SEQUENCE LISTING (971 sequences)

SEQ ID NO: 1 - protein, 9 aa, SARS-COV2
AMDEFIERY

SEQ ID NO: 2 - protein, 10 aa, SARS-COV2
ATEETFKLSY

SEQ ID NO: 3 - protein, 9 aa, SARS-COV2
ATSRTLSYY

SEQ ID NO: 4 - protein, 13 aa, SARS-COV2
CSDKAYKIEELFY

SEQ ID NO: 5 - protein, 9 aa, SARS-COV2
CTDDNALAY

SEQ ID NO: 6 - protein, 10 aa, SARS-COV2
CTDDNALAYY

SEQ ID NO: 7 - protein, 9 aa, SARS-COV2
DTDFVNEFY

SEQ ID NO: 8 - protein, 11 aa, SARS-COV2
FADDLNQLTGY

SEQ ID NO: 9 - protein, 9 aa, SARS-COV2
FIDTKRGVY

SEQ ID NO: 10 - protein, 9 aa, SARS-COV2
FTSDYYQLY

SEQ ID NO: 11 - protein, 9 aa, SARS-COV2
GTDLEGNFY

SEQ ID NO: 12 - protein, 11 aa, SARS-COV2
HTDFSSEIIGY

SEQ ID NO: 13 - protein, 9 aa, SARS-COV2
ISDYDYYRY

SEQ ID NO: 14 - protein, 11 aa, SARS-COV2
ITDVFYKENSY

SEQ ID NO: 15 - protein, 10 aa, SARS-COV2
IVDTVSALVY

SEQ ID NO: 16 - protein, 9 aa, SARS-COV2
KSDGTGTIY

SEQ ID NO: 17 - protein, 13 aa, SARS-COV2
LSDDAVVCFNSTY

SEQ ID NO: 18 - protein, 9 aa, SARS-COV2
LTDEMIAQY

SEQ ID NO: 19 - protein, 8 aa, SARS-COV2
LTENLLLY

SEQ ID NO: 20 - protein, 9 aa, SARS-COV2
LTNDNTSRY

SEQ ID NO: 21 - protein, 10 aa, SARS-COV2
MADQAMTQMY

SEQ ID NO: 22 - protein, 9 aa, SARS-COV2
PTDNYITTY

SEQ ID NO: 23 - protein, 11 aa, SARS-COV2
STDGNKIADKY

SEQ ID NO: 24 - protein, 11 aa, SARS-COV2
STECSNLLLQY

SEQ ID NO: 25 - protein, 8 aa, SARS-COV2
TSDYYQLY

SEQ ID NO: 26 - protein, 10 aa, SARS-COV2
TTDPSFLGRY

SEQ ID NO: 27 - protein, 10 aa, SARS-COV2
TVDSSQGSEY

SEQ ID NO: 28 - protein, 8 aa, SARS-COV2
VTDVTQLY

SEQ ID NO: 29 - protein, 9 aa, SARS-COV2
VTDVTQLYL

SEQ ID NO: 30 - protein, 14 aa, SARS-COV2
VTDVTQLYLGGMSY

SEQ ID NO: 31 - protein, 11 aa, SARS-COV2
VVDDPCPIHFY

SEQ ID NO: 32 - protein, 9 aa, SARS-COV2
VVDKYFDCY

SEQ ID NO: 33 - protein, 9 aa, SARS-COV2
VVDYGARFY

SEQ ID NO: 34 - protein, 11 aa, SARS-COV2
YADVFHLYLQY

SEQ ID NO: 35 - protein, 9 aa, SARS-COV2
ALSKGVHFV

SEQ ID NO: 36 - protein, 9 aa, SARS-COV2
ALWEIQQVV

SEQ ID NO: 37 - protein, 9 aa, SARS-COV2
FLAFVVFLL

SEQ ID NO: 38 - protein, 9 aa, SARS-COV2
FLAHIQWMV

SEQ ID NO: 39 - protein, 9 aa, SARS-COV2
FLLNKEMYL

SEQ ID NO: 40 - protein, 9 aa, SARS-COV2
FLLPSLATV

SEQ ID NO: 41 - protein, 9 aa, SARS-COV2
FLNRFTTTL

SEQ ID NO: 42 - protein, 9 aa, SARS-COV2
ILFTRFFYV

SEQ ID NO: 43 - protein, 9 aa, SARS-COV2
KLIEYTDFA

SEQ ID NO: 44 - protein, 9 aa, SARS-COV2
KLSYGIATV

SEQ ID NO: 45 - protein, 9 aa, SARS-COV2
LLLDDFVEI

SEQ ID NO: 46 - protein, 9 aa, SARS-COV2
LLYDANYFL

SEQ ID NO: 47 - protein, 9 aa, SARS-COV2
NLSDRVVFV

SEQ ID NO: 48 - protein, 9 aa, SARS-COV2
SMWALIISV

SEQ ID NO: 49 - protein, 9 aa, SARS-COV2
TLMNVLTLV

SEQ ID NO: 50 - protein, 9 aa, SARS-COV2
TMADLVYAL

SEQ ID NO: 51 - protein, 9 aa, SARS-COV2
VLWAHGFEL

SEQ ID NO: 52 - protein, 9 aa, SARS-COV2
YLATALLTL

SEQ ID NO: 53 - protein, 9 aa, SARS-COV2
YLDAYNMMI

SEQ ID NO: 54 - protein, 11 aa, SARS-COV2
YLFDESGEFKL

SEQ ID NO: 55 - protein, 9 aa, SARS-COV2
YLNTLTLAV

SEQ ID NO: 56 - protein, 9 aa, SARS-COV2
YLQPRTFLL

SEQ ID NO: 57 - protein, 9 aa, SARS-COV2
YLYALVYFL

SEQ ID NO: 58 - protein, 9 aa, SARS-COV2
ALSKGVHFV

SEQ ID NO: 59 - protein, 9 aa, SARS-COV2
FIAGLIAIV

SEQ ID NO: 60 - protein, 9 aa, SARS-COV2
FLAHIQWMV

SEQ ID NO: 61 - protein, 9 aa, SARS-COV2
FLLNKEMYL

SEQ ID NO: 62 - protein, 9 aa, SARS-COV2
FLLPSLATV

SEQ ID NO: 63 - protein, 9 aa, SARS-COV2
FLNRFTTTL

SEQ ID NO: 64 - protein, 9 aa, SARS-COV2
FLPRVFSAV

SEQ ID NO: 65 - protein, 9 aa, SARS-COV2
FVNEFYAYL

SEQ ID NO: 66 - protein, 9 aa, SARS-COV2
KIADYNYKL

SEQ ID NO: 67 - protein, 9 aa, SARS-COV2
KLSYGIATV

SEQ ID NO: 68 - protein, 9 aa, SARS-COV2
LLYDANYFL

SEQ ID NO: 69 - protein, 9 aa, SARS-COV2
NLSDRVVFV

SEQ ID NO: 70 - protein, 9 aa, SARS-COV2
QMAPISAMV

SEQ ID NO: 71 - protein, 9 aa, SARS-COV2
SLPGVFCGV

SEQ ID NO: 72 - protein, 9 aa, SARS-COV2
TLMNVLTLV

SEQ ID NO: 73 - protein, 9 aa, SARS-COV2
TMADLVYAL

SEQ ID NO: 74 - protein, 9 aa, SARS-COV2
VLNDILSRL

SEQ ID NO: 75 - protein, 9 aa, SARS-COV2
VMVELVAEL

SEQ ID NO: 76 - protein, 9 aa, SARS-COV2
YLATALLTL

SEQ ID NO: 77 - protein, 9 aa, SARS-COV2
YLNTLTLAV

SEQ ID NO: 78 - protein, 9 aa, SARS-COV2
YLQPRTFLL

SEQ ID NO: 79 - protein, 9 aa, SARS-COV2
YLTNDVSFL

SEQ ID NO: 80 - protein, 9 aa, SARS-COV2
ALSKGVHFV

SEQ ID NO: 81 - protein, 9 aa, SARS-COV2
FLAHIQWMV

SEQ ID NO: 82 - protein, 9 aa, SARS-COV2
FLLNKEMYL

SEQ ID NO: 83 - protein, 9 aa, SARS-COV2
FLLPSLATV

SEQ ID NO: 84 - protein, 9 aa, SARS-COV2
FLNGSCGSV

SEQ ID NO: 85 - protein, 9 aa, SARS-COV2
FLNRFTTTL

SEQ ID NO: 86 - protein, 9 aa, SARS-COV2
FLPRVFSAV

SEQ ID NO: 87 - protein, 9 aa, SARS-COV2
GLNDNLLEI

SEQ ID NO: 88 - protein, 9 aa, SARS-COV2
KLKDCVMYA

SEQ ID NO: 89 - protein, 9 aa, SARS-COV2
KLSYGIATV

SEQ ID NO: 90 - protein, 9 aa, SARS-COV2
MLAKALRKV

SEQ ID NO: 91 - protein, 9 aa, SARS-COV2
NLSDRVVFV

SEQ ID NO: 92 - protein, 9 aa, SARS-COV2
RLANECAQV

SEQ ID NO: 93 - protein, 9 aa, SARS-COV2
SLPGVFCGV

SEQ ID NO: 94 - protein, 10 aa, SARS-COV2
TLATHGLAAV

SEQ ID NO: 95 - protein, 9 aa, SARS-COV2
TLIGDCATV

SEQ ID NO: 96 - protein, 9 aa, SARS-COV2
TLMNVLTLV

SEQ ID NO: 97 - protein, 9 aa, SARS-COV2
TMADLVYAL

SEQ ID NO: 98 - protein, 9 aa, SARS-COV2
VLAWLYAAV

SEQ ID NO: 99 - protein, 9 aa, SARS-COV2
VLNDILSRL

SEQ ID NO: 100 - protein, 9 aa, SARS-COV2
YLATALLTL

SEQ ID NO: 101 - protein, 9 aa, SARS-COV2
YLNTLTLAV

SEQ ID NO: 102 - protein, 9 aa, SARS-COV2
YLQPRTFLL

SEQ ID NO: 103 - protein, 9 aa, SARS-COV2
ALSKGVHFV

SEQ ID NO: 104 - protein, 9 aa, SARS-COV2
FLLPSLATV

SEQ ID NO: 105 - protein, 9 aa, SARS-COV2
YLQPRTFLL

SEQ ID NO: 106 - protein, 9 aa, SARS-COV2
ALWEIQQVV

SEQ ID NO: 107 - protein, 9 aa, SARS-COV2
FLAHIQWMV

SEQ ID NO: 108 - protein, 9 aa, SARS-COV2
FLLPSLATV

SEQ ID NO: 109 - protein, 9 aa, SARS-COV2
KIADYNYKL

SEQ ID NO: 110 - protein, 9 aa, SARS-COV2
LLYDANYFL

SEQ ID NO: 111 - protein, 9 aa, SARS-COV2
SLPGVFCGV

SEQ ID NO: 112 - protein, 9 aa, SARS-COV2
TLMNVLTLV

SEQ ID NO: 113 - protein, 9 aa, SARS-COV2
TVYSHLLLV

SEQ ID NO: 114 - protein, 9 aa, SARS-COV2
YLQPRTFLL

SEQ ID NO: 115 - protein, 9 aa, SARS-COV2
YVWKSYVHV

SEQ ID NO: 116 - protein, 9 aa, SARS-COV2
ALSKGVHFV

SEQ ID NO: 117 - protein, 9 aa, SARS-COV2
ALWEIQQVV

SEQ ID NO: 118 - protein, 9 aa, SARS-COV2
FLAHIQWMV

SEQ ID NO: 119 - protein, 9 aa, SARS-COV2
FLLPSLATV

SEQ ID NO: 120 - protein, 9 aa, SARS-COV2
FLPRVFSAV

SEQ ID NO: 121 - protein, 9 aa, SARS-COV2
FVDGVPFVV

SEQ ID NO: 122 - protein, 9 aa, SARS-COV2
LLDDFVEII

SEQ ID NO: 123 - protein, 9 aa, SARS-COV2
SLPGVFCGV

SEQ ID NO: 124 - protein, 9 aa, SARS-COV2
TMADLVYAL

SEQ ID NO: 125 - protein, 9 aa, SARS-COV2
YIDIGNYTV

SEQ ID NO: 126 - protein, 9 aa, SARS-COV2
YLQPRTFLL

SEQ ID NO: 127 - protein, 9 aa, SARS-COV2
ALSKGVHFV

SEQ ID NO: 128 - protein, 9 aa, SARS-COV2
ALWEIQQVV

SEQ ID NO: 129 - protein, 9 aa, SARS-COV2
FLAFVVFLL

SEQ ID NO: 130 - protein, 9 aa, SARS-COV2
FLAHIQWMV

SEQ ID NO: 131 - protein, 9 aa, SARS-COV2
FLLNKEMYL

SEQ ID NO: 132 - protein, 9 aa, SARS-COV2
FLLPSLATV

SEQ ID NO: 133 - protein, 9 aa, SARS-COV2
ILFTRFFYV

SEQ ID NO: 134 - protein, 9 aa, SARS-COV2
KLLEQWNLV

SEQ ID NO: 135 - protein, 9 aa, SARS-COV2
KLSYGIATV

SEQ ID NO: 136 - protein, 9 aa, SARS-COV2
LLYDANYFL

SEQ ID NO: 137 - protein, 9 aa, SARS-COV2
NLIDSYFVV

SEQ ID NO: 138 - protein, 9 aa, SARS-COV2
NLSDRVVFV

SEQ ID NO: 139 - protein, 9 aa, SARS-COV2
SLVKPSFYV

SEQ ID NO: 140 - protein, 9 aa, SARS-COV2
SMWALIISV

SEQ ID NO: 141 - protein, 9 aa, SARS-COV2
TLMNVLTLV

SEQ ID NO: 142 - protein, 9 aa, SARS-COV2
TMADLVYAL

SEQ ID NO: 143 - protein, 9 aa, SARS-COV2
VLWAHGFEL

SEQ ID NO: 144 - protein, 9 aa, SARS-COV2
YLATALLTL

SEQ ID NO: 145 - protein, 9 aa, SARS-COV2
YLQPRTFLL

SEQ ID NO: 146 - protein, 9 aa, SARS-COV2
YLYALVYFL

SEQ ID NO: 147 - protein, 9 aa, SARS-COV2
ALAYYNTTK

SEQ ID NO: 148 - protein, 9 aa, SARS-COV2
ALCTFLLNK

SEQ ID NO: 149 - protein, 9 aa, SARS-COV2
ASMPTTIAK

SEQ ID NO: 150 - protein, 9 aa, SARS-COV2
GVAMPNLYK

SEQ ID NO: 151 - protein, 9 aa, SARS-COV2
GVYFASTEK

SEQ ID NO: 152 - protein, 9 aa, SARS-COV2
GVYYHKNNK

SEQ ID NO: 153 - protein, 9 aa, SARS-COV2
KLFAAETLK

SEQ ID NO: 154 - protein, 9 aa, SARS-COV2
KLFDRYFKY

SEQ ID NO: 155 - protein, 12 aa, SARS-COV2
KMADQAMTQMYK

SEQ ID NO: 156 - protein, 9 aa, SARS-COV2
KMQRMLLEK

SEQ ID NO: 157 - protein, 9 aa, SARS-COV2
KSAGFPFNK

SEQ ID NO: 158 - protein, 9 aa, SARS-COV2
KTFPPTEPK

SEQ ID NO: 159 - protein, 10 aa, SARS-COV2
KTFPPTEPKK

SEQ ID NO: 160 - protein, 9 aa, SARS-COV2
KTIQPRVEK

SEQ ID NO: 161 - protein, 10 aa, SARS-COV2
KVAGFAKFLK

SEQ ID NO: 162 - protein, 9 aa, SARS-COV2
QTFFKLVNK

SEQ ID NO: 163 - protein, 9 aa, SARS-COV2
RLFRKSNLK

SEQ ID NO: 164 - protein, 9 aa, SARS-COV2
RLISMMGFK

SEQ ID NO: 165 - protein, 9 aa, SARS-COV2
RQFHQKLLK

SEQ ID NO: 166 - protein, 9 aa, SARS-COV2
STFNVPMEK

SEQ ID NO: 167 - protein, 9 aa, SARS-COV2
TSFGPLVRK

SEQ ID NO: 168 - protein, 9 aa, SARS-COV2
TTIKPVTYK

SEQ ID NO: 169 - protein, 9 aa, SARS-COV2
VLSGHNLAK

SEQ ID NO: 170 - protein, 9 aa, SARS-COV2
VTNNTFTLK

SEQ ID NO: 171 - protein, 9 aa, SARS-COV2
VTYVPAQEK

SEQ ID NO: 172 - protein, 10 aa, SARS-COV2
VVYRGTTTYK

SEQ ID NO: 173 - protein, 9 aa, SARS-COV2
AIDAYPLTK

SEQ ID NO: 174 - protein, 9 aa, SARS-COV2
AIVSTIQRK

SEQ ID NO: 175 - protein, 9 aa, SARS-COV2
ALCTFLLNK

SEQ ID NO: 176 - protein, 9 aa, SARS-COV2
ASANLAATK

SEQ ID NO: 177 - protein, 9 aa, SARS-COV2
ASMPTTIAK

SEQ ID NO: 178 - protein, 9 aa, SARS-COV2
ATAEAELAK

SEQ ID NO: 179 - protein, 10 aa, SARS-COV2
ATSRTLSYYK

SEQ ID NO: 180 - protein, 9 aa, SARS-COV2
ATVVIGTSK

SEQ ID NO: 181 - protein, 9 aa, SARS-COV2
AVAKHDFFK

SEQ ID NO: 182 - protein, 10 aa, SARS-COV2
AVFDKNLYDK

SEQ ID NO: 183 - protein, 9 aa, SARS-COV2
AVLQSGFRK

SEQ ID NO: 184 - protein, 9 aa, SARS-COV2
GTHWFVTQR

SEQ ID NO: 185 - protein, 9 aa, SARS-COV2
GTLSYEQFK

SEQ ID NO: 186 - protein, 10 aa, SARS-COV2
GTLSYEQFKK

SEQ ID NO: 187 - protein, 9 aa, SARS-COV2
GVAMPNLYK

SEQ ID NO: 188 - protein, 9 aa, SARS-COV2
GVYFASTEK

SEQ ID NO: 189 - protein, 9 aa, SARS-COV2
HVVGPNVNK

SEQ ID NO: 190 - protein, 9 aa, SARS-COV2
IINNTVYTK

SEQ ID NO: 191 - protein, 9 aa, SARS-COV2
KSAGFPFNK

SEQ ID NO: 192 - protein, 9 aa, SARS-COV2
KTFPPTEPK

SEQ ID NO: 193 - protein, 10 aa, SARS-COV2
KTFPPTEPKK

SEQ ID NO: 194 - protein, 9 aa, SARS-COV2
KTIQPRVEK

SEQ ID NO: 195 - protein, 9 aa, SARS-COV2
KTQFNYYKK

SEQ ID NO: 196 - protein, 10 aa, SARS-COV2
KVAGFAKFLK

SEQ ID NO: 197 - protein, 9 aa, SARS-COV2
MTNRQFHQK

SEQ ID NO: 198 - protein, 9 aa, SARS-COV2
QTFFKLVNK

SEQ ID NO: 199 - protein, 9 aa, SARS-COV2
QVVNVVTTK

SEQ ID NO: 200 - protein, 9 aa, SARS-COV2
SASKIITLK

SEQ ID NO: 201 - protein, 10 aa, SARS-COV2
SIINNTVYTK

SEQ ID NO: 202 - protein, 9 aa, SARS-COV2
STFNVPMEK

SEQ ID NO: 203 - protein, 11 aa, SARS-COV2
STMTNRQFHQK

SEQ ID NO: 204 - protein, 9 aa, SARS-COV2
TSFGPLVRK

SEQ ID NO: 205 - protein, 9 aa, SARS-COV2
TTIKPVTYK

SEQ ID NO: 206 - protein, 9 aa, SARS-COV2
TVIEVQGYK

SEQ ID NO: 207 - protein, 9 aa, SARS-COV2
VTNNTFTLK

SEQ ID NO: 208 - protein, 9 aa, SARS-COV2
VTYVPAQEK

SEQ ID NO: 209 - protein, 10 aa, SARS-COV2
VVYRGTTTYK

SEQ ID NO: 210 - protein, 9 aa, SARS-COV2
AYANSVFNI

SEQ ID NO: 211 - protein, 9 aa, SARS-COV2
AYILFTRFF

SEQ ID NO: 212 - protein, 10 aa, SARS-COV2
AYVNTFSSTF

SEQ ID NO: 213 - protein, 9 aa, SARS-COV2
CYFGLFCLL

SEQ ID NO: 214 - protein, 10 aa, SARS-COV2
CYFPLQSYGF

SEQ ID NO: 215 - protein, 9 aa, SARS-COV2
FYLTNDVSF

SEQ ID NO: 216 - protein, 9 aa, SARS-COV2
GYKSVNITF

SEQ ID NO: 217 - protein, 9 aa, SARS-COV2
IYLYLTFYL

SEQ ID NO: 218 - protein, 9 aa, SARS-COV2
IYNDKVAGF

SEQ ID NO: 219 - protein, 11 aa, SARS-COV2
IYTELEPPCRF

SEQ ID NO: 220 - protein, 9 aa, SARS-COV2
KFTDGVCLF

SEQ ID NO: 221 - protein, 9 aa, SARS-COV2
LYDKLVSSF

SEQ ID NO: 222 - protein, 9 aa, SARS-COV2
LYLYALVYF

SEQ ID NO: 223 - protein, 10 aa, SARS-COV2
LYNSASFSTF

SEQ ID NO: 224 - protein, 9 aa, SARS-COV2
MYASAVVLL

SEQ ID NO: 225 - protein, 11 aa, SARS-COV2
MYMGTLSYEQF

SEQ ID NO: 226 - protein, 9 aa, SARS-COV2
NYLKRRVVF

SEQ ID NO: 227 - protein, 9 aa, SARS-COV2
NYMPYFFTL

SEQ ID NO: 228 - protein, 9 aa, SARS-COV2
QYIKWPWYI

SEQ ID NO: 229 - protein, 11 aa, SARS-COV2
RYPANSIVCRF

SEQ ID NO: 230 - protein, 9 aa, SARS-COV2
SYFIASFRL

SEQ ID NO: 231 - protein, 10 aa, SARS-COV2
SYFIASFRLF

SEQ ID NO: 232 - protein, 10 aa, SARS-COV2
SYFVVKRHTF

SEQ ID NO: 233 - protein, 9 aa, SARS-COV2
TFFKLVNKF

SEQ ID NO: 234 - protein, 9 aa, SARS-COV2
TYACWHHSI

SEQ ID NO: 235 - protein, 9 aa, SARS-COV2
TYKPNTWCI

SEQ ID NO: 236 - protein, 13 aa, SARS-COV2
TYLEGSVRVVTTF

SEQ ID NO: 237 - protein, 9 aa, SARS-COV2
VYDPLQPEL

SEQ ID NO: 238 - protein, 9 aa, SARS-COV2
VYFLQSINF

SEQ ID NO: 239 - protein, 9 aa, SARS-COV2
VYIGDPAQL

SEQ ID NO: 240 - protein, 9 aa, SARS-COV2
VYMPASWVM

SEQ ID NO: 241 - protein, 11 aa, SARS-COV2
VYMPASWVMRI

SEQ ID NO: 242 - protein, 9 aa, SARS-COV2
VYSTGSNVF

SEQ ID NO: 243 - protein, 10 aa, SARS-COV2
VYYTSNPTTF

SEQ ID NO: 244 - protein, 9 aa, SARS-COV2
YFIASFRLF

SEQ ID NO: 245 - protein, 9 aa, SARS-COV2
YFTSDYYQL

SEQ ID NO: 246 - protein, 9 aa, SARS-COV2
YFVVKRHTF

SEQ ID NO: 247 - protein, 9 aa, SARS-COV2
YYHTTDPSF

SEQ ID NO: 248 - protein, 9 aa, SARS-COV2
YYKKDNSYF

SEQ ID NO: 249 - protein, 9 aa, SARS-COV2
YYTSNPTTF

SEQ ID NO: 250 - protein, 9 aa, SARS-COV2
AYANSVFNI

SEQ ID NO: 251 - protein, 10 aa, SARS-COV2
AYVNTFSSTF

SEQ ID NO: 252 - protein, 10 aa, SARS-COV2
CYFPLQSYGF

SEQ ID NO: 253 - protein, 9 aa, SARS-COV2
FFASFYYVW

SEQ ID NO: 254 - protein, 9 aa, SARS-COV2
FYLTNDVSF

SEQ ID NO: 255 - protein, 10 aa, SARS-COV2
GYINVFAFPF

SEQ ID NO: 256 - protein, 9 aa, SARS-COV2
GYKSVNITF

SEQ ID NO: 257 - protein, 9 aa, SARS-COV2
IYLYLTFYL

SEQ ID NO: 258 - protein, 9 aa, SARS-COV2
IYNDKVAGF

SEQ ID NO: 259 - protein, 13 aa, SARS-COV2
LYALVYFLQSINF

SEQ ID NO: 260 - protein, 9 aa, SARS-COV2
LYDKLVSSF

SEQ ID NO: 261 - protein, 10 aa, SARS-COV2
LYIIKLIFLW

SEQ ID NO: 262 - protein, 13 aa, SARS-COV2
LYLTFYLTNDVSF

SEQ ID NO: 263 - protein, 9 aa, SARS-COV2
LYLYALVYF

SEQ ID NO: 264 - protein, 10 aa, SARS-COV2
LYNSASFSTF

SEQ ID NO: 265 - protein, 9 aa, SARS-COV2
MYASAVVLL

SEQ ID NO: 266 - protein, 10 aa, SARS-COV2
MYASAVVLLI

SEQ ID NO: 267 - protein, 12 aa, SARS-COV2
MYIFFASFYYVW

SEQ ID NO: 268 - protein, 11 aa, SARS-COV2
MYMGTLSYEQF

SEQ ID NO: 269 - protein, 9 aa, SARS-COV2
NYMPYFFTL

SEQ ID NO: 270 - protein, 10 aa, SARS-COV2
NYMPYFFTLL

SEQ ID NO: 271 - protein, 11 aa, SARS-COV2
NYMPYFFTLLL

SEQ ID NO: 272 - protein, 13 aa, SARS-COV2
NYMPYFFTLLLQL

SEQ ID NO: 273 - protein, 9 aa, SARS-COV2
QYIKWPWYI

SEQ ID NO: 274 - protein, 10 aa, SARS-COV2
QYIKWPWYIW

SEQ ID NO: 275 - protein, 13 aa, SARS-COV2
QYIKWPWYIWLGF

SEQ ID NO: 276 - protein, 12 aa, SARS-COV2
RYLALYNKYKYF

SEQ ID NO: 277 - protein, 11 aa, SARS-COV2
RYPANSIVCRF

SEQ ID NO: 278 - protein, 9 aa, SARS-COV2
TYACWHHSI

SEQ ID NO: 279 - protein, 9 aa, SARS-COV2
TYASALWEI

SEQ ID NO: 280 - protein, 9 aa, SARS-COV2
TYKPNTWCI

SEQ ID NO: 281 - protein, 13 aa, SARS-COV2
TYLEGSVRVVTTF

SEQ ID NO: 282 - protein, 9 aa, SARS-COV2
VYFLQSINF

SEQ ID NO: 283 - protein, 9 aa, SARS-COV2
VYIGDPAQL

SEQ ID NO: 284 - protein, 9 aa, SARS-COV2
VYMPASWVM

SEQ ID NO: 285 - protein, 11 aa, SARS-COV2
VYMPASWVMRI

SEQ ID NO: 286 - protein, 14 aa, SARS-COV2
VYMPASWVMRIMTW

SEQ ID NO: 287 - protein, 10 aa, SARS-COV2
VYSSANNCTF

SEQ ID NO: 288 - protein, 9 aa, SARS-COV2
VYSTGSNVF

SEQ ID NO: 289 - protein, 9 aa, SARS-COV2
VYSVIYLYL

SEQ ID NO: 290 - protein, 11 aa, SARS-COV2
VYSVIYLYLTF

SEQ ID NO: 291 - protein, 10 aa, SARS-COV2
VYYTSNPTTF

SEQ ID NO: 292 - protein, 14 aa, SARS-COV2
YYDSMSYEDQDALF

SEQ ID NO: 293 - protein, 9 aa, SARS-COV2
YYHTTDPSF

SEQ ID NO: 294 - protein, 9 aa, SARS-COV2
YYKKDNSYF

SEQ ID NO: 295 - protein, 10 aa, SARS-COV2
YYSQLMCQPI

SEQ ID NO: 296 - protein, 9 aa, SARS-COV2
YYTSNPTTF

SEQ ID NO: 297 - protein, 10 aa, SARS-COV2
DIYNDKVAGF

SEQ ID NO: 298 - protein, 10 aa, SARS-COV2
DIYNDKVAGF

SEQ ID NO: 299 - protein, 9 aa, SARS-COV2
DTVIEVQGY

SEQ ID NO: 300 - protein, 9 aa, SARS-COV2
DTYNLWNTF

SEQ ID NO: 301 - protein, 9 aa, SARS-COV2
DVFHLYLQY

SEQ ID NO: 302 - protein, 9 aa, SARS-COV2
DVFYKENSY

SEQ ID NO: 303 - protein, 9 aa, SARS-COV2
DVLLPLTQY

SEQ ID NO: 304 - protein, 9 aa, SARS-COV2
DVTDVTQLY

SEQ ID NO: 305 - protein, 9 aa, SARS-COV2
EAIRHVRAW

SEQ ID NO: 306 - protein, 11 aa, SARS-COV2
EAVMYMGTLSY

SEQ ID NO: 307 - protein, 14 aa, SARS-COV2
EAVMYMGTLSYEQF

SEQ ID NO: 308 - protein, 9 aa, SARS-COV2
EIAIILASF

SEQ ID NO: 309 - protein, 9 aa, SARS-COV2
EIKESVQTF

SEQ ID NO: 310 - protein, 9 aa, SARS-COV2
EIVDTVSAL

SEQ ID NO: 311 - protein, 10 aa, SARS-COV2
ETICAPLTVF

SEQ ID NO: 312 - protein, 10 aa, SARS-COV2
ETIQITISSF

SEQ ID NO: 313 - protein, 12 aa, SARS-COV2
ETIQITISSFKW

SEQ ID NO: 314 - protein, 9 aa, SARS-COV2
ETISLAGSY

SEQ ID NO: 315 - protein, 9 aa, SARS-COV2
ETKAIVSTI

SEQ ID NO: 316 - protein, 9 aa, SARS-COV2
ETKCTLKSF

SEQ ID NO: 317 - protein, 9 aa, SARS-COV2
ETTADIVVF

SEQ ID NO: 318 - protein, 10 aa, SARS-COV2
EVARDLSLQF

SEQ ID NO: 319 - protein, 10 aa, SARS-COV2
EVAVKMFDAY

SEQ ID NO: 320 - protein, 10 aa, SARS-COV2
EVVDKYFDCY

SEQ ID NO: 321 - protein, 9 aa, SARS-COV2
FTSDYYQLY

SEQ ID NO: 322 - protein, 9 aa, SARS-COV2
FVFKNIDGY

SEQ ID NO: 323 - protein, 9 aa, SARS-COV2
FVVEVVDKY

SEQ ID NO: 324 - protein, 9 aa, SARS-COV2
GVVQLTSQW

SEQ ID NO: 325 - protein, 9 aa, SARS-COV2
LVAEWFLAY

SEQ ID NO: 326 - protein, 9 aa, SARS-COV2
NTVKSVGKF

SEQ ID NO: 327 - protein, 9 aa, SARS-COV2
QTFSVLACY

SEQ ID NO: 328 - protein, 9 aa, SARS-COV2
QVVDMSMTY

SEQ ID NO: 329 - protein, 9 aa, SARS-COV2
TILDGISQY

SEQ ID NO: 330 - protein, 9 aa, SARS-COV2
VVIPDYNTY

SEQ ID NO: 331 - protein, 9 aa, SARS-COV2
YTPSKLIEY

SEQ ID NO: 332 - protein, 9 aa, SARS-COV2
CVADYSVLY

SEQ ID NO: 333 - protein, 10 aa, SARS-COV2
DTLKEILVTY

SEQ ID NO: 334 - protein, 9 aa, SARS-COV2
DTVIEVQGY

SEQ ID NO: 335 - protein, 9 aa, SARS-COV2
DVFHLYLQY

SEQ ID NO: 336 - protein, 9 aa, SARS-COV2
DVFYKENSY

SEQ ID NO: 337 - protein, 9 aa, SARS-COV2
DVLLPLTQY

SEQ ID NO: 338 - protein, 9 aa, SARS-COV2
DVTDVTQLY

SEQ ID NO: 339 - protein, 9 aa, SARS-COV2
EIAIILASF

SEQ ID NO: 340 - protein, 9 aa, SARS-COV2
EIKESVQTF

SEQ ID NO: 341 - protein, 9 aa, SARS-COV2
EIVDTVSAL

SEQ ID NO: 342 - protein, 11 aa, SARS-COV2
EIVDTVSALVY

SEQ ID NO: 343 - protein, 10 aa, SARS-COV2
ETIQITISSF

SEQ ID NO: 344 - protein, 9 aa, SARS-COV2
ETISLAGSY

SEQ ID NO: 345 - protein, 14 aa, SARS-COV2
ETISLAGSYKDWSY

SEQ ID NO: 346 - protein, 9 aa, SARS-COV2
ETKCTLKSF

SEQ ID NO: 347 - protein, 10 aa, SARS-COV2
EVAVKMFDAY

SEQ ID NO: 348 - protein, 11 aa, SARS-COV2
EVGPEHSLAEY

SEQ ID NO: 349 - protein, 10 aa, SARS-COV2
EVVDKYFDCY

SEQ ID NO: 350 - protein, 9 aa, SARS-COV2
FTSDYYQLY

SEQ ID NO: 351 - protein, 9 aa, SARS-COV2
FVFKNIDGY

SEQ ID NO: 352 - protein, 9 aa, SARS-COV2
FVVEVVDKY

SEQ ID NO: 353 - protein, 9 aa, SARS-COV2
NVIPTITQM

SEQ ID NO: 354 - protein, 9 aa, SARS-COV2
QVVDMSMTY

SEQ ID NO: 355 - protein, 9 aa, SARS-COV2
TILDGISQY

SEQ ID NO: 356 - protein, 11 aa, SARS-COV2
TTYPGQGLNGY

SEQ ID NO: 357 - protein, 9 aa, SARS-COV2
WTAGAAAYY

SEQ ID NO: 358 - protein, 9 aa, SARS-COV2
AIMQLFFSY

SEQ ID NO: 359 - protein, 9 aa, SARS-COV2
ALCEKALKY

SEQ ID NO: 360 - protein, 9 aa, SARS-COV2
CVADYSVLY

SEQ ID NO: 361 - protein, 9 aa, SARS-COV2
EYADVFHLY

SEQ ID NO: 362 - protein, 9 aa, SARS-COV2
FAIGLALYY

SEQ ID NO: 363 - protein, 9 aa, SARS-COV2
FLFVAAIFY

SEQ ID NO: 364 - protein, 9 aa, SARS-COV2
FLTENLLLY

SEQ ID NO: 365 - protein, 9 aa, SARS-COV2
FLYLYALVY

SEQ ID NO: 366 - protein, 9 aa, SARS-COV2
FTSDYYQLY

SEQ ID NO: 367 - protein, 9 aa, SARS-COV2
GVYSVIYLY

SEQ ID NO: 368 - protein, 9 aa, SARS-COV2
HFYWFFSNY

SEQ ID NO: 369 - protein, 9 aa, SARS-COV2
KLFDRYFKY

SEQ ID NO: 370 - protein, 9 aa, SARS-COV2
LVAEWFLAY

SEQ ID NO: 371 - protein, 9 aa, SARS-COV2
MMSAPPAQY

SEQ ID NO: 372 - protein, 9 aa, SARS-COV2
SFYEDFLEY

SEQ ID NO: 373 - protein, 9 aa, SARS-COV2
SMMGFKMNY

SEQ ID NO: 374 - protein, 9 aa, SARS-COV2
VMYMGTLSY

SEQ ID NO: 375 - protein, 9 aa, SARS-COV2
YANRNRFLY

SEQ ID NO: 376 - protein, 9 aa, SARS-COV2
YFKYWDQTY

SEQ ID NO: 377 - protein, 9 aa, SARS-COV2
YIFFASFYY

SEQ ID NO: 378 - protein, 9 aa, SARS-COV2
YILFTRFFY

SEQ ID NO: 379 - protein, 9 aa, SARS-COV2
AMRPNFTIK

SEQ ID NO: 380 - protein, 9 aa, SARS-COV2
ASKIITLKK

SEQ ID NO: 381 - protein, 9 aa, SARS-COV2
ASMPTTIAK

SEQ ID NO: 382 - protein, 9 aa, SARS-COV2
KTFPPTEPK

SEQ ID NO: 383 - protein, 9 aa, SARS-COV2
KTIQPRVEK

SEQ ID NO: 384 - protein, 9 aa, SARS-COV2
KVKYLYFIK

SEQ ID NO: 385 - protein, 9 aa, SARS-COV2
AIMQLFFSY

SEQ ID NO: 386 - protein, 9 aa, SARS-COV2
ATSRTLSYY

SEQ ID NO: 387 - protein, 9 aa, SARS-COV2
AVKTQFNYY

SEQ ID NO: 388 - protein, 9 aa, SARS-COV2
GTFTCASEY

SEQ ID NO: 389 - protein, 9 aa, SARS-COV2
GVYSVIYLY

SEQ ID NO: 390 - protein, 9 aa, SARS-COV2
HSIGFDYVY

SEQ ID NO: 391 - protein, 9 aa, SARS-COV2
HSYFTSDYY

SEQ ID NO: 392 - protein, 9 aa, SARS-COV2
IQYIDIGNY

SEQ ID NO: 393 - protein, 9 aa, SARS-COV2
KIEELFYSY

SEQ ID NO: 394 - protein, 9 aa, SARS-COV2
KLFDRYFKY

SEQ ID NO: 395 - protein, 11 aa, SARS-COV2
KMADQAMTQMY

SEQ ID NO: 396 - protein, 9 aa, SARS-COV2
KMNYQVNGY

SEQ ID NO: 397 - protein, 9 aa, SARS-COV2
KNFKSVLYY

SEQ ID NO: 398 - protein, 9 aa, SARS-COV2
KVNSTLEQY

SEQ ID NO: 399 - protein, 9 aa, SARS-COV2
LTNDNTSRY

SEQ ID NO: 400 - protein, 9 aa, SARS-COV2
LVKPSFYVY

SEQ ID NO: 401 - protein, 9 aa, SARS-COV2
MMSAPPAQY

SEQ ID NO: 402 - protein, 9 aa, SARS-COV2
MSNLGMPSY

SEQ ID NO: 403 - protein, 9 aa, SARS-COV2
RLYYDSMSY

SEQ ID NO: 404 - protein, 11 aa, SARS-COV2
RVAGDSGFAAY

SEQ ID NO: 405 - protein, 11 aa, SARS-COV2
RVFSAVGNICY

SEQ ID NO: 406 - protein, 9 aa, SARS-COV2
RYLALYNKY

SEQ ID NO: 407 - protein, 9 aa, SARS-COV2
SFYEDFLEY

SEQ ID NO: 408 - protein, 9 aa, SARS-COV2
SMMGFKMNY

SEQ ID NO: 409 - protein, 9 aa, SARS-COV2
STNVTIATY

SEQ ID NO: 410 - protein, 9 aa, SARS-COV2
TLKEILVTY

SEQ ID NO: 411 - protein, 9 aa, SARS-COV2
VMYMGTLSY

SEQ ID NO: 412 - protein, 9 aa, SARS-COV2
VVIPDYNTY

SEQ ID NO: 413 - protein, 9 aa, SARS-COV2
VVYRGTTTY

SEQ ID NO: 414 - protein, 9 aa, SARS-COV2
WTAGAAAYY

SEQ ID NO: 415 - protein, 9 aa, SARS-COV2
AVHECFVKR

SEQ ID NO: 416 - protein, 9 aa, SARS-COV2
AVILRGHLR

SEQ ID NO: 417 - protein, 9 aa, SARS-COV2
GTHWFVTQR

SEQ ID NO: 418 - protein, 9 aa, SARS-COV2
GVKHVYQLR

SEQ ID NO: 419 - protein, 9 aa, SARS-COV2
KSNLKPFER

SEQ ID NO: 420 - protein, 9 aa, SARS-COV2
KTPKYKFVR

SEQ ID NO: 421 - protein, 9 aa, SARS-COV2
RIAGHHLGR

SEQ ID NO: 422 - protein, 9 aa, SARS-COV2
RVCGVSAAR

SEQ ID NO: 423 - protein, 9 aa, SARS-COV2
RVKNLNSSR

SEQ ID NO: 424 - protein, 9 aa, SARS-COV2
RVVRSIFSR

SEQ ID NO: 425 - protein, 9 aa, SARS-COV2
RVYANLGER

SEQ ID NO: 426 - protein, 9 aa, SARS-COV2
SVSPKLFIR

SEQ ID NO: 427 - protein, 9 aa, SARS-COV2
SVYAWNRKR

SEQ ID NO: 428 - protein, 9 aa, SARS-COV2
VVSTGYHFR

SEQ ID NO: 429 - protein, 9 aa, SARS-COV2
VYYPDKVFR

SEQ ID NO: 430 - protein, 9 aa, SARS-COV2
AVHFISNSW

SEQ ID NO: 431 - protein, 9 aa, SARS-COV2
KAYNVTQAF

SEQ ID NO: 432 - protein, 9 aa, SARS-COV2
KIADYNYKL

SEQ ID NO: 433 - protein, 9 aa, SARS-COV2
KLAKKFDTF

SEQ ID NO: 434 - protein, 10 aa, SARS-COV2
KLASHMYCSF

SEQ ID NO: 435 - protein, 9 aa, SARS-COV2
KLFDRYFKY

SEQ ID NO: 436 - protein, 10 aa, SARS-COV2
KLFDRYFKYW

SEQ ID NO: 437 - protein, 9 aa, SARS-COV2
KLLHKPIVW

SEQ ID NO: 438 - protein, 9 aa, SARS-COV2
KLMGHFAWW

SEQ ID NO: 439 - protein, 12 aa, SARS-COV2
KLMGHFAWWTAF

SEQ ID NO: 440 - protein, 10 aa, SARS-COV2
KMFDAYVNTF

SEQ ID NO: 441 - protein, 9 aa, SARS-COV2
KMKDLSPRW

SEQ ID NO: 442 - protein, 9 aa, SARS-COV2
KQFDTYNLW

SEQ ID NO: 443 - protein, 9 aa, SARS-COV2
KSHNIALIW

SEQ ID NO: 444 - protein, 9 aa, SARS-COV2
KSYELQTPF

SEQ ID NO: 445 - protein, 10 aa, SARS-COV2
RLFARTRSMW

SEQ ID NO: 446 - protein, 12 aa, SARS-COV2
RLFARTRSMWSF

SEQ ID NO: 447 - protein, 9 aa, SARS-COV2
RLRAKHYVY

SEQ ID NO: 448 - protein, 9 aa, SARS-COV2
RMYIFFASF

SEQ ID NO: 449 - protein, 9 aa, SARS-COV2
RTIKGTHHW

SEQ ID NO: 450 - protein, 9 aa, SARS-COV2
RTIKVFTTV

SEQ ID NO: 451 - protein, 9 aa, SARS-COV2
RTNVYLAVF

SEQ ID NO: 452 - protein, 11 aa, SARS-COV2
RVYSSANNCTF

SEQ ID NO: 453 - protein, 9 aa, SARS-COV2
SMMGFKMNY

SEQ ID NO: 454 - protein, 9 aa, SARS-COV2
DTANPKTPK

SEQ ID NO: 455 - protein, 10 aa, SARS-COV2
ETISLAGSYK

SEQ ID NO: 456 - protein, 11 aa, SARS-COV2
ETKAIVSTIQR

SEQ ID NO: 457 - protein, 10 aa, SARS-COV2
EVVENPTIQK

SEQ ID NO: 458 - protein, 9 aa, SARS-COV2
EVVGDIILK

SEQ ID NO: 459 - protein, 9 aa, SARS-COV2
FTIGTVTLK

SEQ ID NO: 460 - protein, 9 aa, SARS-COV2
HVVGPNVNK

SEQ ID NO: 461 - protein, 9 aa, SARS-COV2
LVIGAVILR

SEQ ID NO: 462 - protein, 9 aa, SARS-COV2
QVVNVVTTK

SEQ ID NO: 463 - protein, 9 aa, SARS-COV2
STFNVPMEK

SEQ ID NO: 464 - protein, 10 aa, SARS-COV2
ETAHSCNVNR

SEQ ID NO: 465 - protein, 12 aa, SARS-COV2
ETFVTHSKGLYR

SEQ ID NO: 466 - protein, 10 aa, SARS-COV2
ETISLAGSYK

SEQ ID NO: 467 - protein, 10 aa, SARS-COV2
EVNSFSGYLK

SEQ ID NO: 468 - protein, 10 aa, SARS-COV2
EVVENPTIQK

SEQ ID NO: 469 - protein, 9 aa, SARS-COV2
EVVGDIILK

SEQ ID NO: 470 - protein, 9 aa, SARS-COV2
FAVSKGFFK

SEQ ID NO: 471 - protein, 9 aa, SARS-COV2
FSSEIIGYK

SEQ ID NO: 472 - protein, 9 aa, SARS-COV2
FTALTQHGK

SEQ ID NO: 473 - protein, 9 aa, SARS-COV2
FTIGTVTLK

SEQ ID NO: 474 - protein, 9 aa, SARS-COV2
GAMDTTSYR

SEQ ID NO: 475 - protein, 10 aa, SARS-COV2
GVAPGTAVLR

SEQ ID NO: 476 - protein, 10 aa, SARS-COV2
HTTDPSFLGR

SEQ ID NO: 477 - protein, 11 aa, SARS-COV2
HVASCDAIMTR

SEQ ID NO: 478 - protein, 10 aa, SARS-COV2
HVGEIPVAYR

SEQ ID NO: 479 - protein, 9 aa, SARS-COV2
LVASIKNFK

SEQ ID NO: 480 - protein, 9 aa, SARS-COV2
LVIGAVILR

SEQ ID NO: 481 - protein, 9 aa, SARS-COV2
NSASFSTFK

SEQ ID NO: 482 - protein, 9 aa, SARS-COV2
QTMLFTMLR

SEQ ID NO: 483 - protein, 9 aa, SARS-COV2
QVVNVVTTK

SEQ ID NO: 484 - protein, 9 aa, SARS-COV2
SASKIITLK

SEQ ID NO: 485 - protein, 9 aa, SARS-COV2
STFNVPMEK

SEQ ID NO: 486 - protein, 9 aa, SARS-COV2
STTTNIVTR

SEQ ID NO: 487 - protein, 9 aa, SARS-COV2
SVSPKLFIR

SEQ ID NO: 488 - protein, 9 aa, SARS-COV2
TSFGPLVRK

SEQ ID NO: 489 - protein, 9 aa, SARS-COV2
TTCCSLSHR

SEQ ID NO: 490 - protein, 9 aa, SARS-COV2
TTFDSEYCR

SEQ ID NO: 491 - protein, 9 aa, SARS-COV2
TTIKPVTYK

SEQ ID NO: 492 - protein, 9 aa, SARS-COV2
TTIVNGVRR

SEQ ID NO: 493 - protein, 9 aa, SARS-COV2
TVIEVQGYK

SEQ ID NO: 494 - protein, 9 aa, SARS-COV2
TVYDDGARR

SEQ ID NO: 495 - protein, 9 aa, SARS-COV2
VTFQSAVKR

SEQ ID NO: 496 - protein, 9 aa, SARS-COV2
VVSTGYHFR

SEQ ID NO: 497 - protein, 9 aa, SARS-COV2
DISGINASV

SEQ ID NO: 498 - protein, 9 aa, SARS-COV2
EAANFCALI

SEQ ID NO: 499 - protein, 9 aa, SARS-COV2
EAFEKMVSL

SEQ ID NO: 500 - protein, 9 aa, SARS-COV2
EAMYTPHTV

SEQ ID NO: 501 - protein, 9 aa, SARS-COV2
ETAQNSVRV

SEQ ID NO: 502 - protein, 9 aa, SARS-COV2
ETFKLSYGI

SEQ ID NO: 503 - protein, 9 aa, SARS-COV2
ETICAPLTV

SEQ ID NO: 504 - protein, 9 aa, SARS-COV2
ETKAIVSTI

SEQ ID NO: 505 - protein, 9 aa, SARS-COV2
FIAGLIAIV

SEQ ID NO: 506 - protein, 9 aa, SARS-COV2
FSASTSAFV

SEQ ID NO: 507 - protein, 9 aa, SARS-COV2
FSYFAVHFI

SEQ ID NO: 508 - protein, 9 aa, SARS-COV2
FTISVTTEI

SEQ ID NO: 509 - protein, 9 aa, SARS-COV2
FTVLCLTPV

SEQ ID NO: 510 - protein, 10 aa, SARS-COV2
FTYASALWEI

SEQ ID NO: 511 - protein, 9 aa, SARS-COV2
FVAAIFYLI

SEQ ID NO: 512 - protein, 9 aa, SARS-COV2
FVNEFYAYL

SEQ ID NO: 513 - protein, 9 aa, SARS-COV2
HTIDGSSGV

SEQ ID NO: 514 - protein, 9 aa, SARS-COV2
MSAFAMMFV

SEQ ID NO: 515 - protein, 9 aa, SARS-COV2
NATNVVIKV

SEQ ID NO: 516 - protein, 9 aa, SARS-COV2
NTASWFTAL

SEQ ID NO: 517 - protein, 9 aa, SARS-COV2
NTFSSTFNV

SEQ ID NO: 518 - protein, 9 aa, SARS-COV2
NTQEVFAQV

SEQ ID NO: 519 - protein, 9 aa, SARS-COV2
NVFAFPFTI

SEQ ID NO: 520 - protein, 9 aa, SARS-COV2
QSSYIVDSV

SEQ ID NO: 521 - protein, 9 aa, SARS-COV2
STSAFVETV

SEQ ID NO: 522 - protein, 9 aa, SARS-COV2
SVAALTNNV

SEQ ID NO: 523 - protein, 9 aa, SARS-COV2
SVVSKVVKV

SEQ ID NO: 524 - protein, 9 aa, SARS-COV2
TTAAKLMVV

SEQ ID NO: 525 - protein, 9 aa, SARS-COV2
TTIQTIVEV

SEQ ID NO: 526 - protein, 9 aa, SARS-COV2
TVASLINTL

SEQ ID NO: 527 - protein, 9 aa, SARS-COV2
TVYSHLLLV

SEQ ID NO: 528 - protein, 9 aa, SARS-COV2
YTACSHAAV

SEQ ID NO: 529 - protein, 9 aa, SARS-COV2
YTMADLVYA

SEQ ID NO: 530 - protein, 9 aa, SARS-COV2
YTNSFTRGV

SEQ ID NO: 531 - protein, 9 aa, SARS-COV2
YTVELGTEV

SEQ ID NO: 532 - protein, 9 aa, SARS-COV2
FPRGQGVPI

SEQ ID NO: 533 - protein, 9 aa, SARS-COV2
IPRRNVATL

SEQ ID NO: 534 - protein, 9 aa, SARS-COV2
KPNELSRVL

SEQ ID NO: 535 - protein, 9 aa, SARS-COV2
VPMEKLKTL

SEQ ID NO: 536 - protein, 9 aa, SARS-COV2
DLKGKYVQI

SEQ ID NO: 537 - protein, 9 aa, SARS-COV2
EAFEKMVSL

SEQ ID NO: 538 - protein, 9 aa, SARS-COV2
LMIERFVSL

SEQ ID NO: 539 - protein, 9 aa, SARS-COV2
TPKYKFVRI

SEQ ID NO: 540 - protein, 9 aa, SARS-COV2
VPMEKLKTL

SEQ ID NO: 541 - protein, 9 aa, SARS-COV2
YLKLRSDVL

SEQ ID NO: 542 - protein, 9 aa, SARS-COV2
YLQPRTFLL

SEQ ID NO: 543 - protein, 10 aa, SARS-COV2
AQFAPSASAF

SEQ ID NO: 544 - protein, 10 aa, SARS-COV2
AQYELKHGTF

SEQ ID NO: 545 - protein, 9 aa, SARS-COV2
ILMTARTVY

SEQ ID NO: 546 - protein, 10 aa, SARS-COV2
KMFDAYVNTF

SEQ ID NO: 547 - protein, 9 aa, SARS-COV2
QLYLGGMSY

SEQ ID NO: 548 - protein, 9 aa, SARS-COV2
RLYYDSMSY

SEQ ID NO: 549 - protein, 10 aa, SARS-COV2
RQKRTATKAY

SEQ ID NO: 550 - protein, 9 aa, SARS-COV2
TLKEILVTY

SEQ ID NO: 551 - protein, 9 aa, SARS-COV2
VLNEKCSAY

SEQ ID NO: 552 - protein, 9 aa, SARS-COV2
VMYMGTLSY

SEQ ID NO: 553 - protein, 9 aa, SARS-COV2
VQMAPISAM

SEQ ID NO: 554 - protein, 9 aa, SARS-COV2
VVYRGTTTY

SEQ ID NO: 555 - protein, 9 aa, SARS-COV2
YLKLTDNVY

SEQ ID NO: 556 - protein, 9 aa, SARS-COV2
YQKVGMQKY

SEQ ID NO: 557 - protein, 9 aa, SARS-COV2
FAVDAAKAY

SEQ ID NO: 558 - protein, 9 aa, SARS-COV2
HVGEIPVAY

SEQ ID NO: 559 - protein, 9 aa, SARS-COV2
ILMTARTVY

SEQ ID NO: 560 - protein, 9 aa, SARS-COV2
MMSAPPAQY

SEQ ID NO: 561 - protein, 9 aa, SARS-COV2
MVMCGGSLY

SEQ ID NO: 562 - protein, 9 aa, SARS-COV2
NVLEGSVAY

SEQ ID NO: 563 - protein, 9 aa, SARS-COV2
QLYLGGMSY

SEQ ID NO: 564 - protein, 9 aa, SARS-COV2
RLYYDSMSY

SEQ ID NO: 565 - protein, 9 aa, SARS-COV2
SIIQFPNTY

SEQ ID NO: 566 - protein, 9 aa, SARS-COV2
TILDGISQY

SEQ ID NO: 567 - protein, 9 aa, SARS-COV2
TLKEILVTY

SEQ ID NO: 568 - protein, 9 aa, SARS-COV2
VLNEKCSAY

SEQ ID NO: 569 - protein, 9 aa, SARS-COV2
VMYMGTLSY

SEQ ID NO: 570 - protein, 9 aa, SARS-COV2
VVIPDYNTY

SEQ ID NO: 571 - protein, 9 aa, SARS-COV2
VVYRGTTTY

SEQ ID NO: 572 - protein, 9 aa, SARS-COV2
YLFDESGEF

SEQ ID NO: 573 - protein, 9 aa, SARS-COV2
YLKLTDNVY

SEQ ID NO: 574 - protein, 10 aa, SARS-COV2
AQFAPSASAF

SEQ ID NO: 575 - protein, 10 aa, SARS-COV2
AQYELKHGTF

SEQ ID NO: 576 - protein, 9 aa, SARS-COV2
GEYSHVVAF

SEQ ID NO: 577 - protein, 9 aa, SARS-COV2
KAYNVTQAF

SEQ ID NO: 578 - protein, 9 aa, SARS-COV2
KKFLPFQQF

SEQ ID NO: 579 - protein, 9 aa, SARS-COV2
KRVDWTIEY

SEQ ID NO: 580 - protein, 9 aa, SARS-COV2
RKAVFISPY

SEQ ID NO: 581 - protein, 9 aa, SARS-COV2
RKGGRTIAF

SEQ ID NO: 582 - protein, 9 aa, SARS-COV2
RLYYDSMSY

SEQ ID NO: 583 - protein, 9 aa, SARS-COV2
VKNGSIHLY

SEQ ID NO: 584 - protein, 9 aa, SARS-COV2
YQKVGMQKY

SEQ ID NO: 585 - protein, 9 aa, SARS-COV2
ATVHTANKW

SEQ ID NO: 586 - protein, 9 aa, SARS-COV2
FTSDYYQLY

SEQ ID NO: 587 - protein, 9 aa, SARS-COV2
KAYNVTQAF

SEQ ID NO: 588 - protein, 9 aa, SARS-COV2
KSHNIALIW

SEQ ID NO: 589 - protein, 9 aa, SARS-COV2
KSVNITFEL

SEQ ID NO: 590 - protein, 9 aa, SARS-COV2
KSYELQTPF

SEQ ID NO: 591 - protein, 9 aa, SARS-COV2
LSFKELLVY

SEQ ID NO: 592 - protein, 9 aa, SARS-COV2
LTYTGAIKL

SEQ ID NO: 593 - protein, 9 aa, SARS-COV2
MSMTYGQQF

SEQ ID NO: 594 - protein, 9 aa, SARS-COV2
RSFIEDLLF

SEQ ID NO: 595 - protein, 9 aa, SARS-COV2
RTIKGTHHW

SEQ ID NO: 596 - protein, 9 aa, SARS-COV2
SSLPSYAAF

SEQ ID NO: 597 - protein, 9 aa, SARS-COV2
VVYRGTTTY

SEQ ID NO: 598 - protein, 10 aa, SARS-COV2
KRFDNPVLPF

SEQ ID NO: 599 - protein, 9 aa, SARS-COV2
KRVDWTIEY

SEQ ID NO: 600 - protein, 9 aa, SARS-COV2
KRWQLALSK

SEQ ID NO: 601 - protein, 9 aa, SARS-COV2
NRFLYIIKL

SEQ ID NO: 602 - protein, 9 aa, SARS-COV2
NRFNVAITR

SEQ ID NO: 603 - protein, 9 aa, SARS-COV2
QRNAPRITF

SEQ ID NO: 604 - protein, 9 aa, SARS-COV2
RRLISMMGF

SEQ ID NO: 605 - protein, 10 aa, SARS-COV2
RRVVFNGVSF

SEQ ID NO: 606 - protein, 9 aa, SARS-COV2
SRYWEPEFY

SEQ ID NO: 607 - protein, 9 aa, SARS-COV2
VRFPNITNL

SEQ ID NO: 608 - protein, 9 aa, SARS-COV2
CPDGVKHVY

SEQ ID NO: 609 - protein, 11 aa, SARS-COV2
FACPDGVKHVY

SEQ ID NO: 610 - protein, 9 aa, SARS-COV2
FAIGLALYY

SEQ ID NO: 611 - protein, 9 aa, SARS-COV2
FAVDAAKAY

SEQ ID NO: 612 - protein, 10 aa, SARS-COV2
FPNITNLCPF

SEQ ID NO: 613 - protein, 9 aa, SARS-COV2
FVVEVVDKY

SEQ ID NO: 614 - protein, 9 aa, SARS-COV2
HVGEIPVAY

SEQ ID NO: 615 - protein, 9 aa, SARS-COV2
IPFAMQMAY

SEQ ID NO: 616 - protein, 11 aa, SARS-COV2
IPIGAGICASY

SEQ ID NO: 617 - protein, 9 aa, SARS-COV2
IPIQASLPF

SEQ ID NO: 618 - protein, 10 aa, SARS-COV2
IPMDSTVKNY

SEQ ID NO: 619 - protein, 10 aa, SARS-COV2
LPFAMGIIAM

SEQ ID NO: 620 - protein, 9 aa, SARS-COV2
LPFFSNVTW

SEQ ID NO: 621 - protein, 9 aa, SARS-COV2
LPFNDGVYF

SEQ ID NO: 622 - protein, 9 aa, SARS-COV2
LPGVYSVIY

SEQ ID NO: 623 - protein, 9 aa, SARS-COV2
LPSLATVAY

SEQ ID NO: 624 - protein, 9 aa, SARS-COV2
LPVNVAFEL

SEQ ID NO: 625 - protein, 9 aa, SARS-COV2
MPYFFTLLL

SEQ ID NO: 626 - protein, 9 aa, SARS-COV2
NVLEGSVAY

SEQ ID NO: 627 - protein, 9 aa, SARS-COV2
QPTESIVRF

SEQ ID NO: 628 - protein, 9 aa, SARS-COV2
SANNCTFEY

SEQ ID NO: 629 - protein, 9 aa, SARS-COV2
TPAFDKSAF

SEQ ID NO: 630 - protein, 9 aa, SARS-COV2
TPSGTWLTY

SEQ ID NO: 631 - protein, 9 aa, SARS-COV2
VASQSIIAY

SEQ ID NO: 632 - protein, 9 aa, SARS-COV2
VPFVVSTGY

SEQ ID NO: 633 - protein, 9 aa, SARS-COV2
VPFWITIAY

SEQ ID NO: 634 - protein, 9 aa, SARS-COV2
VPWDTIANY

SEQ ID NO: 635 - protein, 9 aa, SARS-COV2
YPGQGLNGY

SEQ ID NO: 636 - protein, 9 aa, SARS-COV2
YPNASFDNF

SEQ ID NO: 637 - protein, 8 aa, SARS-COV2
FPFTIYSL

SEQ ID NO: 638 - protein, 10 aa, SARS-COV2
FPFTIYSLLL

SEQ ID NO: 639 - protein, 9 aa, SARS-COV2
HPTQAPTHL

SEQ ID NO: 640 - protein, 9 aa, SARS-COV2
LPVNVAFEL

SEQ ID NO: 641 - protein, 9 aa, SARS-COV2
AELAKNVSL

SEQ ID NO: 642 - protein, 9 aa, SARS-COV2
GEYSHVVAF

SEQ ID NO: 643 - protein, 9 aa, SARS-COV2
NELSRVLGL

SEQ ID NO: 644 - protein, 9 aa, SARS-COV2
SEFDRDAAM

SEQ ID NO: 645 - protein, 9 aa, SARS-COV2
EHFIETISL

SEQ ID NO: 646 - protein, 10 aa, SARS-COV2
FHLDGEVITF

SEQ ID NO: 647 - protein, 9 aa, SARS-COV2
MHAASGNLL

SEQ ID NO: 648 - protein, 9 aa, SARS-COV2
QHEETIYNL

SEQ ID NO: 649 - protein, 10 aa, SARS-COV2
QHEETIYNLL

SEQ ID NO: 650 - protein, 9 aa, SARS-COV2
SHFAIGLAL

SEQ ID NO: 651 - protein, 10 aa, SARS-COV2
SHSQLGGLHL

SEQ ID NO: 652 - protein, 11 aa, SARS-COV2
SHSQLGGLHLL

SEQ ID NO: 653 - protein, 9 aa, SARS-COV2
SHVVAFNTL

SEQ ID NO: 654 - protein, 9 aa, SARS-COV2
THLSVDTKF

SEQ ID NO: 655 - protein, 11 aa, SARS-COV2
YHNESGLKTIL

SEQ ID NO: 656 - protein, 9 aa, SARS-COV2
YHTTDPSFL

SEQ ID NO: 657 - protein, 11 aa, SARS-COV2
AEAELAKNVSL

SEQ ID NO: 658 - protein, 9 aa, SARS-COV2
AEIRASANL

SEQ ID NO: 659 - protein, 10 aa, SARS-COV2
AEIVDTVSAL

SEQ ID NO: 660 - protein, 9 aa, SARS-COV2
AELAKNVSL

SEQ ID NO: 661 - protein, 9 aa, SARS-COV2
AEWFLAYIL

SEQ ID NO: 662 - protein, 9 aa, SARS-COV2
CEFCGTENL

SEQ ID NO: 663 - protein, 9 aa, SARS-COV2
GEAANFCAL

SEQ ID NO: 664 - protein, 10 aa, SARS-COV2
GECPNFVFPL

SEQ ID NO: 665 - protein, 9 aa, SARS-COV2
GETLPTEVL

SEQ ID NO: 666 - protein, 9 aa, SARS-COV2
GEVITFDNL

SEQ ID NO: 667 - protein, 9 aa, SARS-COV2
GEYSHVVAF

SEQ ID NO: 668 - protein, 9 aa, SARS-COV2
HEETIYNLL

SEQ ID NO: 669 - protein, 9 aa, SARS-COV2
HEFCSQHTM

SEQ ID NO: 670 - protein, 9 aa, SARS-COV2
HEGKTFYVL

SEQ ID NO: 671 - protein, 9 aa, SARS-COV2
HEVLLAPLL

SEQ ID NO: 672 - protein, 10 aa, SARS-COV2
LEFGATSAAL

SEQ ID NO: 673 - protein, 10 aa, SARS-COV2
LEYHDVRVVL

SEQ ID NO: 674 - protein, 9 aa, SARS-COV2
NETLVTMPL

SEQ ID NO: 675 - protein, 9 aa, SARS-COV2
QEYADVFHL

SEQ ID NO: 676 - protein, 10 aa, SARS-COV2
REAVGTNLPL

SEQ ID NO: 677 - protein, 10 aa, SARS-COV2
REVGFVVPGL

SEQ ID NO: 678 - protein, 10 aa, SARS-COV2
SELVIGAVIL

SEQ ID NO: 679 - protein, 9 aa, SARS-COV2
SEVGPEHSL

SEQ ID NO: 680 - protein, 9 aa, SARS-COV2
TEEVGHTDL

SEQ ID NO: 681 - protein, 9 aa, SARS-COV2
TEVVGDIIL

SEQ ID NO: 682 - protein, 10 aa, SARS-COV2
YENFNQHEVL

SEQ ID NO: 683 - protein, 11 aa, SARS-COV2
AEAELAKNVSL

SEQ ID NO: 684 - protein, 9 aa, SARS-COV2
AELAKNVSL

SEQ ID NO: 685 - protein, 9 aa, SARS-COV2
AEWFLAYIL

SEQ ID NO: 686 - protein, 9 aa, SARS-COV2
GEAANFCAL

SEQ ID NO: 687 - protein, 9 aa, SARS-COV2
GETLPTEVL

SEQ ID NO: 688 - protein, 9 aa, SARS-COV2
GEYSHVVAF

SEQ ID NO: 689 - protein, 9 aa, SARS-COV2
HEFCSQHTM

SEQ ID NO: 690 - protein, 9 aa, SARS-COV2
HEGKTFYVL

SEQ ID NO: 691 - protein, 9 aa, SARS-COV2
KEIDRLNEV

SEQ ID NO: 692 - protein, 9 aa, SARS-COV2
KENSYTTTI

SEQ ID NO: 693 - protein, 9 aa, SARS-COV2
QEYADVFHL

SEQ ID NO: 694 - protein, 9 aa, SARS-COV2
SEVGPEHSL

SEQ ID NO: 695 - protein, 9 aa, SARS-COV2
VEKGVLPQL

SEQ ID NO: 696 - protein, 9 aa, SARS-COV2
YENFNQHEV

SEQ ID NO: 697 - protein, 10 aa, SARS-COV2
AEWFLAYILF

SEQ ID NO: 698 - protein, 10 aa, SARS-COV2
EEAIRHVRAW

SEQ ID NO: 699 - protein, 9 aa, SARS-COV2
EEFEPSTQY

SEQ ID NO: 700 - protein, 10 aa, SARS-COV2
EELKKLLEQW

SEQ ID NO: 701 - protein, 9 aa, SARS-COV2
GEYSHVVAF

SEQ ID NO: 702 - protein, 10 aa, SARS-COV2
KEIKESVQTF

SEQ ID NO: 703 - protein, 9 aa, SARS-COV2
MEVTPSGTW

SEQ ID NO: 704 - protein, 9 aa, SARS-COV2
QEILGTVSW

SEQ ID NO: 705 - protein, 9 aa, SARS-COV2
QELGKYEQY

SEQ ID NO: 706 - protein, 10 aa, SARS-COV2
QEYADVFHLY

SEQ ID NO: 707 - protein, 9 aa, SARS-COV2
REHEHEIAW

SEQ ID NO: 708 - protein, 9 aa, SARS-COV2
SEFSSLPSY

SEQ ID NO: 709 - protein, 11 aa, SARS-COV2
SEMVMCGGSLY

SEQ ID NO: 710 - protein, 10 aa, SARS-COV2
VENPDILRVY

SEQ ID NO: 711 - protein, 9 aa, SARS-COV2
VENPHLMGW

SEQ ID NO: 712 - protein, 10 aa, SARS-COV2
AEWFLAYILF

SEQ ID NO: 713 - protein, 10 aa, SARS-COV2
EEAIRHVRAW

SEQ ID NO: 714 - protein, 9 aa, SARS-COV2
EEFEPSTQY

SEQ ID NO: 715 - protein, 10 aa, SARS-COV2
GEFKLASHMY

SEQ ID NO: 716 - protein, 9 aa, SARS-COV2
GEYSHVVAF

SEQ ID NO: 717 - protein, 10 aa, SARS-COV2
KEIKESVQTF

SEQ ID NO: 718 - protein, 9 aa, SARS-COV2
MEVTPSGTW

SEQ ID NO: 719 - protein, 9 aa, SARS-COV2
QEILGTVSW

SEQ ID NO: 720 - protein, 9 aa, SARS-COV2
QELGKYEQY

SEQ ID NO: 721 - protein, 10 aa, SARS-COV2
QEYADVFHLY

SEQ ID NO: 722 - protein, 13 aa, SARS-COV2
QEYADVFHLYLQY

SEQ ID NO: 723 - protein, 9 aa, SARS-COV2
REHEHEIAW

SEQ ID NO: 724 - protein, 9 aa, SARS-COV2
SEFSSLPSY

SEQ ID NO: 725 - protein, 8 aa, SARS-COV2
TEISFMLW

SEQ ID NO: 726 - protein, 9 aa, SARS-COV2
TELEPPCRF

SEQ ID NO: 727 - protein, 10 aa, SARS-COV2
VENPDILRVY

SEQ ID NO: 728 - protein, 9 aa, SARS-COV2
VENPHLMGW

SEQ ID NO: 729 - protein, 10 aa, SARS-COV2
AEIRASANLA

SEQ ID NO: 730 - protein, 9 aa, SARS-COV2
AEIVDTVSA

SEQ ID NO: 731 - protein, 9 aa, SARS-COV2
MEIDFLELA

SEQ ID NO: 732 - protein, 9 aa, SARS-COV2
MELPTGVHA

SEQ ID NO: 733 - protein, 9 aa, SARS-COV2
QEAYEQAVA

SEQ ID NO: 734 - protein, 10 aa, SARS-COV2
QESPFVMMSA

SEQ ID NO: 735 - protein, 9 aa, SARS-COV2
REAACCHLA

SEQ ID NO: 736 - protein, 9 aa, SARS-COV2
SEFRVYSSA

SEQ ID NO: 737 - protein, 9 aa, SARS-COV2
TEVPVAIHA

SEQ ID NO: 738 - protein, 9 aa, SARS-COV2
YENAFLPFA

SEQ ID NO: 739 - protein, 10 aa, SARS-COV2
FAFPFTIYSL

SEQ ID NO: 740 - protein, 9 aa, SARS-COV2
FAIGLALYY

SEQ ID NO: 741 - protein, 9 aa, SARS-COV2
FAVDAAKAY

SEQ ID NO: 742 - protein, 9 aa, SARS-COV2
KAYNVTQAF

SEQ ID NO: 743 - protein, 9 aa, SARS-COV2
LAKDTTEAF

SEQ ID NO: 744 - protein, 9 aa, SARS-COV2
LTKHPNQEY

SEQ ID NO: 745 - protein, 9 aa, SARS-COV2
TLKEILVTY

SEQ ID NO: 746 - protein, 9 aa, SARS-COV2
VARDLSLQF

SEQ ID NO: 747 - protein, 9 aa, SARS-COV2
VATSRTLSY

SEQ ID NO: 748 - protein, 9 aa, SARS-COV2
VVIPDYNTY

SEQ ID NO: 749 - protein, 9 aa, SARS-COV2
VVYRGTTTY

SEQ ID NO: 750 - protein, 8 aa, SARS-COV2
YAFEHIVY

SEQ ID NO: 751 - protein, 9 aa, SARS-COV2
YLFDESGEF

SEQ ID NO: 752 - protein, 9 aa, SARS-COV2
YTPSKLIEY

SEQ ID NO: 753 - protein, 9 aa, SARS-COV2
YVNTFSSTF

SEQ ID NO: 754 - protein, 9 aa, SARS-COV2
CPAEIVDTV

SEQ ID NO: 755 - protein, 9 aa, SARS-COV2
DAPAHISTI

SEQ ID NO: 756 - protein, 9 aa, SARS-COV2
FPFTIYSLL

SEQ ID NO: 757 - protein, 9 aa, SARS-COV2
FPLCANGQV

SEQ ID NO: 758 - protein, 10 aa, SARS-COV2
FPLNSIIKTI

SEQ ID NO: 759 - protein, 9 aa, SARS-COV2
FPRGQGVPI

SEQ ID NO: 760 - protein, 9 aa, SARS-COV2
IPTNFTISV

SEQ ID NO: 761 - protein, 10 aa, SARS-COV2
IPYNSVTSSI

SEQ ID NO: 762 - protein, 8 aa, SARS-COV2
LPFAMGII

SEQ ID NO: 763 - protein, 9 aa, SARS-COV2
LPIDKCSRI

SEQ ID NO: 764 - protein, 10 aa, SARS-COV2
LPIDKCSRII

SEQ ID NO: 765 - protein, 9 aa, SARS-COV2
LPLVSSQCV

SEQ ID NO: 766 - protein, 9 aa, SARS-COV2
LPQNAVVKI

SEQ ID NO: 767 - protein, 8 aa, SARS-COV2
LPWNVVRI

SEQ ID NO: 768 - protein, 10 aa, SARS-COV2
LPWNVVRIKI

SEQ ID NO: 769 - protein, 9 aa, SARS-COV2
LPYPDPSRI

SEQ ID NO: 770 - protein, 9 aa, SARS-COV2
MPASWVMRI

SEQ ID NO: 771 - protein, 9 aa, SARS-COV2
MPLKAPKEI

SEQ ID NO: 772 - protein, 8 aa, SARS-COV2
MPLSAPTL

SEQ ID NO: 773 - protein, 9 aa, SARS-COV2
MPLSAPTLV

SEQ ID NO: 774 - protein, 10 aa, SARS-COV2
VPFWITIAYI

SEQ ID NO: 775 - protein, 9 aa, SARS-COV2
VPQEHYVRI

SEQ ID NO: 776 - protein, 9 aa, SARS-COV2
VPYCYDTNV

SEQ ID NO: 777 - protein, 8 aa, SARS-COV2
VPYNMRVI

SEQ ID NO: 778 - protein, 9 aa, SARS-COV2
YPSLETIQI

SEQ ID NO: 779 - protein, 9 aa, SARS-COV2
CPIHFYSKW

SEQ ID NO: 780 - protein, 10 aa, SARS-COV2
DPYEDFQENW

SEQ ID NO: 781 - protein, 9 aa, SARS-COV2
FAMQMAYRF

SEQ ID NO: 782 - protein, 10 aa, SARS-COV2
FAVHFISNSW

SEQ ID NO: 783 - protein, 8 aa, SARS-COV2
FPFTIYSL

SEQ ID NO: 784 - protein, 9 aa, SARS-COV2
FPFTIYSLL

SEQ ID NO: 785 - protein, 10 aa, SARS-COV2
FPFTIYSLLL

SEQ ID NO: 786 - protein, 10 aa, SARS-COV2
FPNITNLCPF

SEQ ID NO: 787 - protein, 9 aa, SARS-COV2
HADQLTPTW

SEQ ID NO: 788 - protein, 9 aa, SARS-COV2
IPFAMQMAY

SEQ ID NO: 789 - protein, 9 aa, SARS-COV2
IPIQASLPF

SEQ ID NO: 790 - protein, 10 aa, SARS-COV2
IPLMYKGLPW

SEQ ID NO: 791 - protein, 9 aa, SARS-COV2
LAAVNSVPW

SEQ ID NO: 792 - protein, 9 aa, SARS-COV2
LPFFSNVTW

SEQ ID NO: 793 - protein, 10 aa, SARS-COV2
LPFFSNVTWF

SEQ ID NO: 794 - protein, 9 aa, SARS-COV2
LPFNDGVYF

SEQ ID NO: 795 - protein, 8 aa, SARS-COV2
LPNNTASW

SEQ ID NO: 796 - protein, 9 aa, SARS-COV2
LPNNTASWF

SEQ ID NO: 797 - protein, 9 aa, SARS-COV2
LPSLATVAY

SEQ ID NO: 798 - protein, 9 aa, SARS-COV2
LPVNVAFEL

SEQ ID NO: 799 - protein, 10 aa, SARS-COV2
LPVNVAFELW

SEQ ID NO: 800 - protein, 12 aa, SARS-COV2
LPYGANKDGIIW

SEQ ID NO: 801 - protein, 9 aa, SARS-COV2
MPASWVMRI

SEQ ID NO: 802 - protein, 12 aa, SARS-COV2
MPASWVMRIMTW

SEQ ID NO: 803 - protein, 9 aa, SARS-COV2
MPYFFTLLL

SEQ ID NO: 804 - protein, 10 aa, SARS-COV2
NPFMIDVQQW

SEQ ID NO: 805 - protein, 9 aa, SARS-COV2
QPTESIVRF

SEQ ID NO: 806 - protein, 9 aa, SARS-COV2
TAFGLVAEW

SEQ ID NO: 807 - protein, 9 aa, SARS-COV2
TASDTYACW

SEQ ID NO: 808 - protein, 10 aa, SARS-COV2
TPLGIDLDEW

SEQ ID NO: 809 - protein, 10 aa, SARS-COV2
VPANSTVLSF

SEQ ID NO: 810 - protein, 9 aa, SARS-COV2
VPFWITIAY

SEQ ID NO: 811 - protein, 9 aa, SARS-COV2
VPWDTIANY

SEQ ID NO: 812 - protein, 9 aa, SARS-COV2
WPWYIWLGF

SEQ ID NO: 813 - protein, 10 aa, SARS-COV2
YPANSIVCRF

SEQ ID NO: 814 - protein, 10 aa, SARS-COV2
YPKLQSSQAW

SEQ ID NO: 815 - protein, 9 aa, SARS-COV2
YPNASFDNF

SEQ ID NO: 816 - protein, 9 aa, SARS-COV2
CPFGEVFNA

SEQ ID NO: 817 - protein, 9 aa, SARS-COV2
FPFNKWGKA

SEQ ID NO: 818 - protein, 9 aa, SARS-COV2
FPLKLRGTA

SEQ ID NO: 819 - protein, 9 aa, SARS-COV2
FPQSAPHGV

SEQ ID NO: 820 - protein, 9 aa, SARS-COV2
FPRGQGVPI

SEQ ID NO: 821 - protein, 9 aa, SARS-COV2
IPIGAGICA

SEQ ID NO: 822 - protein, 9 aa, SARS-COV2
KPTVVVNAA

SEQ ID NO: 823 - protein, 9 aa, SARS-COV2
LPFAMGIIA

SEQ ID NO: 824 - protein, 12 aa, SARS-COV2
LPFFSNVTWFHA

SEQ ID NO: 825 - protein, 8 aa, SARS-COV2
LPFKLTCA

SEQ ID NO: 826 - protein, 9 aa, SARS-COV2
LPFKLTCAT

SEQ ID NO: 827 - protein, 10 aa, SARS-COV2
LPFNDGVYFA

SEQ ID NO: 828 - protein, 10 aa, SARS-COV2
LPSYAAFATA

SEQ ID NO: 829 - protein, 12 aa, SARS-COV2
LPYPDPSRILGA

SEQ ID NO: 830 - protein, 9 aa, SARS-COV2
MPILTLTRA

SEQ ID NO: 831 - protein, 12 aa, SARS-COV2
MPILTLTRALTA

SEQ ID NO: 832 - protein, 9 aa, SARS-COV2
MPLSAPTLV

SEQ ID NO: 833 - protein, 9 aa, SARS-COV2
MPNMLRIMA

SEQ ID NO: 834 - protein, 9 aa, SARS-COV2
MPVCVETKA

SEQ ID NO: 835 - protein, 9 aa, SARS-COV2
SPIFLIVAA

SEQ ID NO: 836 - protein, 9 aa, SARS-COV2
TPLIQPIGA

SEQ ID NO: 837 - protein, 9 aa, SARS-COV2
VPNQPYPNA

SEQ ID NO: 838 - protein, 10 aa, SARS-COV2
YPSARIVYTA

SEQ ID NO: 839 - protein, 9 aa, SARS-COV2
AIKITEHSW

SEQ ID NO: 840 - protein, 13 aa, SARS-COV2
ASFRLFARTRSMW

SEQ ID NO: 841 - protein, 10 aa, SARS-COV2
ASWVMRIMTW

SEQ ID NO: 842 - protein, 9 aa, SARS-COV2
ATVHTANKW

SEQ ID NO: 843 - protein, 8 aa, SARS-COV2
ATYKPNTW

SEQ ID NO: 844 - protein, 14 aa, SARS-COV2
ATYKPNTWCIRCLW

SEQ ID NO: 845 - protein, 13 aa, SARS-COV2
IAIAMACLVGLMW

SEQ ID NO: 846 - protein, 14 aa, SARS-COV2
IAYIICISTKHFYW

SEQ ID NO: 847 - protein, 11 aa, SARS-COV2
ISMDNSPNLAW

SEQ ID NO: 848 - protein, 9 aa, SARS-COV2
KALNLGETF

SEQ ID NO: 849 - protein, 9 aa, SARS-COV2
KATYKPNTW

SEQ ID NO: 850 - protein, 9 aa, SARS-COV2
KAYKIEELF

SEQ ID NO: 851 - protein, 9 aa, SARS-COV2
KAYNVTQAF

SEQ ID NO: 852 - protein, 10 aa, SARS-COV2
KSAGFPFNKW

SEQ ID NO: 853 - protein, 9 aa, SARS-COV2
KSHKPPISF

SEQ ID NO: 854 - protein, 9 aa, SARS-COV2
KSHNIALIW

SEQ ID NO: 855 - protein, 14 aa, SARS-COV2
KSPNFSKLINIIIW

SEQ ID NO: 856 - protein, 9 aa, SARS-COV2
KSYELQTPF

SEQ ID NO: 857 - protein, 13 aa, SARS-COV2
KTTLPVNVAFELW

SEQ ID NO: 858 - protein, 10 aa, SARS-COV2
LSDRELHLSW

SEQ ID NO: 859 - protein, 10 aa, SARS-COV2
LSDRVVFVLW

SEQ ID NO: 860 - protein, 10 aa, SARS-COV2
LTAFGLVAEW

SEQ ID NO: 861 - protein, 10 aa, SARS-COV2
LTNDNTSRYW

SEQ ID NO: 862 - protein, 13 aa, SARS-COV2
LTNDVSFLAHIQW

SEQ ID NO: 863 - protein, 9 aa, SARS-COV2
MACLVGLMW

SEQ ID NO: 864 - protein, 10 aa, SARS-COV2
MSALNHTKKW

SEQ ID NO: 865 - protein, 9 aa, SARS-COV2
RSFIEDLLF

SEQ ID NO: 866 - protein, 8 aa, SARS-COV2
RTFKVSIW

SEQ ID NO: 867 - protein, 9 aa, SARS-COV2
RTIKGTHHW

SEQ ID NO: 868 - protein, 12 aa, SARS-COV2
RTVYDDGARRVW

SEQ ID NO: 869 - protein, 9 aa, SARS-COV2
SALNHTKKW

SEQ ID NO: 870 - protein, 9 aa, SARS-COV2
TAFGLVAEW

SEQ ID NO: 871 - protein, 10 aa, SARS-COV2
TTFTYASALW

SEQ ID NO: 872 - protein, 12 aa, SARS-COV2
TTLPVNVAFELW

SEQ ID NO: 873 - protein, 10 aa, SARS-COV2
VAIKITEHSW

SEQ ID NO: 874 - protein, 9 aa, SARS-COV2
VSFLAHIQW

SEQ ID NO: 875 - protein, 12 aa, SARS-COV2
VTCGTTTLNGLW

SEQ ID NO: 876 - protein, 9 aa, SARS-COV2
FAQDGNAAI

SEQ ID NO: 877 - protein, 9 aa, SARS-COV2
FASEAARVV

SEQ ID NO: 878 - protein, 9 aa, SARS-COV2
FAVDAAKAY

SEQ ID NO: 879 - protein, 9 aa, SARS-COV2
FGADPIHSL

SEQ ID NO: 880 - protein, 9 aa, SARS-COV2
FVSDADSTL

SEQ ID NO: 881 - protein, 9 aa, SARS-COV2
HANEYRLYL

SEQ ID NO: 882 - protein, 9 aa, SARS-COV2
ITFDNLKTL

SEQ ID NO: 883 - protein, 9 aa, SARS-COV2
VADAVIKTL

SEQ ID NO: 884 - protein, 9 aa, SARS-COV2
YADVFHLYL

SEQ ID NO: 885 - protein, 8 aa, SARS-COV2
LFDESGEF

SEQ ID NO: 886 - protein, 9 aa, SARS-COV2
LYDKLVSSF

SEQ ID NO: 887 - protein, 9 aa, SARS-COV2
MFDAYVNTF

SEQ ID NO: 888 - protein, 9 aa, SARS-COV2
MYDPKTKNV

SEQ ID NO: 889 - protein, 9 aa, SARS-COV2
TFDNLKTLL

SEQ ID NO: 890 - protein, 9 aa, SARS-COV2
VYDDGARRV

SEQ ID NO: 891 - protein, 9 aa, SARS-COV2
VYDPLQPEL

SEQ ID NO: 892 - protein, 8 aa, SARS-COV2
FADDLNQL

SEQ ID NO: 893 - protein, 9 aa, SARS-COV2
FGDDTVIEV

SEQ ID NO: 894 - protein, 9 aa, SARS-COV2
FVDGVPFVV

SEQ ID NO: 895 - protein, 9 aa, SARS-COV2
IADKYVRNL

SEQ ID NO: 896 - protein, 9 aa, SARS-COV2
ISDEFSSNV

SEQ ID NO: 897 - protein, 9 aa, SARS-COV2
IVDEPEEHV

SEQ ID NO: 898 - protein, 9 aa, SARS-COV2
IVDTVSALV

SEQ ID NO: 899 - protein, 9 aa, SARS-COV2
KVDGVDVEL

SEQ ID NO: 900 - protein, 9 aa, SARS-COV2
KVDGVVQQL

SEQ ID NO: 901 - protein, 9 aa, SARS-COV2
LSDDAVVCF

SEQ ID NO: 902 - protein, 9 aa, SARS-COV2
MADQAMTQM

SEQ ID NO: 903 - protein, 9 aa, SARS-COV2
MLDMYSVML

SEQ ID NO: 904 - protein, 9 aa, SARS-COV2
SSDNIALLV

SEQ ID NO: 905 - protein, 9 aa, SARS-COV2
TLDSKTQSL

SEQ ID NO: 906 - protein, 9 aa, SARS-COV2
VADAVIKTL

SEQ ID NO: 907 - protein, 9 aa, SARS-COV2
VSDIDITFL

SEQ ID NO: 908 - protein, 9 aa, SARS-COV2
VTDVTQLYL

SEQ ID NO: 909 - protein, 9 aa, SARS-COV2
VVDSYYSLL

SEQ ID NO: 910 - protein, 9 aa, SARS-COV2
VYDPLQPEL

SEQ ID NO: 911 - protein, 9 aa, SARS-COV2
YADVFHLYL

SEQ ID NO: 912 - protein, 9 aa, SARS-COV2
YGDFSHSQL

SEQ ID NO: 913 - protein, 9 aa, SARS-COV2
YIDIGNYTV

SEQ ID NO: 914 - protein, 9 aa, SARS-COV2
YLDAYNMMI

SEQ ID NO: 915 - protein, 9 aa, SARS-COV2
YSDVENPHL

SEQ ID NO: 916 - protein, 9 aa, SARS-COV2
YVDNSSLTI

SEQ ID NO: 917 - protein, 9 aa, SARS-COV2
FASEAARVV

SEQ ID NO: 918 - protein, 9 aa, SARS-COV2
IRQEEVQEL

SEQ ID NO: 919 - protein, 9 aa, SARS-COV2
KRVDWTIEY

SEQ ID NO: 920 - protein, 9 aa, SARS-COV2
NRFLYIIKL

SEQ ID NO: 921 - protein, 9 aa, SARS-COV2
VRFPNITNL

SEQ ID NO: 922 - protein, 9 aa, SARS-COV2
VRIKIVQML

SEQ ID NO: 923 - protein, 9 aa, SARS-COV2
VRSIFSRTL

SEQ ID NO: 924 - protein, 9 aa, SARS-COV2
IRQEEVQEL

SEQ ID NO: 925 - protein, 9 aa, SARS-COV2
KRVDWTIEY

SEQ ID NO: 926 - protein, 9 aa, SARS-COV2
NRFLYIIKL

SEQ ID NO: 927 - protein, 9 aa, SARS-COV2
SRYWEPEFY

SEQ ID NO: 928 - protein, 9 aa, SARS-COV2
VRFPNITNL

SEQ ID NO: 929 - protein, 9 aa, SARS-COV2
YRGTTTYKL

SEQ ID NO: 930 - protein, 9 aa, SARS-COV2
IYNDKVAGF

SEQ ID NO: 931 - protein, 9 aa, SARS-COV2
KRVDWTIEY

SEQ ID NO: 932 - protein, 9 aa, SARS-COV2
NYMPYFFTL

SEQ ID NO: 933 - protein, 9 aa, SARS-COV2
VRFPNITNL

SEQ ID NO: 934 - protein, 9 aa, SARS-COV2
VYMPASWVM

SEQ ID NO: 935 - protein, 9 aa, SARS-COV2
WRNTNPIQL

SEQ ID NO: 936 - protein, 9 aa, SARS-COV2
YYPSARIVY

SEQ ID NO: 937 - protein, 9 aa, SARS-COV2
YYTSNPTTF

SEQ ID NO: 938 - protein, 9 aa, SARS-COV2
FASEAARVV

SEQ ID NO: 939 - protein, 9 aa, SARS-COV2
FAVDAAKAY

SEQ ID NO: 940 - protein, 9 aa, SARS-COV2
FAYANRNRF

SEQ ID NO: 941 - protein, 9 aa, SARS-COV2
FAYTKRNVI

SEQ ID NO: 942 - protein, 9 aa, SARS-COV2
HANEYRLYL

SEQ ID NO: 943 - protein, 9 aa, SARS-COV2
ITFDNLKTL

SEQ ID NO: 944 - protein, 9 aa, SARS-COV2
KAYNVTQAF

SEQ ID NO: 945 - protein, 9 aa, SARS-COV2
VAYFNMVYM

SEQ ID NO: 946 - protein, 9 aa, SARS-COV2
YAKPFLNKV

SEQ ID NO: 947 - protein, 9 aa, SARS-COV2
YVYSRVKNL

SEQ ID NO: 948 - protein, 8 aa, SARS-COV2
FFITGNTL

SEQ ID NO: 949 - protein, 9 aa, SARS-COV2
FYLTNDVSF

SEQ ID NO: 950 - protein, 9 aa, SARS-COV2
IFFITGNTL

SEQ ID NO: 951 - protein, 9 aa, SARS-COV2
IYNDKVAGF

SEQ ID NO: 952 - protein, 9 aa, SARS-COV2
MFDAYVNTF

SEQ ID NO: 953 - protein, 9 aa, SARS-COV2
NYMPYFFTL

SEQ ID NO: 954 - protein, 9 aa, SARS-COV2
SFSASTSAF

SEQ ID NO: 955 - protein, 9 aa, SARS-COV2
SFYEDFLEY

SEQ ID NO: 956 - protein, 9 aa, SARS-COV2
SYSGQSTQL

SEQ ID NO: 957 - protein, 9 aa, SARS-COV2
TYFTQSRNL

SEQ ID NO: 958 - protein, 9 aa, SARS-COV2
VYDPLQPEL

SEQ ID NO: 959 - protein, 9 aa, SARS-COV2
VYIGDPAQL

SEQ ID NO: 960 - protein, 9 aa, SARS-COV2
VYMPASWVM

SEQ ID NO: 961 - protein, 9 aa, SARS-COV2
YFMRFRRAF

SEQ ID NO: 962 - protein, 9 aa, SARS-COV2
YFVVKRHTF

SEQ ID NO: 963 - protein, 9 aa, SARS-COV2
YYHTTDPSF

SEQ ID NO: 964 - protein, 9 aa, SARS-COV2
YYPSARIVY

SEQ ID NO: 965 - protein, 9 aa, SARS-COV2
YYQLYSTQL

SEQ ID NO: 966 - protein, 9 aa, SARS-COV2
YYTSNPTTF

SEQ ID NO: 967 - protein, 9 aa, SARS-COV2
HANEYRLYL

SEQ ID NO: 968 - protein, 9 aa, SARS-COV2
RAMPNMLRI

SEQ ID NO: 969 - protein, 9 aa, SARS-COV2
RTIKVFTTV

SEQ ID NO: 970 - protein, 9 aa, SARS-COV2
TVYSHLLLV

SEQ ID NO: 971 - protein, 9 aa, SARS-COV2
YADVFHLYL

(19) United States
(12) Patent Application Publication
(10) Pub. No.: US 2023/0187026 A1
(43) Pub. Date: Jun. 15, 2023

(54) SYSTEMS AND METHODS FOR AN INTEGRATED PREDICTION METHOD FOR T-CELL IMMUNITY

(71) Applicant: Arizona Board of Regents on Behalf of Arizona State University, Tempe, AZ (US)

(72) Inventors: Karen Anderson, Scottsdale, AZ (US); Abhishek Singharoy, Chandler, AZ (US); Eric Wilson, Tempe, AZ (US)

(21) Appl. No.: 17/924,079

(22) PCT Filed: May 10, 2021
(86) PCT No.: PCT/US2021/031584

§ 371 Date: Nov. 8, 2022

Related U.S. Application Data
(60) Provisional application No. 63/022,078, filed on May 8, 2020.

(51) Int. Cl. G16B 35/20; G16B 40/10; G16B 20/00

(52) U.S. Cl. CPC G16B 35/20; G16B 40/10; G16B 20/00

(57) ABSTRACT

A consensus MHC-I binding and processing prediction workflow, methods and systems are described, for improving T-cell immunity against threats such as viruses and cancer. The methods and systems can also be used to determine population fitness against a target antigen such as a pathogen or cancer.

### CROSS-REFERENCE TO RELATED APPLICATIONS

This application claims priority from Provisional Application No. 63/022,078; filed May 8, 2020, the contents of which are hereby incorporated by reference in their entirety.

### SEQUENCE LISTING

This application contains a Sequence Listing that has been submitted in ASCII format via EFS-Web and is hereby incorporated by reference in its entirety. The ASCII copy is named 684040_sequencelisting_ST25.txt, and is 147 kilobytes in size.

### FIELD

The present disclosure generally relates to predicting peptide ligands for major histocompatibility complex class I (MHC-I) molecules, and in particular, to consensus sequences for MHC-I binding to improve T-cell immunity against threats such as viruses and cancer, related methods for their prediction, and methods for determining population fitness against a threat.

### BACKGROUND

Immunotherapy design is informed by knowing whether a major histocompatibility complex (MHC) Class-I molecule presents a given peptide. MHC-I ligands can be predicted by in silico binding prediction methods. However, prediction performance substantially varies by method, MHC Class-I type, and peptide length. An MHC-I binding prediction method that is robustly sensitive, specific, and accurate could increase the number of candidate epitopes as possible immunotherapy targets.

A need exists for MHC-I binding prediction methods with improved accuracy, sensitivity, and/or specificity, and which can increase the number of candidate epitopes as possible immunotherapy targets.

### SUMMARY

To address the need for improved MHC-I binding prediction methods and systems, one aspect of the present disclosure includes a method for predicting consensus MHC-I binding by one or more candidate peptides to an MHC-I protein expressed by a cell. The method includes the steps of: (a) obtaining or having obtained training data comprising binding affinity data, for each of a plurality of candidate peptides in a data set, wherein each peptide in the data set is identified by mass spectrometry to be presented by a MHC-I protein as expressed in a mono-allelic cell line; (b) training or having trained a plurality of machine learning HLA-peptide presentation prediction models using the training data; and (c) processing data of the plurality of candidate peptides using the plurality of machine learning HLA-peptide presentation prediction models to generate a presentation prediction for each candidate peptide, wherein each presentation prediction is indicative of a likelihood of the candidate peptide binding to an MHC-I protein expressed in the mono-allelic cell line.

The method can further comprise selecting one or more of the candidate peptides for preparing a vaccine composition comprising a polypeptide comprising one or more of the selected peptides. The one or more selected peptides are predicted to be presented by the MHC-I protein expressed in the mono-allelic cell line.

The method can further comprise determining population fitness of a selected population against a target antigen. Population fitness can be determined by factoring observed MHC-I allele preferences for one or more selected target antigen peptides and regional expression of the MHC-I alleles within the selected population. Fitness of a population is inversely associated with observed allele preferences for the selected target antigen peptides. The one or more selected target antigen peptides are predicted to be presented by the MHC-I allele expressed in the mono-allelic cell line.

In some aspects, the method further comprises determining population fitness of a selected population against a target antigen by factoring observed MHC-I allele preferences for selected target antigen peptides and regional expression of the MHC-I alleles within the selected population. Fitness of a population is inversely associated with observed allele preferences for the selected target antigen peptides; and the one or more selected target antigen peptides are predicted to be presented by the MHC-I allele expressed in the mono-allelic cell line.

In a further aspect, the step of processing the data of the plurality of candidate peptides using the plurality of machine learning HLA-peptide presentation prediction models includes: (a) observing or having observed performance of each model on a mass spectrometry (MS) data set of naturally presented MHC-I peptides from a mono-allelic cell lines; and (b) based on the performance, parameterizing allele and algorithm specific score thresholds and expected false detection rates (FDR) for each model.

In one aspect, the training data comprises data relating to a target antigen, which comprises at least two of peptide binding affinity measurements for the target antigen, MHC-peptide stability data and MHC-I pocket architecture. In some aspects, the method determines one or more possible immunotherapy targets.

In another aspect, the present disclosure provides a method for determining population fitness of a selected population against a target antigen. In one aspect, the method includes the steps of: (a) using an ensemble presentation prediction model combining the presentation prediction output of each of a plurality machine learning HLA-peptide presentation prediction models to provide a single presentation prediction for each of a plurality of candidate peptides with respect to the target antigen, wherein the presentation prediction represents the likelihood of the candidate peptide binding to an MHC-I protein expressed in a mono-allelic cell line; (b) selecting from the candidate peptides based on the presentation prediction for each peptide to determine selected peptides; and (c) using the selected peptides to assess the fitness of a selected population against the target antigen, by factoring the observed allele preferences for the selected target antigen peptides and regional expression of those MHC-I alleles within the selected population.

In another aspect of the method, each of the plurality of machine learning HLA-peptide presentation prediction models have a previously demonstrated accuracy of peptide calls for the target antigen. Accuracy may be determined, for example, by generating an ROC curve and determining an area under the curve (AUC) measurement of at least 80, 85, 90 or 95 for at least one allotype.

In another aspect of the disclosed method, the target antigen is SARS-CoV-2 and the plurality of machine learning HLA-peptide presentation prediction models includes at least 2, 3, 4, 5, 6 or all of: (i) MHCflurry-binding_percentile; (ii) MHCflurry_presentation; (iii) netMHC-4.0, (iv) netMHCpan-EL-4.0, (v) netMHCstabpan; (vi) Pick-pocket; and (vii) MixMHCpred. In one aspect, the plurality of machine learning HLA-peptide presentation prediction models includes all of (i) through (vii) and wherein peptide binding affinity measurements for the target antigen are used to predict a binding affinity for the target antigen using MHCflurry-affinity percentile and netMHC-4.0; MixMHCpred, netMHCpan-EL, and MHCflurry-presentation are trained on naturally eluted MHC-I ligands; MHCflurry-presentation incorporates antigen processing prediction; netMHCstabpan is trained on MHC-peptide stability data; and PickPocket is trained on quantitative binding affinity data and extrapolates binding based on MHC-I pocket architecture. Any or all of these steps may be implemented by a computer processor. In one aspect, the candidate peptides are selected from any one or any combination of potential 5-mers, 6-mers, 7-mers, 8-mers, 9-mers, 10-mers, 11-mers, 12-mers, 13-mers, 14-mers, 15-mers, 16-mers, 17-mers, 18-mers, 19mers and 20-mers with respect to the target antigen.

When the method further comprises, a vaccine composition is formulated including a polypeptide having one or more selected peptide sequences selected according to the disclosed methods, or alternatively including a polynucleotide encoding such a polypeptide, wherein the peptides are selected using any of the disclosed methods. In one aspect, the vaccine composition may be a cell-mediated immune vaccine such as a T-cell vaccine.

In some aspects, the target antigen is a cancer antigen. In some aspects, the target antigen is a pathogen. For instance, the pathogen can be a human immunodeficiency virus (HIV), Hepatitis C virus, Dengue virus, or a coronavirus. In one aspect, the target antigen is SARS-CoV2 and the vaccine composition is a SARS-CoV2 vaccine composition.

In other aspects, the present disclosure provides a method of determining population fitness of a selected population against a target antigen. The method comprises the steps of (a) using an ensemble presentation prediction model combining the presentation prediction output of each of a plurality of machine learning HLA-peptide presentation prediction models to provide a single presentation prediction for each of a plurality of candidate peptides with respect to the target antigen, wherein the presentation prediction represents the likelihood of the candidate peptide binding to an MHC-I protein expressed in a mono-allelic cell line; (b) selecting from the candidate peptides based on the presentation prediction for each peptide to determine selected peptides; and (c) using the selected peptides to assess the fitness of a selected population against the target antigen, by factoring observed allele preferences for the selected target antigen peptides and regional expression of those MHC-I alleles within the selected population.

In some aspects, fitness of a population is inversely associated with observed allele preferences for the selected target antigen peptides. In some aspects, each of the plurality of machine learning HLA-peptide presentation prediction models have a previously demonstrated accuracy of peptide calls for the target antigen, wherein accuracy is determined by generating an ROC curve and determining an area under the curve (AUC) measurement of at least 80, 85, 90 or 95 for at least one allotype.

In some aspects, the target antigen can be cancer. The target antigen can also be a pathogen. For instance, the pathogen can be a human immunodeficiency virus (HIV), Hepatitis C virus, Dengue virus, or a coronavirus. The target antigen can be SARS-CoV-2.

In some aspects, the target antigen is SARS-CoV2 and the vaccine composition is a SARS-CoV2 vaccine composition. In some aspects, the plurality of machine learning HLA-peptide presentation prediction models comprises at least 2, 3, 4, 5, 6 or all of: (i) MHCflurry-binding_percentile, (ii) MHCflurry_presentation, (iii) netMHC-4.0, (iv) netMHCpan-EL-4.0, (v) netMHCstabpan, (vi) Pick-pocket, and (vii) MixMHCpred. The plurality of machine learning HLA-peptide presentation prediction models can comprise all of (i) through (vii) and wherein peptide binding affinity measurements are for the target antigen are used to predicting a binding affinity for the target antigen using MHCflurry-affinity_percentile and netMHC-4.0; MixMHCpred, netMHCpan-EL, and MHCflurry-presentation are trained on naturally eluted MHC-I ligands; MHCflurry-presentation incorporates antigen processing prediction; netMHCstabpan is trained on MHC-peptide stability data; and PickPocket is trained on quantitative binding affinity data and extrapolates binding based on MHC-I pocket architecture.

Any of the methods described herein above can be performed by a computer processor

In another aspect, the present disclosure provides a peptide library comprising a plurality of library members is disclosed. Each member of the peptide library is, for example, a 5-20mer peptide having a predetermined likelihood of binding to a target antigen, and is restricted to a predetermined number of common MHC-I alleles, wherein each member is selected from a plurality of candidate peptides based on a presentation prediction for each peptide with respect to the target antigen. The presentation prediction represents the likelihood of the candidate peptide binding to an MHC-I protein expressed in a mono-allelic cell line. The presentation prediction is an output of an ensemble presentation prediction model combining the presentation prediction output of each of a plurality machine learning HLA-peptide presentation prediction models to provide a single presentation prediction for each of a plurality of candidate peptides with respect to the target antigen. In one aspect, the target antigen is SARS-CoV2 and the library includes all potential 8-14mer target antigen peptides restricted to 52 common MHC-I alleles.

In another aspect, the present disclosure also provides a vaccine composition which includes a polypeptide comprising any one or more of library member peptide sequences, or a polynucleotide encoding the polypeptide. The vaccination composition may be, for example, a T-cell vaccine composition. In one aspect, the library may comprise any one or more of the 8-14mer peptides listed in Table A, a polypeptide comprising any one or more of the 8-14mer peptides listed in Table A, or a polynucleotide encoding the polypeptide. In another aspect, the present disclosure provides a method of treating a SARS-COv2 infection in a subject in need thereof, the method including administering any of the disclosed vaccination compositions to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A EnsembleMHC prediction workflow. The EnsembleMHC score algorithm was parametrized using MHC-I peptides observed in mass spectrometry datasets and 100 randomly generated length and protein matched decoy peptides. Diagram showing the observed false detection rate (FDR) distribution at 50% recall for algorithms relative to each HLA.

FIG. 1B EnsembleMHC prediction workflow. The EnsembleMHC score algorithm was parametrized using MHC-I peptides observed in mass spectrometry datasets and 100 randomly generate length and protein matched decoy peptides. Diagram showing the distribution of observed FDRs for each algorithm across all alleles.

FIG. 1C EnsembleMHC prediction workflow. The EnsembleMHC score algorithm was parametrized using MHC-I peptides observed in mass spectrometry datasets and 100 randomly generate length and protein matched decoy peptides. The correlation between individual peptide scores for each algorithm across all alleles.

FIG. 1D EnsembleMHC prediction workflow. The EnsembleMHC score algorithm was parametrized using MHC-I peptides observed in mass spectrometry datasets and 100 randomly generate length and protein matched decoy peptides. The EnsembleMHC workflow for the prediction of SARS-Cov-2 peptides

FIG. 1E EnsembleMHC prediction workflow. Continuation of FIG. 1D.

FIG. 2A EnsembleMHC reveals anomalies in predicted peptide distribution across 52 alleles. EnsembleMHC workflow was used to predict 8-14mer MHC-I peptides for 52 alleles from the entire SARS-CoV-2 proteome.

FIG. 2B EnsembleMHC reveals anomalies in predicted peptide distribution across 52 alleles. EnsembleMHC workflow was used to predict 8-14mer MHC-I peptides for 52 alleles from specifically SARS-CoV-2 structural proteins.

FIG. 2C EnsembleMHC reveals anomalies in predicted peptide distribution across 52 alleles. Distributions of FIG. 2A and FIG. 2B were individually standardized and the relative change in the binding capacity of each allele was calculated by taking the absolute difference of the Z-scores of allele binding capacity with respect to all SARS proteins or SARS structural proteins. Alleles showing a greater than 1 standard deviation increase or decrease change in binding capacity are highlighted in color.

FIG. 2D EnsembleMHC reveals anomalies in predicted peptide distribution across 52 alleles. The MHC-I peptide density of each SARS-CoV-2 protein was calculated as the percentage of amino acids within that protein that appear within a unique predicted peptide. Red lines indicate structural proteins.

FIG. 3A EnsembleMHC population score correlates with observed death rate. The correlation between EnsembleMHC population score with respect to all SARS-CoV-2 proteins and deaths per million were calculated at each day starting from the day a country passed a particular death milestone ranging from 1 reported death to 100 reported deaths (line color). The days from each start point were normalized, and correlations that were shown to be statistically significant are colored with a red point.

FIG. 3B EnsembleMHC population score correlates with observed death rate. The correlation between EnsembleMHC population score with respect to structural proteins and deaths per million were calculated at each day starting from the day a country passed a particular death milestone ranging from 1 reported death to 100 reported deaths (line color). The days from each start point were normalized, and correlations that were shown to be statistically significant are colored with a red point.

FIG. 3C EnsembleMHC population score correlates with observed death rate. The correlations between the EnsembleMHC score based on structural proteins and death rate were shown for countries meeting the 50 confirmed death threshold. The correlation between deaths per million rank (min rank=least number of deaths max rank=most deaths) and EnsembleMHC population score rank (min rank=lowest score max rank=highest score) at day 1. Correlation coefficients and p values were assigned using spearman's rank correlation.

FIG. 3D EnsembleMHC population score correlates with observed death rate. The correlations between the EnsembleMHC score based on structural proteins and death rate were shown for countries meeting the 50 confirmed death threshold. The correlation between deaths per million rank (min rank=least number of deaths max rank=most deaths) and EnsembleMHC population score rank (min rank=lowest score max rank=highest score) at day 5. Correlation coefficients and p values were assigned using spearman's rank correlation.

FIG. 3E EnsembleMHC population score correlates with observed death rate. The correlations between the EnsembleMHC score based on structural proteins and death rate were shown for countries meeting the 50 confirmed death threshold. The correlation between deaths per million rank (min rank=least number of deaths max rank=most deaths) and EnsembleMHC population score rank (min rank=lowest score max rank=highest score) at day 10. Correlation coefficients and p values were assigned using spearman's rank correlation.

FIG. 3F EnsembleMHC population score correlates with observed death rate. The correlations between the EnsembleMHC score based on structural proteins and death rate were shown for countries meeting the 50 confirmed death threshold. The correlation between deaths per million rank (min rank=least number of deaths max rank=most deaths) and EnsembleMHC population score rank (min rank=lowest score max rank=highest score) at day 15. Correlation coefficients and p values were assigned using spearman's rank correlation.

FIG. 3G EnsembleMHC population score correlates with observed death rate; day 1. The correlations between the EnsembleMHC score based on structural proteins and death rate were shown for countries meeting the 50 confirmed death threshold. The countries at each time point were partitioned into an upper or lower half based on the observed EnsembleMHC population score. P values were determined by Mann-Whitney U test.

FIG. 3H EnsembleMHC population score correlates with observed death rate; day 5. The correlations between the EnsembleMHC score based on structural proteins and death rate were shown for countries meeting the 50 confirmed death threshold. The countries at each time point were partition into an upper or lower half based on the observed EnsembleMHC population score. P values were determined by Mann-Whitney U test.

FIG. 3I EnsembleMHC population score correlates with observed death rate; day 10. The correlations between the EnsembleMHC score based on structural proteins and death rate were shown for countries meeting the 50 confirmed death threshold. The countries at each time point were partition into an upper or lower half based on the observed EnsembleMHC population score. P values were determined by Mann-Whitney U test.

FIG. 3J EnsembleMHC population score correlates with observed death rate; day 15. The correlations between the EnsembleMHC score based on structural proteins and death rate were shown for countries meeting the 50 confirmed death threshold. The countries at each time point were partition into an upper or lower half based on the observed EnsembleMHC population score. P values were determined by Mann-Whitney U test.

FIG. 4A Protein origin of predicted SRAS-CoV-2 peptides. The localization of predicted MHC-I peptides derived from SARS-CoV-2 structural proteins was determined by mapping the peptides back to the reference sequence. The frequency of each amino acid for the SARS-CoV-2 E structural proteins appearing in one of the 160 predicted peptides.

FIG. 4B Protein origin of predicted SRAS-CoV-2 peptides. The localization of predicted MHC-I peptides derived from SARS-CoV-2 structural proteins was determined by mapping the peptides back to the reference sequence. The frequency of each amino acid for the SARS-CoV-2 N structural proteins appearing in one of the 160 predicted peptides.

FIG. 4C Protein origin of predicted SRAS-CoV-2 peptides. The localization of predicted MHC-I peptides derived from SARS-CoV-2 structural proteins was determined by mapping the peptides back to the reference sequence. The frequency of each amino acid for the SARS-CoV-2 M structural proteins appearing in one of the 160 predicted peptides.

FIG. 4D Protein origin of predicted SRAS-CoV-2 peptides. The localization of predicted MHC-I peptides derived from SARS-CoV-2 structural proteins was determined by mapping the peptides back to the reference sequence. The frequency of each amino acid for the SARS-CoV-2 S structural proteins appearing in one of the 160 predicted peptides.

FIG. 4E Protein origin of predicted SRAS-CoV-2 peptides. The localization of predicted MHC-I peptides derived from SARS-CoV-2 structural proteins was determined by mapping the peptides back to the reference sequence. The number of polymorphisms appearing at each position in the E protein structural sequence determined from the alignment of 104 reported SARS-CoV-2 sequences.

FIG. 4F Protein origin of predicted SRAS-CoV-2 peptides. The localization of predicted MHC-I peptides derived from SARS-CoV-2 structural proteins was determined by mapping the peptides back to the reference sequence. The number of polymorphisms appearing at each position in the N protein structural sequence determined from the alignment of 104 reported SARS-CoV-2 sequences.

FIG. 4G Protein origin of predicted SRAS-CoV-2 peptides. The localization of predicted MHC-I peptides derived from SARS-CoV-2 structural proteins was determined by mapping the peptides back to the reference sequence. The number of polymorphisms appearing at each position in the M protein structural sequence determined from the alignment of 104 reported SARS-CoV-2 sequences.

FIG. 4H Protein origin of predicted SRAS-CoV-2 peptides. The localization of predicted MHC-I peptides derived from SARS-CoV-2 structural proteins was determined by mapping the peptides back to the reference sequence. The number of polymorphisms appearing at each position in the S protein structural sequence determined from the alignment of 104 reported SARS-CoV-2 sequences.

FIG. 4I Protein origin of predicted SRAS-CoV-2 peptides. The localization of predicted MHC-I peptides derived from SARS-CoV-2 structural proteins was determined by mapping the peptides back to the reference sequence. The predicted peptides were mapped onto the solved structures for the envelope protein. Red regions indicate an enrichment of predicted peptides and blue regions indicate a depletion of predicted peptides.

FIG. 4J Protein origin of predicted SRAS-CoV-2 peptides. The localization of predicted MHC-I peptides derived from SARS-CoV-2 structural proteins was determined by mapping the peptides back to the reference sequence. The predicted peptides were mapped onto the predicted structure for the nucleocapsid protein. Red regions indicate an enrichment of predicted peptides and blue regions indicate a depletion of predicted peptides.

FIG. 4K Protein origin of predicted SRAS-CoV-2 peptides. The localization of predicted MHC-I peptides derived from SARS-CoV-2 structural proteins was determined by mapping the peptides back to the reference sequence. The predicted peptides were mapped onto the predicted structure for the membrane protein. Red regions indicate an enrichment of predicted peptides and blue regions indicate a depletion of predicted peptides.

FIG. 4L Protein origin of predicted SRAS-CoV-2 peptides. The localization of predicted MHC-I peptides derived from SARS-CoV-2 structural proteins was determined by mapping the peptides back to the reference sequence. Spike protein. Red regions indicate an enrichment of predicted peptides and blue regions indicate a depletion of predicted peptides.

FIG. 5A is a flowchart showing a parameterization aspect of the system of FIGS. 1A-1E.

FIG. 5B is a continuation of flowchart of FIG. 5A, showing a parameterization aspect of the system of FIG. 1A-FIG. 1E.

FIG. 6A Data processing EnsembleMHC population score calculation. The overview of the data processing steps for the global MHC-I allele frequency data and its application in the calculation the EnsembleMHC population score with respect to all SARS-CoV-2 proteins and SARS-CoV-2 structural proteins.

FIG. 6B Continuation of data processing EnsembleMHC population score calculation of FIG. 6A.

FIG. 7A Results of MHC allele frequency data filters and distribution of MHC-I breadth and depth. The number of countries remain after each filtering step.

FIG. 7B Results of MHC allele frequency data filters and distribution of MHC-I breadth and depth. The distribution of the number of alleles described at 4-digit resolution for countries with at least 1 reported coronavirus case (top panel), and the distribution of total number of genotyped individuals in those countries (bottom panel).

FIG. 8A EnsembleMHC population score and deaths per million correlation using different allele frequency accounting methods. The effect on the reported correlation with respect to alternative allele frequency accounting methods (methods). The aggregation of allele frequencies within a particular country by taking the sample weighted mean of reported frequencies for a given allele.

FIG. 8B EnsembleMHC population score and deaths per million correlation using different allele frequency accounting methods. The effect on the reported correlation with respect to alternative allele frequency accounting methods (methods). Normalizing the allele count with respect to all detected alleles in a given population.

FIG. 8C EnsembleMHC population score and deaths per million correlation using different allele frequency accounting methods. The effect on the reported correlation with respect to alternative allele frequency accounting methods (methods). Normalizing allele count with respect to only alleles supported by EnsembleMHC.

FIG. 9A Justification of upper limit for death threshold. The mean statistical power of the resulting correlation of EnsembleMHC population at different death thresholds by day zero. The red line indicates the selected upper limit of analysis of 100 days.

FIG. 9B Justification of upper limit for death threshold. The number of countries remaining at day seven from different death thresholds.

FIG. 10A Justification of nonparametric correlation analysis. The use of nonparametric correlation analysis, namely spearman's rho, is justified by the non-normality of the underlying data. EnsembleMHC population scores for all SARS-CoV-2 proteins and structural proteins were calculated for 10,000 simulated countries. Allele frequencies for simulated countries were generated by randomly sampling an observed allele frequency for each of the considered 52 alleles and renormalizing to ensure the sum of allele frequencies were equal to one. Q-Q plots for nonstructural SARS-CoV-2 proteins EnsembleMHC population score demonstrate positive skew.

FIG. 10B Justification of nonparametric correlation analysis. The use of nonparametric correlation analysis, namely spearman's rho, is justified by the non-normality of the underlying data. EnsembleMHC population scores for all SARS-CoV-2 proteins and structural proteins were calculated for 10,000 simulated countries. Allele frequencies for simulated countries were generated by randomly sampling an observed allele frequency for each of the considered 52 alleles and renormalizing to ensure the sum of allele frequencies were equal to one. Q-Q plots for structural SARS-CoV-2 proteins EnsembleMHC population score demonstrate positive skew.

FIG. 10C Justification of nonparametric correlation analysis. The use of nonparametric correlation analysis, namely spearman's rho, is justified by the non-normality of the underlying data. EnsembleMHC population scores for all SARS-CoV-2 proteins and structural proteins were calculated for 10,000 simulated countries. Allele frequencies for simulated countries were generate by randomly sampling an observed allele frequency for each of the considered 52 alleles and renormalizing to ensure the sum of allele frequencies were equal to one. The QQ plot for all reported deaths per Million indicates a very strong positive skew.

FIG. 11A The effect of different correlation methods on the relationship between EnsembleMHC score and deaths per million. The correlation between EnsembleMHC population score with respect to all SARS-CoV-2 proteins (left panel) or structural proteins (right panel) and deaths per million were calculated at each day starting from the day a country passed a particular death milestone using Pearson's correlation. The days from each start point were normalized, and correlations that were shown to be statistically significant are colored with a red point.

FIG. 11B The effect of different correlation methods on the relationship between EnsembleMHC score and deaths per million. The correlation between EnsembleMHC population score with respect to all SARS-CoV-2 proteins (left panel) or structural proteins (right panel) and deaths per million were calculated at each day starting from the day a country passed a particular death milestone using Spearman's rho. The days from each start point were normalized, and correlations that were shown to be statistically significant are colored with a red point.

FIG. 11C The effect of different correlation methods on the relationship between EnsembleMHC score and deaths per million. The correlation between EnsembleMHC population score with respect to all SARS-CoV-2 proteins (left panel) or structural proteins (right panel) and deaths per million were calculated at each day starting from the day a country passed a particular death milestone using Kendall's tau. The days from each start point were normalized, and correlations that were shown to be statistically significant are colored with a red point.

FIG. 12A Analysis of statistical power for each correlation method. The statistical power of each reported correlation between EnsembleMHC population score with respect to all SARS-CoV-2 proteins (left column) or structural proteins (right column) and deaths per million were calculated at each day starting from the day a country passed a particular death milestone using Pearson's correlation. The days from each start point were normalized, and correlations that were shown to be statistically significant are colored with a red point. The orange line indicates a power threshold of 80%.

FIG. 12B Analysis of statistical power for each correlation method. The statistical power of each reported correlation between EnsembleMHC population score with respect to all SARS-CoV-2 proteins (left column) or structural proteins (right column) and deaths per million were calculated at each day starting from the day a country passed a particular death milestone using Spearman's rho. The days from each start point were normalized, and correlations that were shown to be statistically significant are colored with a red point. The orange line indicates a power threshold of 80%.

FIG. 12C Analysis of statistical power for each correlation method. The statistical power of each reported correlation between EnsembleMHC population score with respect to all SARS-CoV-2 proteins (left column) or structural proteins (right column) and deaths per million were calculated at each day starting from the day a country passed a particular death milestone using Kendall's tau. The days from each start point were normalized, and correlations that were shown to be statistically significant are colored with a red point. The orange line indicates a power threshold of 80%.

FIG. 12D Analysis of statistical power for each correlation method. The statistical power of each reported correlation between EnsembleMHC population score with respect to all SARS-CoV-2 proteins (left column) or structural proteins (right column) and deaths per million were calculated at each day starting from the day a country passed a particular death milestone using Pearson's correlation. The days from each start point were normalized, and correlations that were shown to be statistically significant are colored with a red point. The orange line indicates a power threshold of 80%. The line plot on the left shows the proportion of points achieving a PPV value of greater than 95% at different threshold for pre study odd (R) for the spearman correlation as carried out in the specification below.

FIG. 13A Contribution of each algorithm in EnsembleMHC to the total number of predicted peptides. The UpSet plot shows the contribution of each algorithm to the 658 unique SARS-CoV-2 peptides identified by EnsembleMHC. The top bar plot indicates the number of unique peptides identified by the combination of algorithms shown by the points and segments under each bar.

FIG. 13B Contribution of each algorithm in EnsembleMHC to the total number of predicted peptides. The bars indicate the total number of peptides identified by each algorithm.

FIG. 14A EnsembleMHC peptide FDR distribution and length distributions of predicted SARS-CoV-2 MHC-I peptides. The distribution of peptide FDRs for the 9,712 peptides before the application of the peptide FDR filter. The redline indicates an FDR level of 5%.

FIG. 14B EnsembleMHC peptide FDR distribution and length distributions of predicted SARS-CoV-2 MHC-I peptides. The length distribution of peptides identified from the entire SARS-CoV-2 proteome.

FIG. 14C EnsembleMHC peptide FDR distribution and length distributions of predicted SARS-CoV-2 MHC-I peptides. (A) The distribution of peptide FDRs for the 9,712 peptides before the application of the peptide FDR filter. The redline indicates an FDR level of 5%. The length distribution of peptides identified from SARS-CoV-2 structural protein (C).

FIG. 15A-FIG. 15G Logo plots for the identified peptides from the SARS proteome. Logo plots were generated for MHC alleles with at least 5 peptides identified by EnsembleMHC prediction of all SARS-Cov-2 proteins.

FIG. 16A The distribution of peptide source proportion by allele. The proportion of peptides derived from each SARS-CoV-2 protein for each MHC-I allele. Each point indicates for each protein representing a different MHC-I allele.

FIG. 16B The distribution of peptide source proportion by allele. The proportion of peptides derived from either structural proteins or non-structural proteins. MHC-I alleles in both plots showing greater than 2 standard deviations are labeled.

FIG. 17A Bootstrapping analysis of EnsembleMHC score and deaths per million correlations at the 50 deaths threshold. The bar plot indicates the proportion of bootstrap iteration for each condition that produced a statistically significant value.

FIG. 17 Bootstrapping analysis of EnsembleMHC score and deaths per million correlations at the 50 deaths threshold. The correlations observed in the EnsembleMHC score and deaths per million correlations at the 50 deaths threshold were calculated over 1000 bootstrap iterations (True). In each bootstrap iteration, 50% of the countries were dropped from the analysis. The incidence of spurious statistically significant correlations was simulated by repeating the same bootstrap procedure but with randomized EnsembleMHC scores (scrambled). The green line indicates a PPV value of greater than 95%.

FIG. 17 Bootstrapping analysis of EnsembleMHC score and deaths per million correlations at the 50 deaths threshold. The correlations observed in the EnsembleMHC score and deaths per million correlations at the 50 deaths threshold were calculated over 1000 bootstrap iterations (True). In each bootstrap iteration, 50% of the countries were dropped from the analysis. The incidence of spurious statistically significant correlations was simulated by repeating the same bootstrap procedure but with randomized EnsembleMHC scores (scrambled).

FIG. 18A-FIG. 18H is a series of graphical representations showing that individual algorithms are unable to recreate the correlation reported by the system of FIG. 1A-FIG. 1E.

FIG. 19 is a diagram showing a computing system for use with the system of FIG. 1.

FIG. 20A. Application of the EnsembleMHC prediction algorithm. The EnsembleMHC prediction algorithm was used to recover MHC-I peptides from 10 tumor sample datasets. The average precision and recall for EnsembleMHC and each component algorithm were calculated across all 10 tumor samples. Peptide identification by each algorithm was based on commonly used restrictive (strong) or permissive (strong and weak) binding affinity thresholds.

FIG. 20B. Application of the EnsembleMHC prediction algorithm. The EnsembleMHC prediction algorithm was used to recover MHC-I peptides from 10 tumor sample datasets. The F1 score of each algorithm was calculated for all tumor samples. Each algorithm is grouped into 1 of 4 categories: binding affinity represented by percentile score (blue), binding affinity represented by predicted peptide half-maximal inhibitory concentration (1050) value (green), MHC-1 presentation prediction (orange), and EnsembleMHC (brown). The heat map colors indicate the value of the observed F1 score (color bar) for a given algorithm (y axis) on a particular dataset (x axis). Warmer colors indicate higher F1 scores, and cooler colors indicate lower F1 scores. The average F1 score for each algorithm across all of the samples is shown in the marginal bar plot.

FIG. 20C. Application of the EnsembleMHC prediction algorithm. The EnsembleMHC prediction algorithm was used to recover MHC-I peptides from 10 tumor sample datasets. The schematic for the application of the EnsembleMHC predication algorithm to identify SARS-CoV-2 MHC-I peptides.

FIG. 20D. Application of the EnsembleMHC prediction algorithm. The EnsembleMHC prediction algorithm was used to recover MHC-I peptides from 10 tumor sample datasets. Continuation of the schematic for the application of the EnsembleMHC predication algorithm to identify SARS-CoV-2 MHC-I peptides of FIG. 20C.

FIG. 21A. Prediction of SARS-CoV-2 peptides across 52 common MHC-I alleles (first and second panels). The EnsembleMHC workflow was used to predict MHC-I peptides for 52 alleles from the entire SARS-CoV-2 proteome.

FIG. 21B. Prediction of SARS-CoV-2 peptides across 52 common MHC-I alleles (first and second panels). The EnsembleMHC workflow was used to predict MHC-I peptides for 52 alleles from the SARS-CoV-2 structural proteins (E, S, N, and M).

FIG. 21C. Prediction of SARS-CoV-2 peptides across 52 common MHC-I alleles (FIG. 21A and FIG. 21B). The peptide fractions for both protein sets were calculated by dividing the number of peptides assigned to a given allele by the total number of identified peptides for that protein set. Each line indicates the change in peptide fraction observed by a given allele when comparing the viral peptide-MHC allele distribution for the full SARS-CoV-2 proteome or structural proteins. Alleles showing a change of greater than the median peptide fraction, X=0.015, are highlighted in color.

FIG. 22A. Predicted total epitope load within a population inversely correlates with mortality. SARS-CoV-2 structural protein-based EnsembleMHC population (EMP) scores were assigned to 23 countries (Table B), and correlated with observed mortality rate (deaths per million; Table C). The correlation coefficient is presented as a function of time. Individual country mortality rate data were aligned by truncating each dataset to start after a minimum threshold of deaths was observed in a given country (line color). The Spearman's rank correlation coefficient between structural protein EMP score and SARS-CoV-2 mortality rate was calculated every day following day 0 for each of the minimum death thresholds. Due to the differing lengths of time series analysis at each minimum death threshold, the number of days was normalized to improve visualization. Thus, normalized day 0 represents the day when qualifying countries recorded at least the number of deaths indicated by the minimum death threshold; normalized day 1 represents the final time point at which a correlation was measured. Correlations that were shown to be statistically significant (p % 0.05) are indicated by a red point. The p values were corrected using the Benjamini-Hochberg procedure relative to the number of tests performed for each death threshold.

FIG. 22B-FIG. 22F. Predicted total epitope load within a population inversely correlates with mortality. The correlations between the structural protein EMP score (y axis) and deaths per million (x axis) were shown for countries meeting the 50 minimum deaths threshold at days 1, 6, 12, 17, and 22. Correlation coefficients and p values were assigned using Spearman's rank correlation and the shaded region signifies the 95% confidence interval. Due to Spearman's rank correlation only considering data rank, deaths per million and EMP score were converted to ascending rank values (low rank=low values, high rank=high values) to improve visualization of the measured relationship. Red points indicate a country that has an EMP rank that is less than the median EMP rank of all countries at that day, and blue points indicate a country with an EMP rank that is greater than the median EMP rank. The p values were corrected using the Benjamini-Hochberg procedure relative to the number of tests performed for each death threshold.

FIG. 22G FIG. 22K. Predicted total epitope load within a population inversely correlates with mortality. The countries at each day were partitioned into an upper or a lower half based on the median observed EMP rank. Therefore, countries with an EMP rank greater than the median group EMP score were assigned to the upper half (red) and the remaining countries were assigned to the lower half (blue). p values were determined by the Mann-Whitney U test. The presented boxplots are in the style of Tukey (box defined by 25%, 50%, and 75% quantiles, and whiskers ±1.5 3 IQR). The increasing gap between the red and the blue boxplots indicates a greater discrepancy in the number of deaths per million between the 2 groups. The p values were corrected using the Benjamini-Hochberg procedure relative to the number of tests performed for each death threshold.

FIG. 23A-FIG. 23C. Analysis of other SARS-CoV-2 covariates with observed SARS-CoV-2 population mortality and development of an integrative model. A total of 12 covariates associated with SARS-CoV-2 mortality on the individual patient level were assessed for correlation with population-level mortality (Table C). The correlation of each country-level covariate was determined at each time point after a minimum death threshold was met (line color). The x axis represents the number of days (normalized) following when a minimum death threshold was met, and the y axis indicates the observed effect size for that covariate at a given time point. Correlations achieving statistical significance are colored with a red dot.

FIG. 23D-FIG. 23H. Analysis of other SARS-CoV-2 covariates with observed SARS-CoV-2 population mortality and development of an integrative model. All possible combinations of covariates were used to fit a linear model. The top 10 models, ranked by median adjusted R² (red bars), were identified. The proportion of regressions performed by that model that were found to be statistically significant (F-test p value % 0.05) are represented by the blue bars.

FIG. 24A. EnsembleMHC Parameterization workflow and viral peptide analysis, related to FIG. 20. EnsembleMHC Parametrization workflow.

Figure S1: FIG. 24B EnsembleMHC Parameterization workflow and viral peptide analysis, related to FIG. 20. The EnsembleMHC score algorithm was parameterized using high quality mass spectrometry-detected MHC-I peptides paired with a 100-fold excess of randomly generated decoy peptides. Each bar represents the distribution of algorithm-specific false detection rates (n=7) at that MHC allele.

FIG. 24C EnsembleMHC Parameterization workflow and viral peptide analysis, related to FIG. 20. A density plot of the observed FDRs for each algorithm across all alleles (n=52).

FIG. 24D EnsembleMHC Parameterization workflow and viral peptide analysis, related to FIG. 20. The correlation between individual peptide scores for each algorithm across all alleles was calculated using Pearson correlation. Warmer colors indicate a higher level of correlation while cooler colors indicate lower correlation.

FIG. 24E EnsembleMHC Parameterization workflow and viral peptide analysis, related to FIG. 20. Matthew's correlation coefficient was calculated for each algorithm. Warm colors indicate higher MCC while cooler colors indicate lower MCC. The average MCC for each algorithm is represented by the bar plot on the right margin.

FIG. 24F-FIG. 24G EnsembleMHC Parameterization workflow and viral peptide analysis, related to FIG. 20A-20C. The effect of different peptide^{FDR} cutoff thresholds on the results reported in FIG. 20A-20C was evaluated for a range of 0.01-1. The peptide^{FDR} selected for use in this study is highlighted in red.

FIG. 24H EnsembleMHC Parameterization workflow and viral peptide analysis, related to FIG. 20. The analysis reported in FIGS. 20A and 20B were repeated with additional comparisons to consensus-based MHC-I netMHCcons prediction algorithm.

FIG. 24I-FIG. 24J EnsembleMHC Parameterization workflow and viral peptide analysis, related to FIG. 20A-20C. The analysis reported in FIGS. 20A and 20B were repeated with additional comparisons to consensus-based MHC-I netCTLpan prediction algorithms.

FIG. 24K EnsembleMHC Parameterization workflow and viral peptide analysis, related to FIG. 20A-20C. The positive predictive value of each algorithm was calculated with respect to ability to identify immunogenic peptides derived from Hepatitis-C genome polyprotein, Dengue virus genome polyprotein, and the HN-1 POL-GAG protein when selecting n number of top scoring peptides.

FIG. 25A Data processing and EnsembleMHC population score calculation workflow, related to FIG. 20A-20C. The overview of the data processing steps used on the global MHC-I allele frequency data and the calculation of the EnsembleMHC population score with respect to the full SARS-CoV-2 proteome and SARS-CoV-2 structural proteins (FIGS. 25B-C).

FIG. 25B Data processing and EnsembleMHC population score calculation workflow, related to FIG. 20A-20C. Plot illustrating MHC-typing breadth and depth variation by showing the distribution of the total number of MHC-I allele reported at 1-digit resolution in 86 countries. A FND=Allele Frequency Net Database.

FIG. 25C Data processing and EnsembleMHC population score calculation workflow, related to FIG. 20A-20C. Plot showing the distribution of the number of MHC-genotyped individuals in the set of countries with at least 1 reported coronavirus case. A FND=Allele Frequency Net Database.

FIG. 26A Characteristics of peptides predicted by EnsembleMHC, related to FIGS. 21A and 21B. The UpSet plot shows the contribution of each individual component algorithm to the 658 unique SA RS-CoV-2 peptides identified by EnsembleMHC. The top bar plot indicates the number of unique peptides identified by the combination of algorithms shown by the points and segments 16 located under each bar. The bar plot on the left-hand side of the plot indicates the total number of peptides identified by each algorithm.

FIG. 26B Characteristics of peptides predicted by EnsembleMHC, related to FIGS. 21A and 21B. The peptide^{FDR} distribution of the 9,712 SARS-CoV-2 peptides that fell with the score threshold of at least one component algorithm. The red line indicates an peptide^{FDR} level of <5% h.

FIG. 26C Characteristics of peptides predicted by EnsembleMHC, related to FIGS. 21A and 21B. The length distribution of the 108 high-coincidence peptides identified from SA RS-CoV-2 structural proteins.

FIG. 26D Characteristics of peptides predicted by EnsembleMHC, related to FIGS. 21A and 21B. The length distribution of the 658 high-coincidence peptides identified from full SA RS-CoV-2 proteome.

FIG. 26E-FIG. 26J Characteristics of peptides predicted by EnsembleMHC, related to FIGS. 21A and 21B. Logo plots were generated for MHC alleles with at least 5 peptides identified by EnsembleMHC. Peptides shorter than 9 amino acids had random amino acid inserted into a non-anchor position while peptides longer than 9 amino acids had a random non-anchor position deleted. Large amino acid character height indicates a high frequency of that amino acid at that position. Amino acids are colored residue type.

FIG. 27A-FIG. 27D Molecular origin of predicted SARS-CoV-2 structural protein MHC-I peptides and impact of sequence polymorphism, related to FIGS. 21A and 21B. The predicted SARS-CoV-2 structural protein MHC-I peptides were mapped onto the solved structures for the envelope and spike proteins, and the predicted structures for the nucleocapsid and membrane proteins. Red highlighted regions indicate an enrichment of predicted peptides while blue regions indicate a depletion of predicted peptides.

FIG. 27E-FIG. 27G Molecular origin of predicted SARS-CoV-2 structural protein MHC-I peptides and impact of sequence polymorphism, related to FIGS. 21A and 21B. The incidence of protein sequence mutations (colored bar) and the frequency of that position in one of the 108 SARS-CoV-2 structural protein peptides (black bars) were calculated for 102,148 SARS-CoV-2 sequence variants. Lower left panel, all potential mutations arising in one of the 108 peptides identified by EnsembleMHC were evaluated for changes in binding affinity (peptide^{FDR}>0.05). Lower right panel, the overall frequency of mutations impacting EnsembleMHC-predicted peptides with light blue indicating deleterious mutations, and dark blue indicating neutral mutations.

FIG. 28A Comparison of entire SARS-CoV-2 EnsembleMHC population score and structural protein EnsembleMHC population score, related to FIG. 22A-22C. The correlations between EnsembleMHC population score based on the full SARS-CoV-2 proteome (left) or only SARS-CoV-2 structural proteins (right).

FIG. 28B Comparison of entire SARS-CoV-2 EnsembleMHC population score and structural protein EnsembleMHC population score, related to FIG. 22A-22C. The difference in the proportions of significant p-values and PPV between the full SARS-CoV-2 proteome (left) and SARS-CoV-2 structural proteins (right) (not corrected for multiple testing).

FIG. 28C Comparison of entire SARS-CoV-2 EnsembleMHC population score and structural protein EnsembleMHC population score, related to FIG. 22A-22C. The SARS-CoV-2 peptide-MHC allele distribution resulting from uniform allele sampling. These distributions were used as the partner distributions for the Kolmogorov-Smirnov test described in the results.

FIG. 28D Comparison of entire SARS-CoV-2 EnsembleMHC population score and structural protein EnsembleMHC population score, related to FIG. 22A-22C. 62 (57%) EnsembleMHC-identified SARS-CoV-2 structural protein peptides were included for testing in 4 different studies.

FIG. 28E Comparison of entire SARS-CoV-2 EnsembleMHC population score and structural protein EnsembleMHC population score, related to FIG. 22A-22C. The summary of immunogenicity status of tested EnsembleMHC peptides across all studies. These summaries were split into two groups. Total validated indicates the total number of experimentally validated peptides while total validated (no pools) indicates the number of experimentally validated peptides excluding those only tested in peptide pools. This distinction was made due to the potential of peptide pools to obscure which tested peptide is truly responsible for the observed immune response.

FIG. 28F-FIG. 28H Comparison of entire SARS-CoV-2 EnsembleMHC population score and structural protein EnsembleMHC population score, related to FIG. 22A-22F. Each individual plot shows the 95% confidence interval (shaded region) for the correlations between EMP scores based on the entire SARS-CoV-2 proteome (red) or SARS-CoV-2 structural proteins (blue) and observed deaths per million for different starting minimum death thresholds (indicated by number above plot).

FIG. 29A-FIG. 29C Justification of statistical tests, related to STAR Methods. The correlation between EnsembleMHC population score with respect to all SARS-CoV-2 proteins (left column) or SARS-CoV-2 structural proteins (right columns) and deaths per million using Pearson's r (top), Spearman's rho (middle), and Kendall's tau (bottom). Correlations that were shown to be statistically significant are colored with a red point.

FIG. 29D-FIG. 29F Justification of statistical tests, related to STAR Methods. The statistical power of each reported correlation. Correlations that were shown to be statistically significant are colored with a red point. The orange line indicates a power threshold of 80%.

FIG. 29G-FIG. I Justification of statistical tests, related to STAR Methods. The effect of different allele frequency normalization techniques on the reported correlations between SARS-CoV-2 mortality and EMP scores based on the full SARS-CoV-2 proteome (left column) or SARS-CoV-2 structural proteins (right column). Top panel, the aggregation of allele frequencies within a particular country by taking the sample-weighted mean of reported frequencies for the 52 selected MHC-I alleles. Middle panel, normalizing allele count with respect to all detected alleles in a given population. Bottom panel, normalizing allele count with respect to only the 52 select alleles.

FIG. 29J-FIG. 29L Justification of statistical tests, related to STAR Methods. QQ plots were generated from the respective distributions of the full proteome EnsembleMHC population scores, structural protein EnsembleMHC population scores, and deaths per million. To provide more descriptive distributions, EnsembleMHC population scores based on the full SARS-CoV-2 proteome and SARS-CoV-2 structural proteins were calculated for 10,000 simulated countries. Allele frequencies for simulated countries were generated by randomly sampling an observed allele frequency for each of the 52 alleles and re-normalizing to ensure the sum of allele frequencies were equal to one. Points falling outside of the blue lines indicate non-normal data skewing.

FIG. 29M Justification of statistical tests, related to STAR Methods. The mean statistical power of all resulting correlations between EnsembleMHC population scores and observed deaths per million at different minimum reported death thresholds. The red line indicates a minimum death threshold of 100 deaths by day 0, the selected upper limit for analysis.

FIG. 30A Robustness of EMP score correlation analysis, related to FIG. 22A-22C. 1,000 sub-sampling iterations were performed by randomly selecting 108 peptides from the full SARS-CoV-2 proteome that passed the 5% peptide^{FDR} filter. The correlation between the population EMP score produced by each sub-sampled set of peptides and observed deaths per million were plotted (grey lines). The correlation distribution observed for identified SARS-CoV-2 structural protein peptides (black line), all SARS-CoV-2 proteins (red line), and the median correlation distribution across all subsampling iterations (green line) were plotted for comparison.

FIG. 30B Robustness of EMP score correlation analysis, related to FIG. 22A-22C. Kullback-Leibler divergence was calculated for the correlation distribution of each down sample iteration relative to either the correlation distribution of the all peptide group (AP) or the structural peptide group (SP).

FIG. 30C Robustness of EMP score correlation analysis, related to FIG. 22A-22C. The MHC-I allele assessment of peptides that passed an individual algorithm binding affinity thresholds were shuffled prior to peptide^{FDR} filtering. The red points indicate correlations with a p-value <5%.

FIG. 30D Robustness of EMP score correlation analysis, related to FIG. 22A-22C. The impact of varying peptide^{FDR} cutoff threshold on the shuffled MHC data set. For each peptide^{FDR} cutoff threshold (x-axis), the upper bound of the shaded region indicates the 75th percentile, the lower bound indicates the 25th percentile, and the solid line indicates the median.

FIG. 30E-FIG. 30L Robustness of EMP score correlation analysis, related to FIG. 22A-22C. Population SARS-CoV-2 binding capacities using only single algorithms were correlated to observed deaths per million. For each algorithm, the population SARS-CoV-2 binding capacity was calculated from the resulting viral peptide-MHC allele distribution using restrictive MHC-I binding affinity cutoffs (<0.5% for binding percentile scores, top 0.5% MHCflurry presentation score, and <50 nm for PickPocket). Red points indicate a PPV >95%.

FIG. 31A-FIG. 31B Addition of structural protein EMP score significantly improves linear model fit to observed deaths per million, related to FIG. 23A-23B. Linear models were constructed using either a single risk factor (yellow) or a combination of a risk factor and structural protein EMP scores (green). The x-axis indicates the number of normalized days from when a minimum death threshold was met (line color), and the y-axis indicates the observed adjusted R2 value.

FIG. 31E-FIG. 31F Addition of structural protein EMP score significantly improves linear model fit to observed deaths per million, related to FIG. 23A-23B. A summary of results obtained from single feature linear models (top panel, yellow) or the combination models (bottom panel, green). The red bars indicate the median R2 value achieved by that model and the blue bars indicate the proportion of regressions that were found to be significant (F-test <0.05).

### DETAILED DESCRIPTION

The present disclosure provides a novel consensus MHC-I binding and processing prediction workflow method, referred to herein as EnsembleMHC. The disclosed prediction workflow method integrates seven different prediction algorithms, parameterized on high quality mass spectrometry data. The disclosure demonstrates recovery of peptides by the workflow method at a confidence level unattainable by each algorithm alone. The workflow method can be applied to predict all potential short, e.g. 5-mer to 20-mer peptides restricted to 52 common MHC-I alleles. The resulting predictions from EnsembleMHC can be used, for example, to predict a population fitness score based on total epitope load, for example against cancer cells or pathogens.

Limited information is available on immunogenic MHC-I restricted T cell epitopes for SARS-CoV-2, though studies exist regarding the immunogenicity of peptides derived from SARS-CoV and MERS-CoV. Accordingly, throughout the instant disclosure, the SARS-Cov-2 virus is used as an example for developing the methods and systems of the instant disclosure. For SARS-CoV, immunogenic T cell epitopes have been identified in the S, N, M, and E protein following the 2002-03 outbreak. The majority of these immunogenic targets were HLA-A2 restricted CD8+ T cell epitopes located in the spike protein, with fewer epitopes studied from the nucleocapsid protein. It is generally considered that epitopes in the M and E protein are less immunogenic and in lower frequency than that of the S and N protein, although systematic studies have been limited. Peptides derived from the non-structural polyprotein 1a have been used to generate IFN-producing memory CD8+ T cells from patients with SARS-CoV, and immunogenic epitopes from other non-structural or accessory proteins have been investigated as possible vaccine targets. As in other instances of vaccine development, the process is slowed by the limits on identifying large numbers of candidate epitopes at one time.

Using methods and systems described herein with SARS-CoV-2 as an example of a target antigen, 108 peptides derived from SARS-CoV-2 structural proteins were identified that are potential high value targets SARS-CoV-2 binding, and thus potentially for T-cell vaccine development, based on their predictive binding, expression, and sequence conservation in isolates. The workflow method is applied to predict all potential 8-14mer SARS-CoV-2 peptides restricted to 52 common MHC-I alleles. Additionally, using SARS-CoV-2 as an example of a target antigen, the resulting predictions from EnsembleMHC are used to predict a population fitness score based on total epitope load against the SARS-Cov-2 virus. A strong inverse correlation is observed, of total epitope load and the survival rate (fitness of a population) from SARS-CoV-2 across 21 countries, suggesting that population fitness may be shaped by the presentation of SARS-CoV-2 peptides to the immune system.

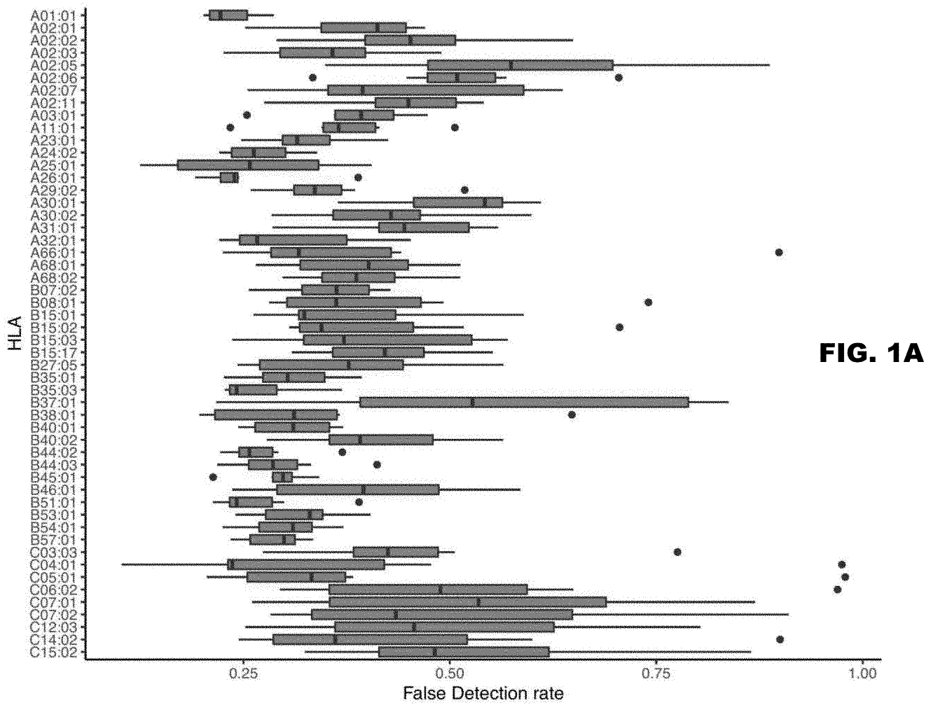

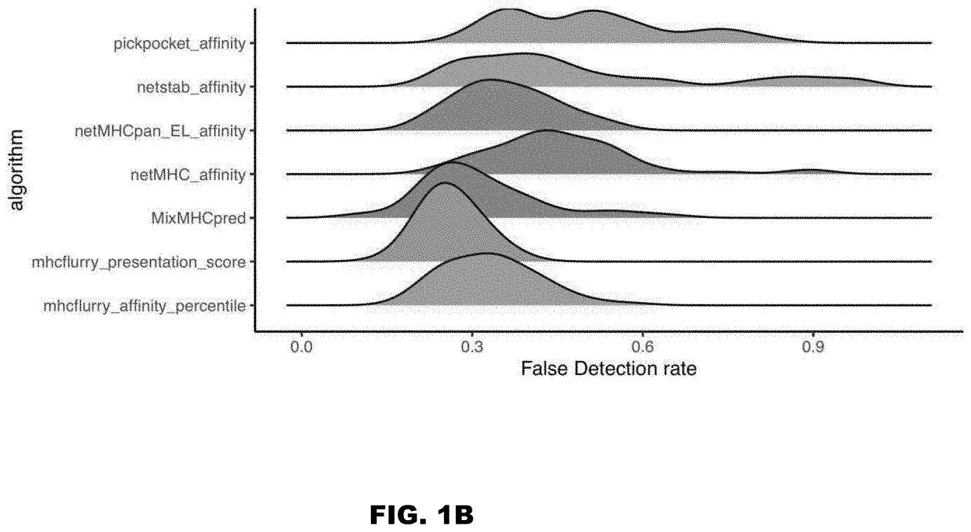

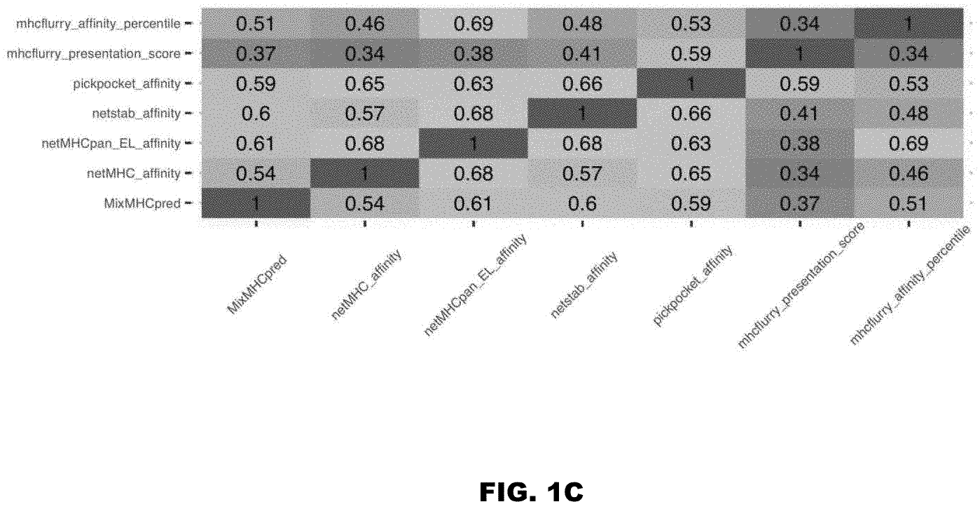

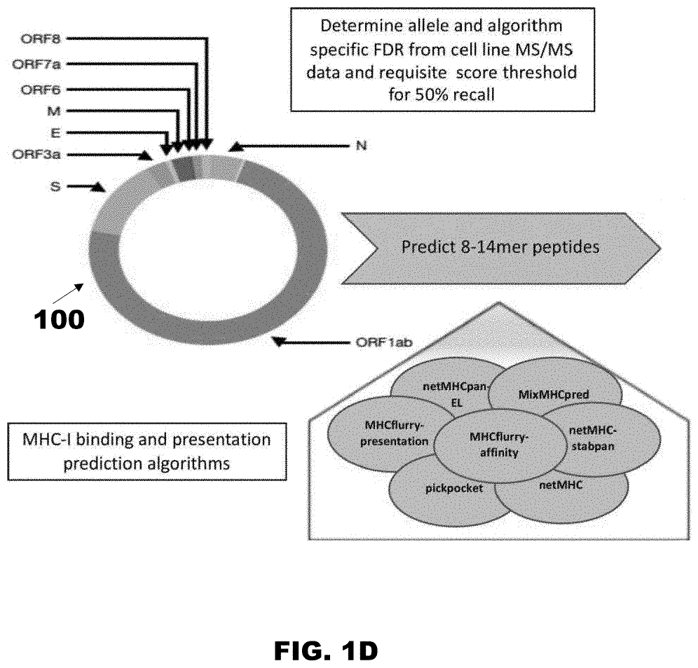

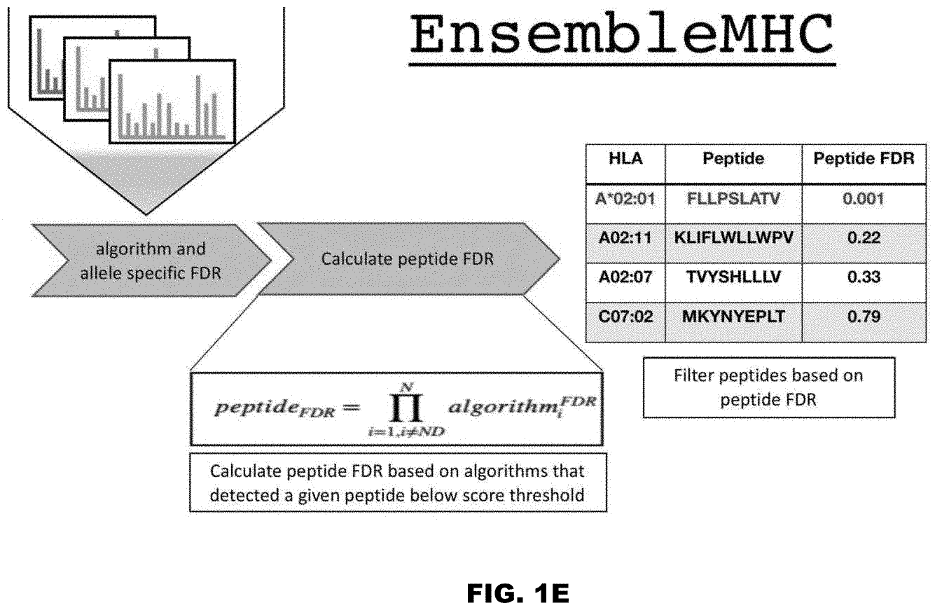

Referring to the drawings, aspects of a consensus MHC-I binding and processing prediction workflow for improving T-cell immunity against threats such as pathogens and cancer, referred to herein as EnsembleMHC, are illustrated and generally indicated as 100 in FIGS. 1-18.

### I. EnsembleMHC Prediction Workflow

EnsembleMHC Source Binding and Processing Prediction Algorithms

EnsembleMHC 100 incorporates MHC-I binding and processing prediction from 7 publicly available prediction algorithms: MHCflurry-binding_percentile, MHCflurry_presentation, netMHC-4.0, netMHCpan-EL-4.0, netMHCstabpan, Pick-pocket, and MixMHCpred. Algorithms were selected on the criteria of providing a free academic license, bash command line integration, and demonstrated accuracy for the prediction of SARS-CoV-2 peptides.

Each of the selected algorithms predict distinct components of MHC-I binding and antigen processing. MHCflurry-affinity_percentile and netMHC-4.0 predict binding affinity based on quantitative peptide binding affinity measurements. MixMHCpred, netMHCpan-EL, and MHCflurry-presentation are trained on naturally eluted MHC-I ligands and in the case of MHCflurry-presentation incorporated antigen processing prediction. netMHCstabpan is trained on MHC-peptide stability data. PickPocket is trained on quantitative binding affinity data and extrapolates binding based on MHC-I pocket architecture

Parametrization of EnsembleMHC Using Mass Spectrometry Data

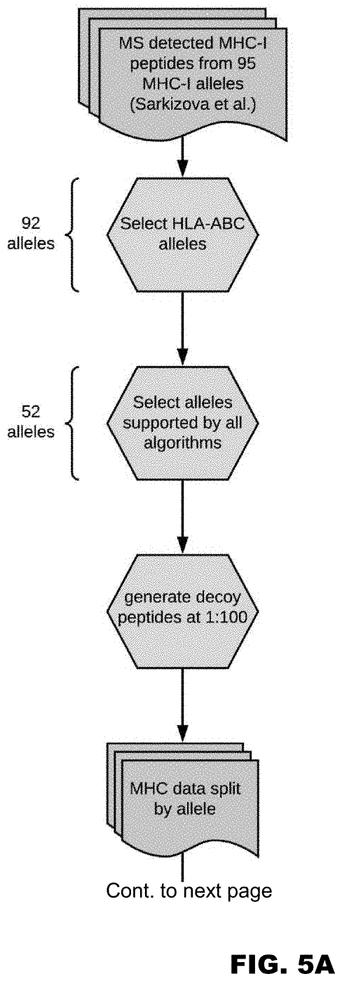

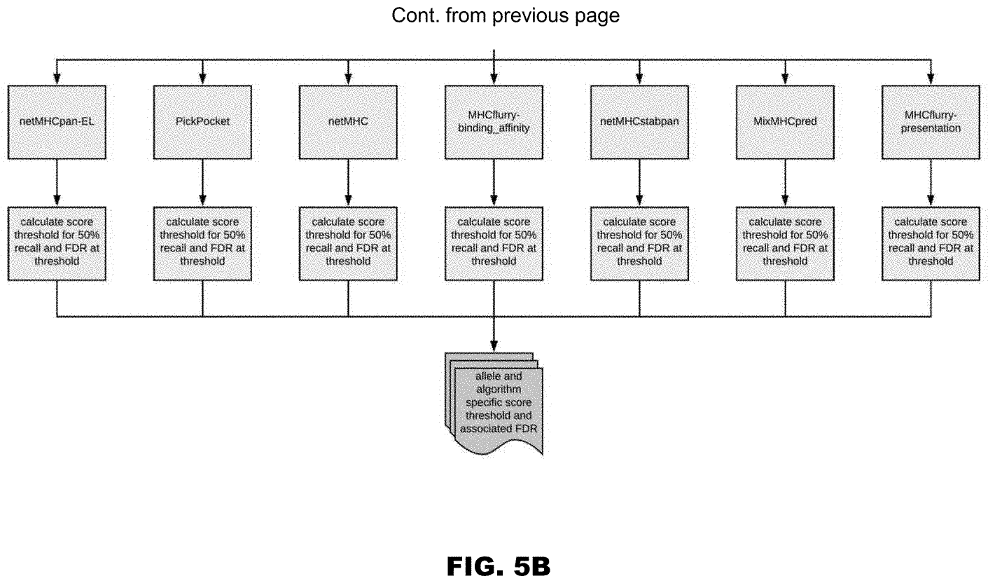

The main advantage of EnsembleMHC 100 is its ability to combine multiple disparate MHC-I binding and processing algorithms to improve accuracy and confidence of peptide calls unattainable by the use of any single algorithm. This is accomplished through the parameterization of allele and algorithm specific score thresholds and expected false detection rates (FDR), determined by observed performance on a comprehensive and high-quality mass spectrometry (MS) data set of naturally presented MHC-I peptides from mono-allelic cell lines. This particular dataset was selected as it is the largest single laboratory MS-based characterization of MHC-I peptides derived from monoallelic cell lines. This approach significantly reduces the number of artifacts introduced by differences in peptide isolation methods, mass spectrometry acquisition, and convolution of peptides in multiallelic cell lines. An overview of the EnsembleMHC 100 parameterization is provided in FIGS. 5A-5B.

Fifty-two common MHC-I alleles were selected for parameterization based on inclusion in the MS dataset and prediction support by all the individual algorithms. Each target peptide (observed in the MS dataset) was paired with 100 length-matched randomly sampled decoy peptides (not observed in the MS dataset) derived from the same source proteins. This decoy generation strategy minimizes bias toward protein expression by generating peptides from the same proteins that produced detectable peptides. If a protein was less than 100 amino acids in length, then every potential peptide from that protein was extracted.

Each of the seven algorithms were then used to predict the binding affinity or binding status independently for all of the 52 selected allele datasets. At each allele, the score threshold for a particular algorithm to achieve 50%

Application of EnsembleMHC for the Prediction of SARS-CoV-2 MHC-I Peptides

Epitope predictions for the SARS-CoV-2 proteome were performed using the reference sequence MN908947.3. All potential 8-14mer peptides (n=67,207) were derived from the open reading frames in the reported proteome, and each peptide was evaluated by the EnsembleMHC workflow 100. For each algorithm, all peptides that failed to reach the identified score threshold at the specified allele were filtered out. The resulting peptides were then aggregated, and the confidence of each peptide prediction, peptide^{FDR}, is represented by the product of the observed allele relative FDRs for each of the algorithms that detected a given peptide. This relationship is given by Equation (1):

$${peptide}^{FDR} = {\prod }_{i = 1 , i \neq ND}^{N} {algorithm}_{i}^{FDR} \qquad ( 1 )$$

Where N is the number of MHC-I binding and processing algorithm, ND represents an algorithm that did not detect a given peptide, and algorithm^{FDR} represents the FDR of the N^{th} algorithm.

The resulting calculation is the joint probability that all of the MHC-I binding and processing algorithms that detected a particular peptide did so in error, and therefore returns a false positive probability for that peptide. Peptides that were assigned a false positive probability of less than or equal to 5% were selected for inclusion in the predicted peptide set. An overview of the application of EnsembleMHC 100 for the prediction of SARS-CoV-2 peptides is shown in FIG. 1.

Protein Structure Visualizations and Polymorphism Analysis

Polymorphism analysis of SARS-CoV-2 structural was performed using 4,455 full length protein sequences obtained from the National Center for Biotechnology Information (NCBI). Proteins were visualized using VMD. Structures for the E (5×29) and S (6vxx) protein were obtained from the Protein Data Bank and predicted protein structures for M and N protein were obtained from iTasser.

### II. Application of EnsembleMHC to Determine Population Fitness Against SARS-CoV-2

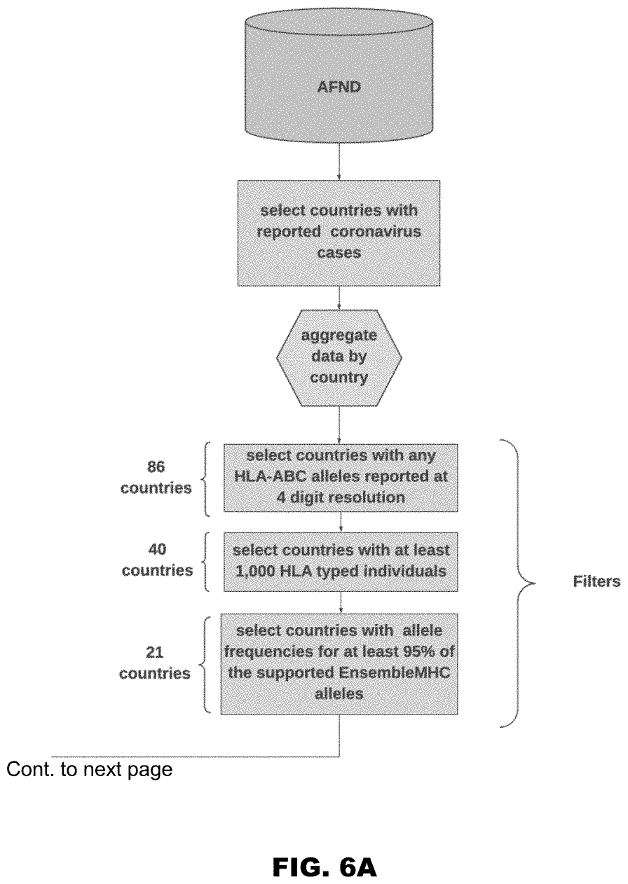

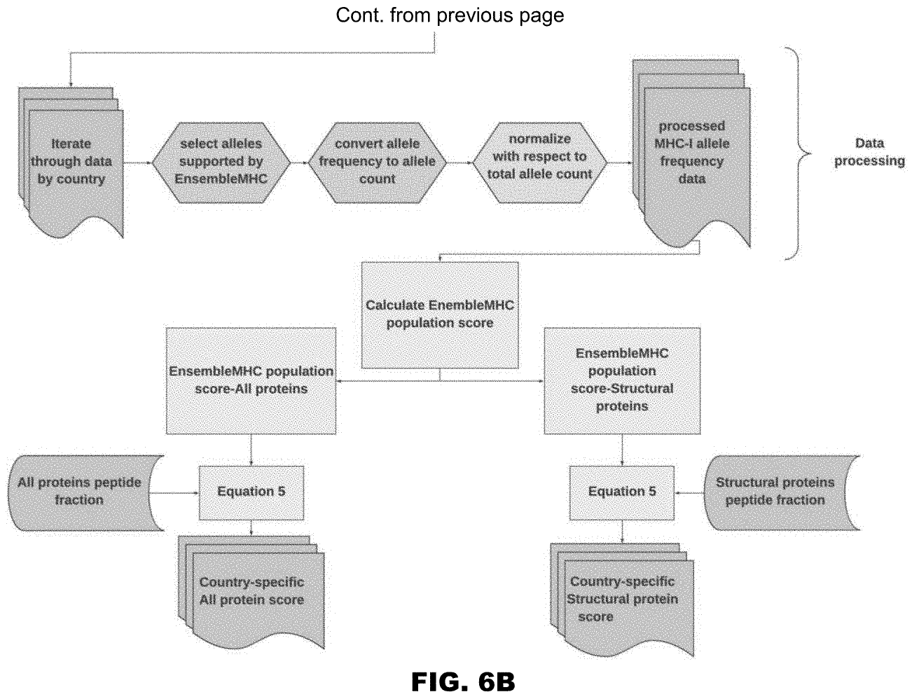

The peptides identified by EnsembleMHC 100 were used to assess the fitness of a given population against the SARS-CoV-2 virus by factoring the observed allele preferences for the predicted SARS-CoV-2 peptides as well as regional expression of those MHC-I alleles within a given population. The workflow is summarized in FIG. 6.

Population-Wide MHC-I Frequency Estimates by Country

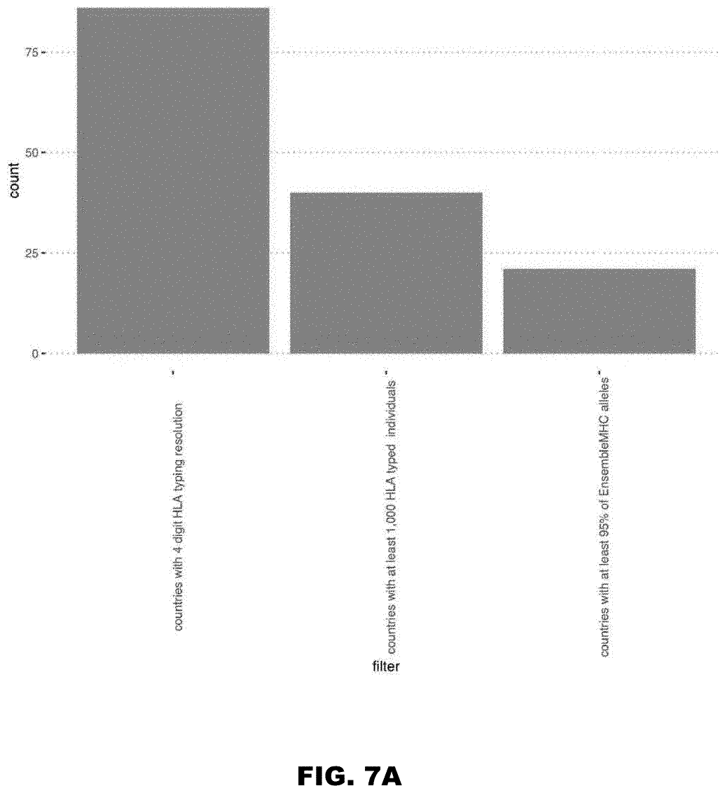

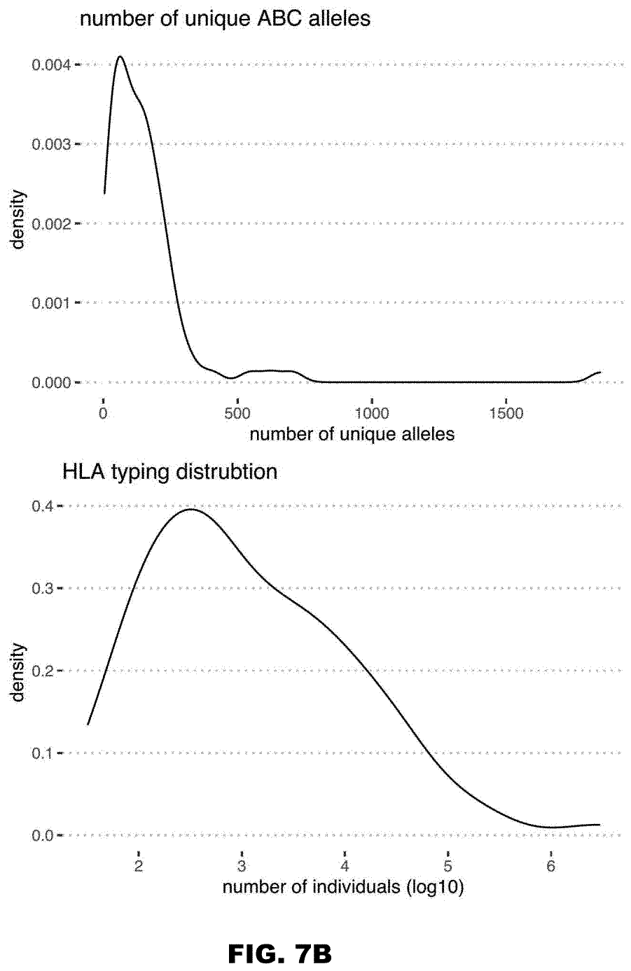

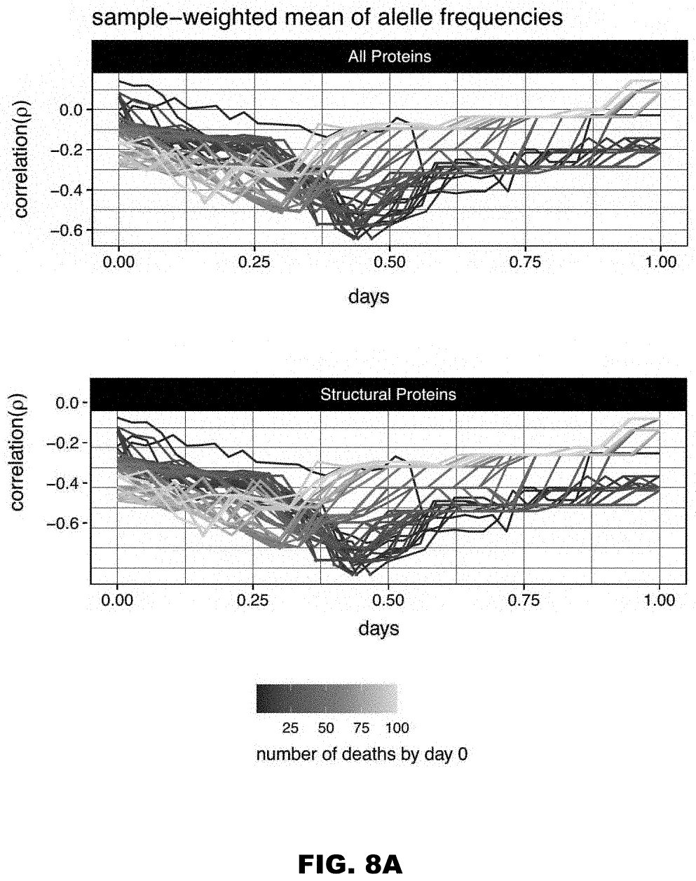

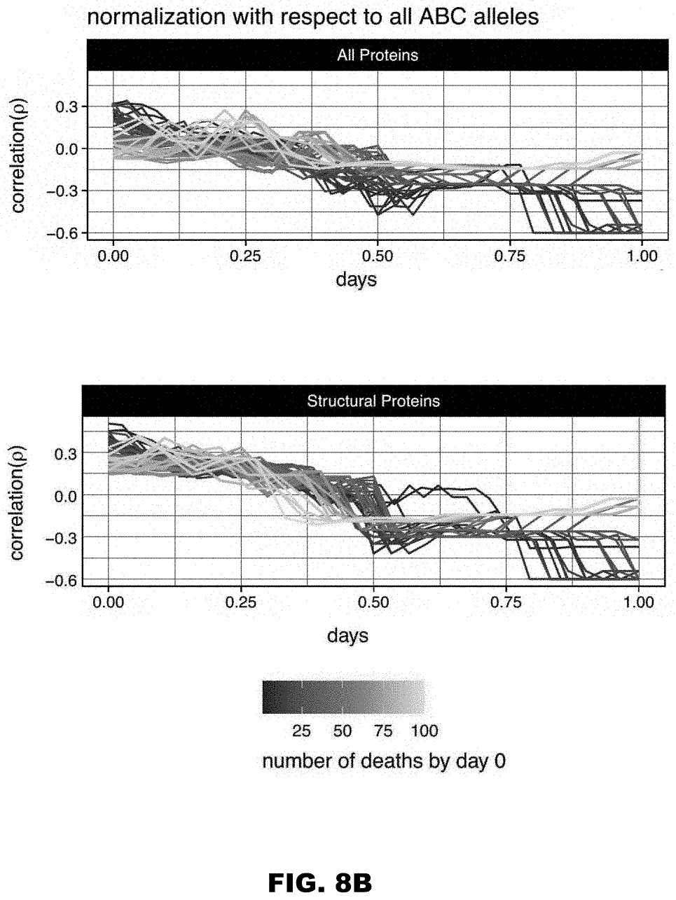

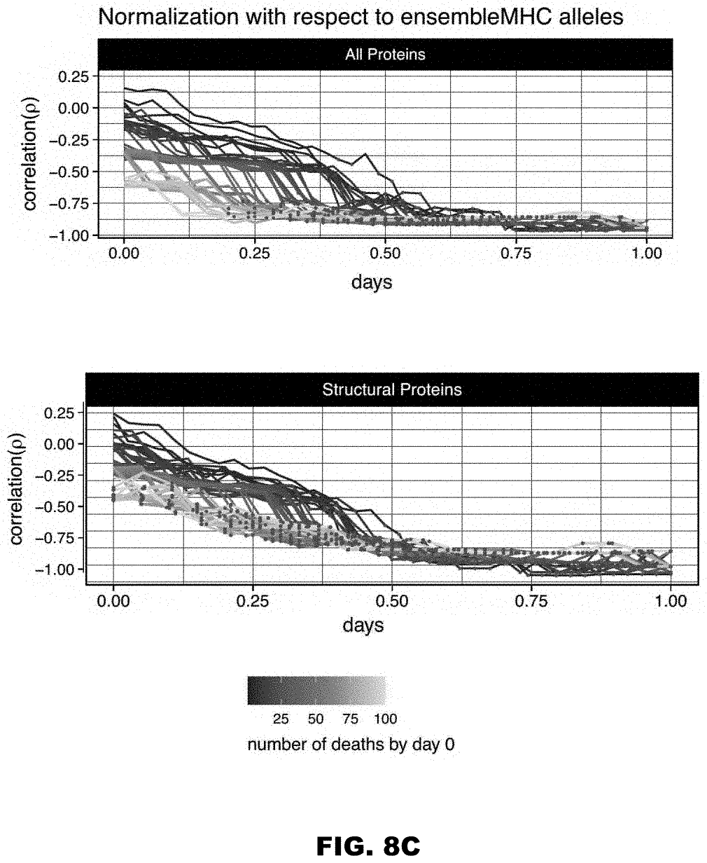

The selection of countries included in the EnsembleMHC population fitness assessment was based on several criteria regarding the underlying MHC-I allele data for that country (FIGS. 7 and 8). The MHC-I allele frequency data used in the instant model was obtained from the Allele Frequency Net Database (AFND), and these frequencies were aggregated by country. However, the currently available population-based MHC-I frequency data has specific limitations and variances, which are addressed as follows:

Ethnic communities within countries. In instances where the MHC-I allele frequencies would pertain to more than one community (e.g. a Chinese minority in Germany), the reported frequencies were counted towards both contributing groups. For example, the MHC-I frequency data pertaining to the Chinese minority in Germany would be factored into the MHC-I frequencies for both China and Germany. In doing so, this treatment resolves both ancestral and demographic MHC-I allele frequencies.

Quality of MHC data within countries. MHC-typing breadth is defined as the diversity of identified MHC-I alleles within a population of communities, and its depth as the ability to accurately achieve 4-digit MHC-I genotype resolution. High variability was observed in both the MHC-I genotyping breadth and depth (FIG. 7). Consequently, additional filter-measures were introduced to capture potential sources of variance within the analyzed cohort of countries. The thresholds for filtering the country-wide MHC-I allele data were set based on meeting two inclusion criteria: 1) HLA genotyping of at least 1000 individuals have been performed in that population, avoiding skewing of allele frequencies due to small sample size. 2) MHC-I allele frequency data for at least 95% of the 52 MHC-I alleles for which the EnsembleMHC 100 was parametrized to predict, controlling for the breadth of HLA characterization within a population and ensuring full power of the EnsembleMHC workflow 100.

Normalization of HLA Data

Analogous to past work on HIV, the focus of this study was to uncover potential differences in SARS-CoV-2 MHC-I presentation dynamics induced by these selected alleles within a population. Accordingly, the MHC-I allele frequency data was carefully processed in order to maintain differences in the expression of selected alleles, while minimizing the effect of confounding variables.

The MHC-I allele frequency data for a given population was first filtered to the 52 selected alleles. These allele frequencies were then converted to the theoretical total number of copies of that allele within the population (allele count) following equation (2):

allele cow=af×2×n (2)

where of is the observed allele frequency in a population and n is the sample size, both available at AFND. The allele count is then normalized with respect to the total allele count of selected 52 alleles within that population (equation 3).

$$norm allele count = \frac{{\sum }_{i = 1}^{52} allele {count}_{i}}{\sum allele count} \qquad ( 3 )$$

where i is one of the 52 selected alleles. This normalization can overcome the potential bias towards the unseen alleles that are either not well characterized or not supported by EnsembleMHC 100 as would be seen using allele frequency accounting techniques (e.g. sample weighted mean of selected allele frequencies or normalization with respect to all observed alleles with a population (FIG. 8A-FIG. 8C)). The binding capacity for SARS-CoV-2 peptides for these unseen alleles cannot be accurately determined using the EnsembleMHC workflow 100.

EnsembleMHC Population Score

The predicted ability of a given population to present SARS-CoV-2 derived peptides was assessed by calculating the EnsembleMHC Population (EMP) score. After the HLA data filtering steps, 21 countries were included in the analysis. The calculation of the EnsembleMHC population score is as follows:

$$EMP score = \frac{{\sum }_{i = 1}^{52} pf \times norm allele {count}_{i}}{{N}_{norm allele count \neq 0}} \qquad ( 4 )$$

Where norm allele count is the observed normalized allele count for a given allele in a population, Nₙₒᵣₘ allele count 6=0 is the number of selected alleles detected in the population, and pf is the peptide fraction or the fraction of total predicted peptides expected to be presented by that allele within the total set of predicted peptides

Death Rate-Presentation Correlation

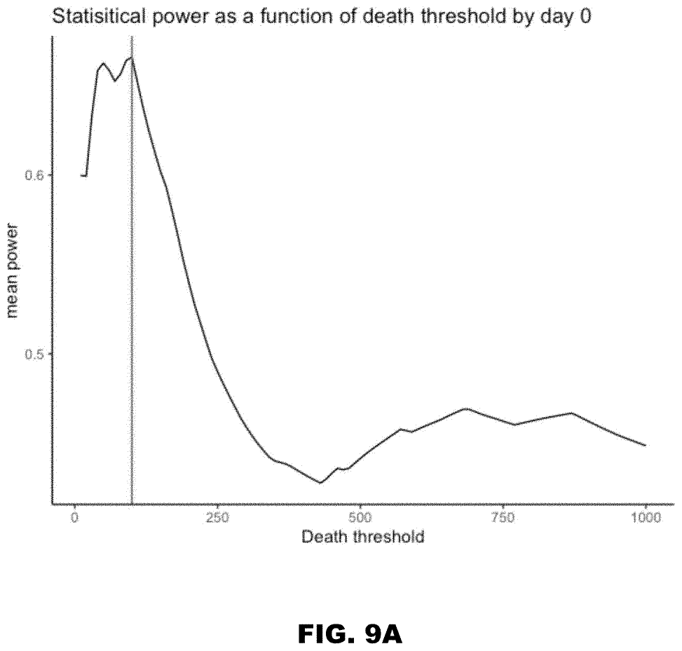

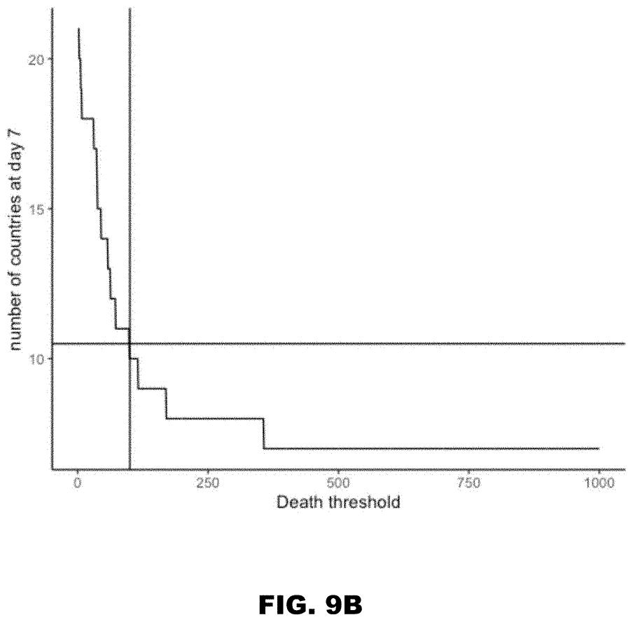

The correlation between the EMP score and the observed deaths per million within the cohort of selected countries was calculated as a function of time. SARS-Cov-2 data was obtained from Johns Hopkins University Center for Systems Science and Engineering. The temporal variations in occurrence of community spread observed in different countries were accounted for by rescaling the time series data relative to when a certain death threshold was met in a country. For example, if the analyzed death threshold was 10 deaths, then day 0 for all considered countries would be when that country met or surpassed 10 deaths. This analysis was performed for thresholds of 1-100 total deaths by day 0, and correlations were calculated at each day sequentially from day 0 until there were fewer than 6 countries remaining at that time point. The upper-limit of 100-deaths was chosen to ensure availability of death-rate data on at least 50% of the countries for a minimum of 7 days starting from day 0. Additionally, a steep decline in average statistical power is observed after day 100 (FIG. 9A and FIG. 9B).

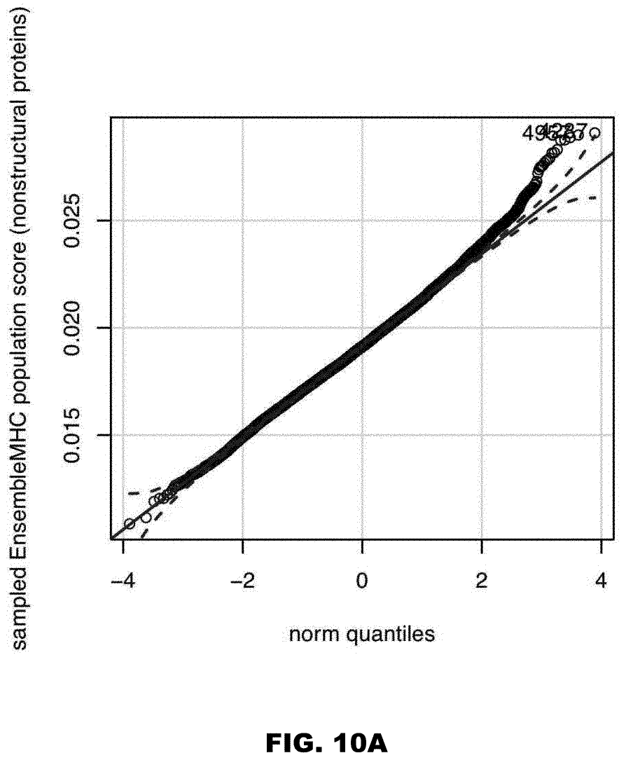

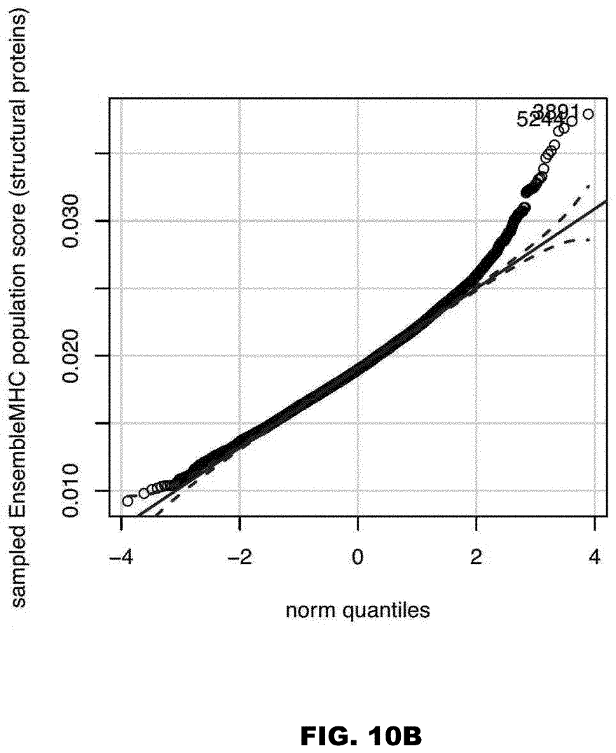

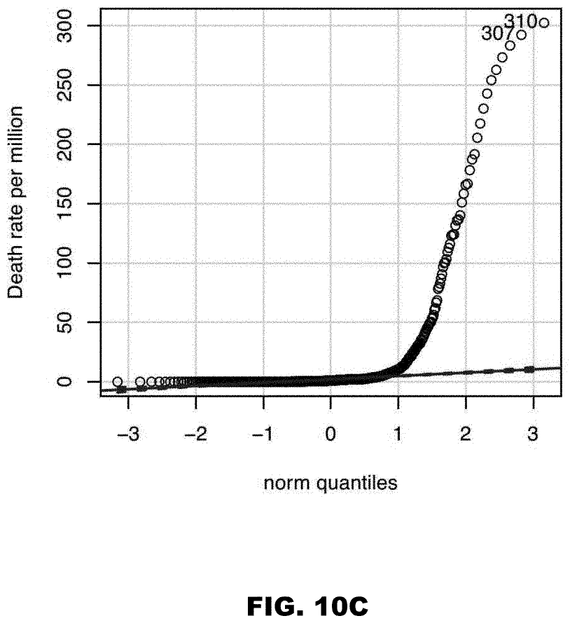

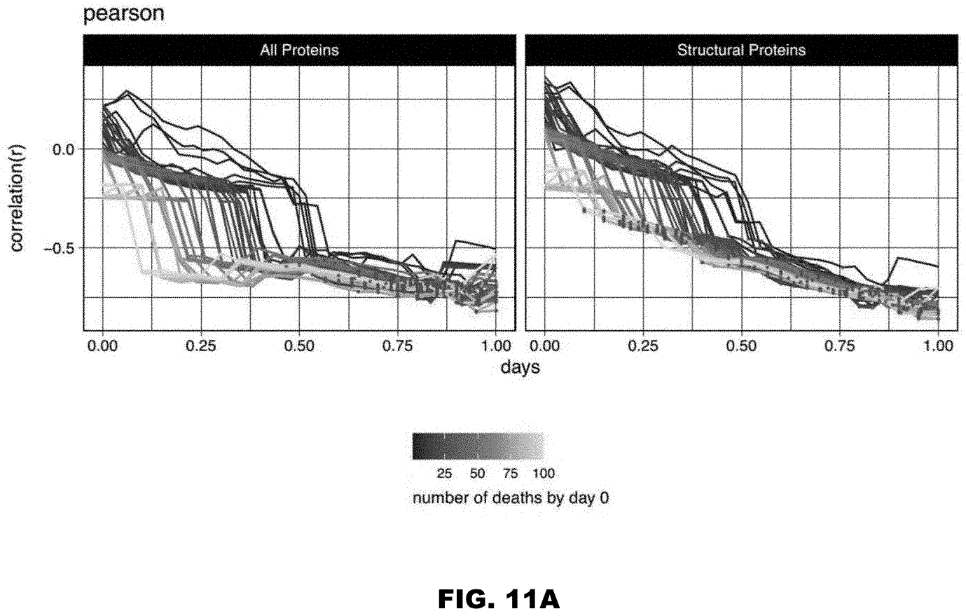

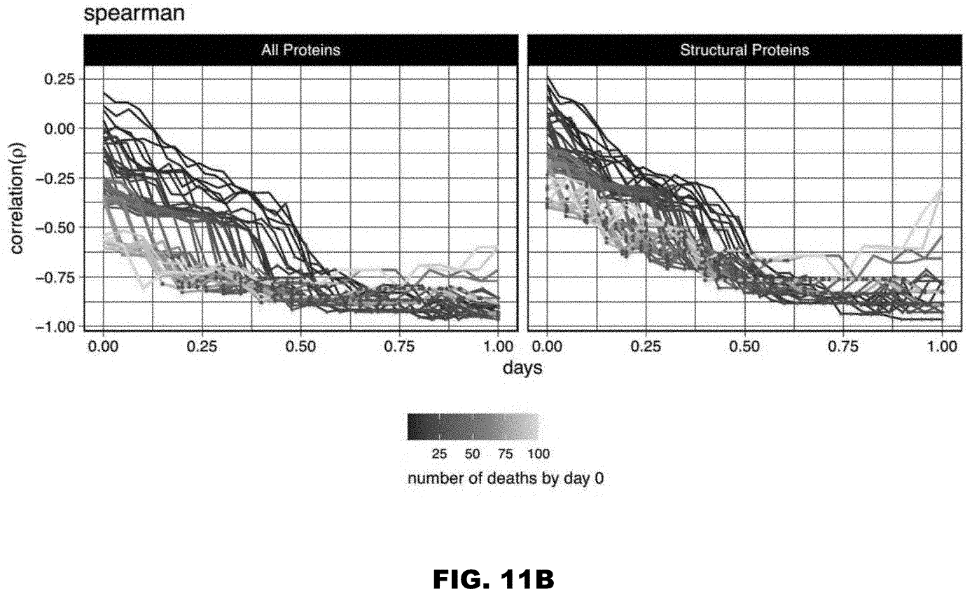

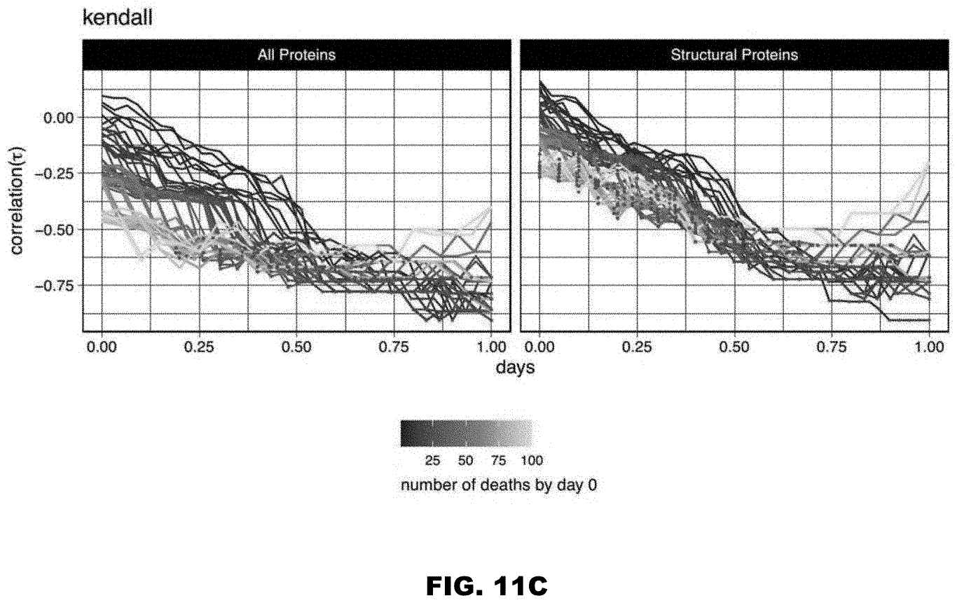

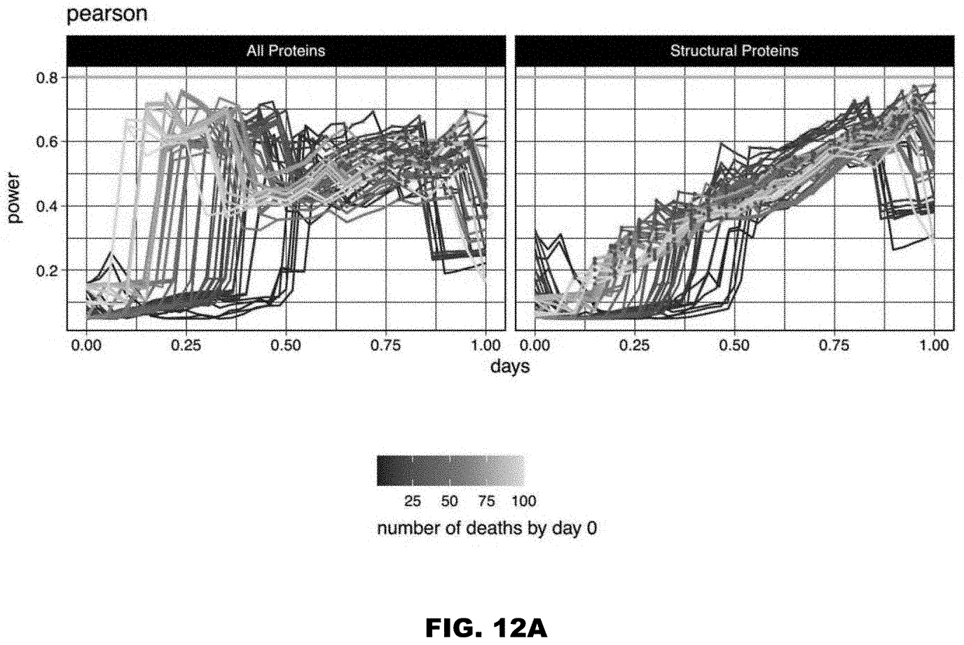

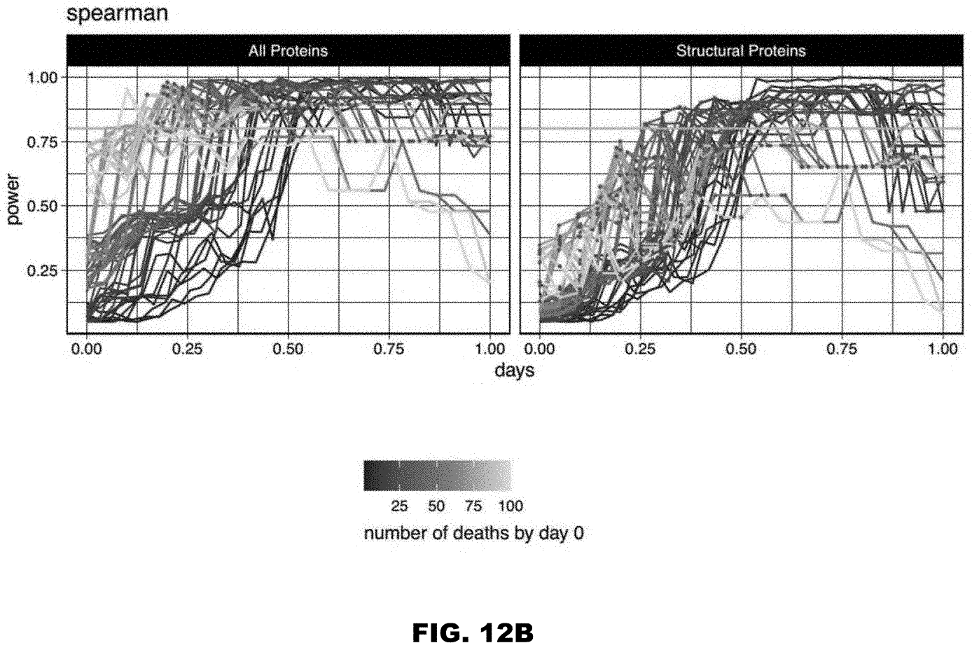

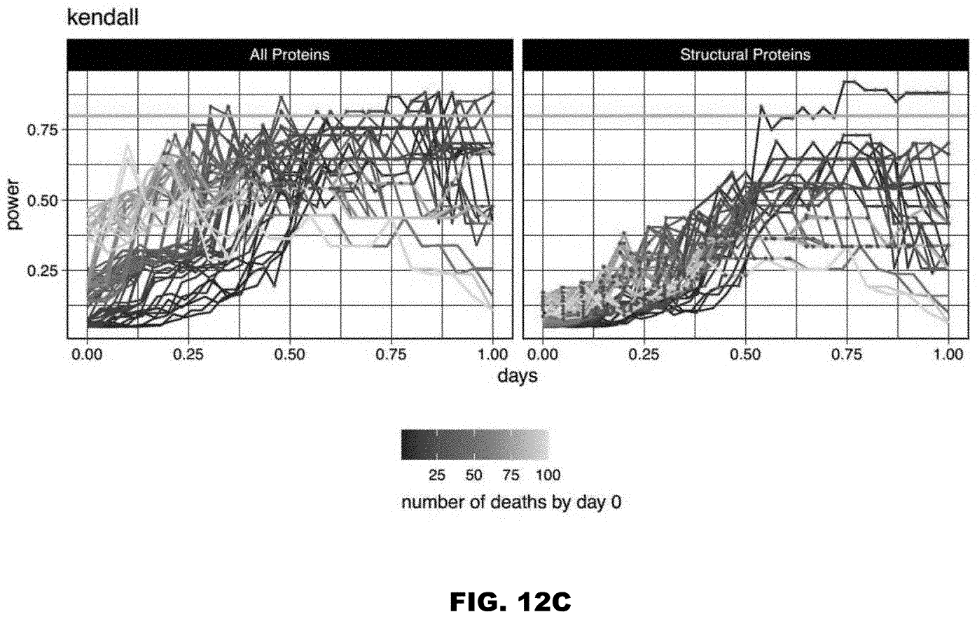

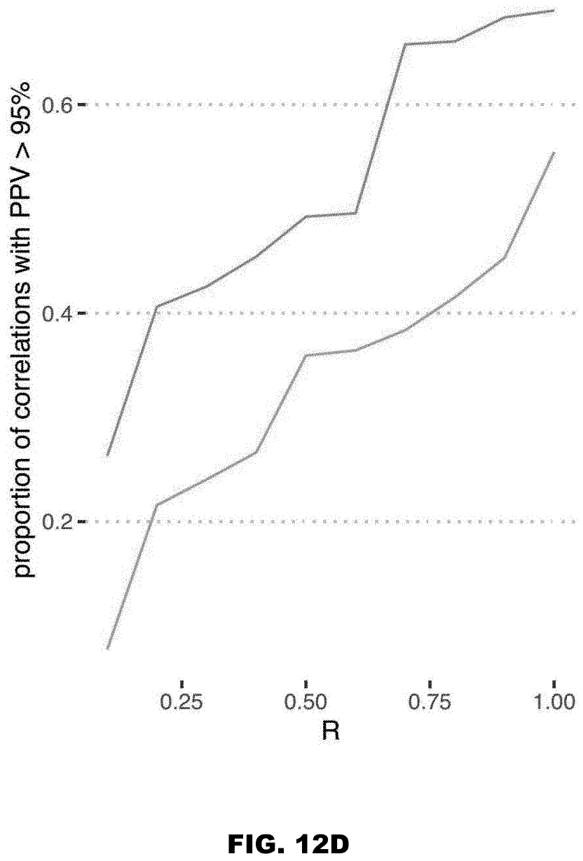

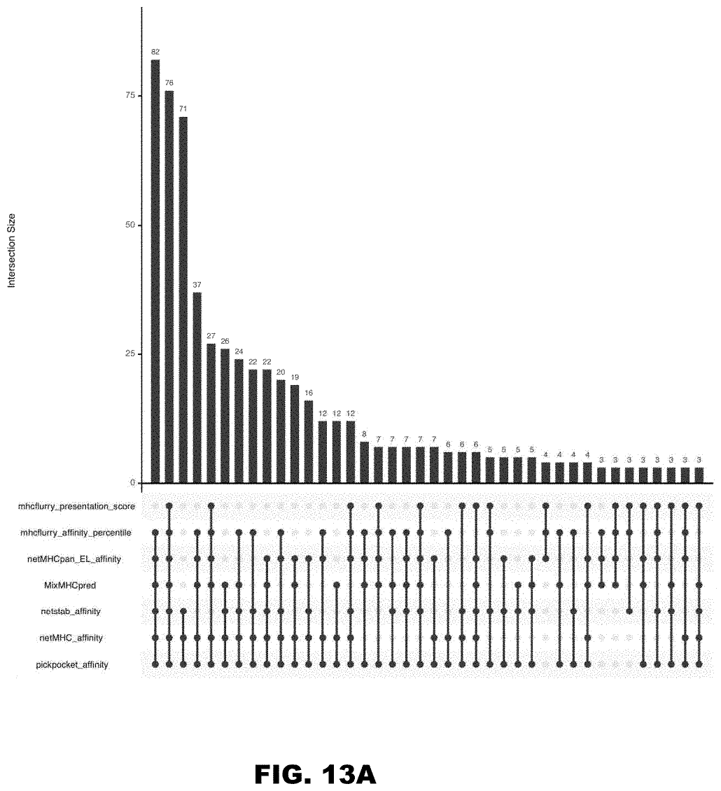

The time death correlation was computed using Spearman's rank correlation coefficient. This method was chosen due to the small sample size and non-normality of the underlying data (FIG. 10A-FIG. 10C). The reported correlations of EMP score and deaths per million using other correlation methods can be seen in supplemental figures (FIGS. 11A-FIG. 11B). The low observed statistical power of the obtained correlations, due to the small sample size, was accounted for by calculating the Positive Predictive Value (PPV) using the following equation (5):

$$PPV = \frac{1 - β \times R}{1 - β \times R + α} \qquad ( 5 )$$

where 1−β is the statistical power of a given correlation, R is the pre-study odds, and a is the significance level. A PPV value of ≥95% is analogous to a p value of ≤0.05. Due to an unknown pre-study odd, R was set to 1 in the reported correlations. The proportion of reported correlations with a PPV of 95% at different R values can be seen in FIG. 12A-FIG. 12D. The significance of partitioning high risk and low risk countries based on median EMP score was determined using Mann-Whitney U-test

### Results

EnsembleMHC Workflow

EnsembleMHC 100 is an integrated MHC-I binding and presentation prediction algorithm leveraging 7 publicly available MHC-I analysis tools trained on multiple distinct properties covering predicted peptide binding affinity, MHC-peptide stability, antigen processing, and binding pocket structural features. Previous work has established the benefits of ensemble algorithms towards improving the quality of MHC-I binding predictions. The EnsembleMHC workflow 100 was parameterized using mass spectrometry peptide data of naturally presented MHC-I peptides eluted from 52 HLA-A, B and C alleles. In line with these results, EnsembleMHC 100 addresses two major bottlenecks faced in MHC-I ligand prediction: identification of a score threshold by which to select peptides, and the minimization of false positives occurring within a determined score threshold

It has been established that a global score threshold for binding affinity is inefficient at recovering observed peptides across diverse HLA alleles and allele specific thresholds, based on putative binding capacity significantly improve results. EnsembleMHC 100 expands upon this observation by dynamically setting an allele-specific score threshold for each prediction algorithm at which 50% of target peptides can be expected to be recovered, as well as assigning the expected false detection rate at that threshold

These qualities of EnsembleMHC 100 produce two desirable traits. First, it determines an allele specific score threshold for each algorithm at which a known quantity of peptides can be expected to be successfully presented on the cell surface. Second, it allows for confidence level assignment of each peptide call made by each algorithm (Methods). The measured allele- and algorithm-specific FDR is shown in FIG. 1. The average deviation between FDRs for each algorithm across all alleles was found to 0.12 with C*4:01 showing the highest FDR deviation of 0.291 and A*01:01 showing the lowest deviation of 0.033. Overall, all seven algorithms exhibited a similar distribution of FDR values however, analysis of individual peptide score correlations between algorithm indicated only a moderate level of score correlation (mean=0.603). This indicated that, while the overall performance of each of the included algorithms was comparable, there was a diversity in individual peptide calls by each algorithm, supporting an integrated approach to peptide selection. An overview of the EnsembleMHC 100 pipeline is presented in FIG. 1.

EnsembleMHC Predictions Reveals Unequal Peptide-Allele Distributions Between the SARS-CoV-2 Proteome and SARS-CoV-2 Viral Capsid Proteins

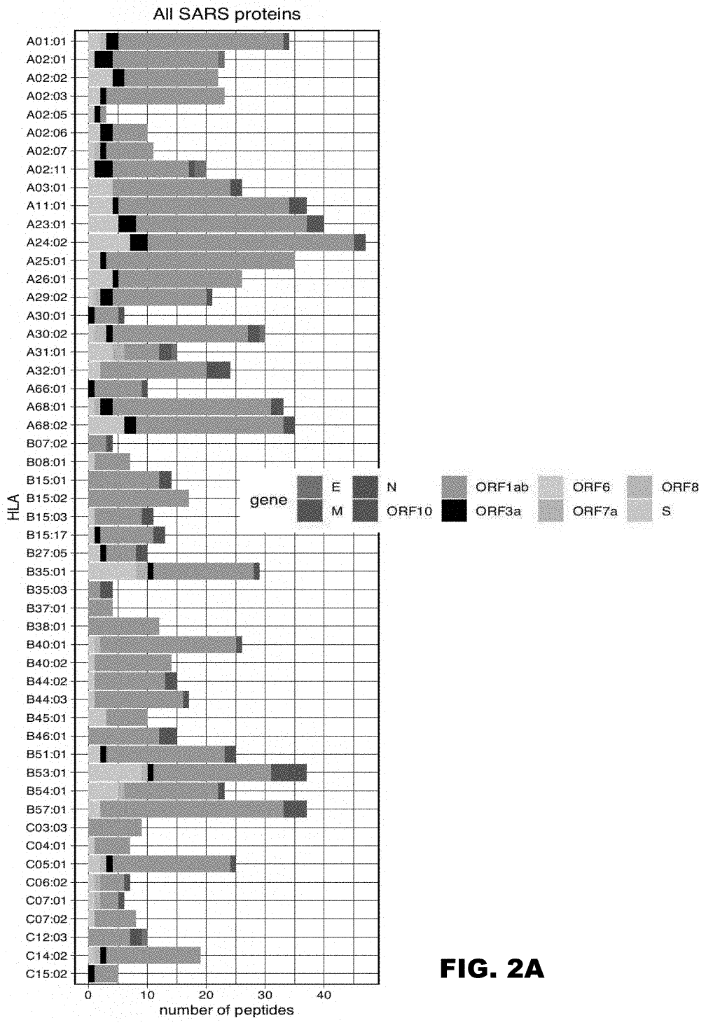

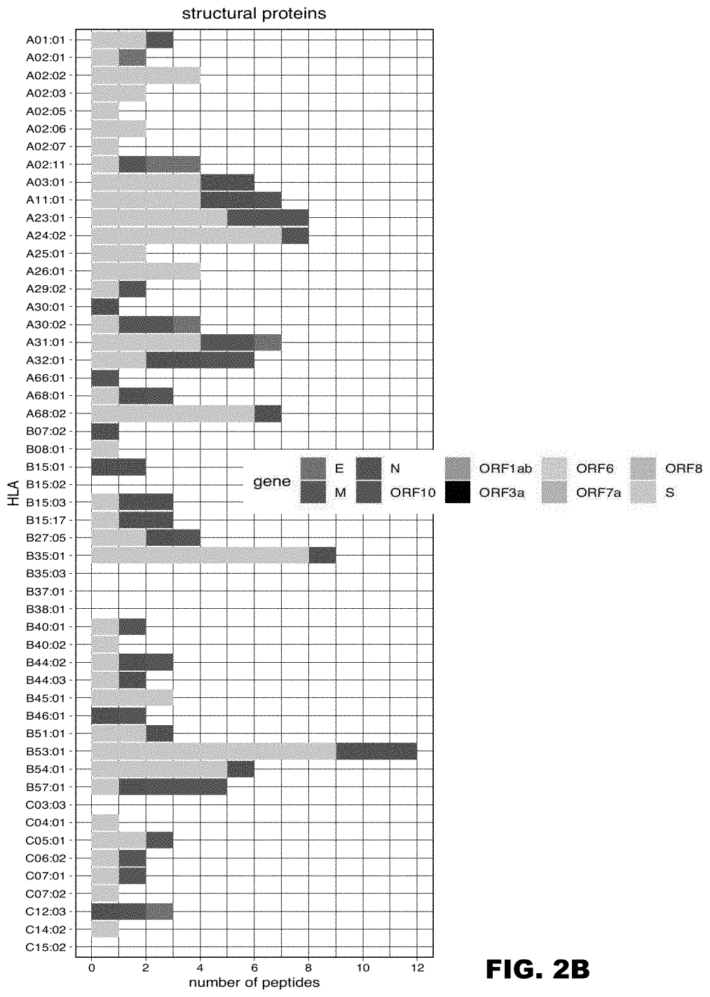

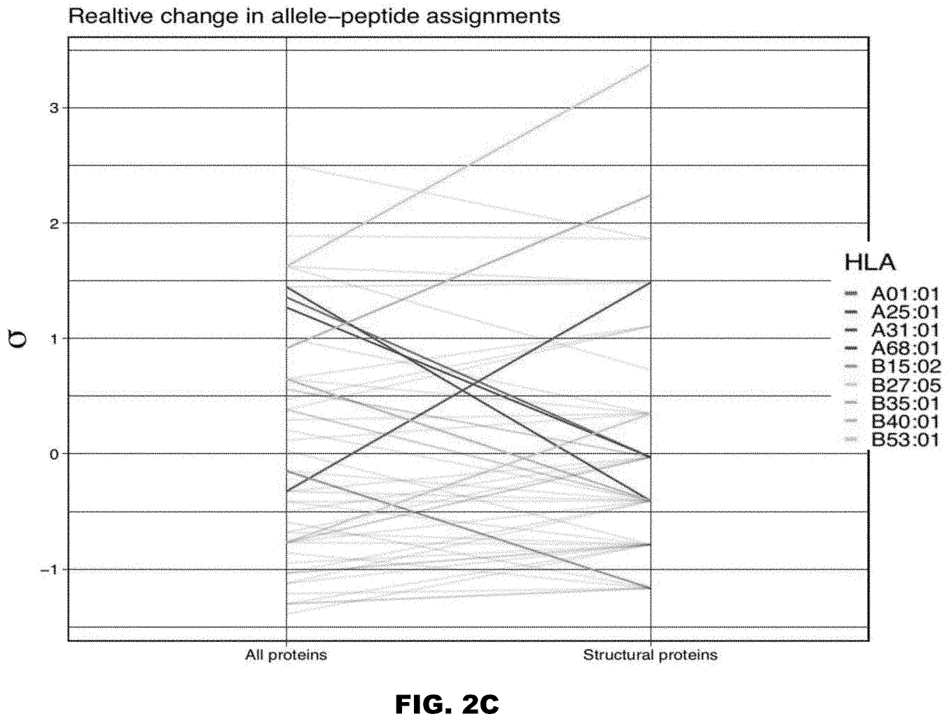

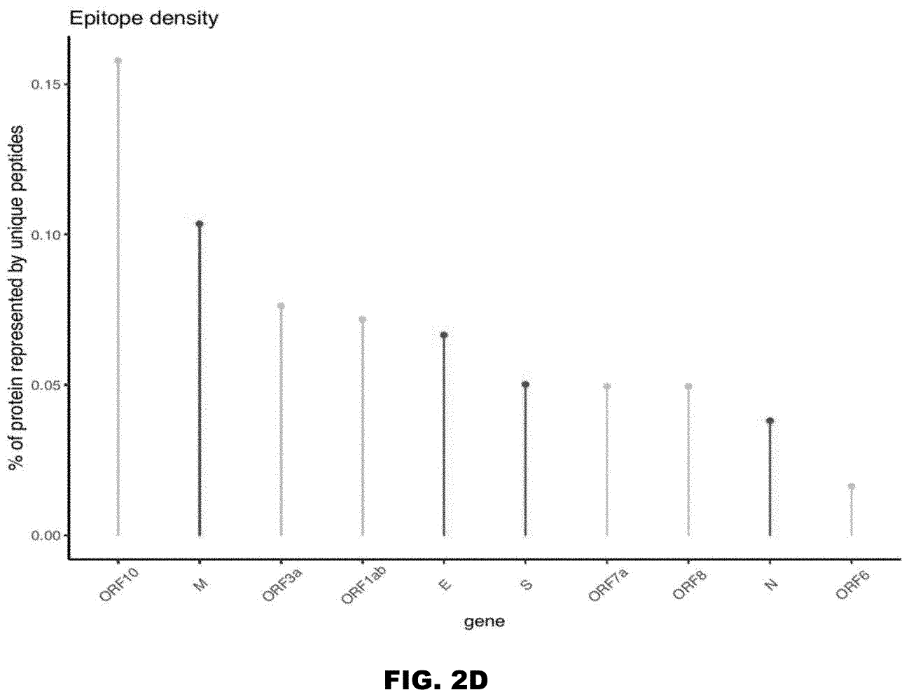

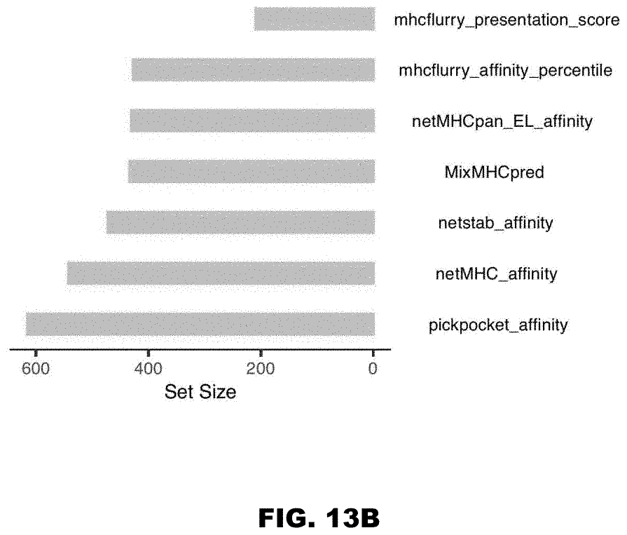

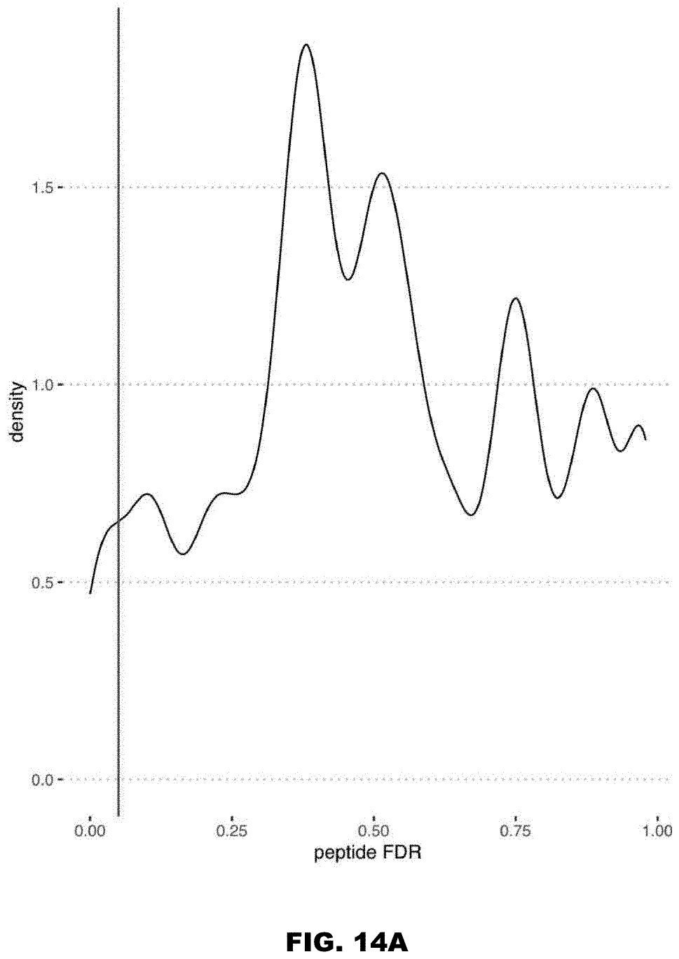

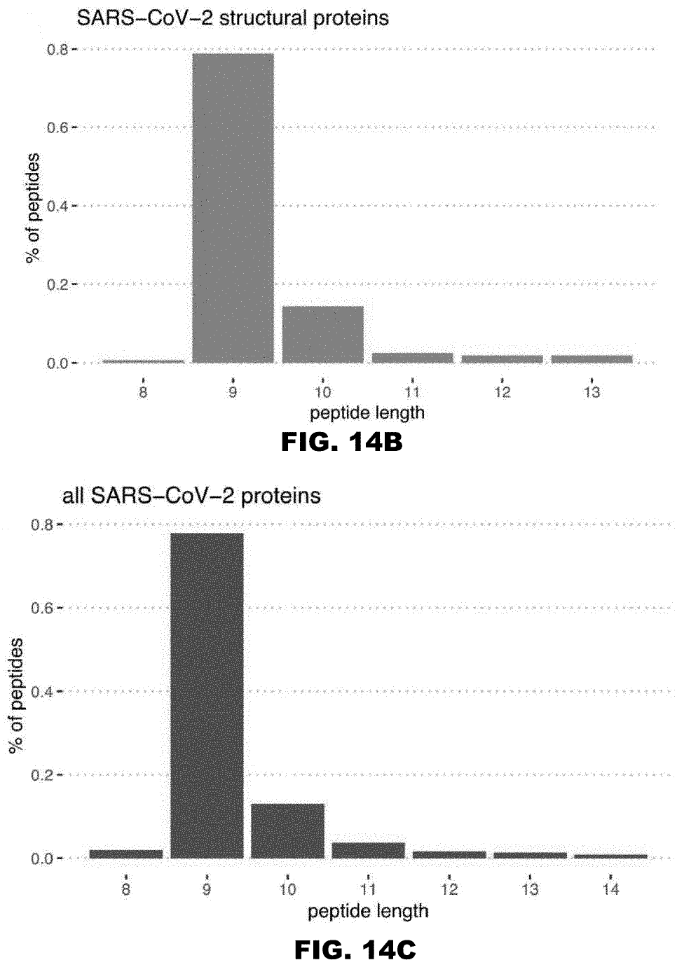

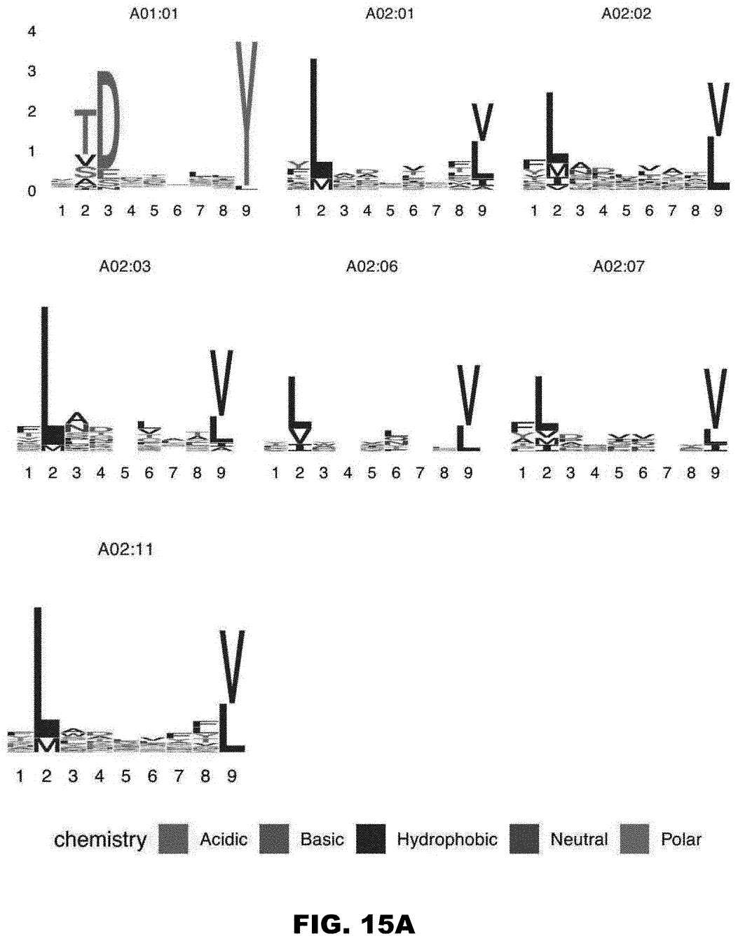

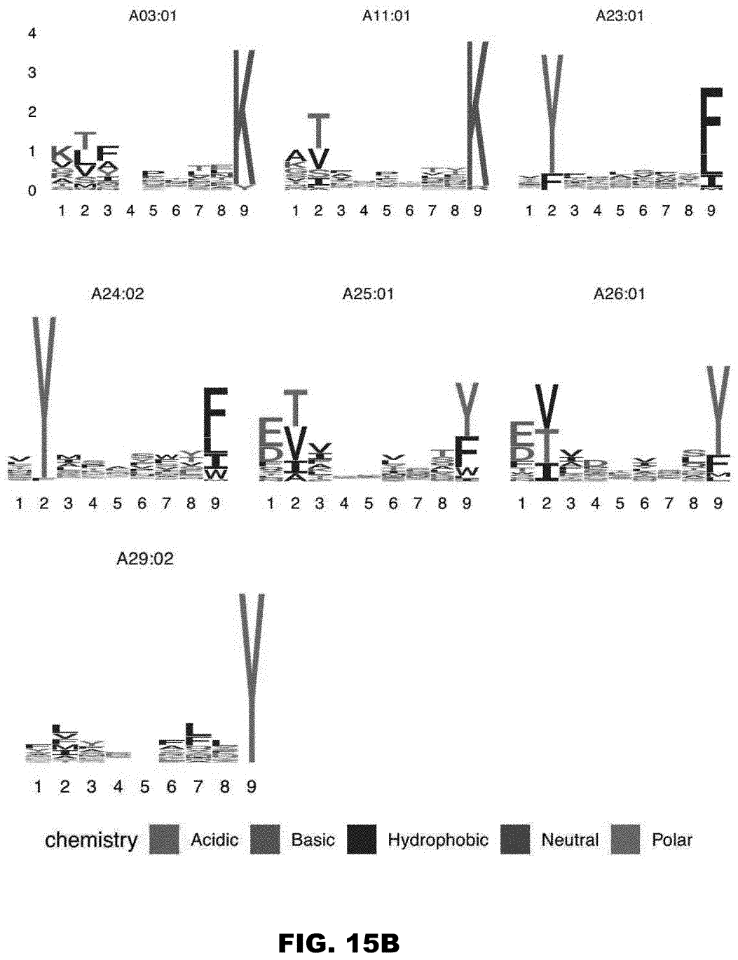

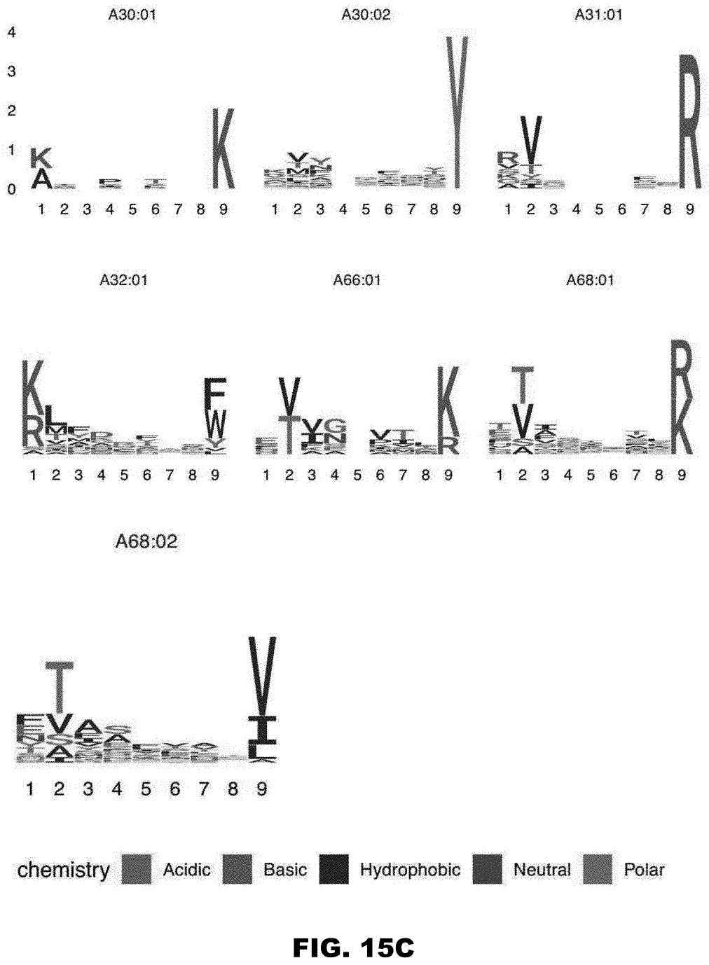

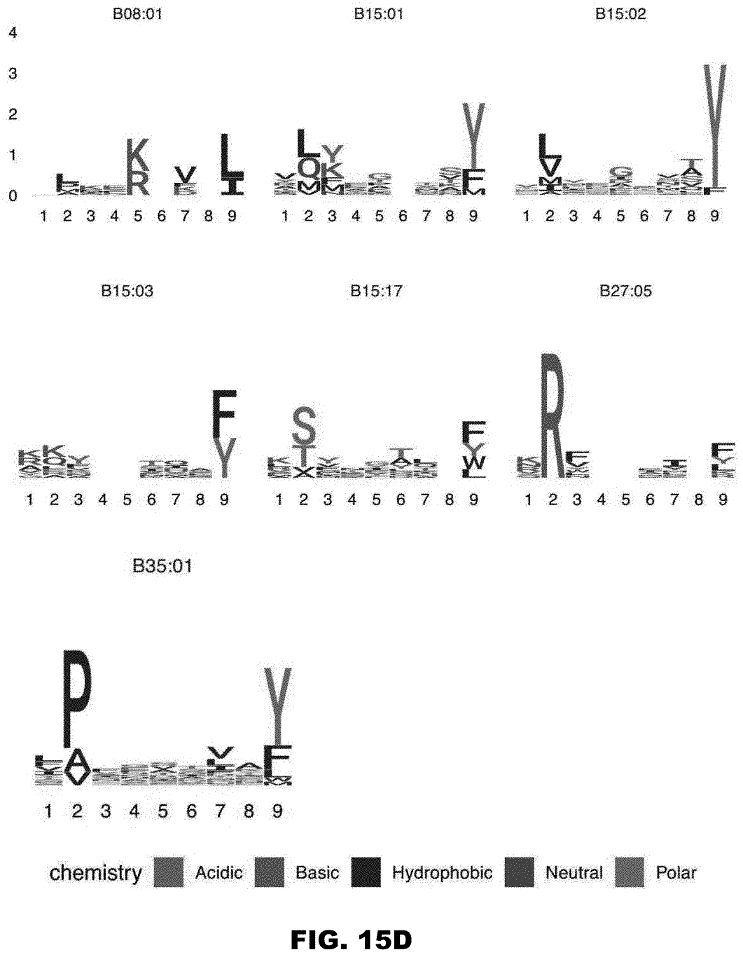

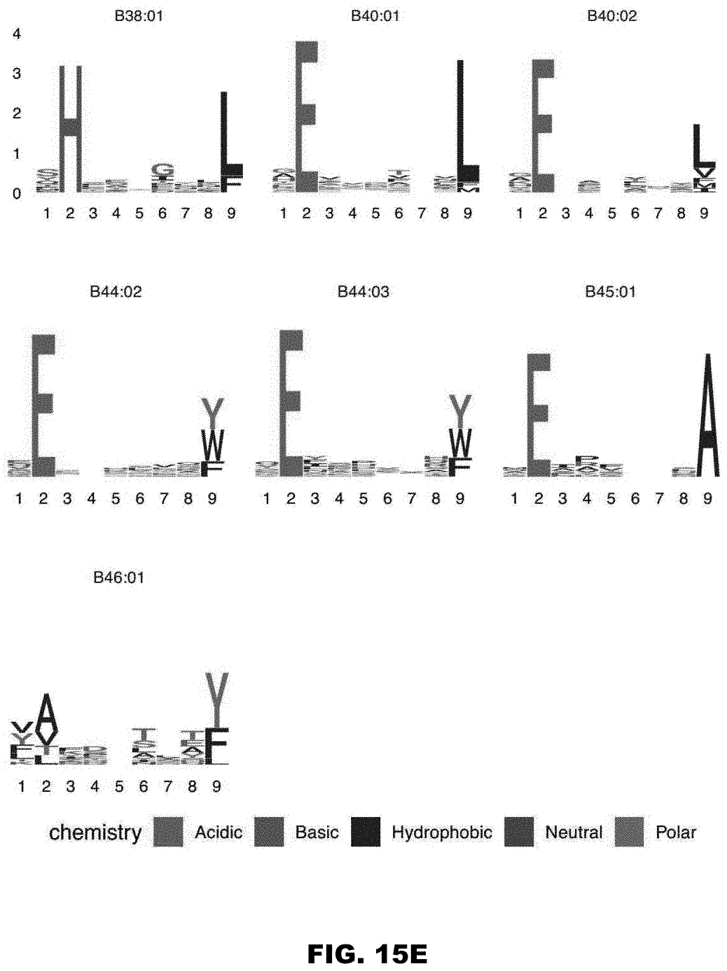

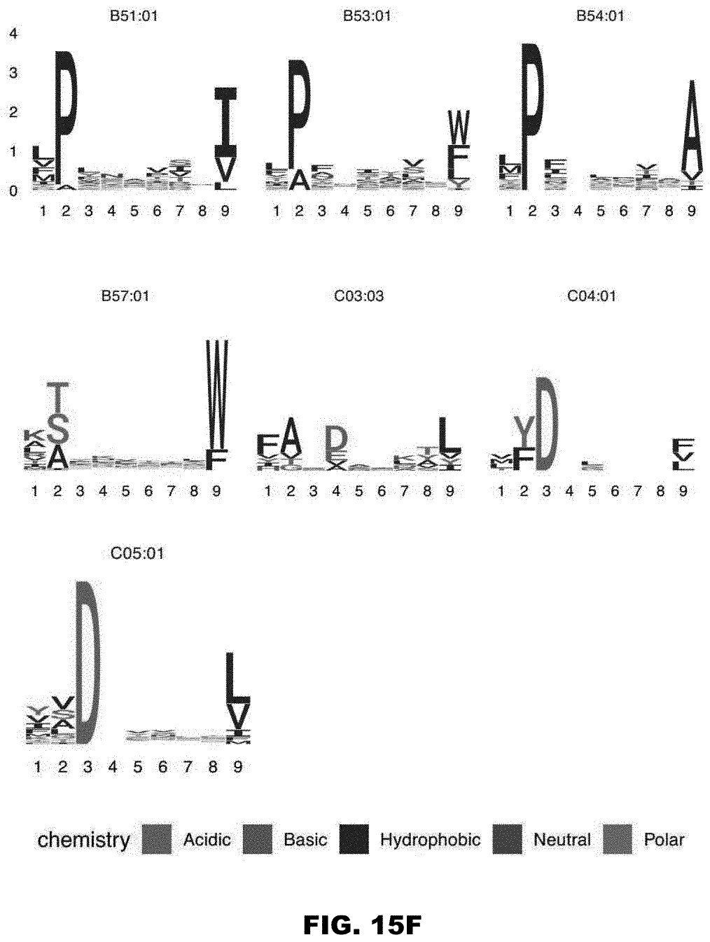

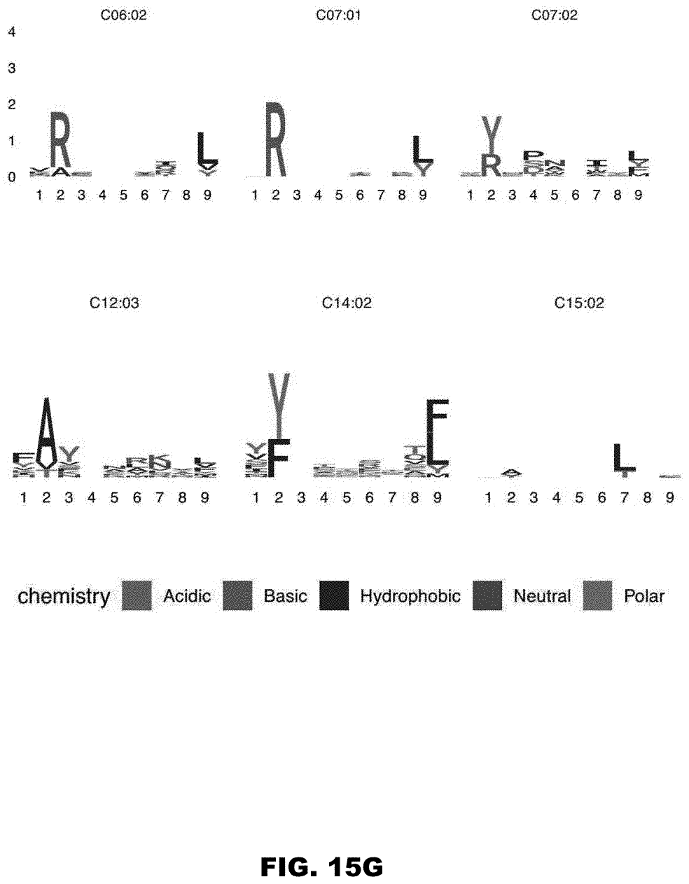

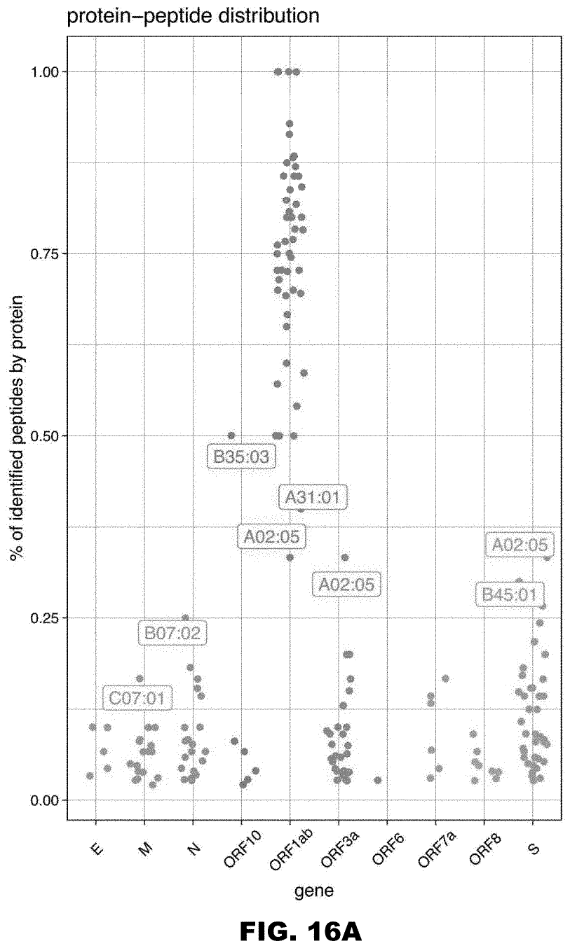

MHC-I peptides derived from SARS-CoV-2 proteome were predicted and prioritized using the EnsembleMHC workflow 100. A total of 67,207 potential 8-14mer viral peptides were evaluated for each of the considered MHC-I alleles. After filtering the pool of candidate peptides at the 5% peptide FDR threshold, the number potential peptides were reduced to 971 (658 unique peptides) (FIG. 13). The distribution of these peptides assigned to each allele for the entire SARS-CoV-2 proteome is presented in FIG. 2. There was an average of 18.6 peptides per allele with a maximum of 47 peptides (HLA-A*24:02), a minimum of 3 peptides (HLA-A*02:05), and a standard deviation (SD) of 11.3 peptides/per allele. In support of the quality of the identified peptides, the length distribution of predicted peptides adheres to expected MHC-I peptide distributions (FIG. 14) and reflect known peptide binding motifs (FIG. 15).

The high expression, relative conservation, and reduced search space of SARS-CoV-2 viral capsid structural proteins (S, E, M, and N) makes MHC-I binding peptides derived from these proteins especially high-value targets for T cell-based vaccine development. FIG. 2 describes the distribution of peptide-allele assignments originating from the four structural proteins. This assignment analysis markedly reduces the number of total predicted peptides to 160 (108 unique peptides) peptides, in proportion to the 20% amino acid content of the structural proteins compared to the entire SARS proteome. The average number of peptides per allele for specific SARS-CoV-2 structural proteins was found to be 3.1 with a maximum of 12 peptides (HLA-B*53:01), a minimum of 0 (HLA*15:02, B*35:03, B*38:01, C*03:03, C*15:02), and a SD of 2.6 peptides.

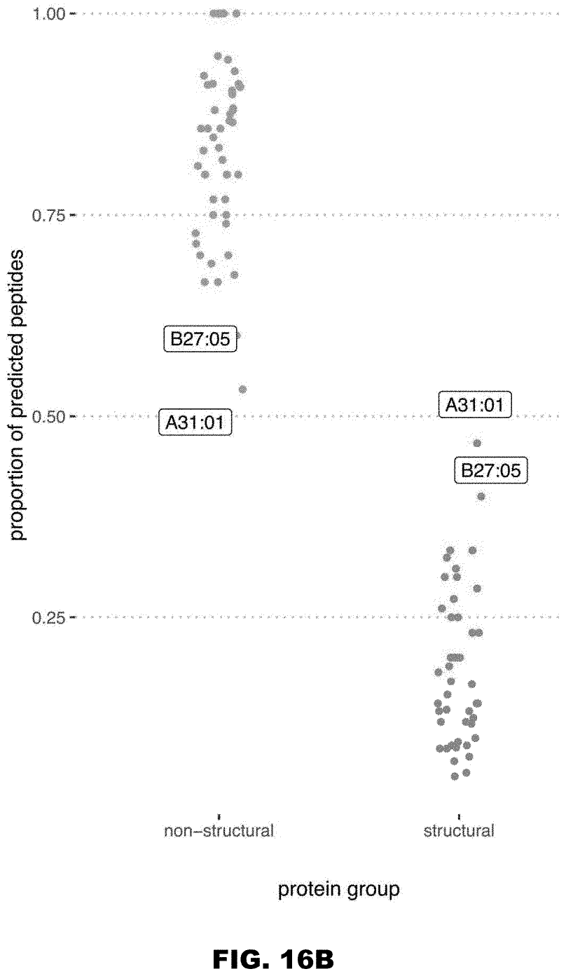

The SARS-CoV-2 structural protein allele-peptide distribution was found to be more variable with a coefficient of variance (SD/mean) of 0.83 compared to 0.61 for all SARS-CoV-2 proteins. The larger coefficient of variance indicates greater extremes in the number of peptides assigned to each allele, supporting the existence of a potential allele bias for the presentation of SARS-CoV-2 structural proteins. To better assess this potential bias, the relative changes in the peptide-allele assignment between all SARS-CoV-2 proteins and SARS-CoV-2 structural proteins was visualized. In the absence of allele bias towards SARS-CoV-2 structural proteins, the allele-peptide assignment distribution for structural proteins would be expected to be similar to the entire proteome, permitting slight fluctuations due to the restriction of the potential peptide pool. Subsequently, the average peptide-allele distribution change between all SARS proteins and the subset of structural proteins was relatively small (mean=−0.5 SDs) with 82% of the alleles remaining within one SD of the All SARS proteome allele-peptide distribution. However, 9 alleles demonstrated change of greater than one SD in relative peptide count between the two protein sets. The greatest decrease in the number of predicted peptides for a given allele was observed for A*25:01 (1.85 SDs), and the greatest increase was seen with A*31:01 (1.81 SDs). Furthermore, a similar bias was observed in the distribution of allele-specific proportions of peptides derived from structural vs non-structural proteins (FIG. 16).

The density of predicted peptides within a given protein is an important consideration for therapeutic targeting, as proteins with more unique epitopes are less likely to experience immunological escape. The epitope density of each antigen was calculated by taking the number of unique peptides per antigen and dividing it by the length of the protein (FIG. 1). The ORF10 protein showed the highest epitope density (13.1%) with 5 predicted peptides for a protein of length of 38, while the membrane protein showed the highest density for structural proteins (10.3%) with 23 unique peptides for a protein that is 222 amino acids in length. Of the structural proteins, the nucleocapsid protein N, which has a high frequency of hydrophilic amino acids, had a low epitope density of only 3.8%. Since the majority of these HLA alleles select for peptides with at least one hydrophobic anchor, epitopes within the N protein are predicted to be relatively rare. In contrast, the nucleocapsid protein has more multiallelic epitopes, as 37.5% of the peptides are predicted to bind multiple HLA, and one peptide is predicted to bind 6 different HLA

The presented results support two conclusions. First, there is an uneven distribution of high confidence predicted SARS-CoV-2 MHC-I peptides across a diverse panel of 52 common alleles. Second, there is a significant rearrangement of in the peptide-allele distribution of predicted MHC-I peptides originating from SARS-CoV-2 structural proteins. Taken together, these conclusions provide preliminary evidence of MHC-I allele bias in the presentation of SARS-CoV-2 peptides

Total Population Epitope Load Inversely Correlates with Reported Population Fitness

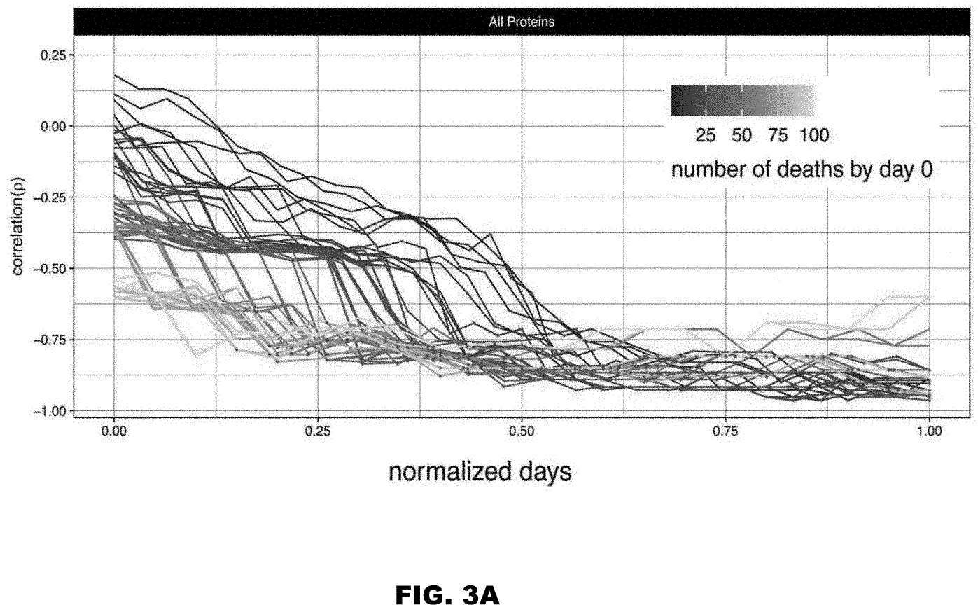

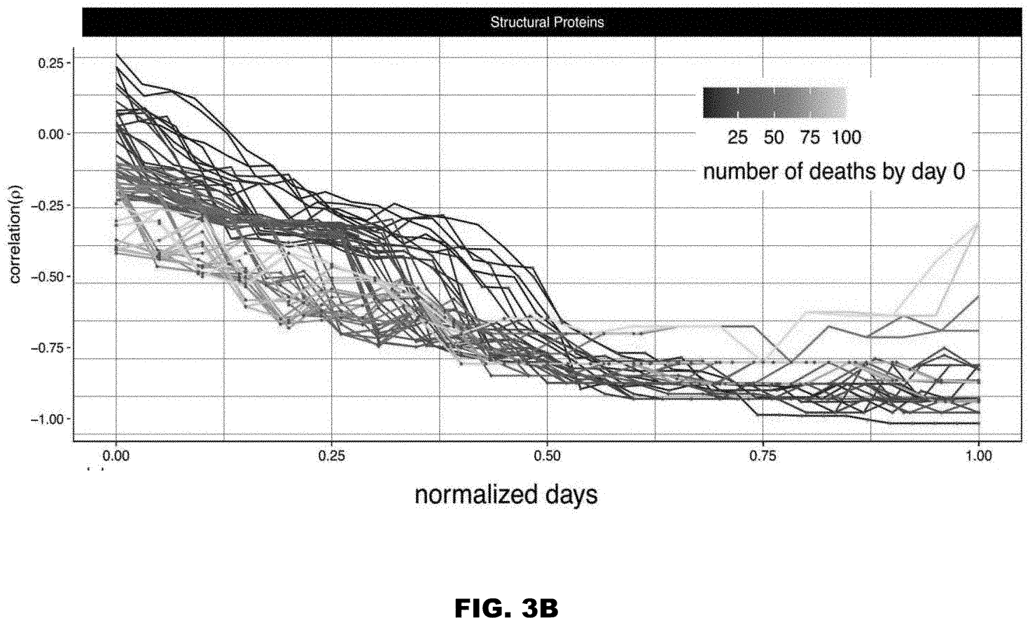

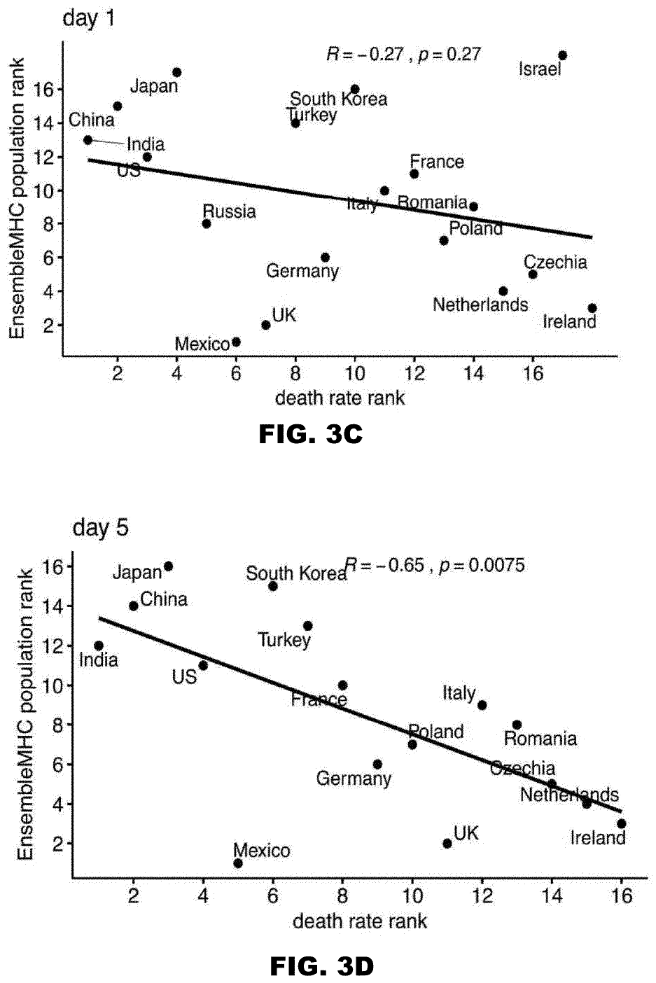

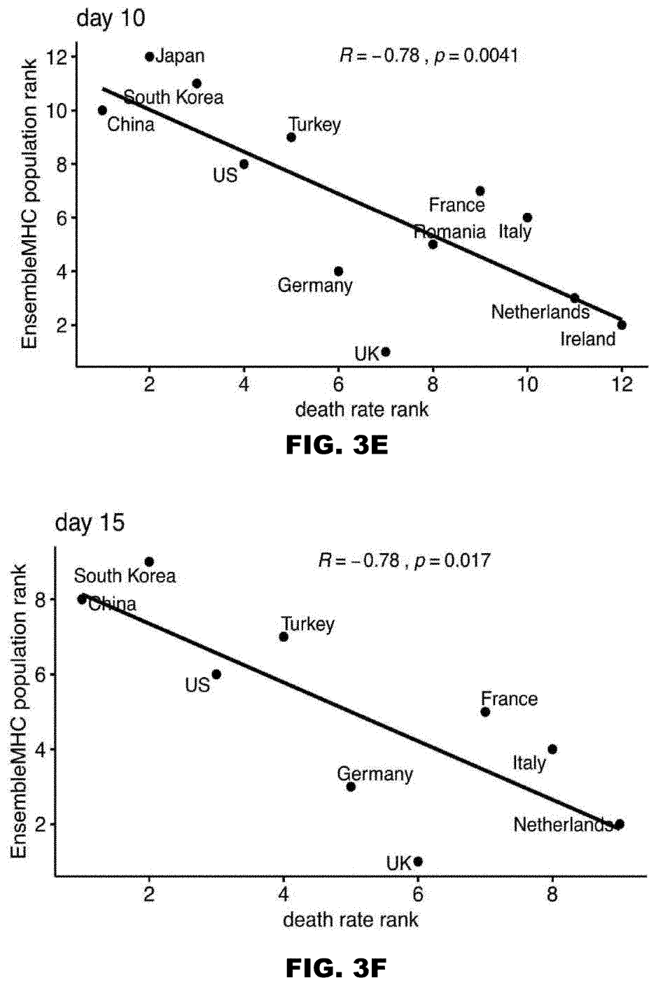

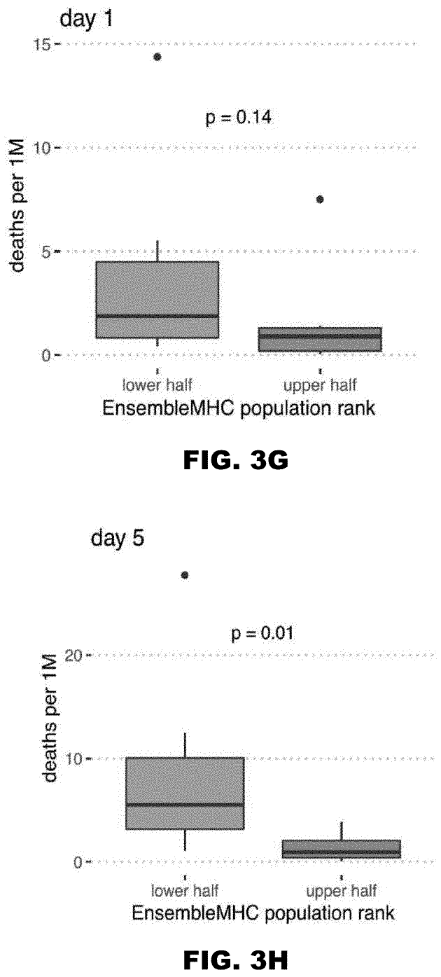

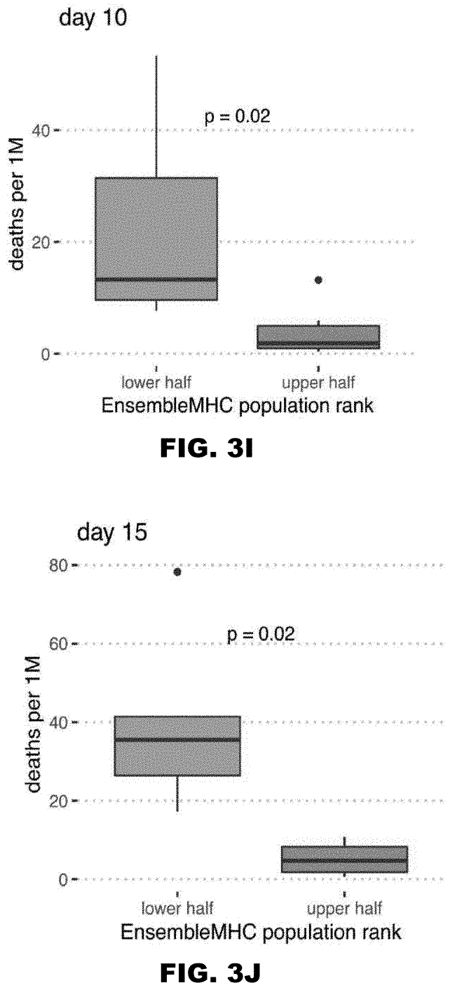

The high variability in total epitope load per allele has several clinical implications. In cancer immunology, total epitope load (the number of novel potential MHC-I binding epitopes in a tumor) is strongly associated with the response to immunotherapy, and to the presence of pre-existing cytotoxic T cell immunity. For viral immunity, certain MHC-I alleles are strongly associated with long term control of chronic viruses such as HIV. An uneven distribution of peptide-allele assignments was observed across the 52 common MHC-I alleles, both for the entire SARS-CoV-2 proteome and for the structural proteins. To determine if the described anomalies in peptide allele assignment could be predictive of population fitness against SARS-CoV-2, the correlation of the EnsembleMHC population score (EMP) with the reported deaths per million for 21 countries as a function of time was analyzed (FIG. 3).

FIG. 2 shows results of the correlation analysis for the EMP score based on the entire SARS-Cov-2 proteome (left panel) or restricted to only SARS-Cov-2 structural proteins (right panel). Both analyses demonstrated an overall negative correlation between EMP score and deaths per million that decreased as time progressed with a mean correlation of −0.61 and −0.71 for structural protein population score and all proteome population score respectively. Significance testing of each correlation revealed that 66% of correlations attained a p-value of <0.05 with correlations based on EMP scoring of structural proteins (76%) demonstrating a 30% higher proportion of statistically significant correlations over the EMP score based on all SARS-CoV-2 proteins (57%). Due to relatively low statistical power of the obtained correlations (63.8% of correlations below 80% power), the positive predictive value for each correlation was calculated (FIG. 14). The resulting proportions of correlations with a positive predictive value of greater than 95% were similar to the observed significant p-value proportions with 62% of all correlation, 69% of structural EMPs and 55% all proteins EMPs. This similarity in results for p-values and PPV suggests an overall true correlation.

To further capture the dynamics of EMP score based on the presentation of SARS-CoV-2 structural proteins and the observed deaths per million, FIG. 3 depicts the correlation between EnsembleMHC structural protein score and deaths per million at the median death threshold (50 deaths) for days 1, 5, 10, and 15 days. FIG. 3 also shows the correlation at the selected time points with all days excluding day 1 reaching statistical significance.

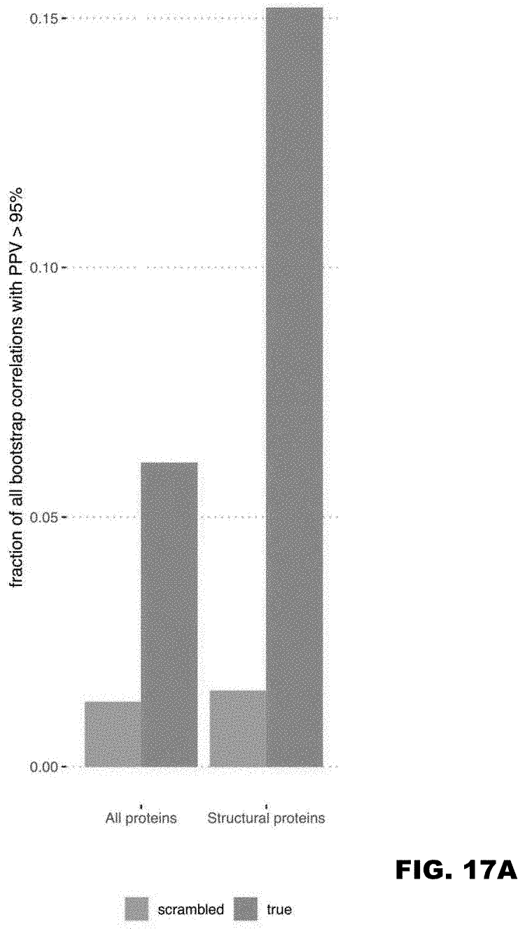

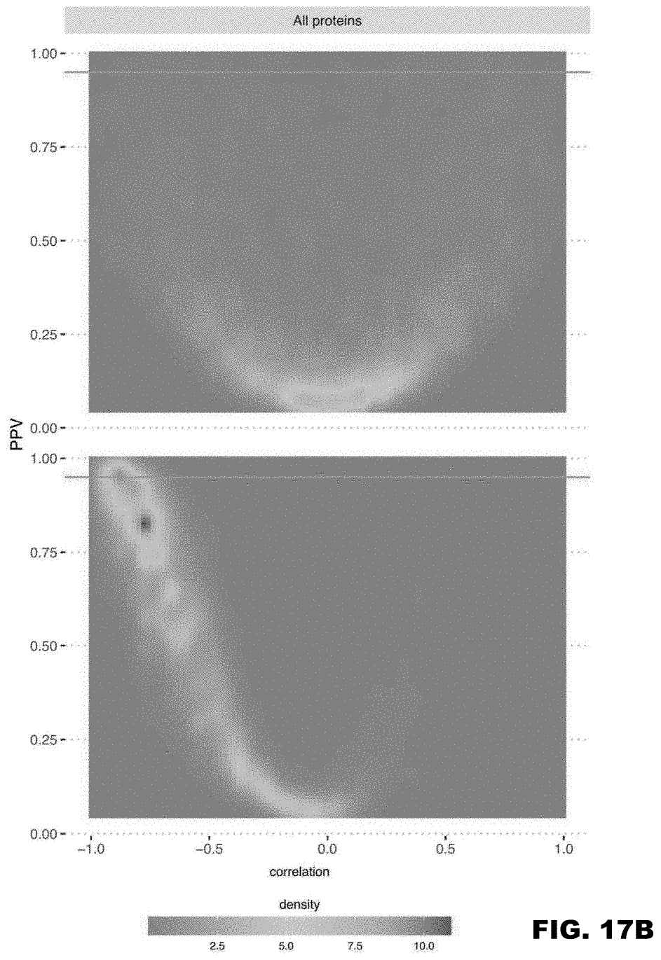

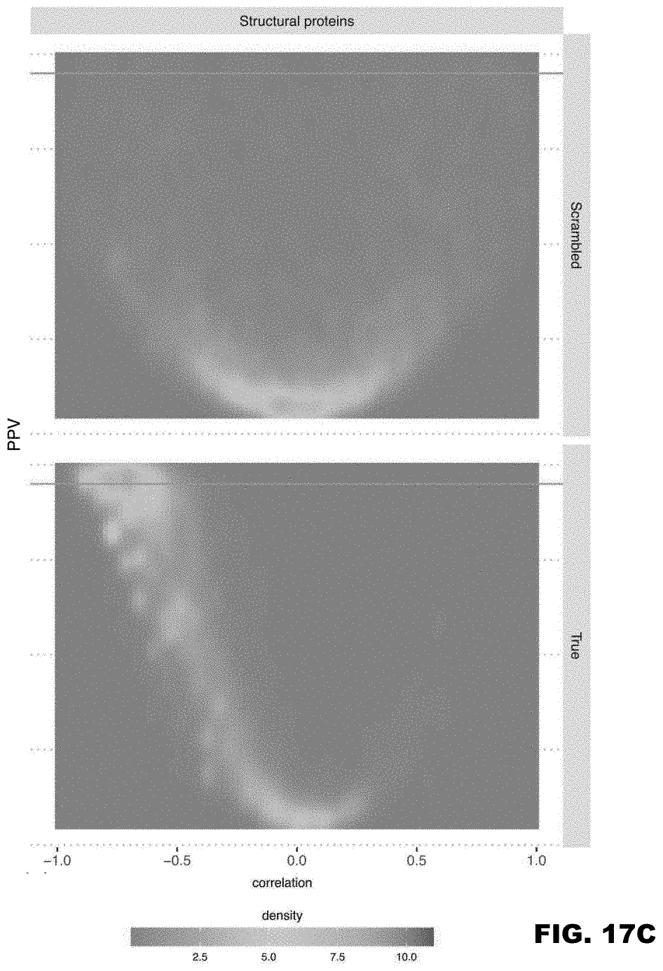

To assess the ability for the EMP score based on structural proteins to partition countries into high and low risk populations, countries were grouped based on having an EMP score higher or lower than the median observed EMP score. Similarly, with the exception of day 1, all other days showed a significant difference between observed deaths per million based on EnsembleMHC population score grouping. The robustness of the correlation calculations was determined by measuring the correlations from bootstrap sampling of half of the countries using the 50-death threshold with either the structural protein based population score, or a randomized assignment of observed structural protein population scores (FIG. 17). The significantly reduced sample size decreased the proportion of bootstrapped correlations with PPV values of greater than 95% to 11%. However, the correlations attained from the EMP score based on structural proteins produced 10-fold higher number of significant correlations than random correlations and 2.5-fold higher than all proteins.

Death threshold and time-dependent analysis of the EMP correlations reveal several trends. First, the mean correlation coefficient decreases, and the proportion of statistically significant correlations increases as a function of death threshold. Second, the mean correlation decreases and stabilizes when comparing points before the halfway point (mean=−0.64, sd=0.15) and after the halfway point (mean=−0.83, sd=0.05) of each time series analysis respective to the selected death threshold.

In summary, three observations are made. First, there is evidence of a statistically significant inverse correlation of EMP score and observed deaths per million. Second, there is evidence that this relationship is primarily driven by the presentation of SARS-CoV-2 structural proteins. Finally, there is the potential to separate high and low risk populations based on EMP score

Peptides Identified by the Presentation Score Function Identify High Value Target Regions

The EMP score based on structural proteins indicated that observed deaths per million may be primarily shaped by the presentation of MHC-I peptides derived from SARS-Cov-2 structural proteins. To gain additional insight into these predicted peptides, the identified structural peptides were mapped back into their originating protein sequence (FIG. 3). Analysis of the frequency of positions within source protein sequences that appeared in a predicted peptide uncovered regions that are enriched for targetable peptides. To address the potential for predicted peptides to be disrupted by polymorphisms, 4,455 full length protein coding sequences from SARS-Cov-2 structural proteins were aligned, and the number of unique polymorphisms were calculated at each position (FIG. 2). This analysis revealed that MHC-I peptides derived from structural proteins were unlikely to be impacted by polymorphism with the average number of polymorphic residues between all sequences being 6.2%. Furthermore, the overall sequence conservation across all sequenced proteins was high with all proteins showing greater than 99.99% conservation. The structural implications of these MHC-I peptide hotspots were explored by mapping predicted MHC-I peptides onto existing protein structures for the envelope and spike protein and the predicted structures for the nucleocapsid and membrane protein. Of particular note, the peptides mapped to the envelope protein revealed an enrichment of peptide located in the transmembrane portion of the channel. This localization indicates that peptides predicted from the envelope protein are less likely to allow an avenue for viral escape due to the invariance of such regions for function. In terms of peptide localization, density, and sequence invariance, the membrane protein appears as the most appealing target. However, further interpretation of the structural impacts and importance of these peptides will require a solved structure

### Discussion

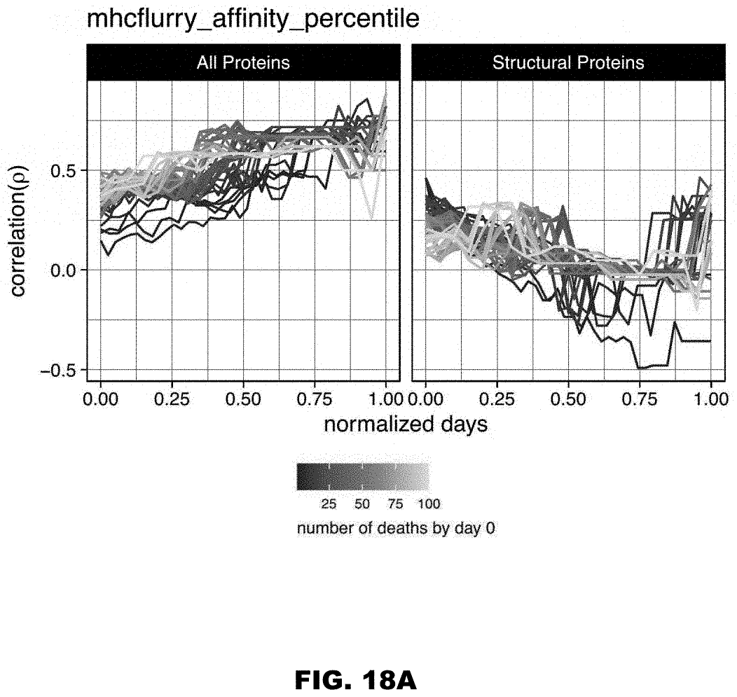

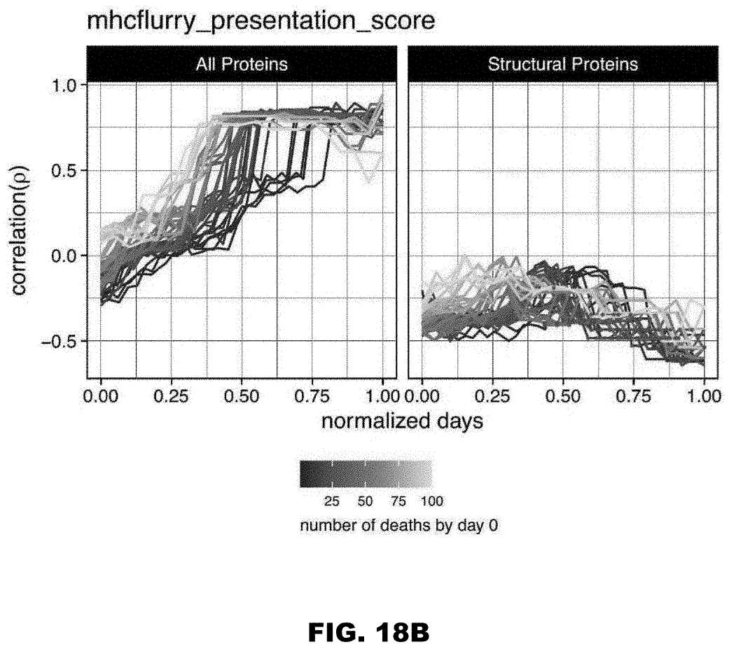

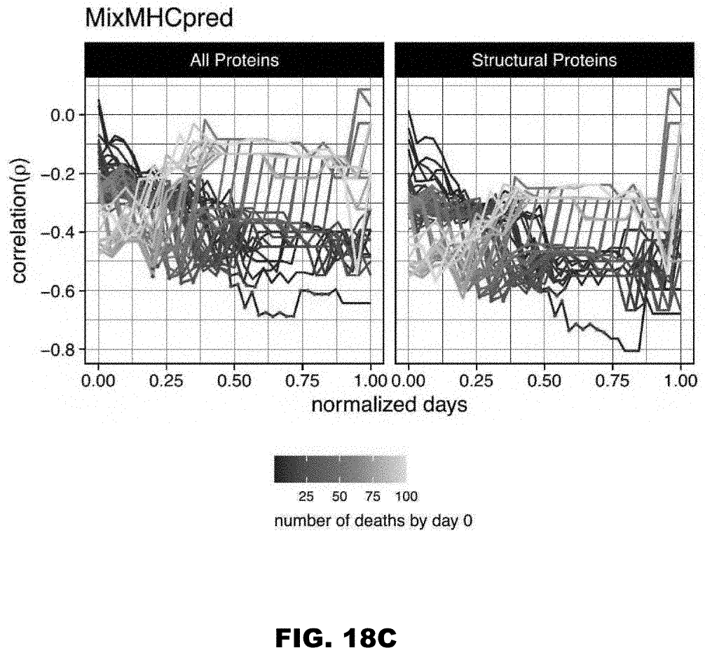

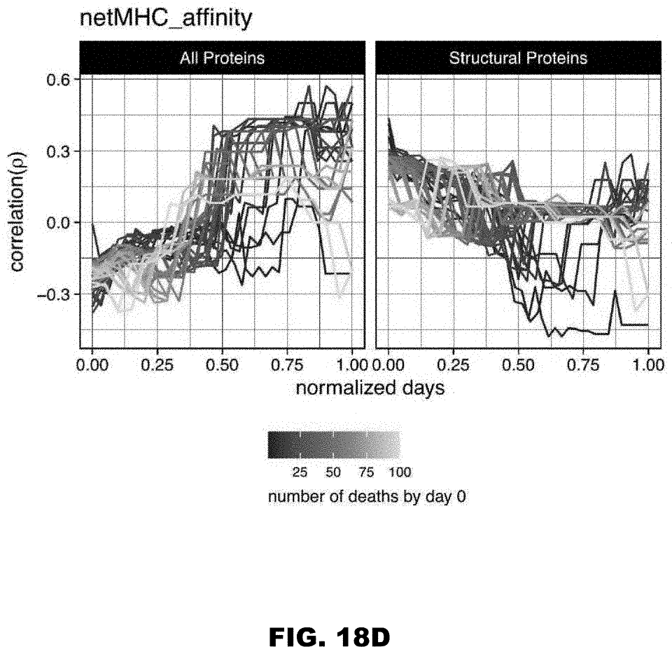

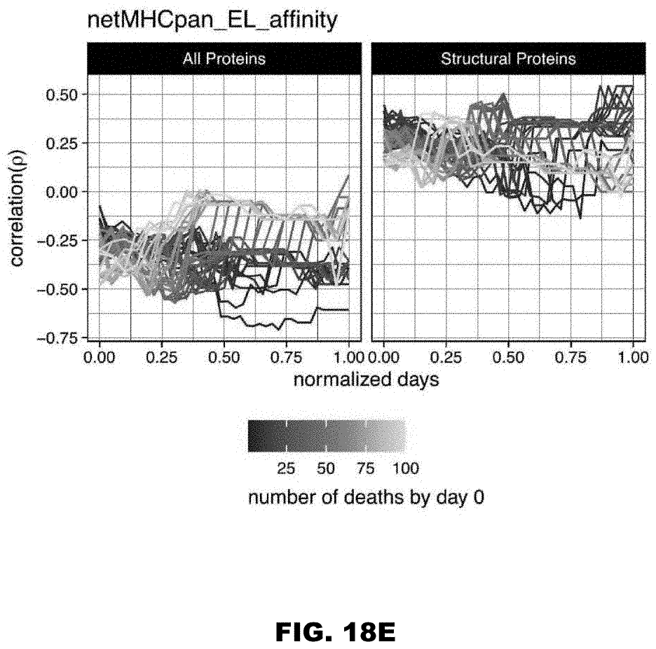

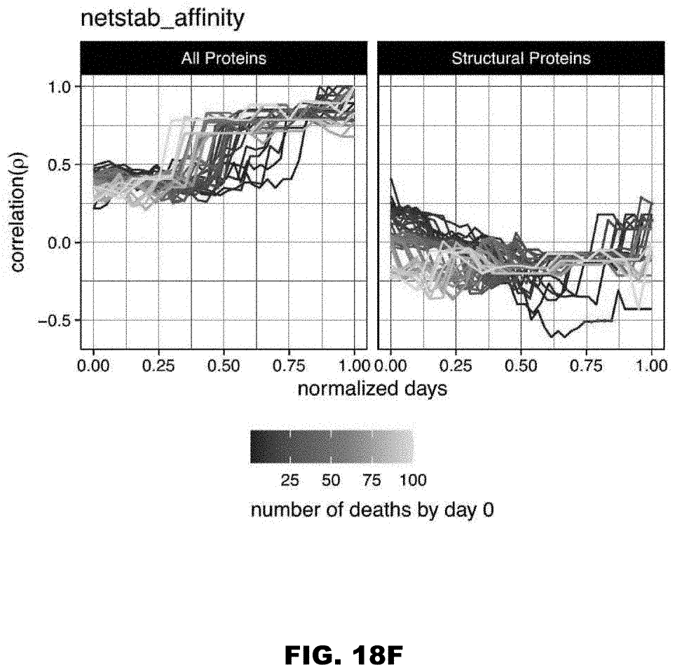

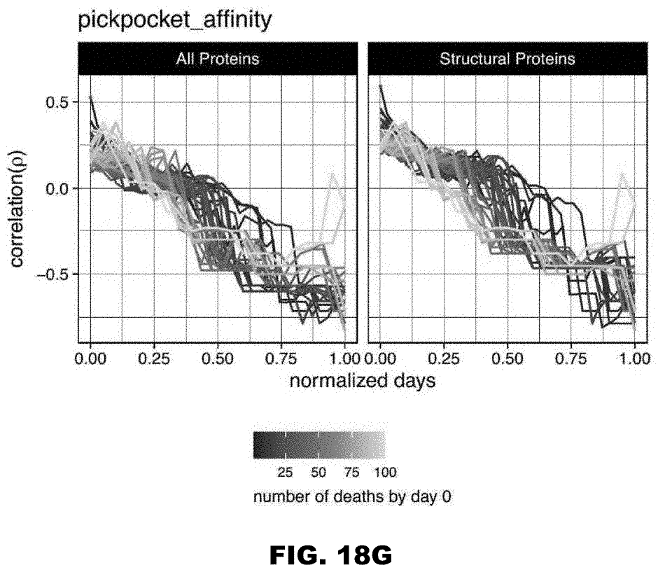

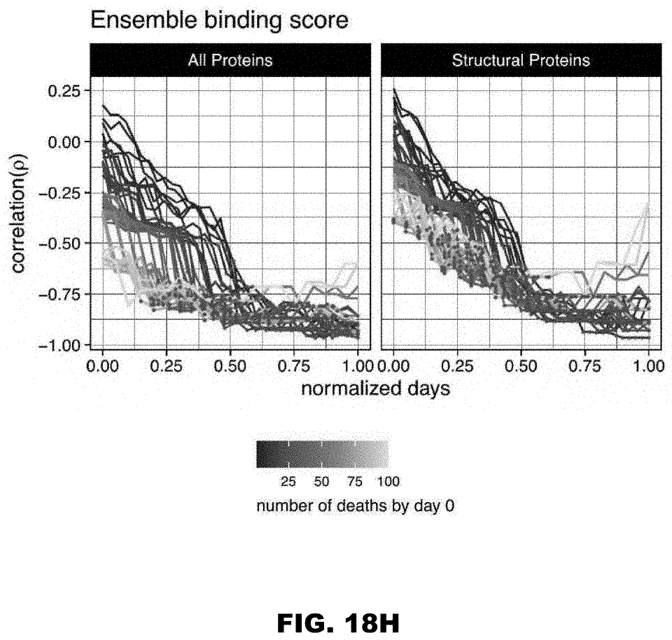

EnsembleMHC workflow 100 was developed using SARS-CoV-2 as an example target antigen. The EnsembleMHC workflow 100 was then utilized to identify SARS-CoV-2 MHC-I peptides with high value for CB8⁺ T cell based therapies, the novel. This workflow leverages seven MHC-I binding and processing algorithms to perform an ensemble-based prediction. The EnsembleMHC 100 improves specificity of peptide calls through the data guided assignment of algorithm and allele specific score thresholds and peptide call confidence. These values are then combined in order to filter peptide candidates and apply stringent FDR control to each identified peptide. The use of several commonly used global score thresholds were unable to recreate the observed relationship between population SARS-CoV-2 peptide binding capacity and observed death rate with the same level of statistical significance as EnsembleMHC 100 (FIG. 18).

The EnsembleMHC workflow 100 was used to predict 8-14mer peptides derived from SARS-CoV-2 proteins for a panel of 52 common MHC-I alleles resulting in the identification of 658 unique peptides. Analysis of the peptide-allele assignment distribution revealed a notable disparity in the number of peptides assigned to each allele indicating a potential presentation capacity hierarchy for SARS-CoV-2 peptides (FIG. 2). Due to the previously reported observation of long-lived memory T cells to SARS-CoV-1 structural proteins, the peptide-allele assignment distribution was assessed with respect to only structural proteins resulting in the isolation of 108 unique peptides. The peptide-allele assignment distribution for structural proteins was shown to be more variable with a 30% higher coefficient of variance compared to all proteins and dramatic shift in relative binding capacity was noted between the two protein groups. The observed discrepancies in allele-peptide assignment distribution support two main conclusions. First, there is evidence of an allele bias towards the presentation of high confidence of SARS-CoV-2 proteins. Second, the disparity in peptide-allele assignment distribution is amplified when considering only SARS-CoV-2 structural proteins. The second conclusion is particularly interesting as recent analysis of potential SARS-CoV-2 MHC-I peptides have been largely focused on the prediction of all SARS-CoV-2 proteins while previous evidence of long lasting memory T cell response supports the particular importance of structural proteins.

The collective population binding capacity for SARS-CoV-2 peptides was assessed as a possible explanation for observation of the disparate impact of the SARS-CoV-2 pandemic in different global populations. This potential relationship was assessed by calculating the correlation between the EnsembleMHC population score, based on individual allele binding capacity and population frequency, and the observed deaths per million in 21 countries as a function of time. It was shown that the correlation between both the EnsembleMHC population score based on all SARS-CoV-2 proteins and SARS-CoV-2 structural proteins produced strongly negative correlations supporting the hypothesis that enhanced presentation of SARS-CoV-2 proteins improves overall outcome (FIG. 3). Interestingly, it was observed that the EnsembleMHC population score based on the presentation of SARS-CoV-2 structural proteins produced a substantial improvement in the number of statistically significant correlations. Additionally, it was demonstrated that high and low risk countries could be identified through partitioning of countries based on structural protein EnsembleMHC population score. This supports the hypothesis that productive SARS-CoV-2 responses are predominantly driven by the presentation of peptides derived from structural proteins. Additional analysis of death threshold and time series dependence indicated that the correlation between EnsembleMHC population score and deaths rate was stronger and generally more significant when starting the correlation analysis from higher death thresholds and later time points. This observation supports the inference that presentation of SARS-CoV-2 peptides are more likely to shape an observed death per million once the virus becomes embedded in a population.

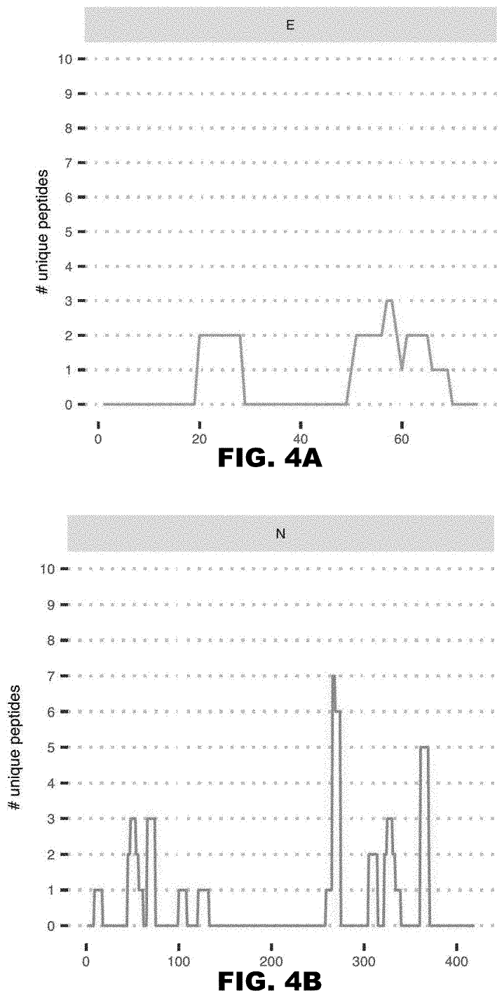

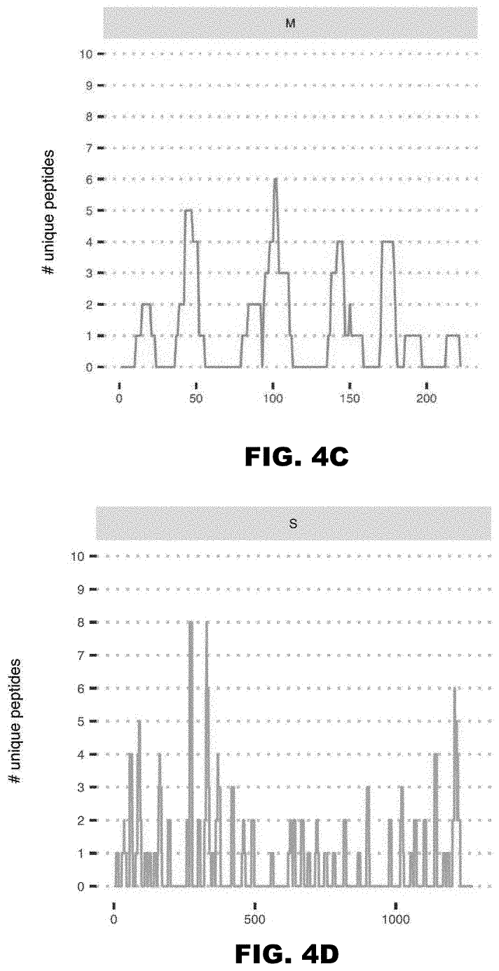

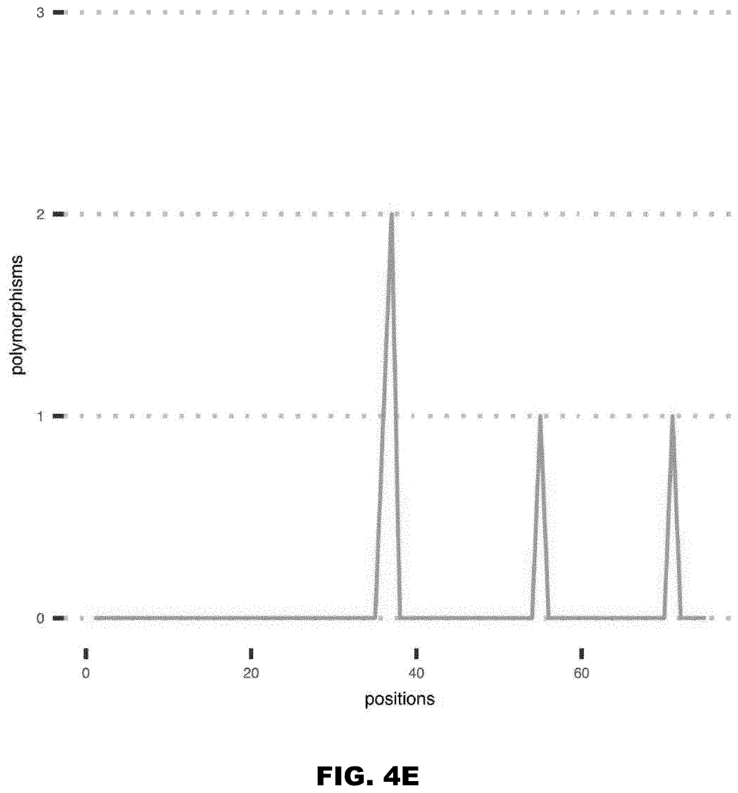

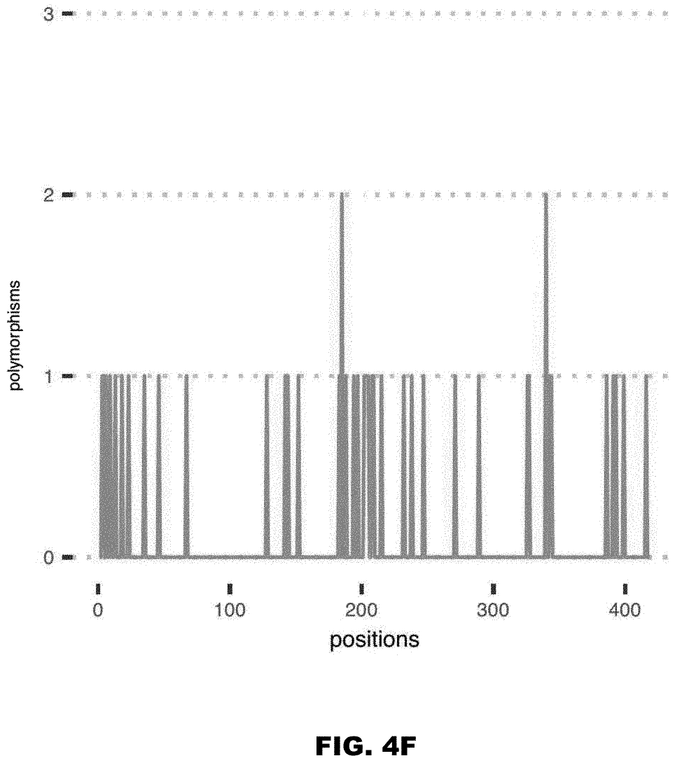

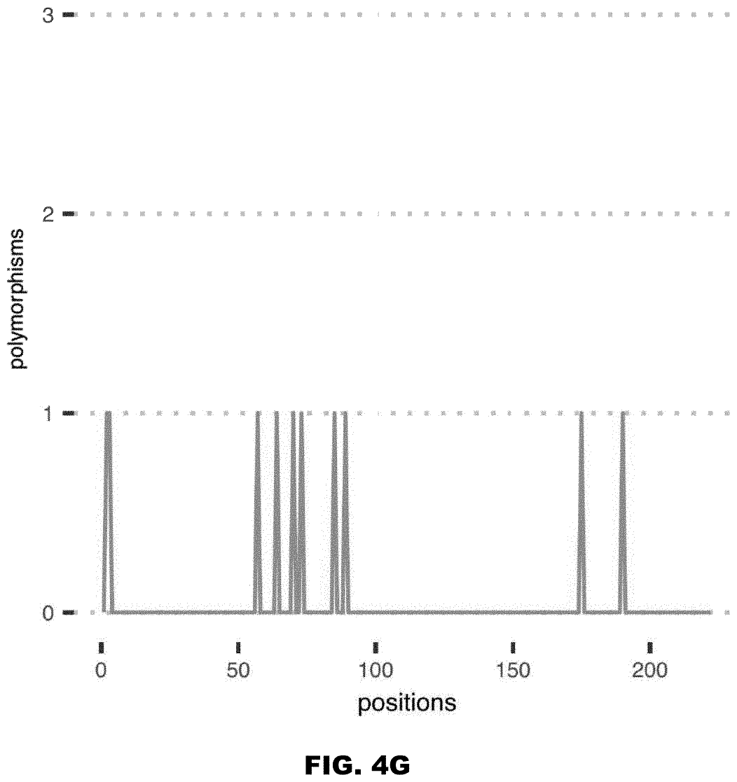

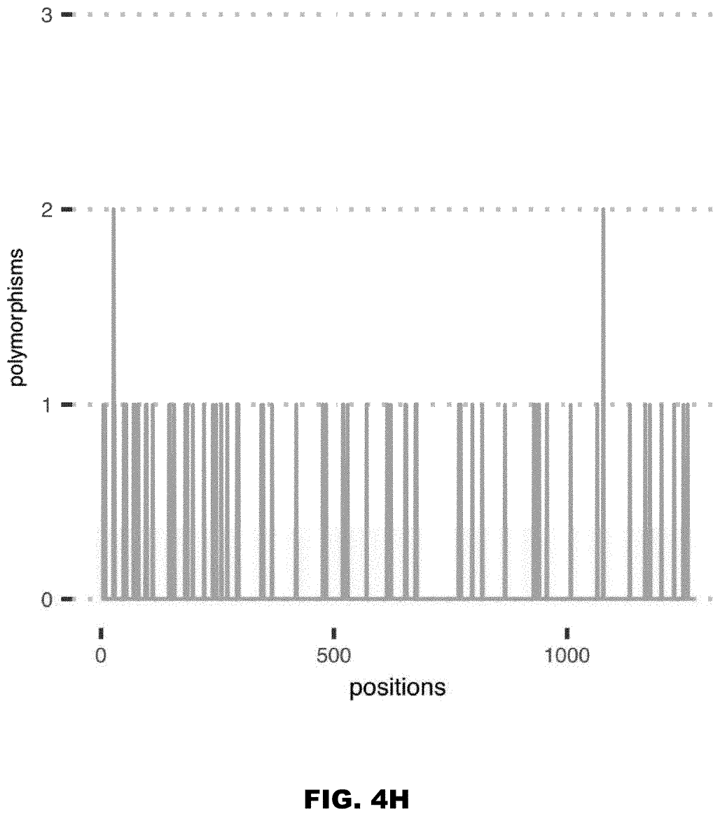

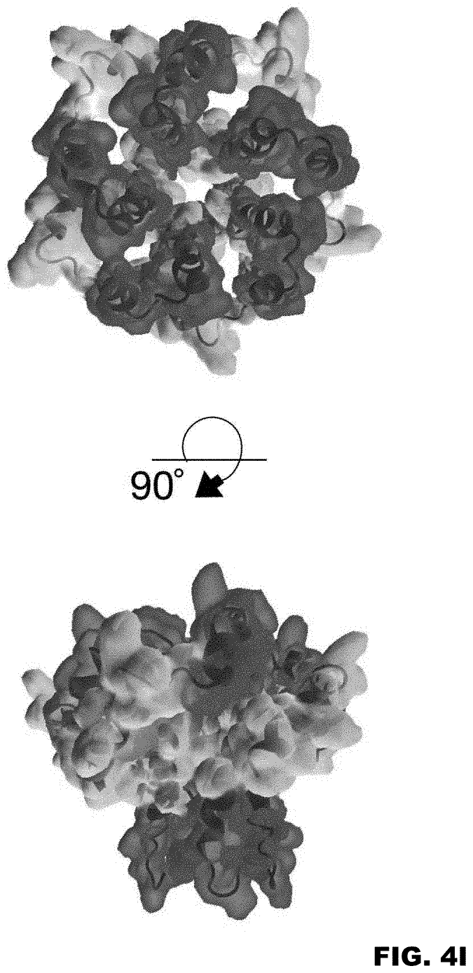

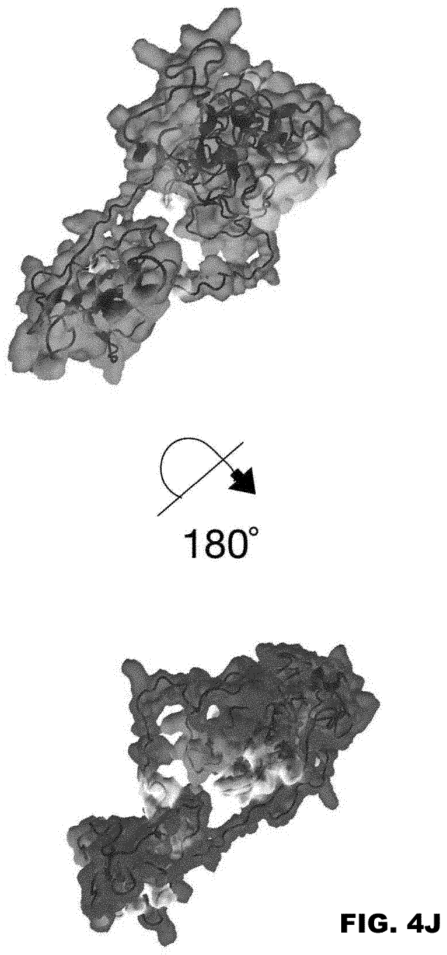

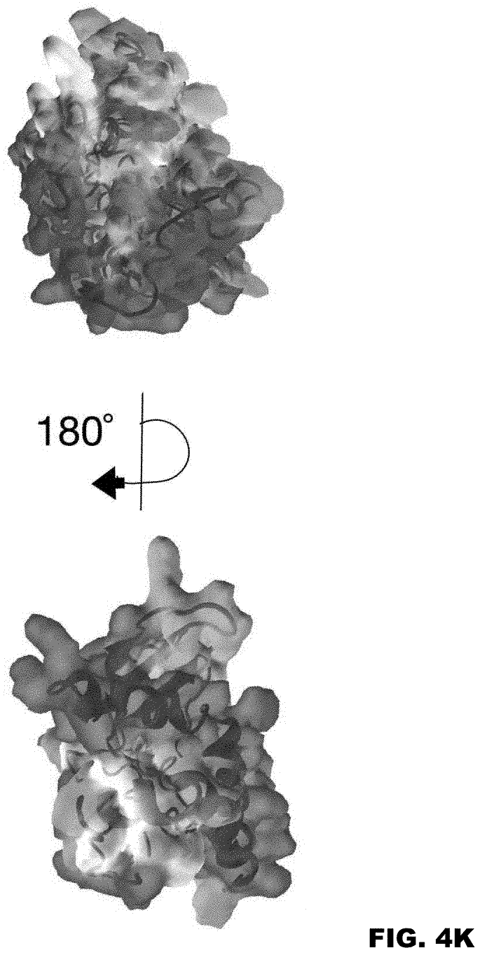

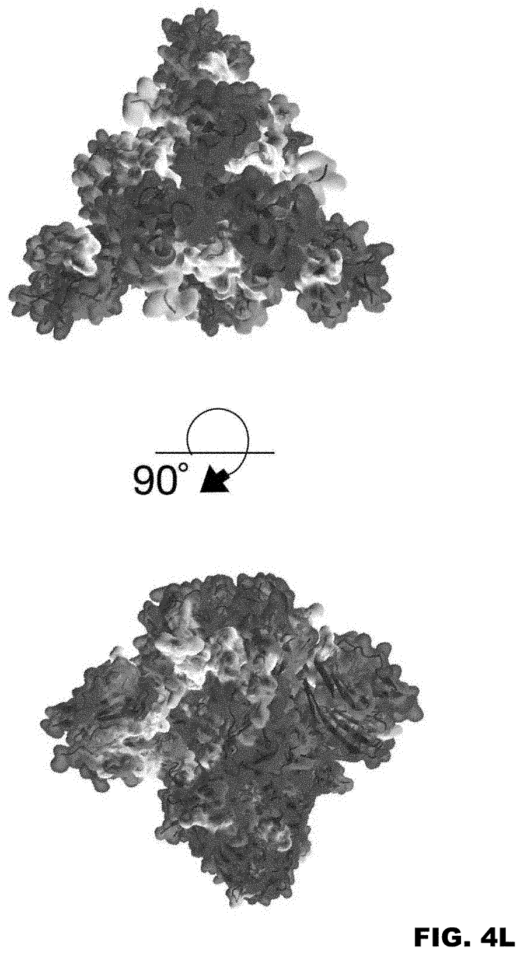

The implied importance of SARS-CoV-2 structural proteins for immune response prompted the analysis of molecular origin. It was revealed that predicted peptides derived from SARS-CoV-2 structural proteins originate from regions enriched for predicted MHC-I peptides (FIG. 4). Furthermore, it was determined that a majority of these peptides are unlikely to be impacted from observed polymorphism in viral protein sequence.

The ability for MHC genotype to shape patient outcome has been well studied in the context of HIV infections. Similarly, MHC-outcome associations have been reported in SARS-CoV-1. A study of a Taiwanese and Hong Kong cohort of patients with SARS-CoV found that HLA-B*07:03 and HLA-B*46:01 were linked to increased susceptibility while HLA-Cw*15:02 were linked to increased resistance. However, such associations did not remain after statistical correction and it is still unclear if MHC-outcome associations in SARS-CoV-1 are applicable to SARS-CoV-2 Recently, a comprehensive prediction of SARS-CoV-2 MHC-I peptides indicated a depletion of a high affinity binding peptides for HLA-B*46:01 potentially supporting a similar association in SARS-CoV-2. However due to the use of global binding affinity thresholds, it remains unclear if reported results represent a true depletion or are an artifact of variation in binding capacity between diverse alleles. Accordingly, when using allele specific binding capacity thresholds, as used by EnsembleMHC 100, an obvious depletion of peptides for HLA-B*46:01 was not observed. These conflicting results are likely a product of the underlying complexity of CD8⁺ immunity. While overall epi-tope load has been associated with viral control, the quality of the presented peptide is also a factor. Future assessment of the immunogenicity and biochemical analysis of binding affinity for the predicted peptides will likely resolve this uncertainty, and provide finer grain resolution of MHC-outcome association.

Another factor to consider is the overall system dynamics of immune response to SARS-CoV-2 infections. It has been shown that the innate immune system also provides a response to SARS-CoV-2. However, overstimulation of the innate immune response has been implicated as the driving cause of mortality via the generation of “cytokine storms”. The initiation of “cytokine storms” has been shown to be deleterious to T cell response with an observed inverse correlation between cytokines associated with this state and T cell levels as well as expression of T cell dysfunction markers. These observations support the underlying hypothesis that patients with a higher likelihood of broad T cell responses are better protected as research has shown that the occurrence of “cytokine storms” diminishes with robust T cell responses.

The workflow used in this study does have certain limitations potentially affecting the overall generalization of results especially concerning peptide presentation capacity and disease outcome. First, this model assumes fidelity of reported SARS-CoV-2 deaths and MHC-I allele frequencies. Second, the presented model does not account for additional external factors (e.g. social distance, governmental policies). However, recent SIR models have indicated that unless the implementation of preventative polices are perfectly timed, they are unlikely to decrease the overall incidence of disease. Additionally, due to nature of the EnsembleMHC 100 parameterization, only a subset of alleles is considered. However, the selected allele represents some of the most common global MHC-I alleles, and while there is large observed variance in the MHC-I protein sequence, the variation in unique peptide binding motifs is much less. Future iterations of EnsembleMHC 100 will be expanded to a larger set of alleles through the use of structure-based clustering of MHC alleles.

In summary, the present disclosure identifies a set of high confidence SARS-CoV-2 peptides that provide a valuable starting point for experimental validation. The predicted peptides are shown to form a variable distribution across a diverse panel of 52 MHC-I alleles, and a population score function is provided, based on the binding capacity of each individual alleles and regional frequencies provides a strong and statistically significant correlation with observed mortality. Furthermore, the present disclosure highlights the potential importance of peptides derived from viral structural proteins and shows that these peptides originate from enriched regions.

### III. Target Antigens

Although the method for selecting peptide sequences for preparing a vaccine composition described in Section I above was developed using the SARS-COV2 pathogen as a target antigen, the method can also be used to identify peptides derived from other pathogens, cancer antigens, and immune modulation antigens, among others. Non-limiting examples of immune modulation antigens are antigens for which the method of the instant disclosure can be used to select peptide sequences within gene therapy vectors that are likely to induce anti-therapeutic immune response. For example, gene therapy using adenoviral or other viral vectors, as well as therapy with CRISPR/Cas9 induce anti-therapeutic immune response.

(a) Pathogens

Non-limiting examples of pathogens for which the method of the instant disclosure can be used to select peptide sequences for preparing a vaccine composition include infectious microbes such as virus, bacteria, parasites and fungi and fragments thereof. Examples of infectious virus include, but are not limited to: Retroviridae (e.g. human immunodeficiency viruses, such as HIV-1 (also referred to as HTLV-III, LAV or HTLV-III/LAV, or HIV-III, and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, pseudorabies virus, cytomegalovirus (CMV), herpes virus; Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Non-limiting examples of bacterial pathogens include Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacterpyloris, Borelia burgdorferi, Legionella pneumophilia, Mycobacteria sps (e.g., M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcusfaecalis, Streptococcus bovis, Streptococcus (anaerobic sps.), Streptococcus pneumoniae, pathogenic Campylobacter sp., Enterococcus sp., Haemophilus infuenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

Examples of pathogens also include, but are not limited to, infectious fungi that infect mammals, and more particularly humans. Examples of infectious fungi include, but are not limited to, Cryptococcus neoformans, Histoplasma capsulatum, Coccidioides immitis, Blastomyces dermatitidis, Chlamydia trachomatis, Candida albicans. Examples of infectious parasites include Plasmodium such as Plasmodium falciparum, Plasmodium malariae, Plasmodium ovale, and Plasmodium vivax. Other infectious organisms (i.e., protists) include Toxoplasma gondii.

Other medically relevant microorganisms that serve as antigens in mammals, and more particularly humans, are described extensively in the literature, e.g., see C. G. A Thomas, Medical Microbiology, Bailliere Tindall, Great Britain 1983, the entire contents of which is hereby incorporated by reference. In addition to the treatment of infectious human diseases, the compositions and methods of the present invention are useful for treating infections of non-human mammals. Many vaccines for the treatment of non-human mammals are disclosed in Bennett, K. Compendium of Veterinary Products, 3rd ed. North American Compendiums, Inc., 1995.

(b) Cancers

In some aspects, the disease condition is cancer or a neoplasm. The neoplasm can be malignant or benign, the cancer can be primary or metastatic; the neoplasm or cancer can be early stage or late stage. Non-limiting examples of neoplasms or cancers that can be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In other aspects, the disease condition is an immune system condition. Non-limiting examples include diseases associated with a weak immune system (primary immune deficiency), a disease associated with a weakened system (acquired immune deficiency), an immune system that is too active (allergic reactions), or an autoimmune disease. Non-liming examples of immune system conditions include severe combined immunodeficiency (SCID), rheumatoid arthritis, osteoarthritis, Chron's disease, angiofibroma, ocular diseases (e.g., retinal vascularisation, diabetic retinopathy, age-related macular degeneration, macular degeneration, etc.), obesity, Alzheimer's disease, restenosis, autoimmune diseases, allergy, asthma, endometriosis, atherosclerosis, vein graft stenosis, peri-anastomatic prothetic graft stenosis, prostate hyperplasia, chronic obstructive pulmonary disease, psoriasis, inhibition of neurological damage due to tissue repair, scar tissue formation (and can aid in wound healing), multiple sclerosis, inflammatory bowel disease, infections, particularly bacterial, viral, retroviral or parasitic infections (by increasing apoptosis), pulmonary disease, neoplasm, Parkinson's disease, transplant rejection (as an immunosuppressant), septic shock, etc.

### IV. Computing Device

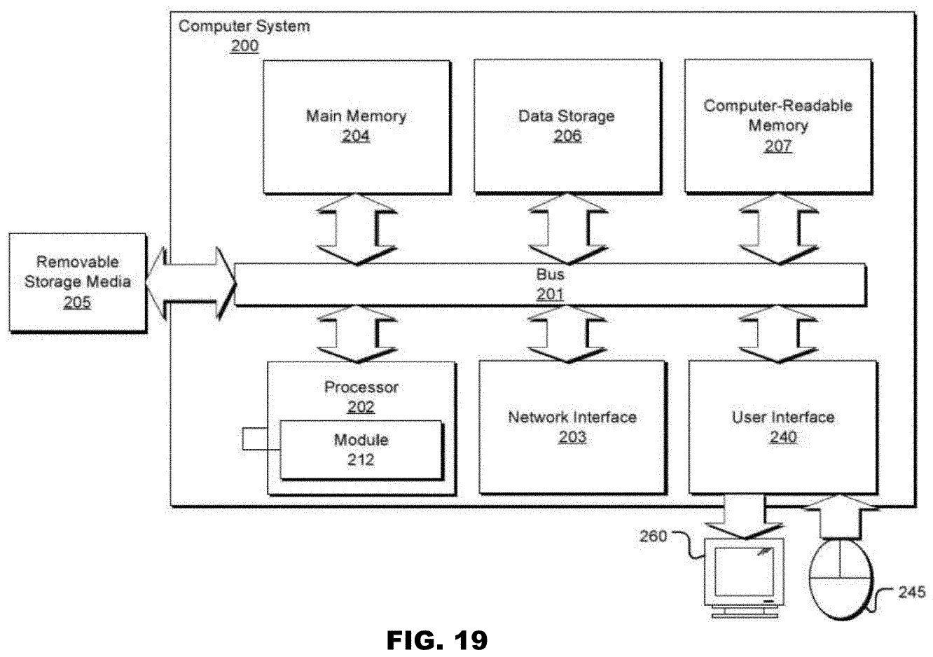

Referring to FIG. 19, a computing device 200 is illustrated which may be configured, via one or more of an application 211 or computer-executable instructions, to execute functionality described herein. More particularly, in some aspects, aspects of the functionality/methods herein may be translated to software or machine-level code, which may be installed to and/or executed by the computing device 200 such that the computing device 200 is configured to execute functionality described herein. It is contemplated that the computing device 200 includes any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 200 includes various hardware components, such as a processor 202, a main memory 204 (e.g., a system memory), and a system bus 201 that couples various components of the computing device 200 to the processor 202. The system bus 201 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 200 may further include a variety of memory devices and computer-readable media 207 that includes removable/non-removable media and volatile/nonvolatile media and/or tangible media, but excludes transitory propagated signals. Computer-readable media 207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media includes wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 204 includes computer storage media in the form of volatile/nonvolatile memory such as read only memory (ROM) and random access memory (RAM). A basic input/output system (BIOS), containing the basic routines that help to transfer information between elements within the computing device 200 (e.g., during start-up) is typically stored in ROM. RAM typically contains data and/or program modules that are immediately accessible to and/or presently being operated on by processor 202. Further, data storage 206 in the form of Read-Only Memory (ROM) or otherwise may store an operating system, application programs, and other program modules and program data.

The data storage 206 may also include other removable/non-removable, volatile/nonvolatile computer storage media. For example, the data storage 206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media includes magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 200.

A user may enter commands and information through a user interface 240 (displayed via a monitor 260) by engaging input devices 245 such as a tablet, electronic digitizer, a microphone, keyboard, and/or pointing device, commonly referred to as mouse, trackball or touch pad. Other input devices 245 includes a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 245 are in operative connection to the processor 202 and may be coupled to the system bus 201, but may be connected by other interface and bus structures, such as a parallel port, game port or a universal serial bus (USB). A monitor 260 or other type of display device may also be connected to the system bus 201. The monitor 260 may also be integrated with a touch-screen panel or the like.

The computing device 200 may be implemented in a networked or cloud-computing environment using logical connections of a network interface 203 to one or more remote devices, such as a remote computer. The remote computer may be a personal computer, a server, a router, a network PC, a peer device or other common network node, and typically includes many or all of the elements described above relative to the computing device 200. The logical connection includes one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 200 may be connected to a public and/or private network through the network interface 203. In such aspects, a modem or other means for establishing communications over the network is connected to the system bus 201 via the network interface 203 or other appropriate mechanism. A wireless networking component including an interface and antenna may be coupled through a suitable device such as an access point or peer computer to a network. In a networked environment, program modules depicted relative to the computing device 200, or portions thereof, may be stored in the remote memory storage device.

Certain aspects are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some aspects, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering aspects in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In aspects in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein includes desktop computers, laptops, tablets e-readers, personal digital assistants, smartphones, gaming devices, servers, and the like. The computing devices may access computer-readable media that include computer-readable storage media and data transmission media. In some aspects, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

Attached hereto are Addendum A and Addendum B which are incorporated by reference in their entirety.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

### V. Peptide Library

A further aspect of the present disclosure provides a peptide library. The peptide library comprises a plurality of library members, wherein each member is a 5-20mer peptide having a predetermined likelihood of binding to a target antigen, and is restricted to a predetermined number of common MHC-I alleles. Each member is selected from a plurality of candidate peptides based on a presentation prediction for each peptide with respect to the target antigen, wherein the presentation prediction represents the likelihood of the candidate peptide binding to an MHC-I protein expressed in a mono-allelic cell line. The presentation prediction is an output of an ensemble presentation prediction model combining the presentation prediction output of each of a plurality of machine learning HLA-peptide presentation prediction models to provide a single presentation prediction for each of a plurality of candidate peptides with respect to the target antigen. The peptide members can be the 8-14mer peptides of Table A. The presentation prediction models can be as described in Section I herein above.

Using the peptide library would accelerate the identification of targets for T cell vaccine development, based on their predictive binding, expression, and sequence conservation in isolates. As used herein, the term “library” refers to a collection of entities, such as, for example, peptides. A library may comprise at least two, at least three, at least four, at least five, at least ten, at least 25, at least 50, at least 102, at least 103, at least 104, at least 105, at least 106, at least 107, at least 108, at least 109, or more different entities (e.g., peptides, nucleic acids). Libraries provided herein comprise a plurality of library members and libraries of nucleic acid compositions each encoding peptide member of the library. In some aspects, a library refers to a collection of nucleic acids that are propagatable, e.g., through a process of clonal amplification. Library entities can be stored, maintained, or contained separately or as a mixture.

In some aspects, the target antigen is SARS-CoV2. When the target antigen is SARS-CoV2, the library comprises all potential 8-14mer target antigen peptides restricted to 52 common MHC-I alleles. In some aspects, the peptide library comprises the 8-14mer peptides of Table A.

### VI. Vaccine Composition

An additional aspect of the present disclosure provides a vaccine composition comprising a polypeptide comprising one or more peptide sequences identified using a method for selecting peptides described in Section I above. A vaccine composition generally comprises an adjuvant. Adjuvants, such as aluminum hydroxide or aluminum phosphate, can be added to the vaccine composition to further increase the ability of the vaccine to trigger, enhance, or prolong an immune response. The vaccine compositions may further comprise additional components known in the art to improve the immune response to a vaccine, such as T cell co-stimulatory molecules or antibodies, such as anti-CTLA4. Additional materials, such as cytokines, chemokines, and bacterial nucleic acid sequences naturally found in bacteria, like CpG, are also potential vaccine adjuvants. In an aspect, a vaccine composition of the invention further comprises alum adjuvant in addition to peptide adjuvants of the invention.

A vaccine can comprise a pharmaceutically acceptable carrier or excipient. Such a carrier may be any solvent or solid material for encapsulation that is non-toxic to the inoculated host and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients, as well as formulations for parenteral and nonparenteral drug delivery, are set forth in Remington's Pharmaceutical Sciences 19th Ed. Mack Publishing (1995). When used for administering via the bronchial tubes, the vaccine is preferably presented in the form of an aerosol.

A vaccine composition comprising a peptide antigen of the invention can optionally comprise one or more possible additives, such as carriers, preservatives, stabilizers, adjuvants in addition to peptide adjuvants of the invention, and other substances.

The dosages of a vaccine composition of the invention can and will vary depending on the adjuvant composition, the peptide antigen, the pathogen, and the intended host, as will be appreciated by one of skill in the art. Generally speaking, the dosage need only be sufficient to elicit a protective immune response in a majority of hosts. Routine experimentation may readily establish the required dosage. Administering multiple dosages may also be used as needed to provide the desired level of protective immunity.

A vaccine composition of the invention may also be a commercially available vaccine composition, wherein the commercially available vaccine composition is supplemented with a peptide antigen of the disclosure.

When the target antigen is SARS-CoV2, a vaccine composition can be prepared, wherein the composition comprises a peptide comprising any one or more of the library member peptide sequences described herein above in Section IV, or a polynucleotide encoding the polypeptide.

### VII. Methods of Treating

An additional aspect of the present disclosure provides methods of treating or preventing a viral infection in a subject in need thereof. The method comprises administering to the subject a vaccination composition described in Section VI herein above. wherein the target antigen is a viral antigen. Antigens can be as described in Section V herein above.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Total Predicted MHC-1 Epitope Load is Inversely Associated with Population Mortality from SARS-CoV-2

### Summary

Polymorphisms in MHC-I protein sequences across human populations significantly affect viral peptide binding capacity, and thus alter T cell immunity to infection. In the present study, the relationship between observed SARS-CoV-2 population mortality and the predicted viral binding capacities of 52 common MHC-I alleles was assessed. Potential SARS-CoV-2 MHC-I peptides are identified using a consensus MHC-I binding and presentation prediction algorithm called EnsembleMHC. Starting with nearly 3.5 million candidates, a few hundred highly probable MHC-I peptides were resolved. By weighing individual MHC allele-specific SARS-CoV-2 binding capacity with population frequency in 23 countries, a strong inverse correlation between predicted population SARS-CoV-2 peptide binding capacity and mortality rate was observed. The computations reveal that peptides derived from the structural proteins of the virus produce a stronger association with observed mortality rate, highlighting the importance of S, N, M, and E proteins in driving productive immune responses.

### Introduction

In December 2019, the novel coronavirus, severe acute respiratory syndrome-coronavirus-2 (SARS-CoV-2) was identified from a cluster of cases of pneumonia in Wuhan, China. With >73.1 million cases and >1.6 million deaths, the viral spread was declared a global pandemic by the World Health Organization. Due to its high rate of transmission and unpredictable severity, there is an immediate need for information surrounding the adaptive immune response toward SARS-CoV-2.

A robust T cell response is integral for the clearance of corona-viruses and the generation of lasting immunity. The potential role of T cells for coronavirus clearance has been supported by the identification of immunogenic CD8+ T cell epitopes in the S (Spike), N (Nucleocapsid), M (Membrane), and E (Envelope) proteins. In addition, SARS-CoV-specific CD8+-T cells have been shown to provide long-lasting immunity, with memory CD8+T cells being detected up to 17 years post-infection. The specifics of the T cell response to SARS-CoV-2 is still evolving. However, a recent screening of SARS-CoV-2 peptides revealed that a majority of the CD8+-T cell immune response is targeted toward viral structural proteins (N, M, S).

A successful CD8+-T cell response is contingent on the efficient presentation of viral protein fragments by major histocompatibility complex I (MHC-I) proteins. MHC-I molecules bind and present peptides derived from endogenous proteins on the cell surface for CD8+-T cell interrogation. The MHC-I protein is highly polymorphic, with amino acid substitutions within the peptide binding groove drastically altering the composition of presented peptides. Consequently, the influence of MHC genotype to shape patient outcome has been well studied in the context of viral infections. For coronaviruses, there have been several studies of the association of MHC with disease susceptibility. A study of a Taiwanese and Hong Kong cohort of patients with SARS-CoV found that the MHC-I alleles HLA (histocompatibility leukocyte antigen)-B*07:03 and HLA-B*46:01 were linked to increased susceptibility, while HLA-Cw*15:02 was linked to increased resistance. However, some of the reported associations did not remain after statistical correction, and it is still unclear whether MHC-outcome associations reported for SARS-CoV are applicable to SARS-CoV-2. Recently, a comprehensive prediction of SARS-CoV-2 MHC-I peptides indicated a relative depletion of high-affinity binding peptides for HLA-B*46:01, hinting at a similar association profile in SARS-CoV-2. More important, it remains elusive whether such a depletion of putative high-affinity peptides affect patient outcomes to SARS-CoV-2 infections.

The lack of large-scale genomic data linking individual MHC genotypes and outcomes from SARS-CoV-2 infections precludes a similar analysis as performed for SARS-CoV. Therefore, the inventors endeavored to assess the relationship between the predicted SARS-CoV-2 binding capacity of a population and the observed SARS-CoV-2 mortality rate. Historically, MHC-I prediction algorithms have been characterized by a high false positive rate, particularly when predicting peptides that are naturally presented. To minimize false positives and identify the highest-confidence SARS-CoV-2 MHC-I peptides, a consensus algorithm, called EnsembleMHC, and predicted MHC-I peptides for a panel of 52 common MHC-I alleles was developed. This prediction workflow integrates seven different algorithms that have been parameterized on high-quality mass spectrometry data and provides a confidence level for each identified peptide. The distribution of the number of high-confidence peptides assigned to each allele was used to assess a country-specific SARS-CoV-2 binding capacity, called the EnsembleMHC population (EMP) score, for 23 countries (for selection criteria, please refer to the STAR Methods). This score was derived by weighing the individual binding capacities of the 52 MHC-I alleles by their endemic frequencies. A strong inverse correlation was noted between the EMP score and observed population SARS-CoV-2 mortality. Furthermore, the correlation is demonstrated to become stronger when considering EMP scores based solely on SARS-CoV-2 structural proteins, underlining their potential importance in driving a robust immune response. Based on their predicted binding affinity, expression, and sequence conservation in viral isolates, 108 peptides derived from SARS-CoV-2 structural proteins were identified that are high-value targets for CD8+-T cell vaccine development (see peptide sequences in bold in Table A).

### Results

EnsembleMHC Workflow Offers More Precise MHC-I Presentation Predictions than Individual Algorithms

The accurate assessment of differences in SARS-CoV-2 binding capacities across MHC-I allelic variants requires the isolation of MHC-I peptides with a high probability of being presented. EnsembleMHC provides the requisite precision through the use of allele- and algorithm-specific score thresholds and peptide confidence assignment.

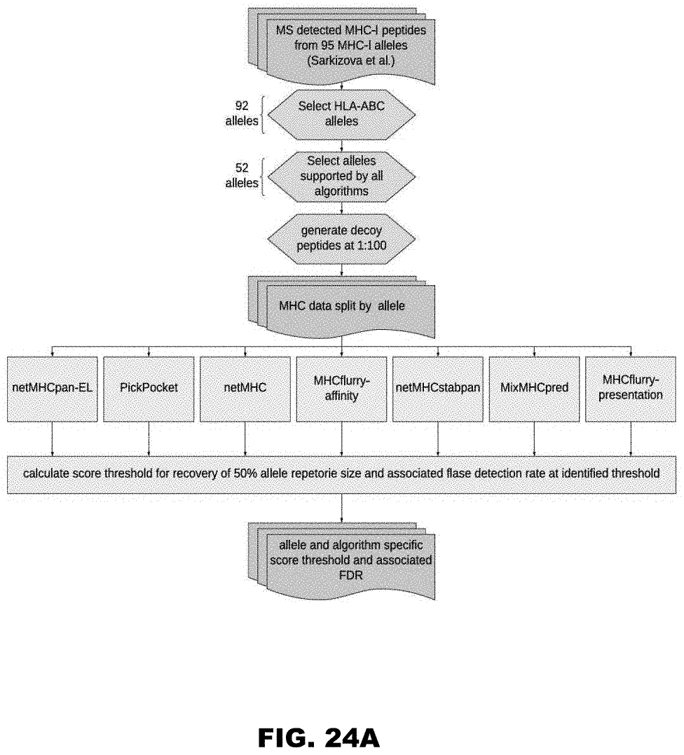

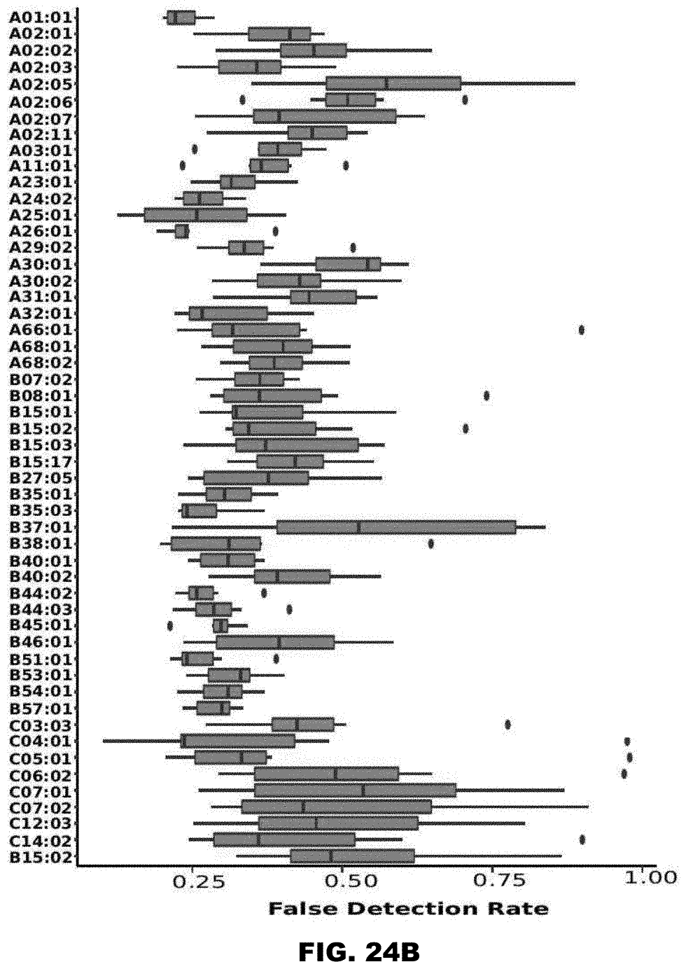

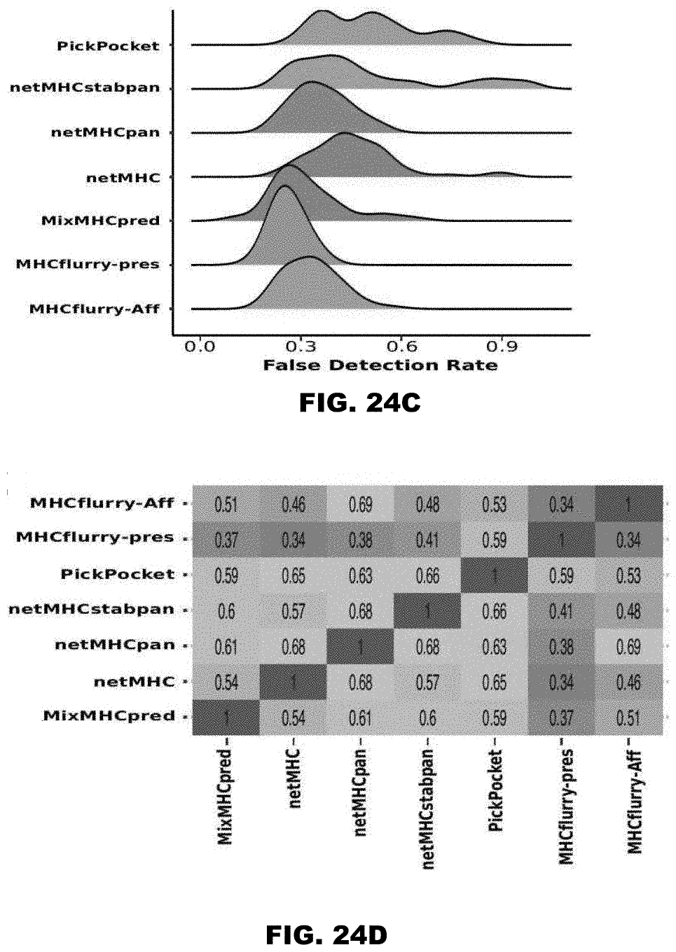

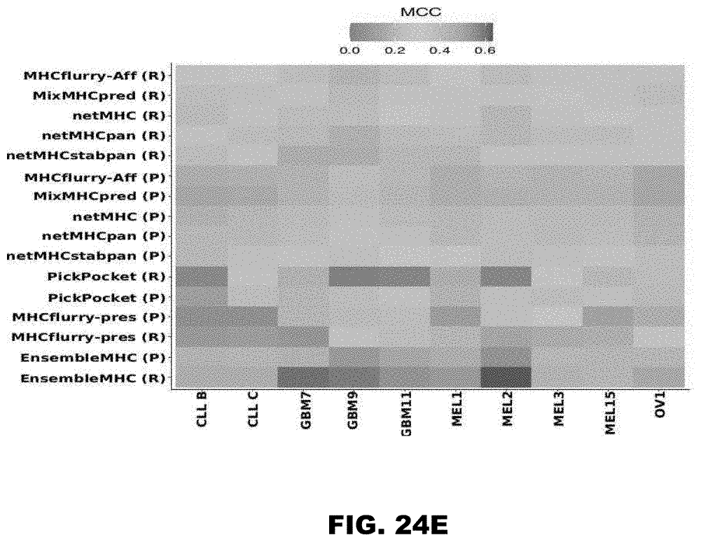

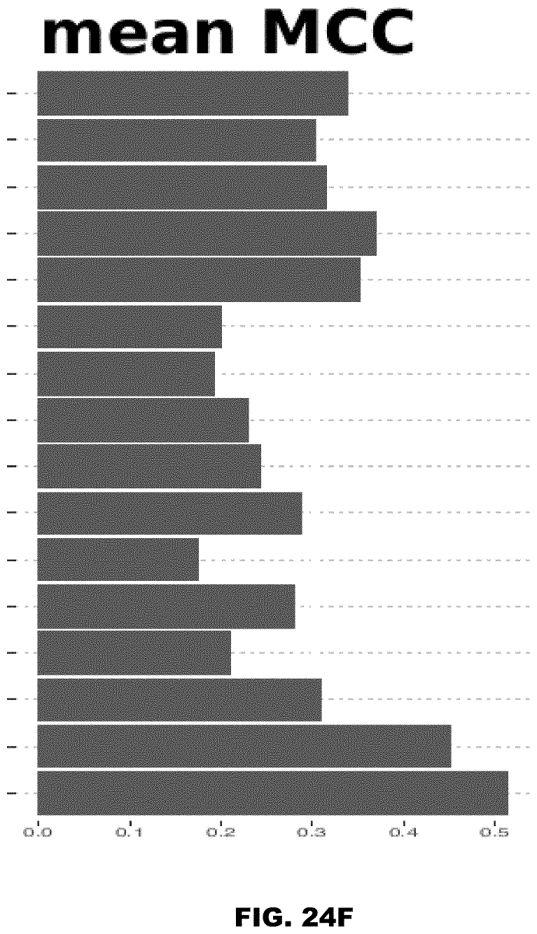

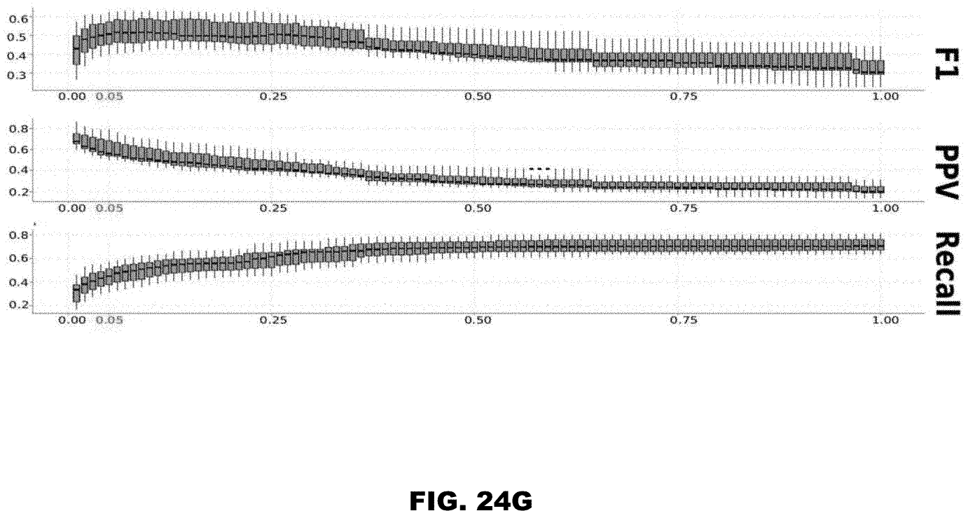

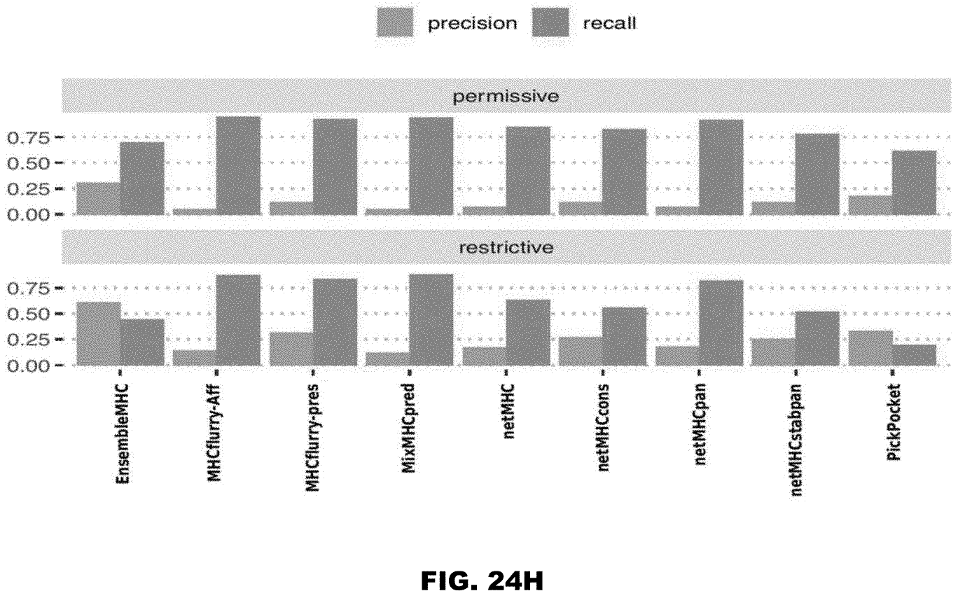

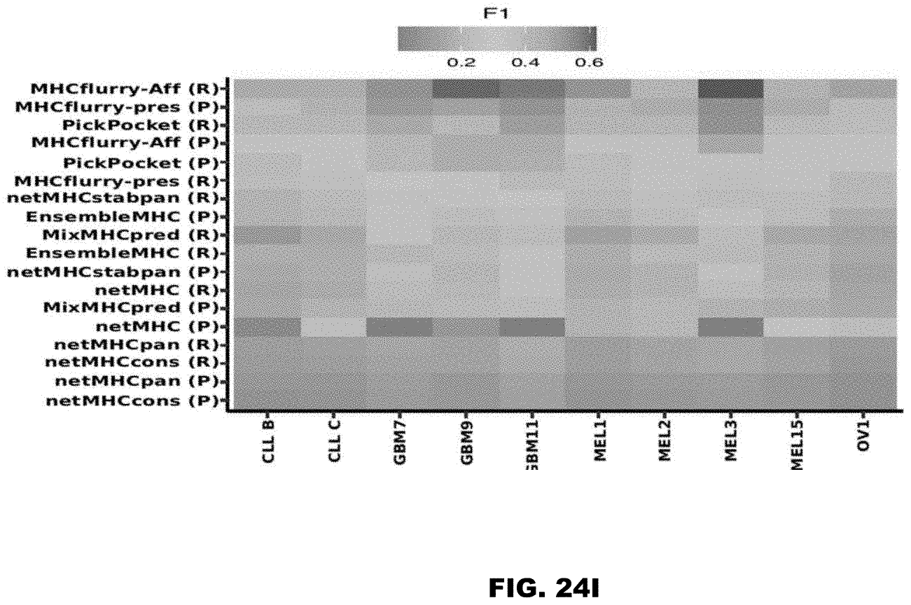

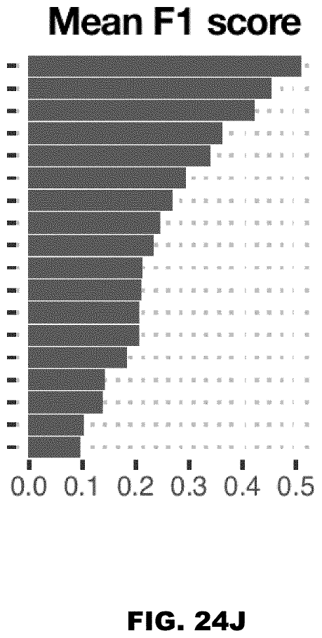

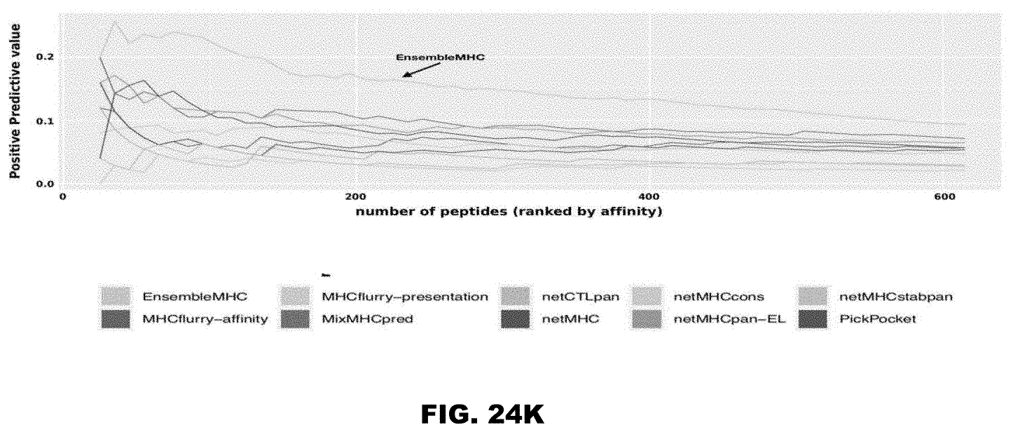

MHC-I alleles substantially vary in both peptide binding repertoire size and median binding affinity. The EnsembleMHC workflow addresses this inter-allele variation by identifying peptides based on MHC allele- and algorithm-specific binding affinity thresholds. These thresholds were set by benchmarking each of the 7 component algorithms against 52 single MHC allele peptide datasets. Each dataset consists of mass spectrometry-confirmed MHC-I peptides that have been naturally presented by a model cell line expressing 1 of the 52 select MHC-I alleles. These experimentally validated peptides, denoted target peptides, were supplemented with a 100-fold excess of decoy peptides. Decoys were generated by randomly sampling peptides that were not detected by mass spectrometry, but were derived from the same protein sources as a detected target peptide. Algorithm- and allele-specific binding affinity thresholds were then identified through the independent application of each component algorithm to all of the MHC allele datasets. For every dataset and algorithm combination, the target and decoy peptides were ranked by predicted binding affinity to the MHC allele defined by that dataset. Then, an algorithm-specific binding affinity threshold was set to the minimum score needed to isolate the highest affinity peptides commensurate to 50% of the observed allele repertoire size (FIG. 24A). The observed allele repertoire size was defined as the total number of target peptides within a given single MHC allele dataset. Therefore, if a dataset had 1,000 target peptides, the top 500 highest affinity peptides would be selected, and the algorithm-specific threshold would be set to the predicted binding affinity of the 500th peptide. This parameterization method resulted in the generation of a customized set of allele- and algorithm-specific binding affinity thresholds in which an expected quantity of peptides can be recovered.

Consensus MHC-I prediction typically require a method for combining outputs from each individual component algorithm into a composite score. This composite score is then used for peptide selection. EnsembleMHC identifies high-confidence peptides based on filtering by a quantity called peptide^{FDR} (Equation 1; see below). During the identification of allele- and algorithm-specific binding affinity thresholds, the empirical false detection rate (FDR) of each algorithm was calculated. This calculation was based on the proportion of target to decoy peptides isolated by the algorithm-specific binding affinity threshold. A peptide^{FDR} is then assigned to each individual peptide by taking the product of the empirical FDRs of each algorithm that identified that peptide for the same MHC-I allele. Analysis of the parameterization process revealed that the overall performance of each included algorithm was comparable, and there was diversity in individual peptide calls by each algorithm, supporting an integrated approach to peptide confidence assessment (FIGS. 24B-24D). Peptide identification by EnsembleMHC was performed by selecting all of the peptides with a peptide^{FDR}≤5%.

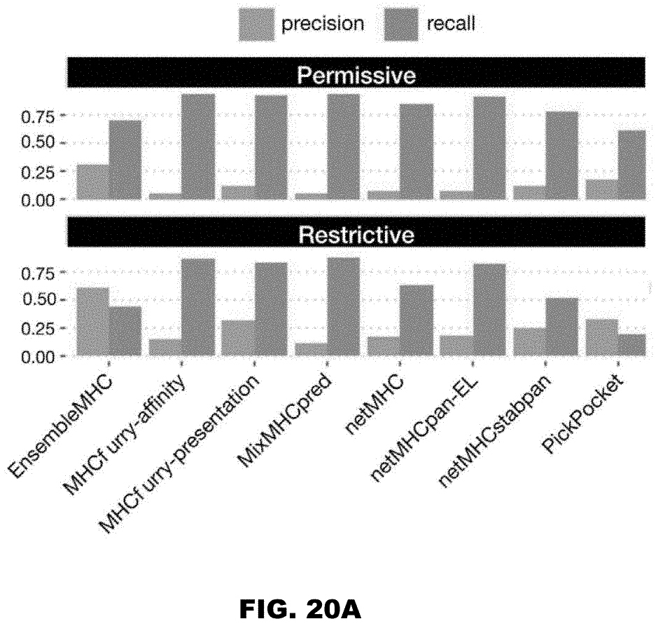

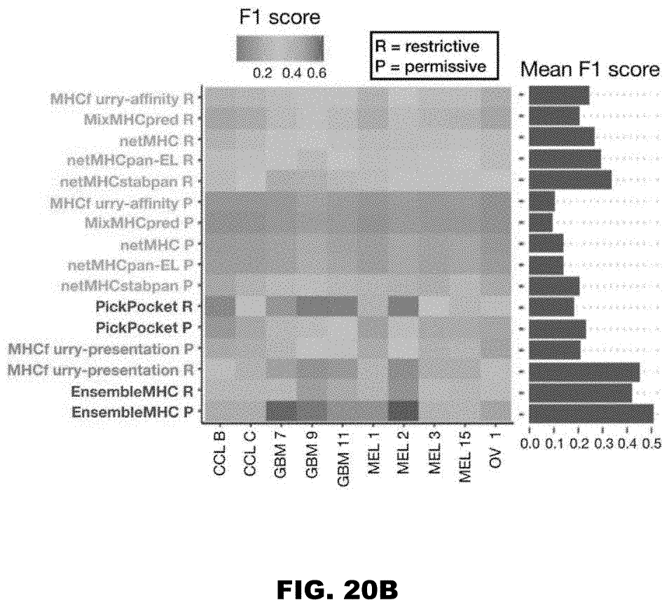

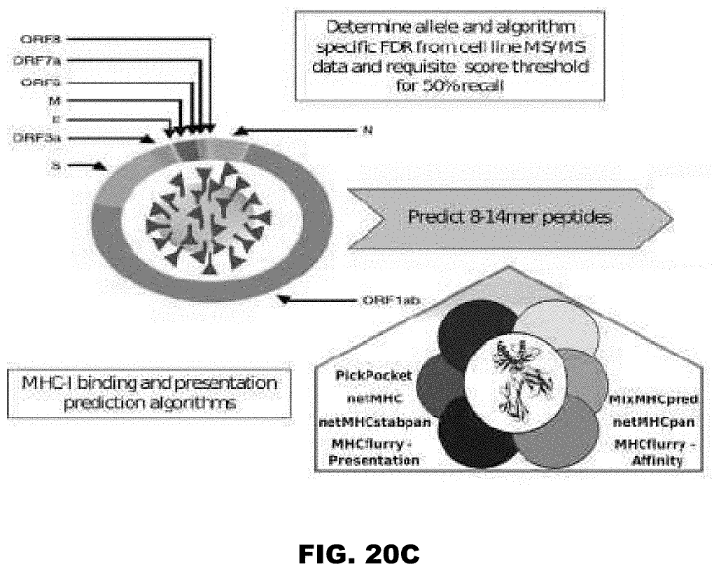

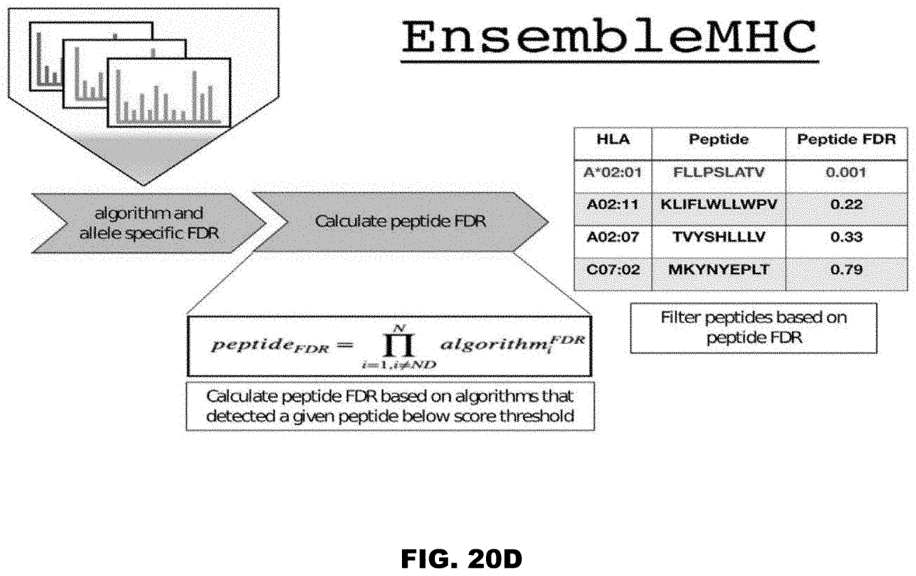

The efficacy of peptide^{FDR} as a filtering metric was determined through the prediction of naturally presented MHC-I peptides derived from 10 tumor samples (FIGS. 20A-20C). Similar to the single MHC allele datasets, each tumor sample dataset consisted of mass spectrometry-detected target peptides and a 100-fold excess of decoy peptides. The performance of EnsembleMHC was assessed via comparison with individual component algorithms. Peptide identification by each algorithm was based on a restrictive or permissive binding affinity threshold (FIG. 20A). For the component algorithms, the permissive and restrictive thresholds correspond to commonly used binding affinity cutoffs for the identification of weak and strong binders, respectively. The performance of each algorithm on the 10 datasets was evaluated through the calculation of the empirical precision, recall, and F1 score.

The average precision and recall of each algorithm across all tumor samples demonstrated an inverse relationship (FIG. 20A). In general, restrictive binding affinity thresholds produced higher precision at the cost of poorer recall. When comparing the precision of each algorithm at restrictive thresholds, EnsembleMHC demonstrated a 3.4-fold improvement over the median precision of individual component algorithms. EnsembleMHC also produced the highest F1 score, with an average of 0.51, followed by MHCflurry-presentation, with an F1 score of 0.45, both of which are 1.5- to 2-fold higher than the rest of the algorithms (FIG. 20B). This result was shown to be robust across a range of peptide FDR cutoff thresholds (FIG. 24E), alternative performance metrics (FIG. 24F), and other consensus-based prediction algorithms (FIGS. 24G and 24H). Furthermore, EnsembleMHC demonstrated the ability to more efficiently prioritize peptides with experimentally established immunogenicity from the Hepatitis-C genome polyprotein, the Dengue virus genome polyprotein, and the HIV-1 POL-GAG protein (FIG. 24I). These results demonstrate the enhanced precision of EnsembleMHC over individual component algorithms when using common binding affinity thresholds.

In summary, the EnsembleMHC workflow offers two desirable features. First, it determines allele-specific binding affinity thresholds for each algorithm at which a known quantity of peptides is expected to be successfully presented on the cell surface. Second, it assigns a confidence level to each peptide call made by each algorithm. These traits enhance the ability to identify MHC-I peptides with a high probability of successful cell surface presentation.

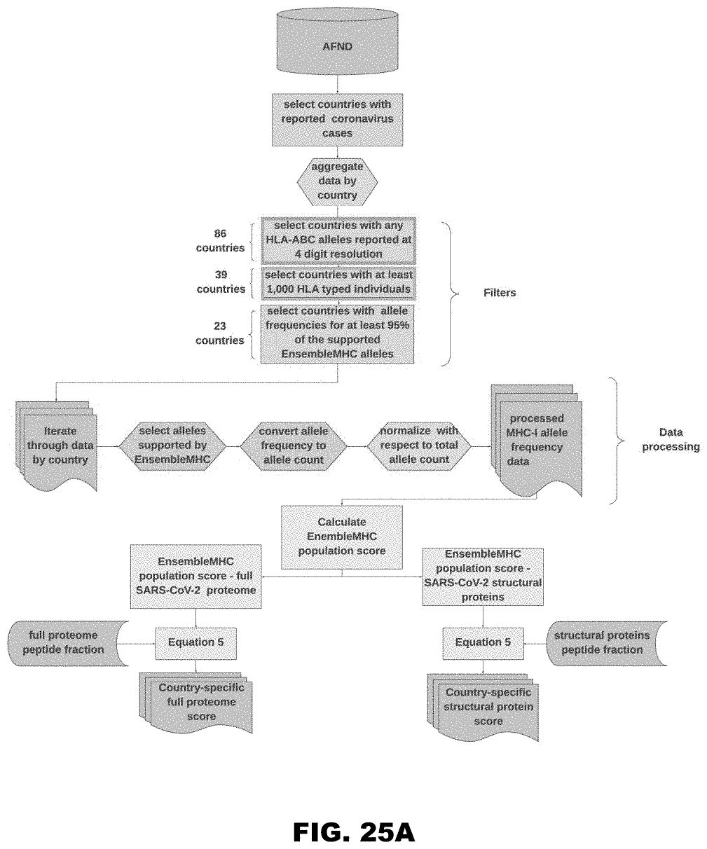

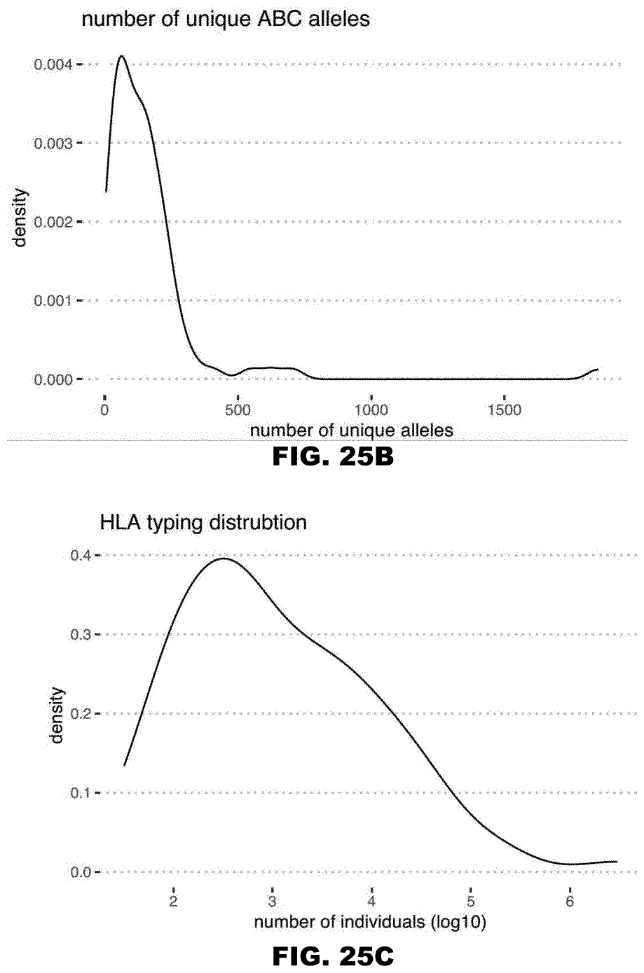

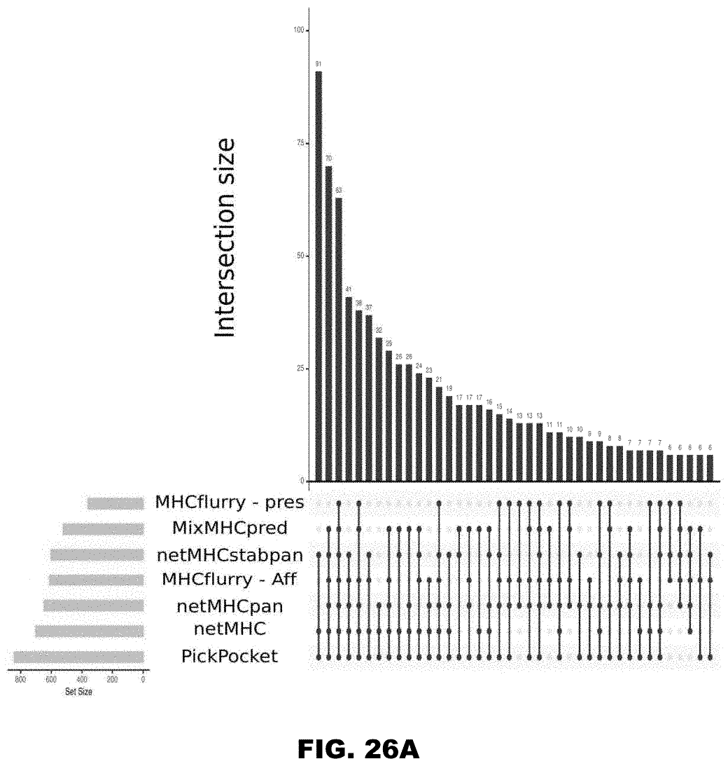

EnsembleMHC was used to identify MHC-I peptides for the SARS-CoV-2 virus (FIG. 20C). The resulting identification of high-confidence SARS-CoV-2 peptides allows for the characterization of alleles that are enriched or depleted for predicted MHC-I peptides. The resulting distribution of allele-specific SARS-CoV-2 binding capacities will then be weighed by the normalized frequencies of the 52 alleles (FIG. 25; Equation 5 and Equation 6; see below) in 23 countries to determine the population-specific SARS-CoV-2 binding capacity or EMP score (Equation 7; see below). The potential impact of varying population SARS-CoV-2 binding capacities on disease out-comes can then be assessed by correlating population SARS-CoV-2 mortality rates with EMP scores.

### The MHC-1 Peptide-Allele Distribution for SARS-CoV-2 Structural Proteins is Especially Disproportionate.

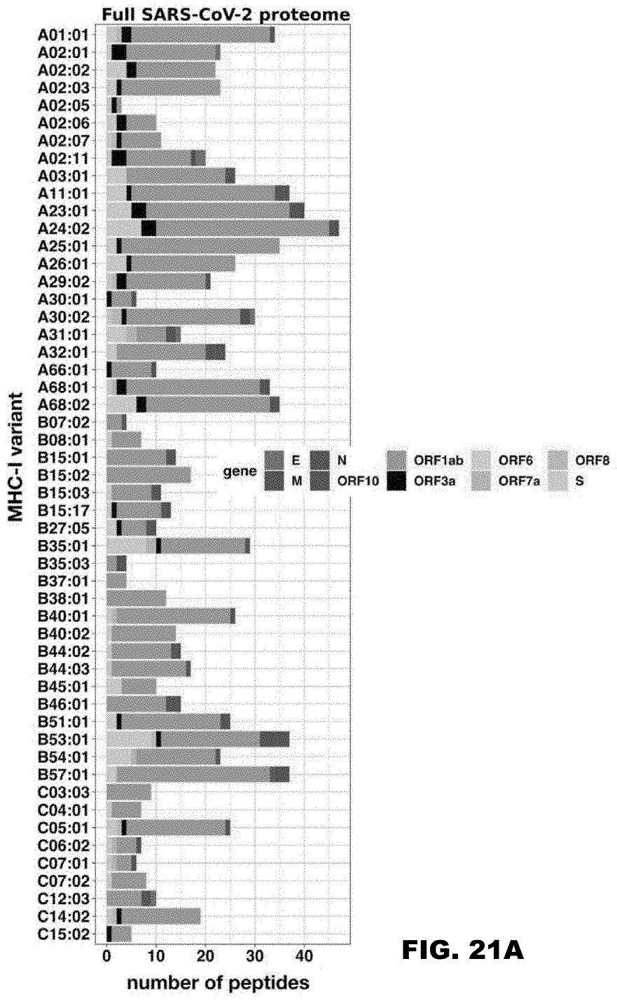

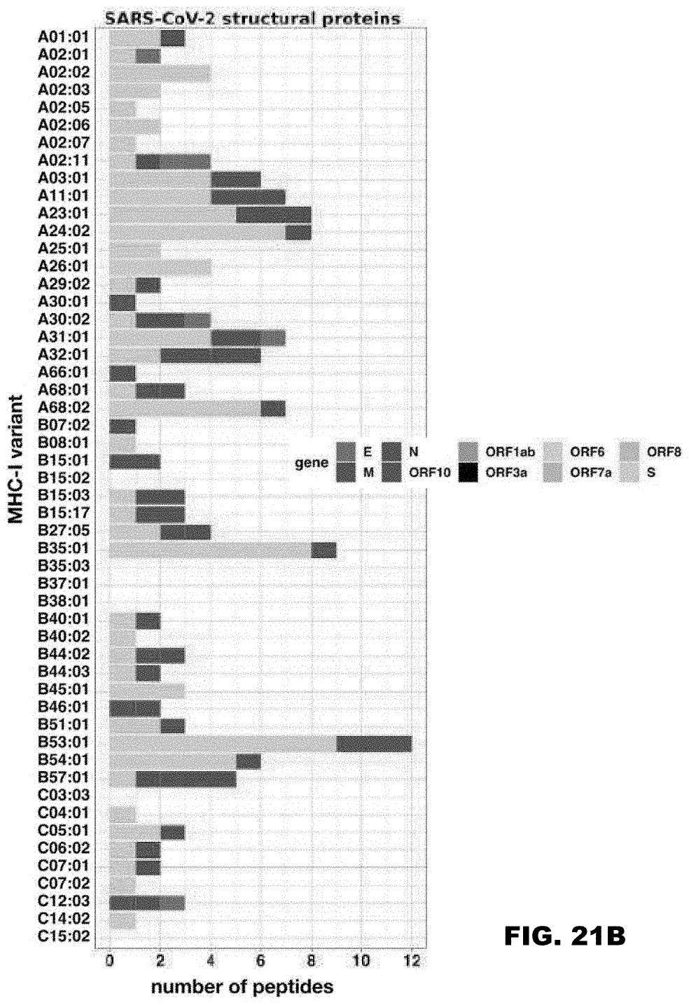

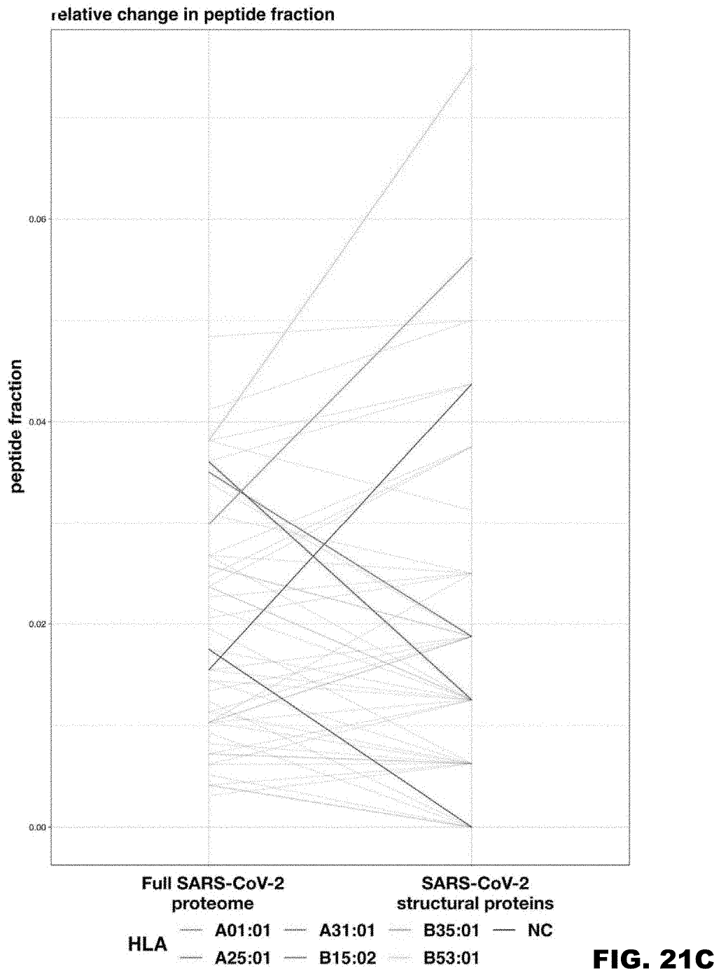

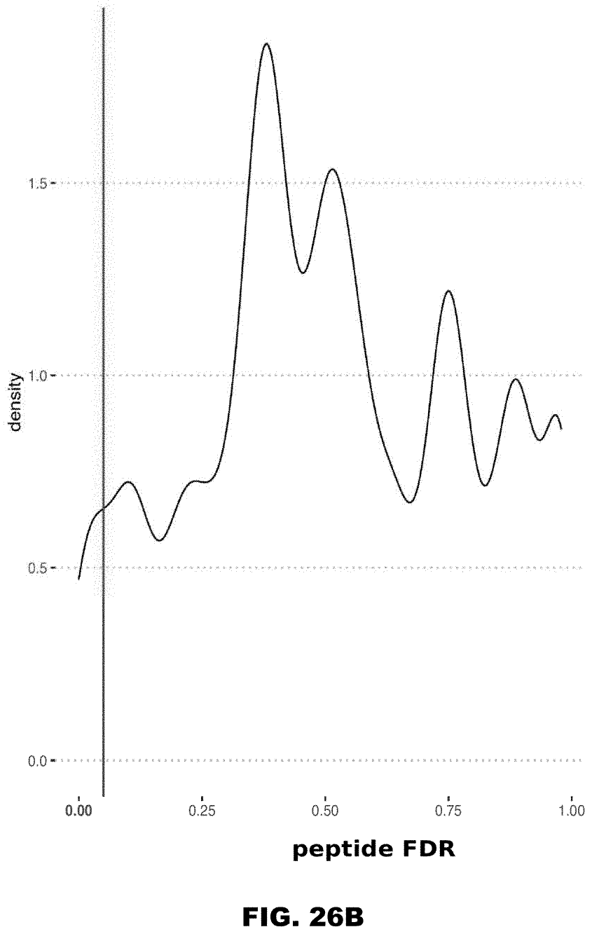

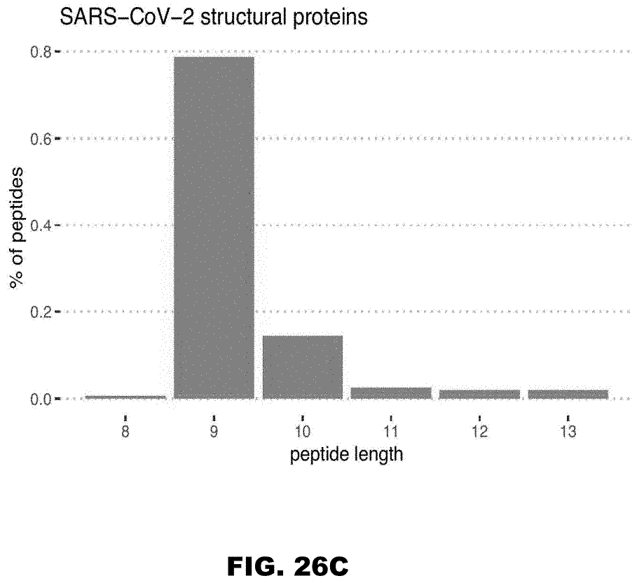

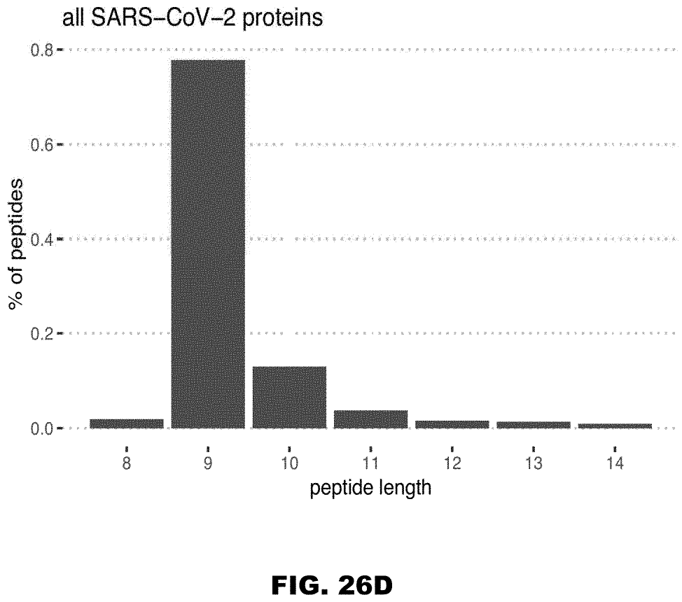

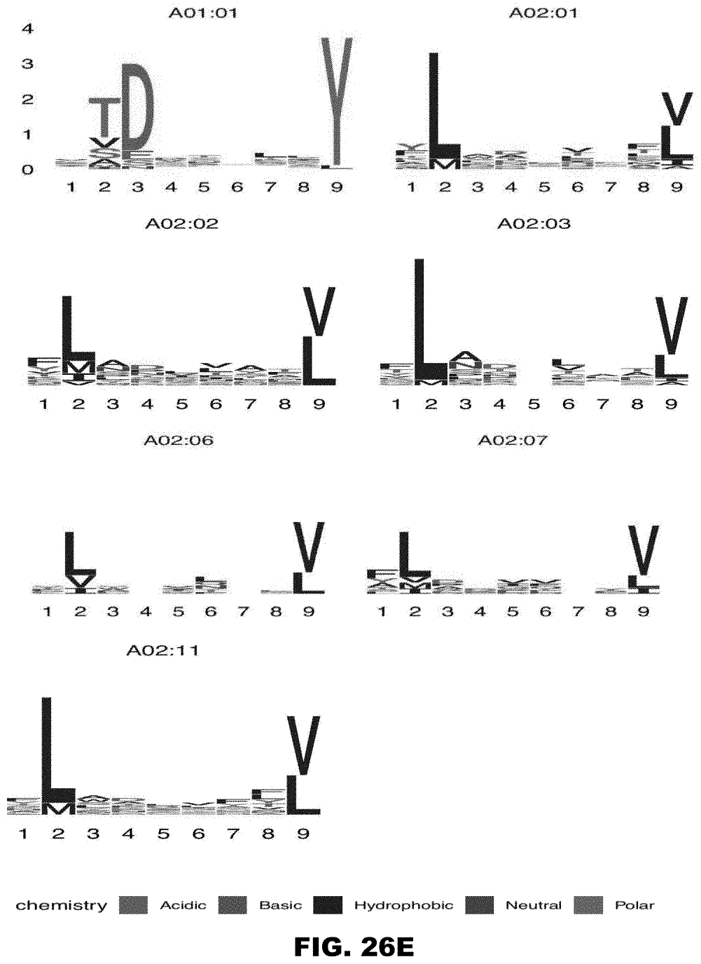

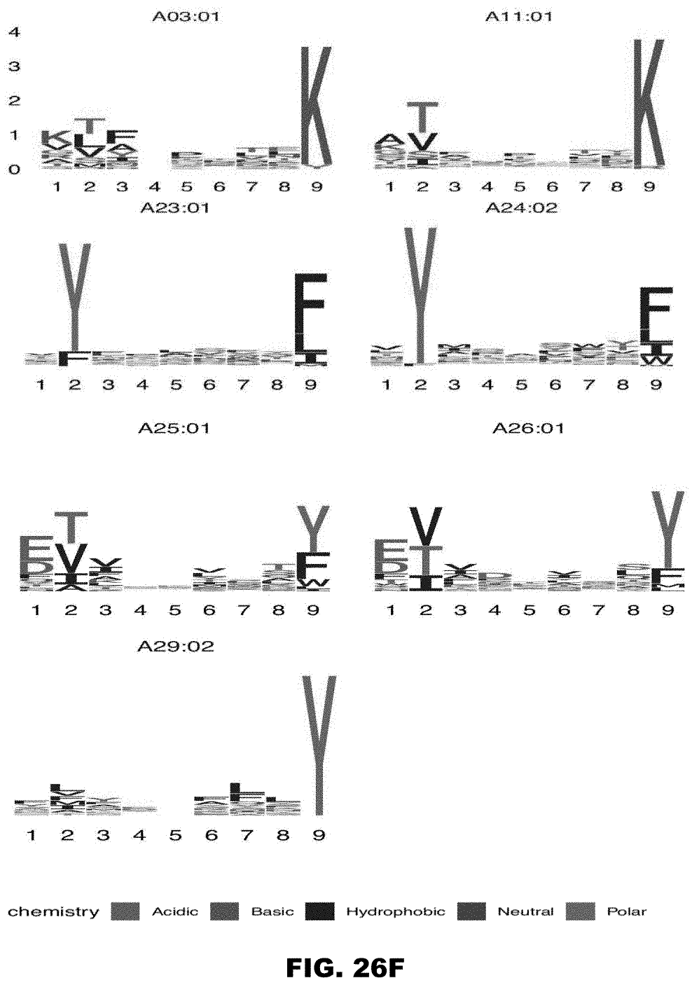

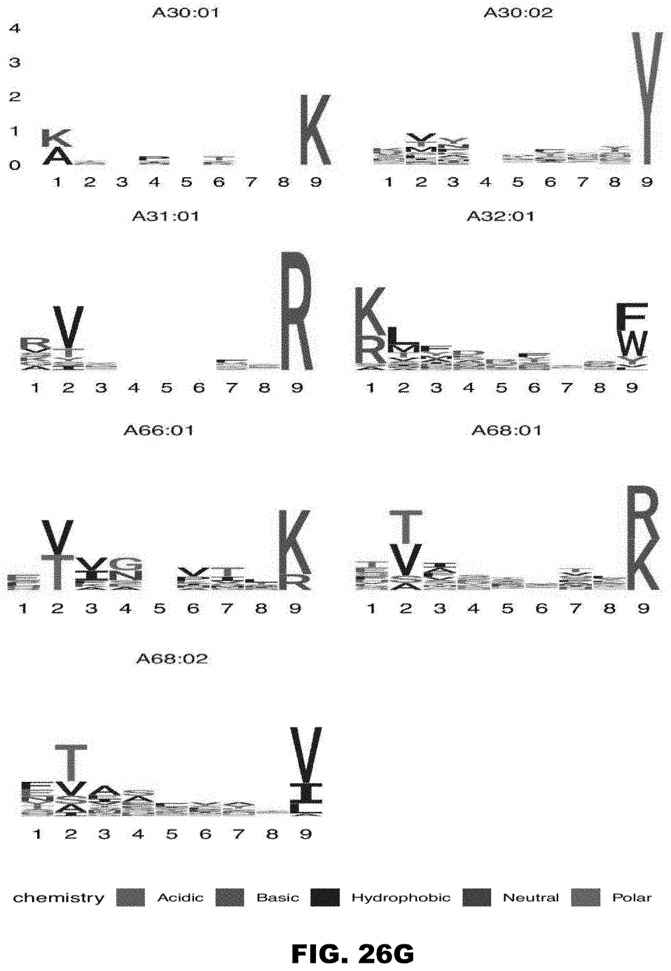

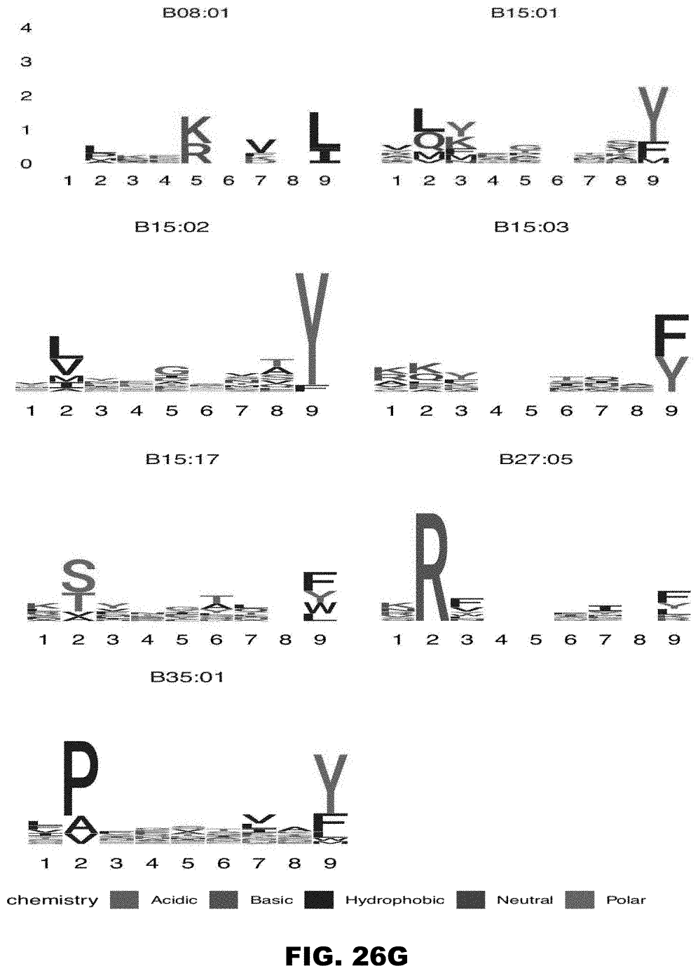

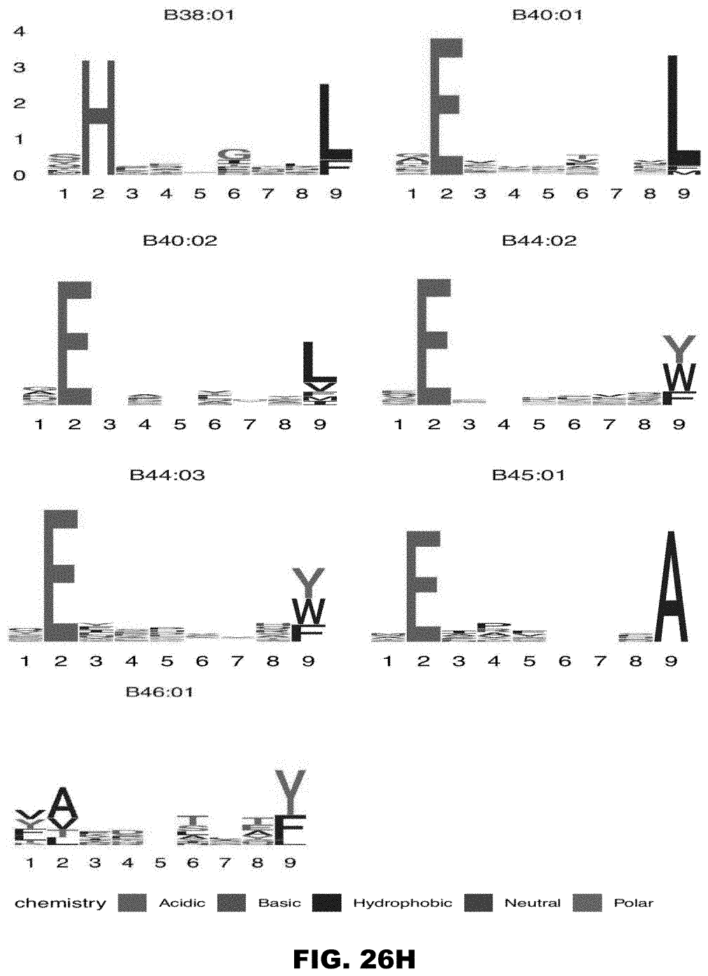

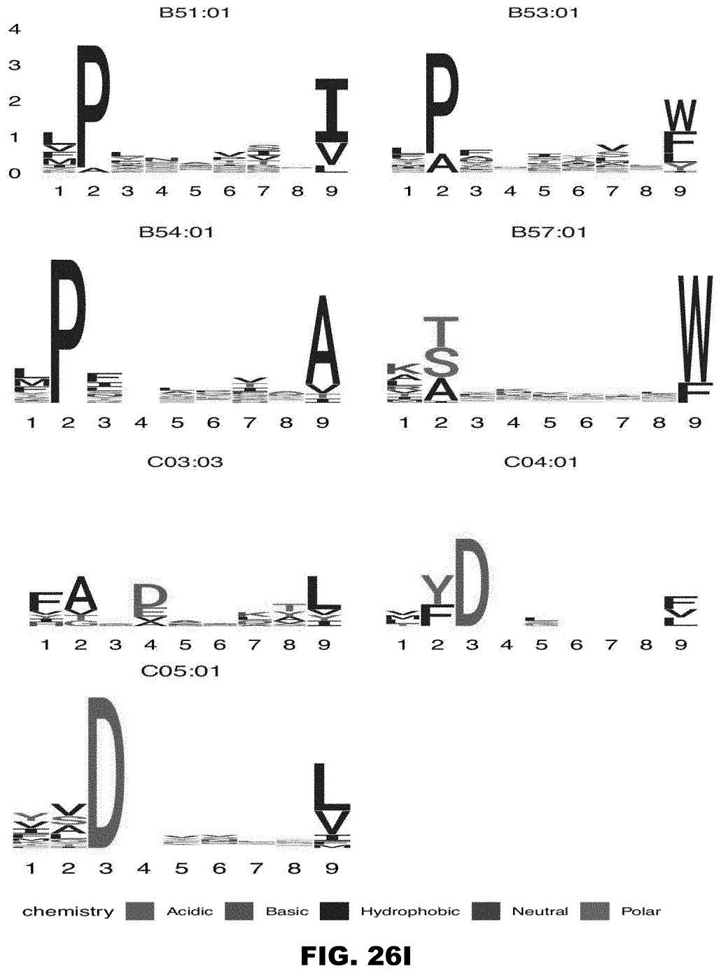

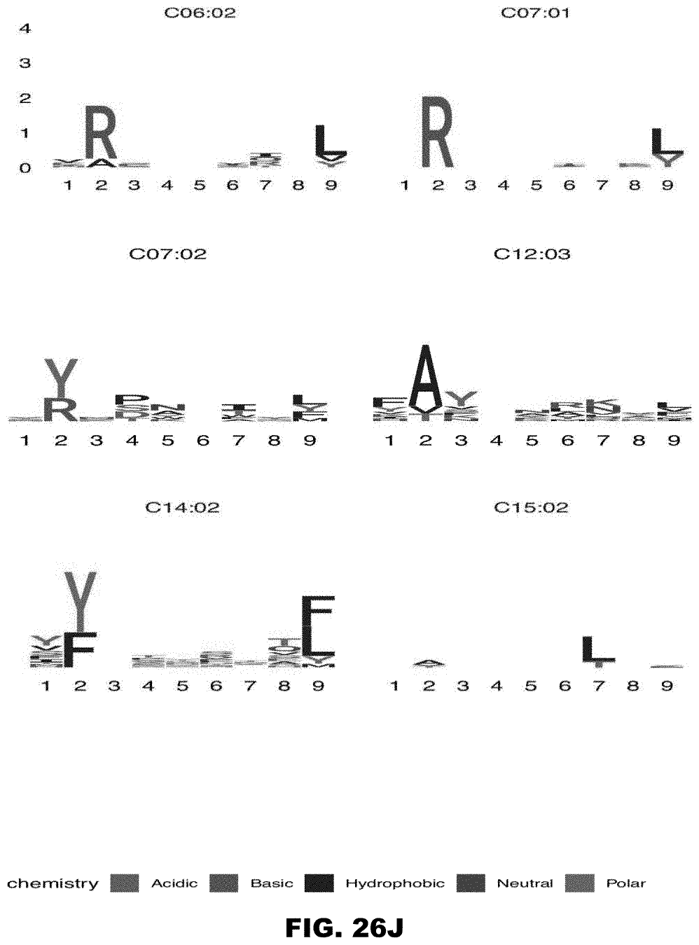

MHC-I peptides derived from the SARS-CoV-2 proteome were predicted and prioritized using EnsembleMHC. A total of 67,207 potential 8- to 14-mer viral peptides were evaluated for each of the considered MHC-I alleles. After filtering the pool of candidate peptides at the 5% peptide^{FDR} threshold, the number of potential peptides was reduced from 3.49 million to 971 (658 unique peptides) (FIGS. 26A and 26B, Table A). Illustrated in FIG. 21A, the viral peptide-MHC allele (or peptide-allele) distribution for high-confidence SARS-CoV-2 peptides was determined by assigning the identified peptides to their predicted MHC-I alleles. There was a median of 16 peptides per allele, with a maximum of 47 peptides (HLA-A*24:02), a minimum of 3 peptides (HLA-A*02:05), and an interquartile range (IQR) of 16 peptides. Quality assurance of the predicted peptides was performed by computing the peptide length frequencies and binding motifs. The predicted peptides were found to adhere to expected MHC-I peptide lengths28, with 78% of the peptides being 9 amino acids in length, 13% being 10 amino acids in length, and 8% of peptides accounting for the remaining lengths (FIGS. 26C and 26D). Similarly, logo plots generated from predicted peptides were found to closely reflect reference peptide binding motifs for considered alleles (FIG. 26E). Overall, the EnsembleMHC prediction platform demonstrated the ability to isolate a short list of potential peptides that adhere to expected MHC-I peptide characteristics.

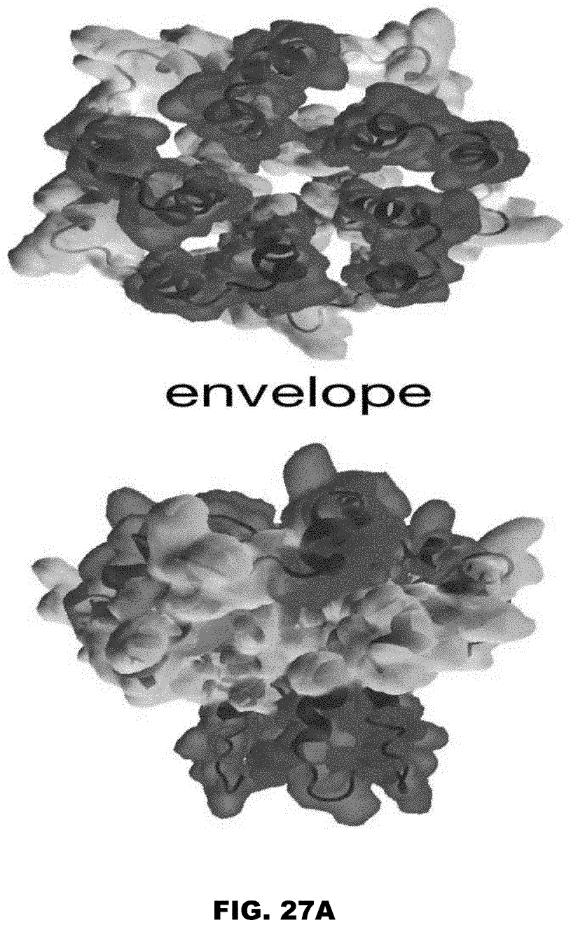

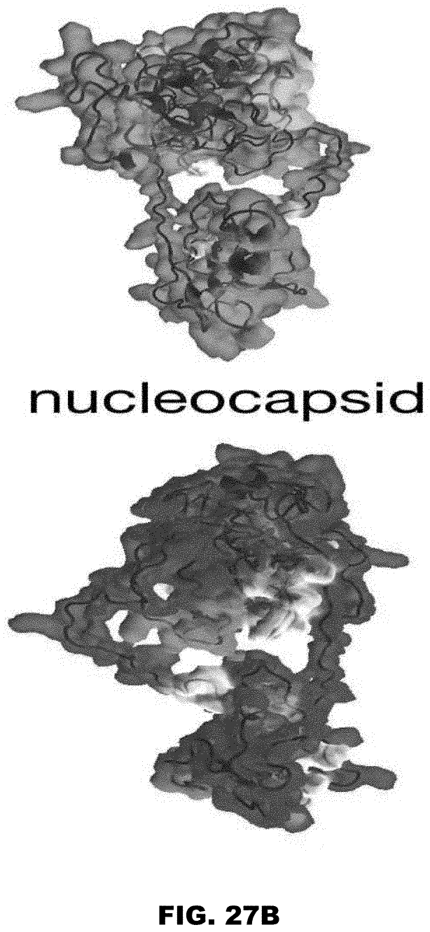

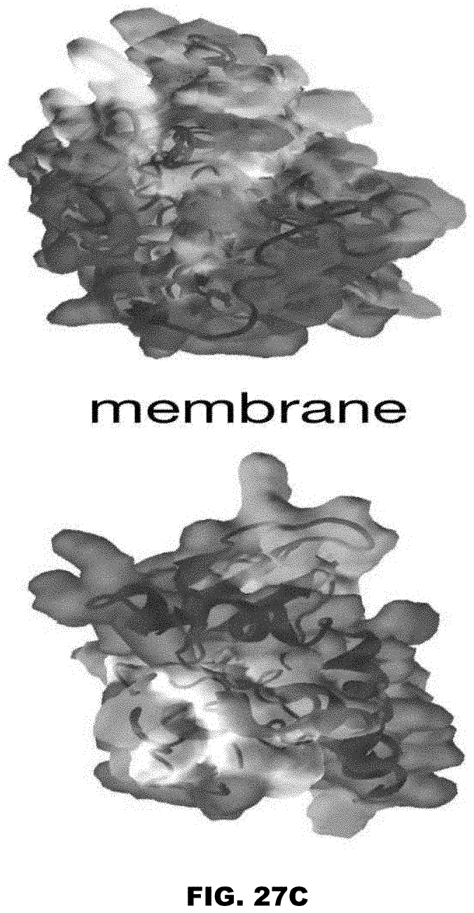

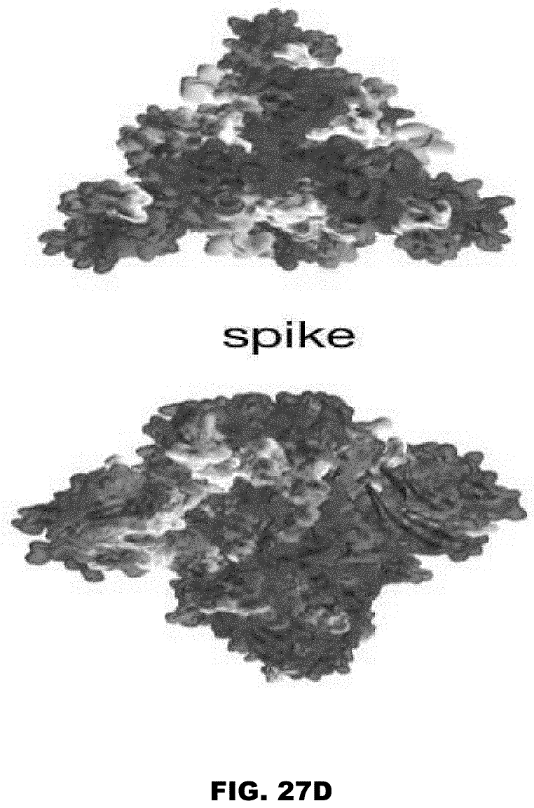

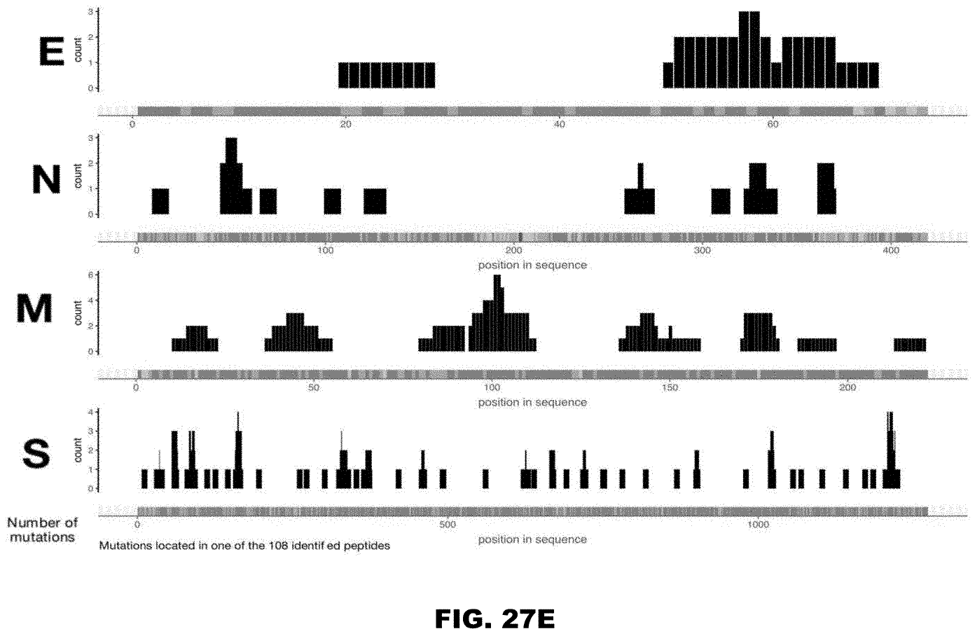

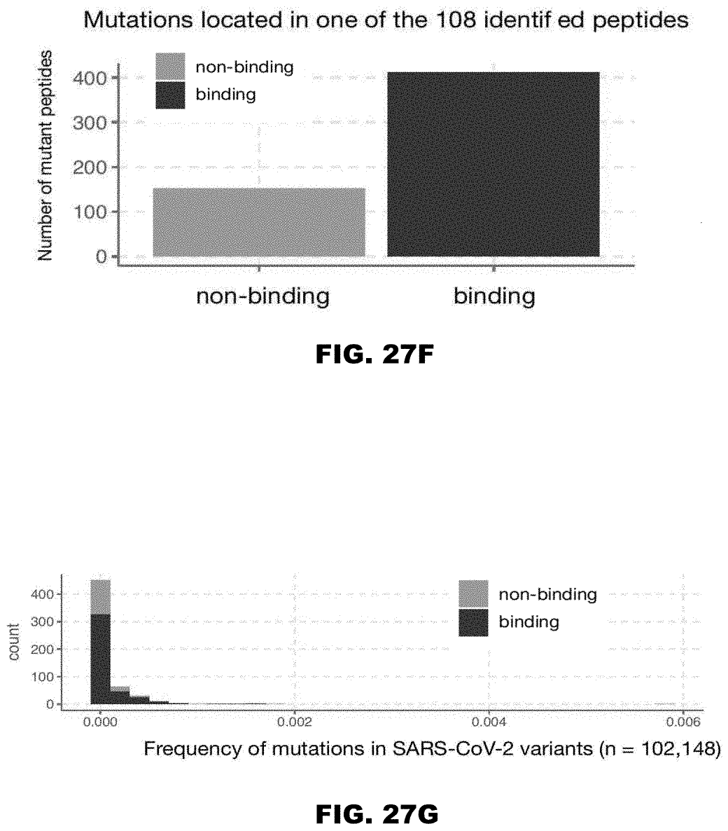

The high expression, relative conservation, and reduced search space of SARS-CoV-2 structural proteins (S, E, M, and N) make MHC-I binding peptides derived from these proteins high-value targets for CD8+-T cell-based vaccine development. FIG. 21B describes the peptide-allele distribution for predicted MHC-I peptides originating from the four structural proteins. This analysis markedly reduces the number of considered peptides from 658 to 108 (Table A). The median number of predicted SARS-CoV-2 structural peptides assigned to each MHC-I allele was found to be 2, with a maximum of 12 peptides (HLAB*53:01), a minimum of 0 peptides (HLA-B*15:02, B*35:03, B*38:01, C*03:03, C*15:02), and an IQR of 3 peptides. Analysis of the molecular source of the identified SARS-CoV-2 structural protein peptides revealed that they originate from enriched regions that are highly conserved (FIGS. 27A and 27B). This indicates that such peptides would be ideal candidates for targeted therapies as they are unlikely to be disrupted by mutation, and several peptides can be targeted using minimal stretches of the source protein. Consideration of the MHC-I peptides derived only from SARS-CoV-2 structural proteins reduces the number of potential peptides to a condensed set of high-value targets that is amenable to experimental validation.

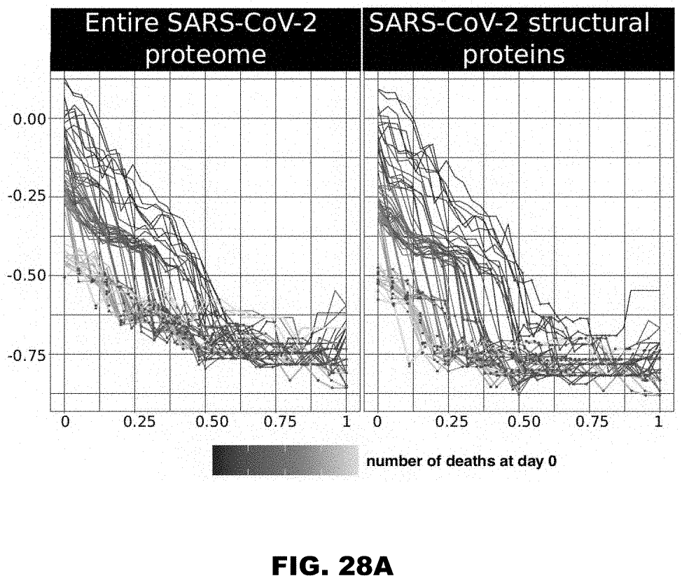

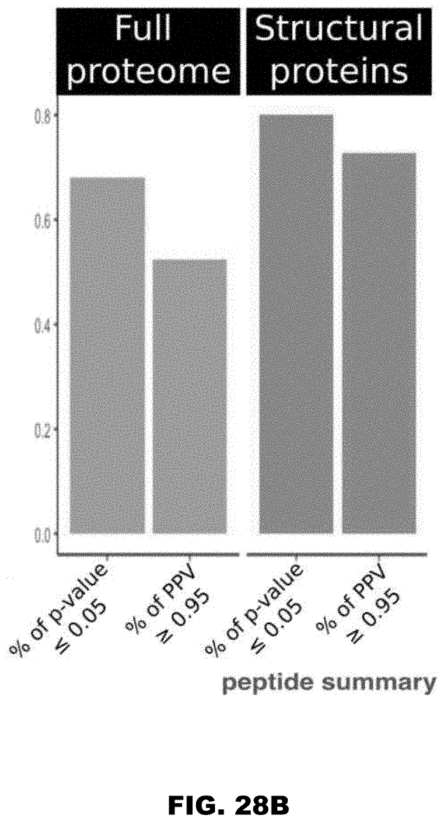

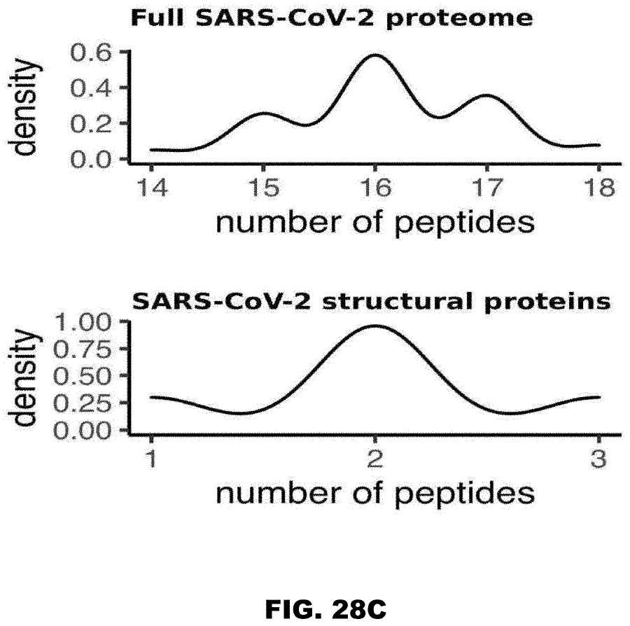

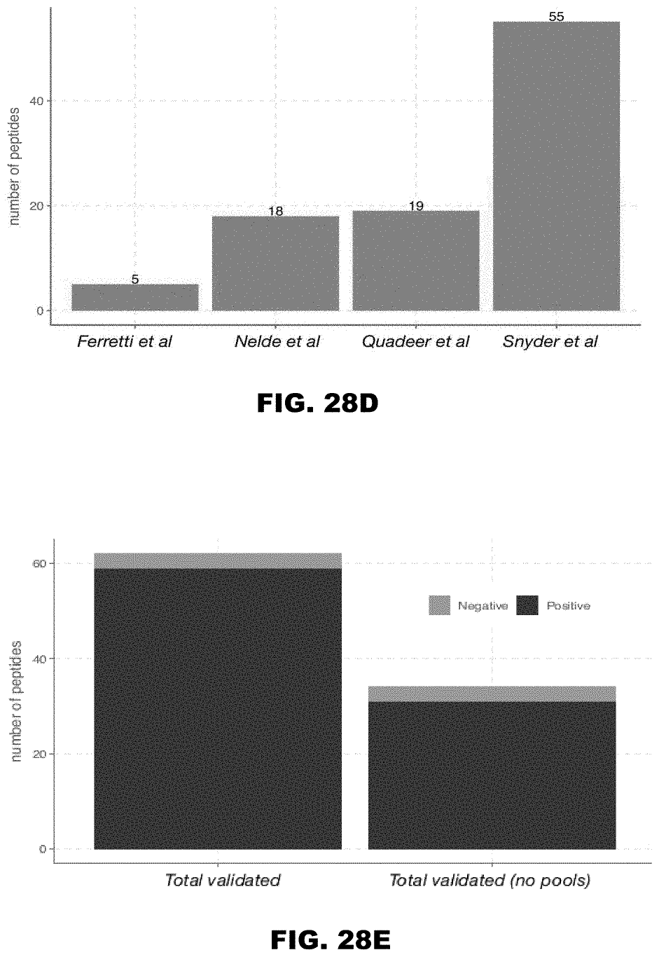

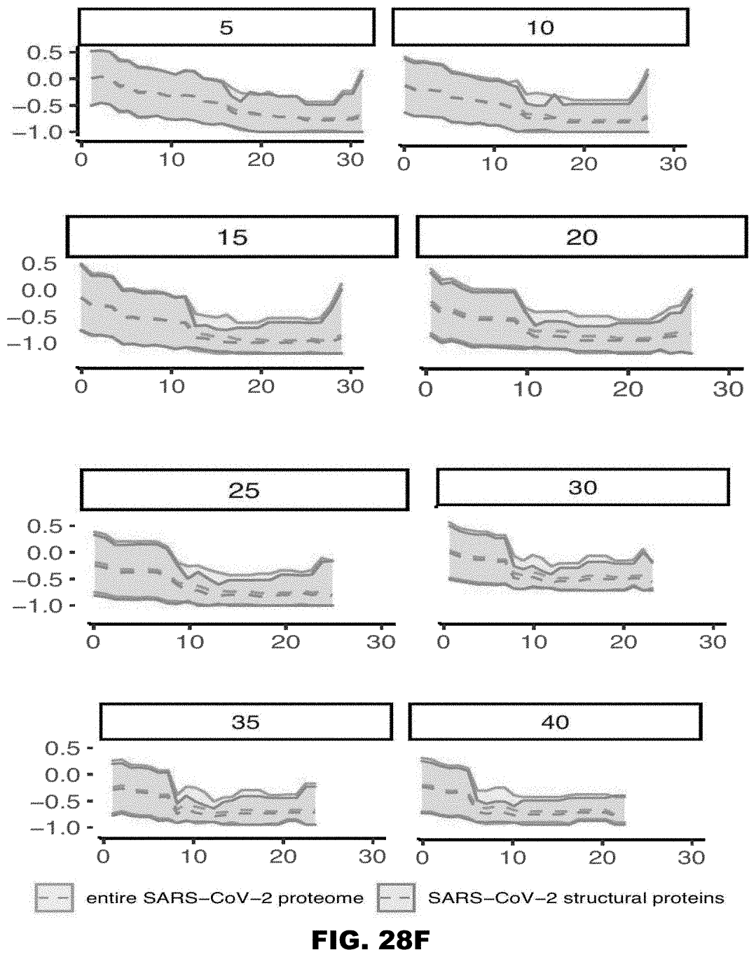

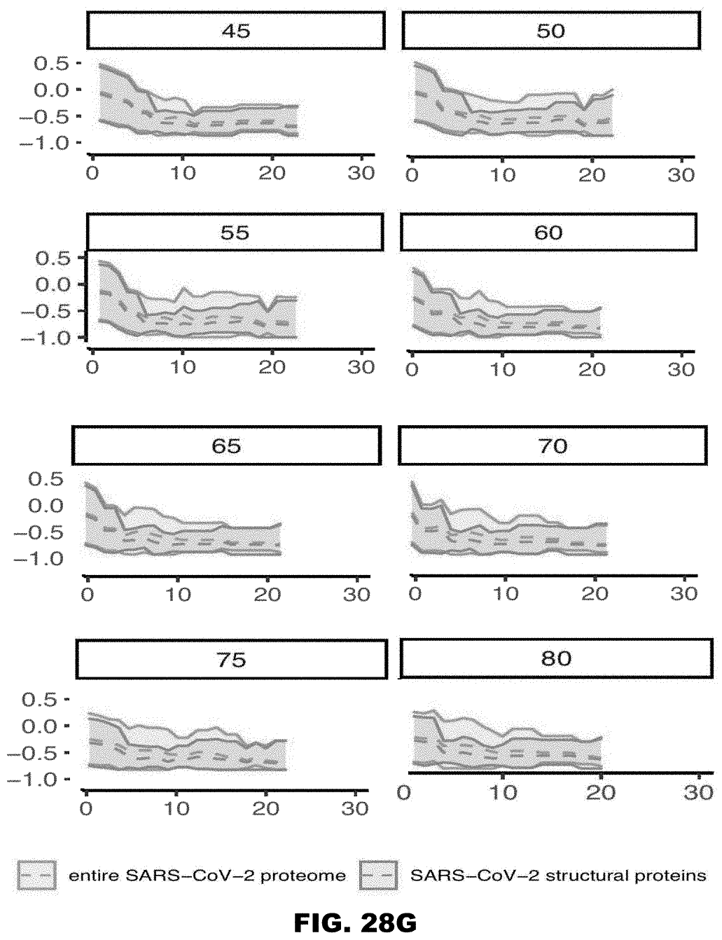

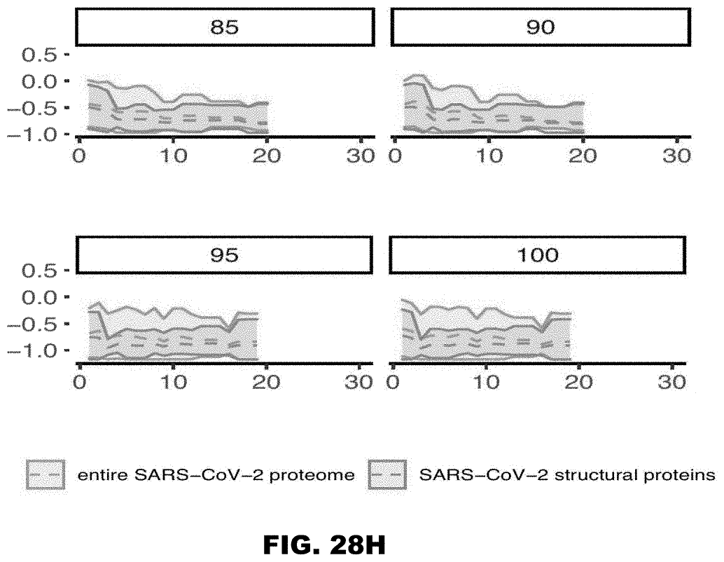

Both the peptide-allele distributions, namely the ones derived from the full SARS-CoV-2 proteome, and those from the structural proteins were found to significantly deviate from an even distribution of predicted peptides as is apparent in FIGS. 21A and 21B and reflected in the Kolmogorov-Smirnov test p values (FIG. 28A-28F; full proteome=5.673e 7 and structural proteins=1.45e 2). These results support a potential allele-specific hierarchy for SARS-CoV-2 peptide presentation.

To determine whether the MHC-I binding capacity hierarchy was consistent between the full SARS-CoV-2 proteome and SARS-CoV-2 structural proteins, the relative changes in the observed peptide fraction (number of peptides assigned to an allele/total number of peptides) between the two protein sets was visualized (FIG. 21C). A total of 6 alleles demonstrated changes greater than the median peptide fraction (X=0.015) when comparing the 2 protein sets. The greatest decrease in peptide fraction was observed for A*25:01 (1.52 times the median peptide fraction), and the greatest increase was seen with B*53:01 (2.38 times the median peptide fraction). Furthermore, the resulting SARS-CoV-2 structural protein peptide-allele distribution was found to be more variable than the distribution derived from the full SARS-CoV-2 proteome, with a quartile coefficient of dispersion of 0.6 compared to 0.44, respectively. This indicates that peptides derived from SARS-CoV-2 structural proteins experience larger relative inter-allele binding capacity discrepancies than peptides derived from the full SARS-CoV-2 proteome. These results indicate a potential MHC-I binding capacity hierarchy that is more pronounced for SARS-CoV-2 structural proteins.

Total Population Epitope Load Inversely Correlates with Reported Death Rates from SARS-CoV-2

The documented importance of MHC-I peptides derived from SARS-CoV-2 structural proteins, coupled with the observed MHC allele binding capacity hierarchy and the high immunogenicity rate of SARS-CoV-2 structural protein MHC-I peptides identified by EnsembleMHC (FIG. 28D), prompts a potential relationship between MHC-I genotype and infection outcome. However, due to the absence of MHC genotype data for SARS-CoV-2 patients, this relationship was assessed at the population level by correlating predicted country-specific SARS-CoV-2 binding capacity (or EMP score) with observed SARS-CoV-2 mortality.

EMP scores were determined for 23 countries (Tables B and C) by weighing the individual binding capacities of 52 common MHC-I alleles by their normalized endemic expression (FIG. 25). Every country in the cohort is assigned two separate EMP scores—one calculated with respect to the 108 unique SARS-CoV-2 structural protein peptides (structural protein EMP) and the other with respect to the 658 unique peptides derived from the full SARS-CoV-2 proteome (full proteome EMP). The EMP score corresponds to the average predicted SARS-CoV-2 binding capacity of a select population. Therefore, individuals in a country with a high EMP score would be expected, on average, to present more SARS-CoV-2 peptides to CD8+-T cells than would individuals from a country with a low EMP score. The resulting EMP scores were then correlated with observed SARS-CoV-2 mortality (deaths per million) as a function of time (January-April 2020). Temporal variance in community spread within the cohort of countries was corrected by truncating the SARS-CoV-2 mortality dataset for each country to start after a certain minimum death threshold was met. For example, if the minimum death threshold was 50, then day 0 would be when each country reported at least 50 deaths. The number of countries included in each correlation decreases as the number of days increases, due to discrepancies in the length of time that each country met a given minimum death threshold (Table S3). Therefore, the correlation between EMP score and SARS-CoV-2 mortality was only estimated at time points at which there were at least 8 countries. The 8-country threshold was chosen because it is the minimum sample size needed to maintain sufficient power when detecting large effect sizes (r>0.85). The strength of the relationship between EMP score and SARS-CoV-2 mortality was determined using Spear-man's rank-order correlation (for details concerning the choice of statistical tests, please refer to STAR Methods). Accordingly, both EMP scores and SARS-CoV-2 mortality data were converted into ascending ranks, with the lowest rank indicating the minimum value and the highest rank indicating the maximum value. For instance, a country with an EMP score rank of 1 and death per million rank of 23 would have the lowest predicted SARS-CoV-2 binding capacity and the highest level of SARS-CoV-2-related mortality. Using the described paradigm, the structural protein EMP score and the full proteome EMP score were correlated with SARS-CoV-2-related deaths per million for 23 countries.

TABLE B
Countries
China
Japan
South Korea
US
France
Germany
India
Italy
Russia
UK
Iran
Israel
Croatia
Romania
Netherlands
Mexico
Ireland
Czechia
Morocco

TABLE C

| Factor | Abbreviation | Description |
|---|---|---|
| % of population ≥ 65 | 65 | Percentage of the population that |
| years | | is 65 years of age or older (2020). |
| Average BMI | Avg. BMI | The age-standardized average |
| | | population body mass index |
| | | (2016). |
| Cardiovascular disease | CD | The deaths per million due to |
| | | cardiovascular disease (2016). |
| Chronic obstructive | COPD | The deaths per million due to |
| pulmonary disease | | complications from chronic |
| | | obstructive pulmonary disease |
| | | (2016). |
| Diabetes mellitus | DM | The deaths per million due to |
| | | complications from diabetes |
| | | mellitus (2016). |
| High blood pressure | BP | The age-standardized percentage |
| | | of the population with a systolic |
| | | blood pressure ≥ 140 or diastolic |
| | | blood pressure ≥ 90 (2015). |
| Obesity prevalent | OBS | The age-standardized percentage |
| | | of the population with a BMI ≥ 30 |
| | | (2016). |
| Overweight prevalent | OVW | The age-standardized percentage |
| | | of the population with a BMI ≥ 25 |
| | | (2016). |
| Structural protein EMP | SP | The SARS-CoV-2 structural |
| score | | protein presentation score. |
| % of GDP spent on | GDP | Current health expenditure (CHE) |
| health care | | as percentage of gross domestic |
| | | product (2017). |
| % of total gov. | GGHE | General government expenditure |
| expenditure on health | | on health as a percentage of total |
| care | | (2014). |
| % of population that is | SEX | The proportion of the total |
| female | | population that is female (2020). |

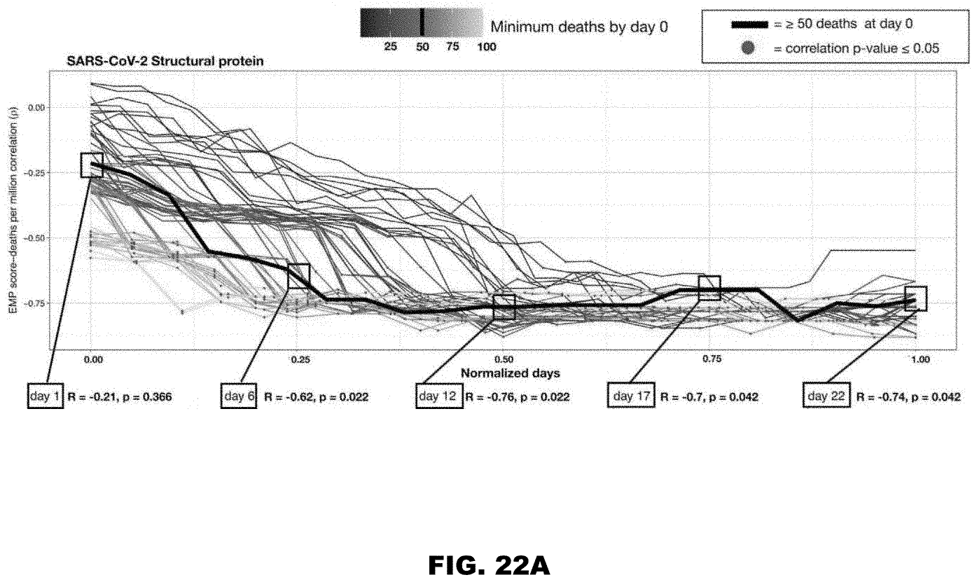

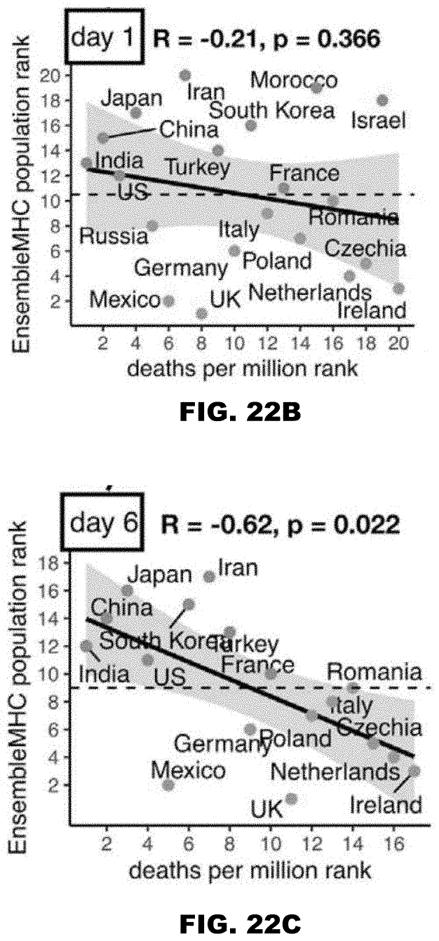

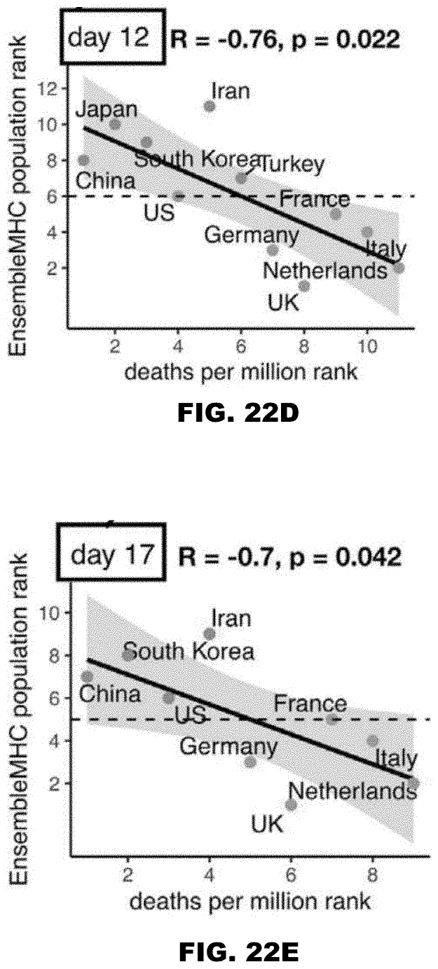

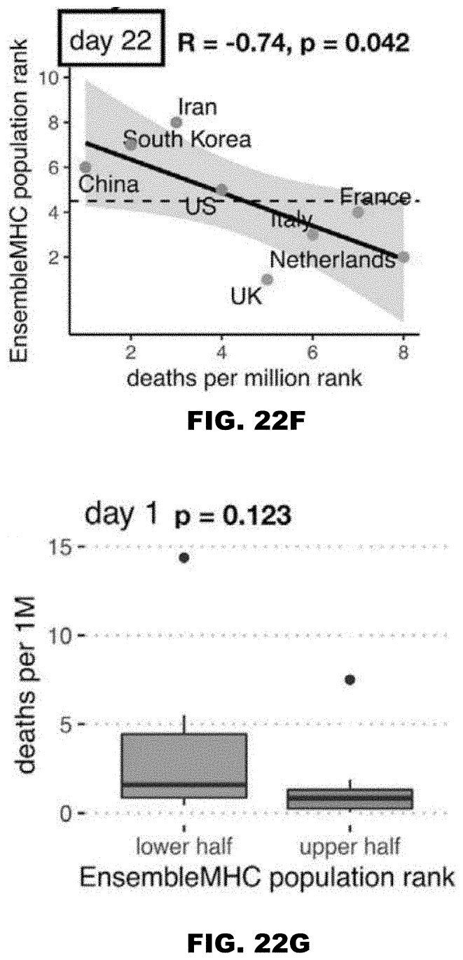

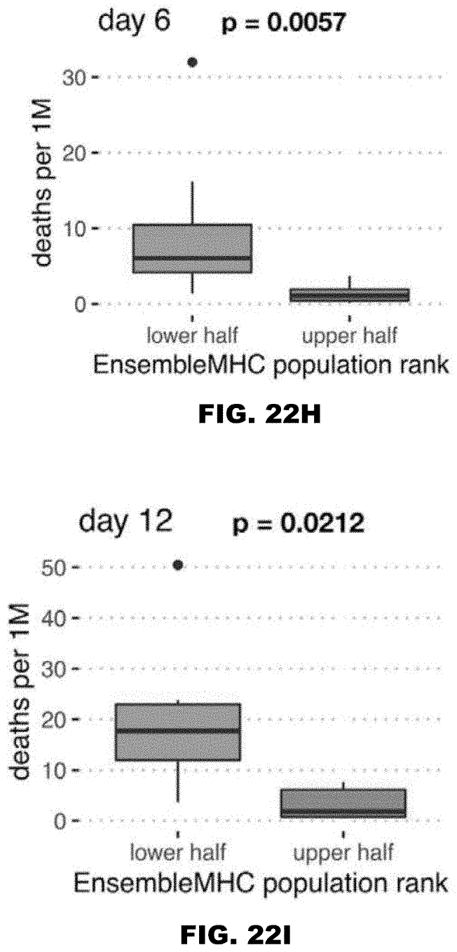

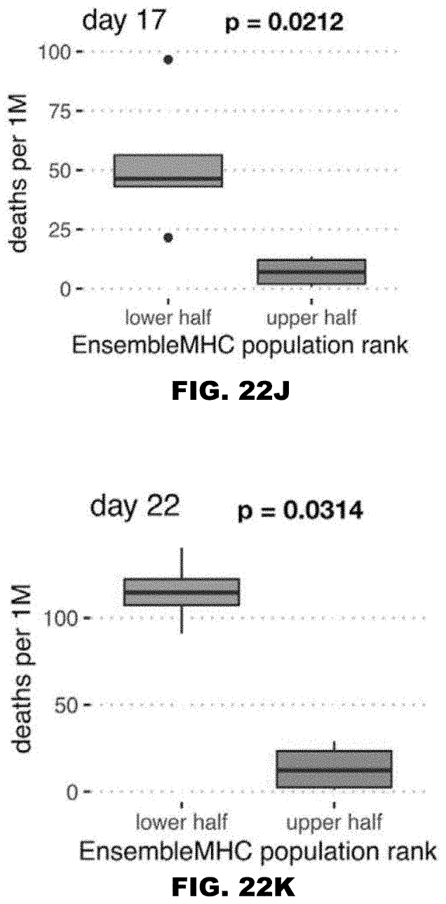

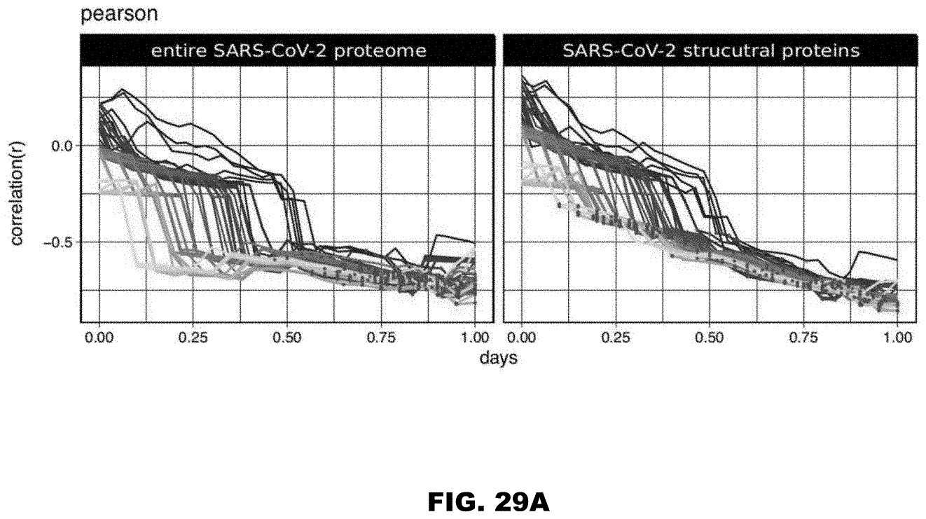

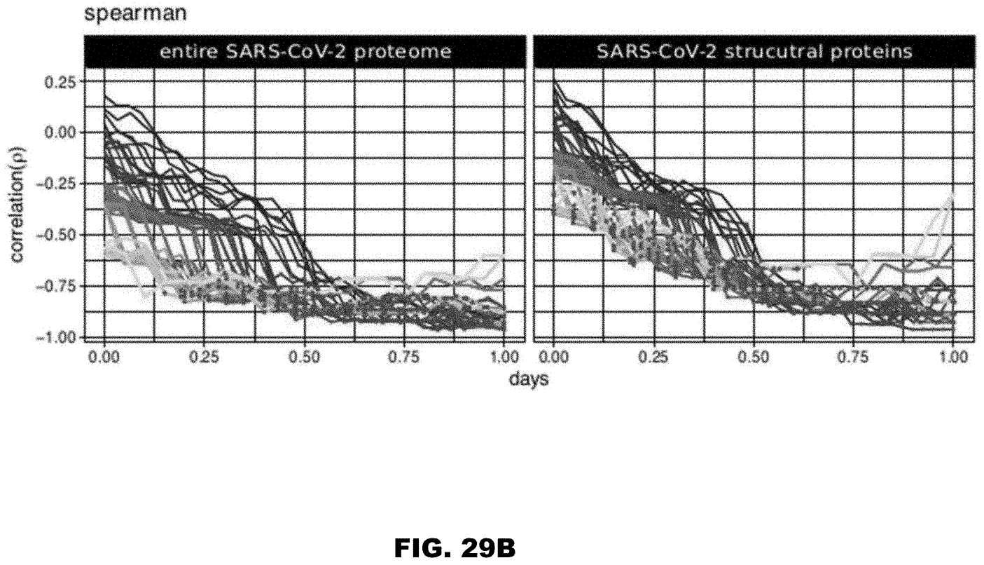

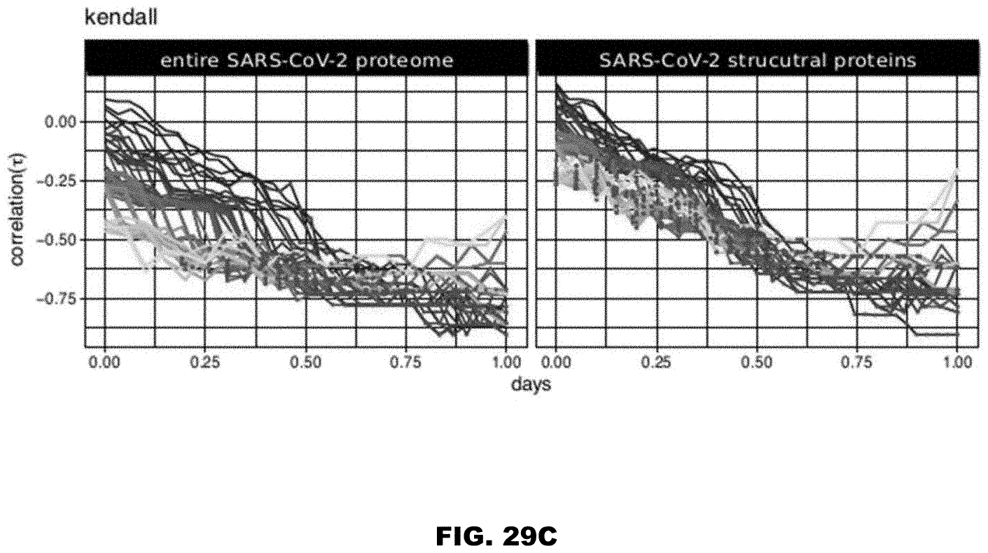

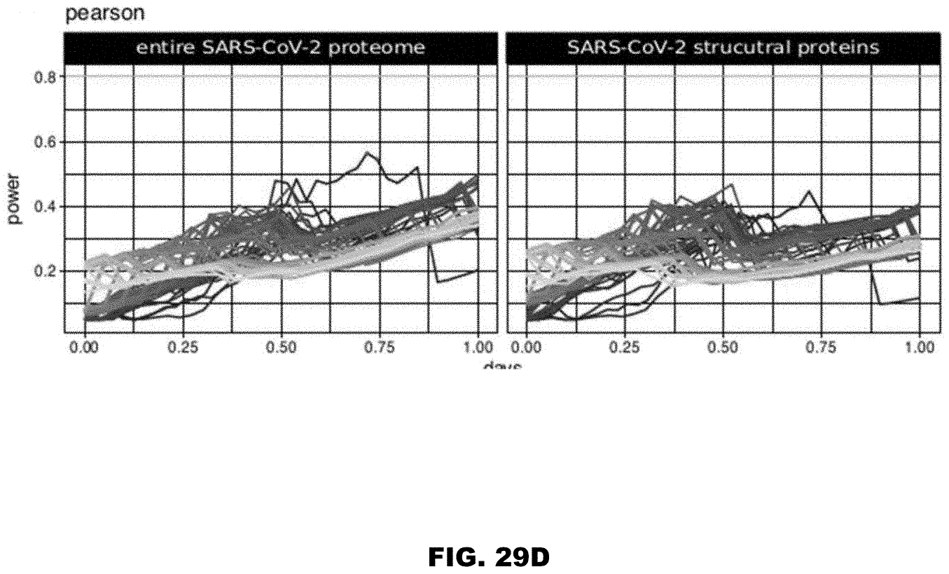

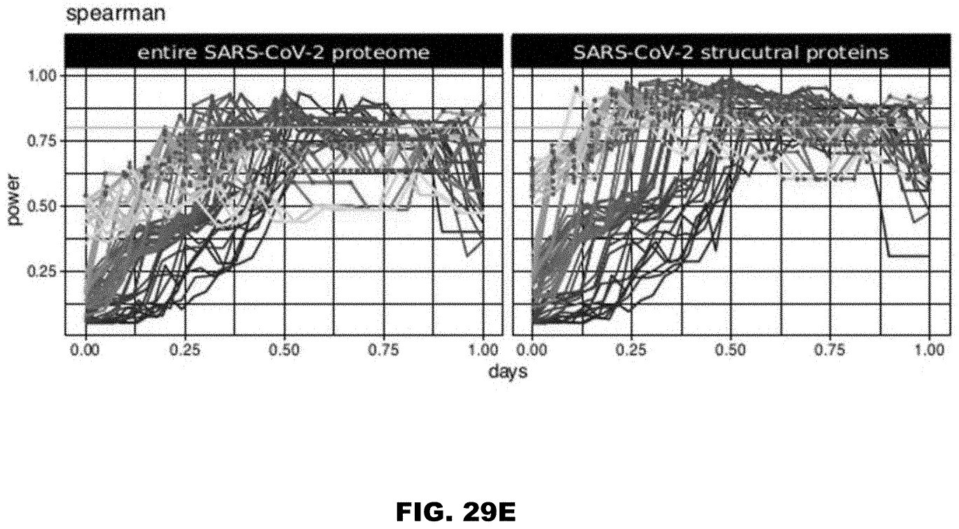

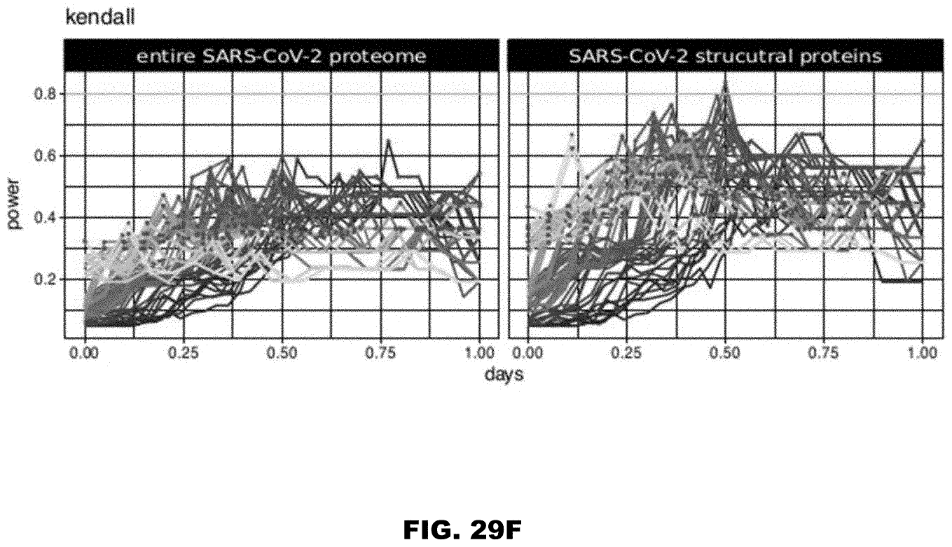

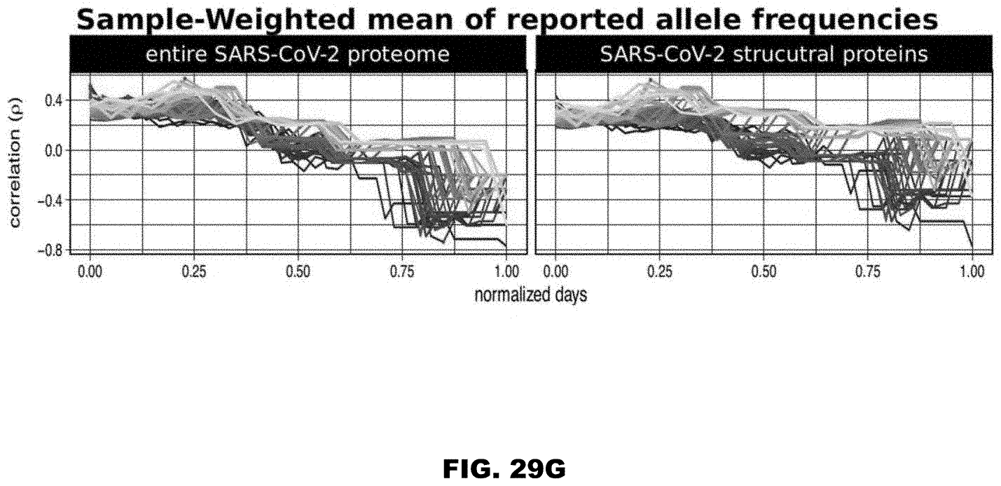

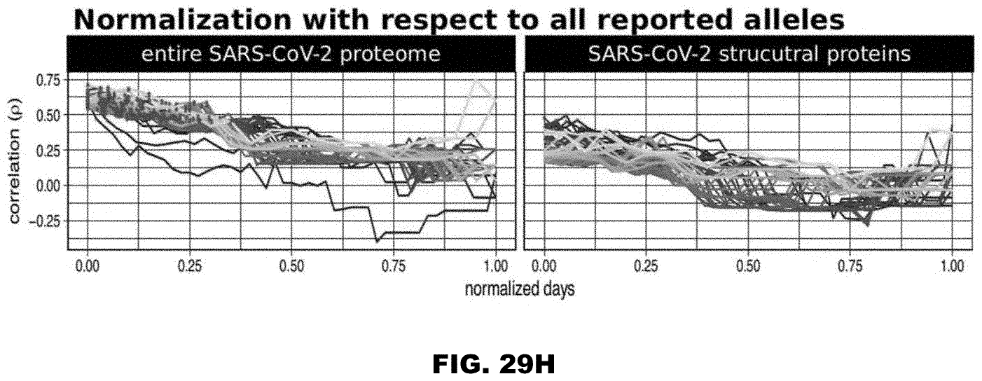

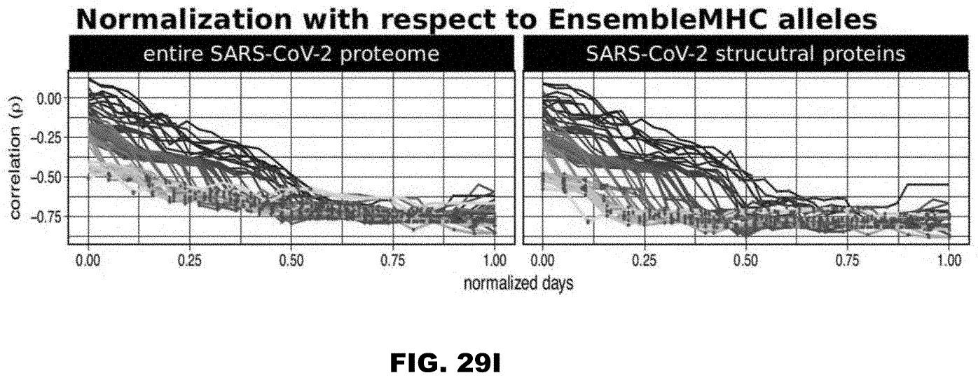

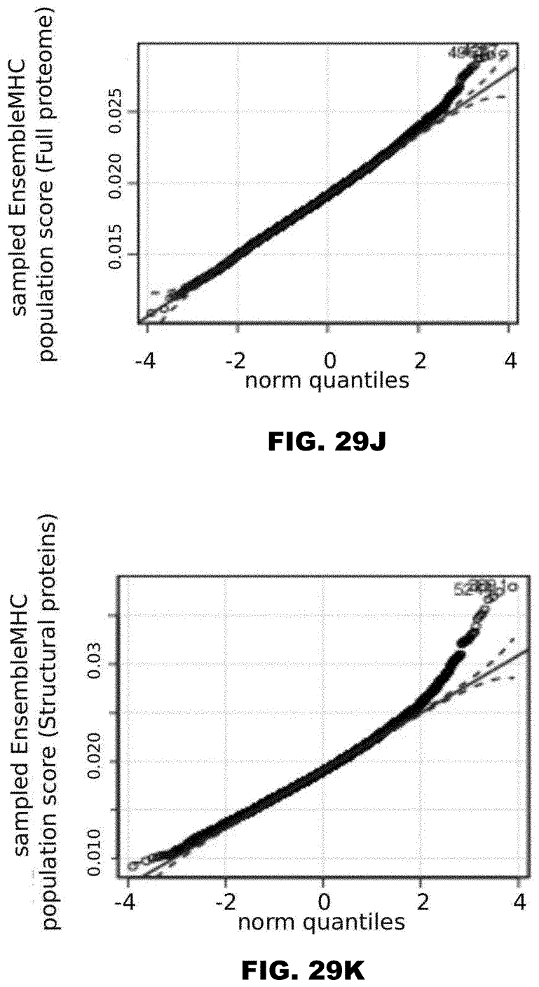

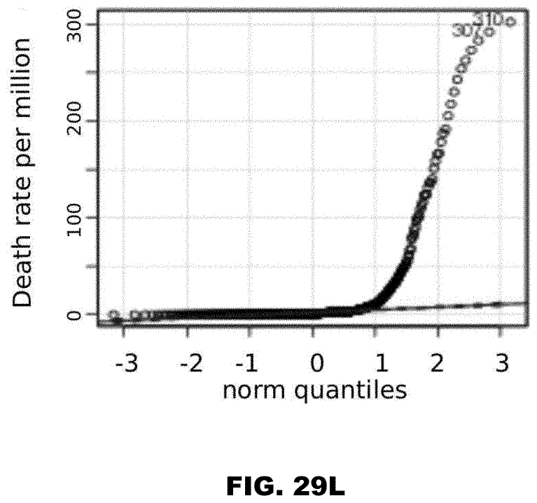

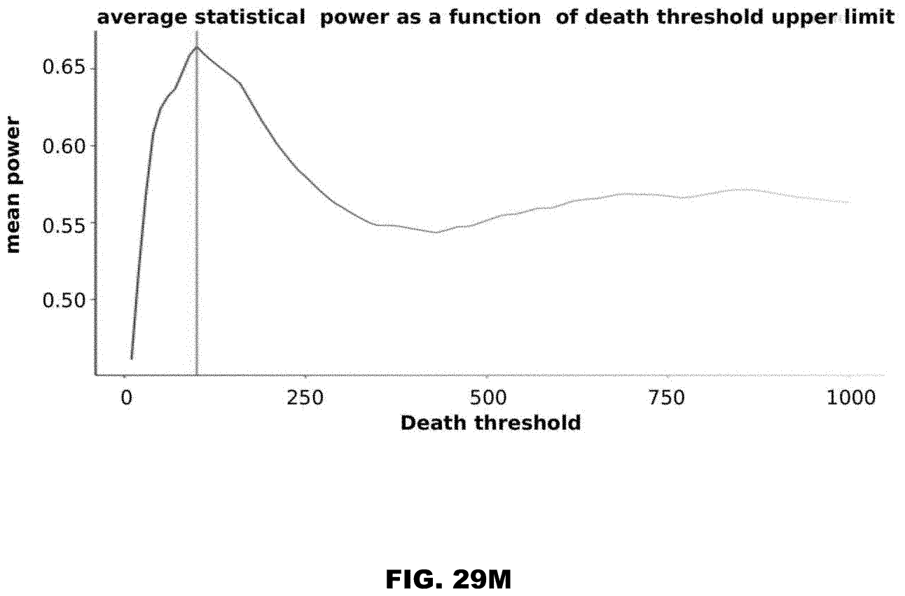

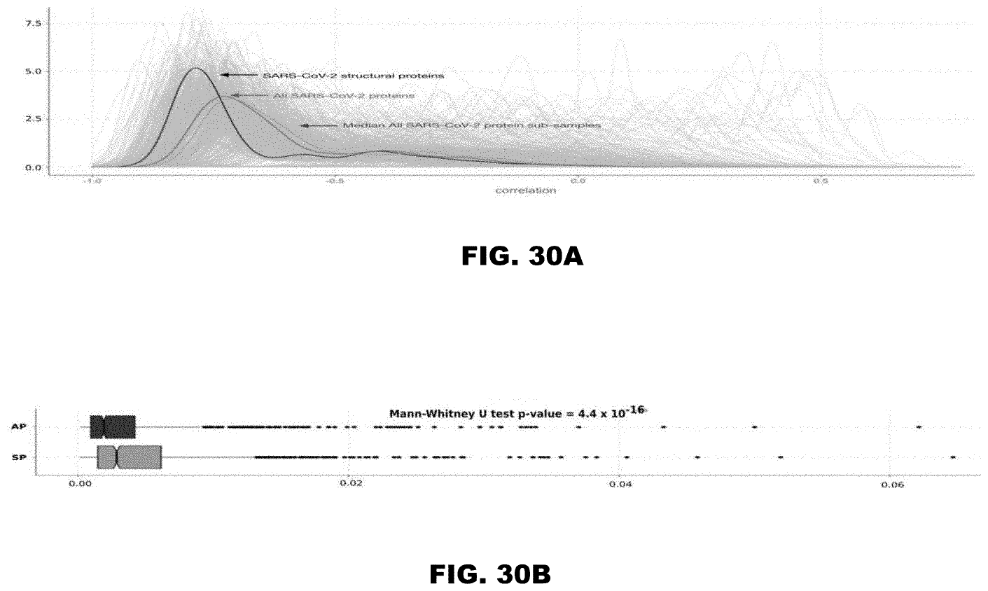

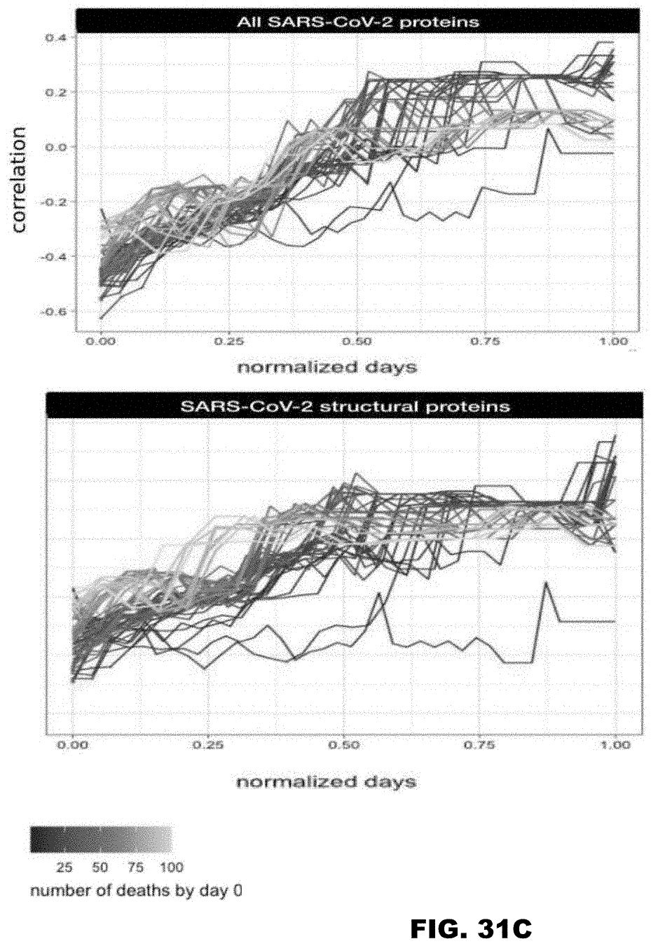

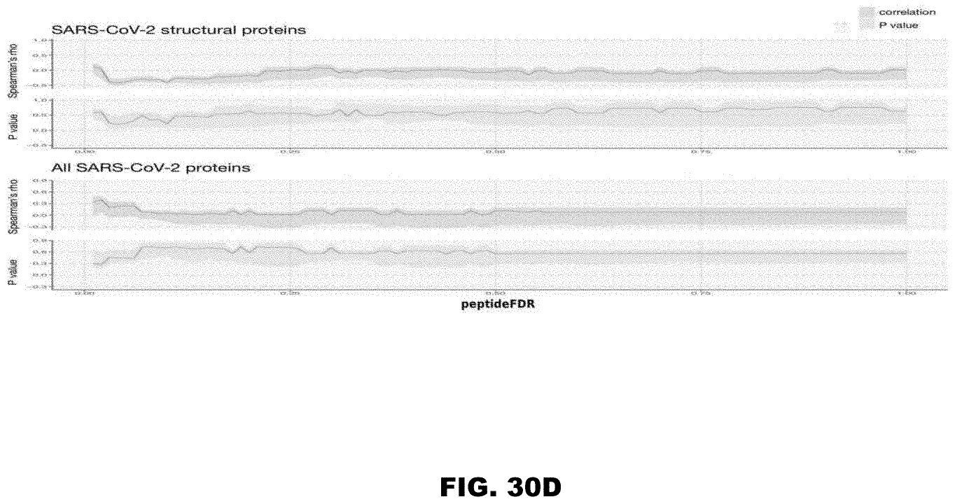

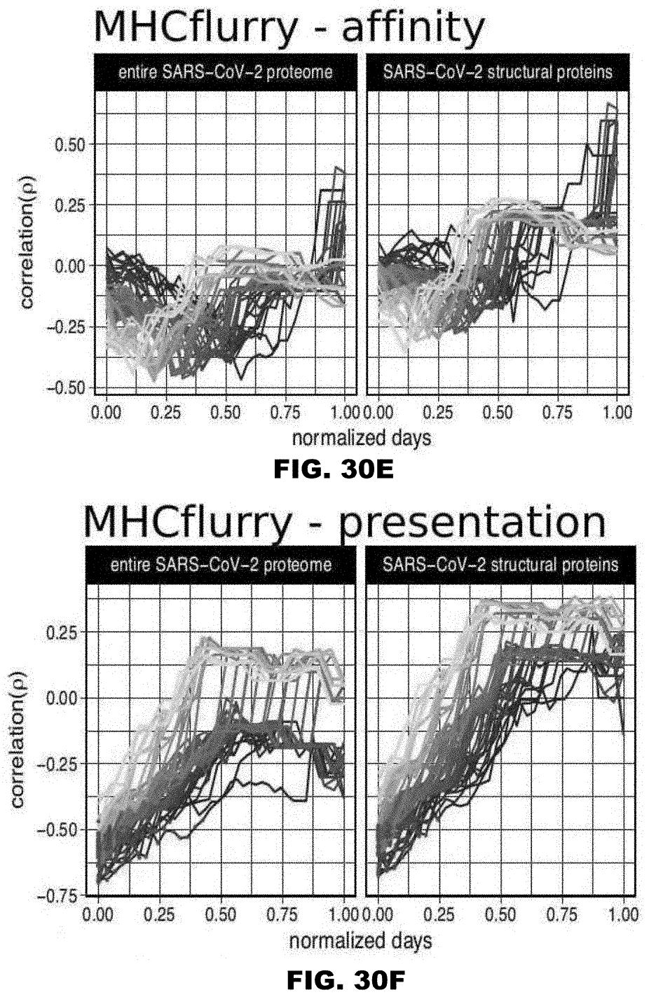

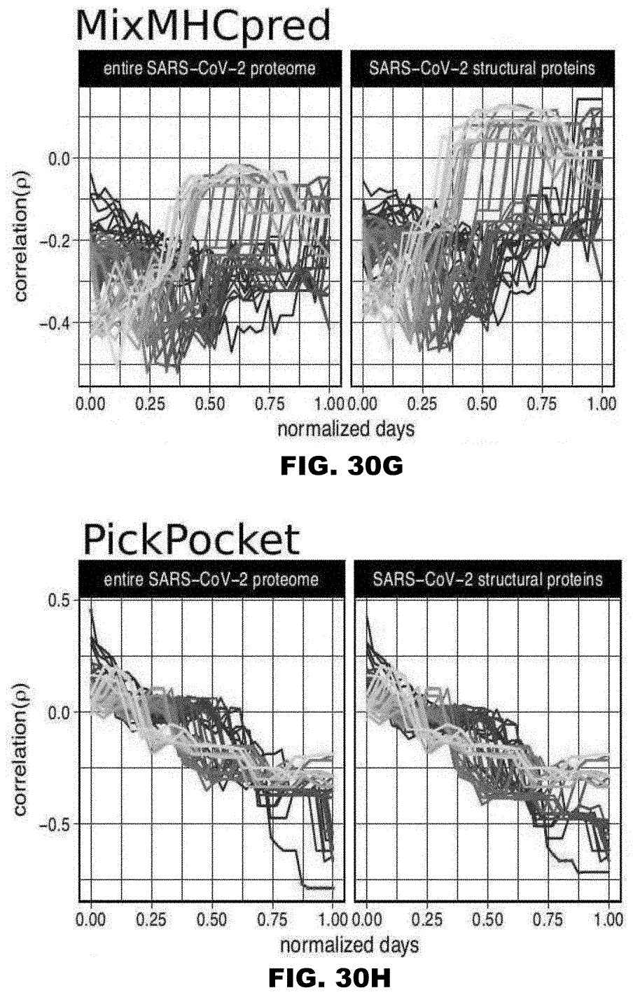

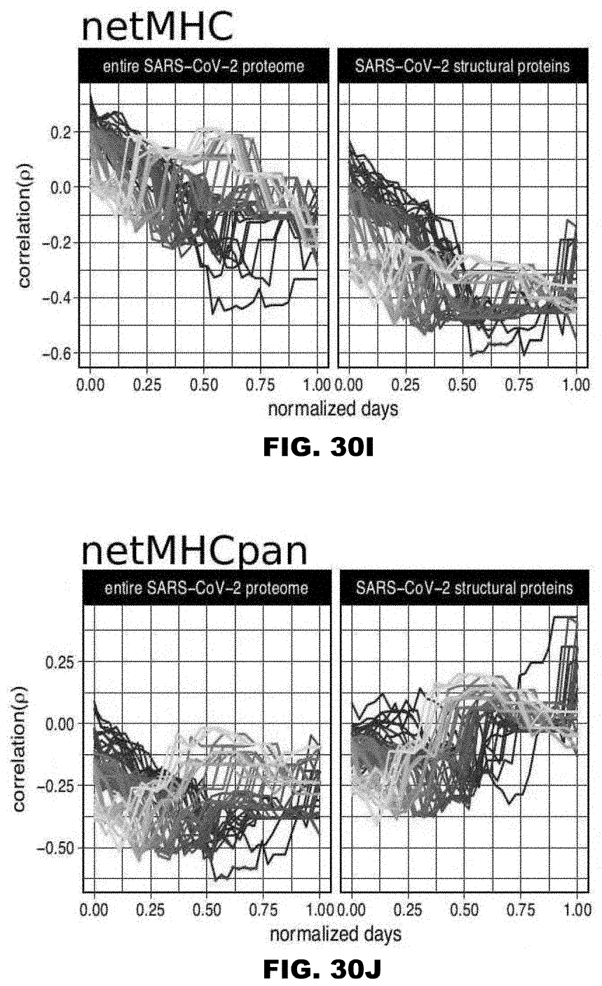

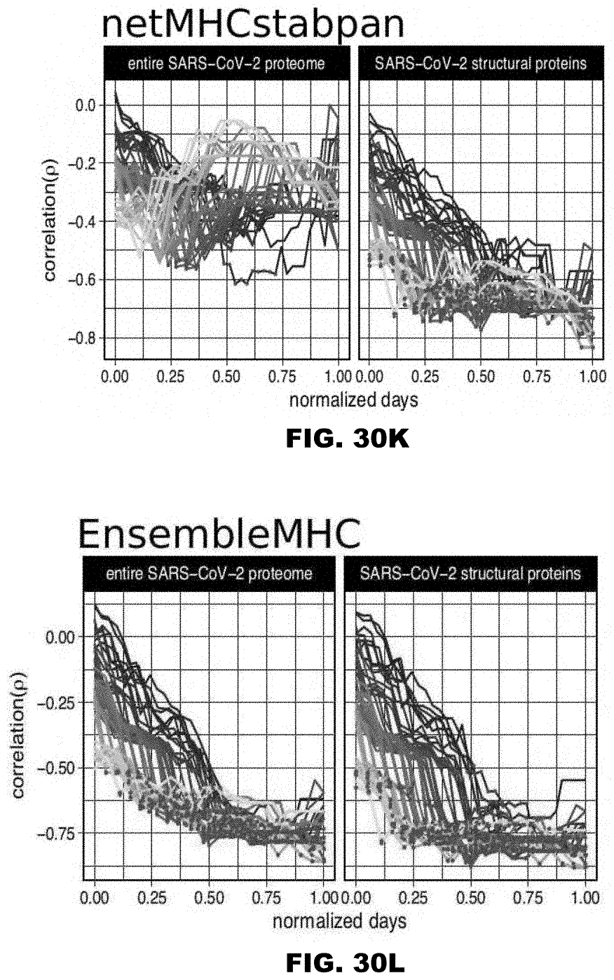

Total predicted population SARS-CoV-2 binding capacity exhibited a strong inverse correlation with observed deaths per million. This relationship was found to be true for correlations based on the structural protein EMP (FIG. 22A) and full proteome EMP (FIG. 28A) scores, with mean effect sizes of 0.66 and 0.60, respectively. Significance testing of the correlations produced by both EMP scores revealed that the majority of re-ported correlations are statistically significant, with 63% attaining a p % 0.05. Correlations based on the structural protein EMP score demonstrated a 23% higher proportion of statistically significant correlations compared to the full proteome EMP score (74% versus 51%). Furthermore, correlations for EMP scores based on structural proteins produced narrower 95% confidence intervals (FIG. 28B; Table S3). Due to relatively low statistical power of the obtained correlations (FIG. 29), the positive predictive value (PPV) for each correlation (Equation 8; see below) was calculated. The resulting proportions of correlations with a PPV of R 95% were similar to the observed significant p value proportions, with 62% of all measured correlations, 72% of structural protein EMP score correlations, and 52% of full proteome EMP score correlations (FIG. 28B). The similar proportions of significant p values and PPVs supports that an overall true association is being captured. Furthermore, analysis of similar-size peptide sets sampled from the full SARS-CoV-2 proteome revealed that the observed distinction between the correlations produced by the two protein groups are unlikely to be due to differences in peptide set sizes (FIGS. 30A and 30B).

Finally, the reported correlations did not remain after randomizing the allele assignment of predicted peptides before peptide^{FDR} filtering (FIGS. 30C and 30D) through the use of any individual algorithm (FIG. 30E). This indicates that the observed relationship is contingent on the high-confidence peptide-allele distribution identified by EnsembleMHC. These data demonstrate that the MHC-I allele hierarchy characterized by EnsembleMHC is inversely associated with SARS-CoV-2 population mortality, and that the relationship becomes stronger when considering only the presentation of SARS-CoV-2 structural proteins.

The ability to use the structural protein EMP score to identify high- and low-risk populations was assessed using the median minimum death threshold (50 deaths) at evenly spaced time points (FIG. 22A, squares). All of the correlations, with the exception of day 1, were found to be significant, with an average effect size of 0.71 (FIG. 22B). Next, the countries at each day were partitioned into a high or low group based on whether their assigned EMP score was higher or lower than the median observed EMP score FIG. 22C). The resulting groups demonstrated a statistically significant difference in the median deaths per million between countries with low structural protein EMP scores and countries with high structural protein EMP scores. In addition, it was observed that deaths per million increased much more rapidly in countries with low structural protein EMP scores. These results indicate that the structural protein EMP score may be useful for assessing population risk from SARS-CoV-2 infections.

In summary, several important observations were made. First, there is a strong inverse correlation between predicted population SARS-CoV-2 binding capacity and observed deaths per million. This finding suggests that outcome to SARS-CoV-2 may be tied to total epitope load. Second, the correlation between predicted epitope load and population mortality is stronger for SARS-CoV-2 structural MHC-I peptides. This suggests that CD8+-T cell-mediated immune response may be driven primarily by the recognition of epitopes derived from these proteins, a finding supported by recent T cell epitope mapping of SARS-CoV-2. Finally, the EMP score can separate countries within the considered cohort into high- or low-risk populations.

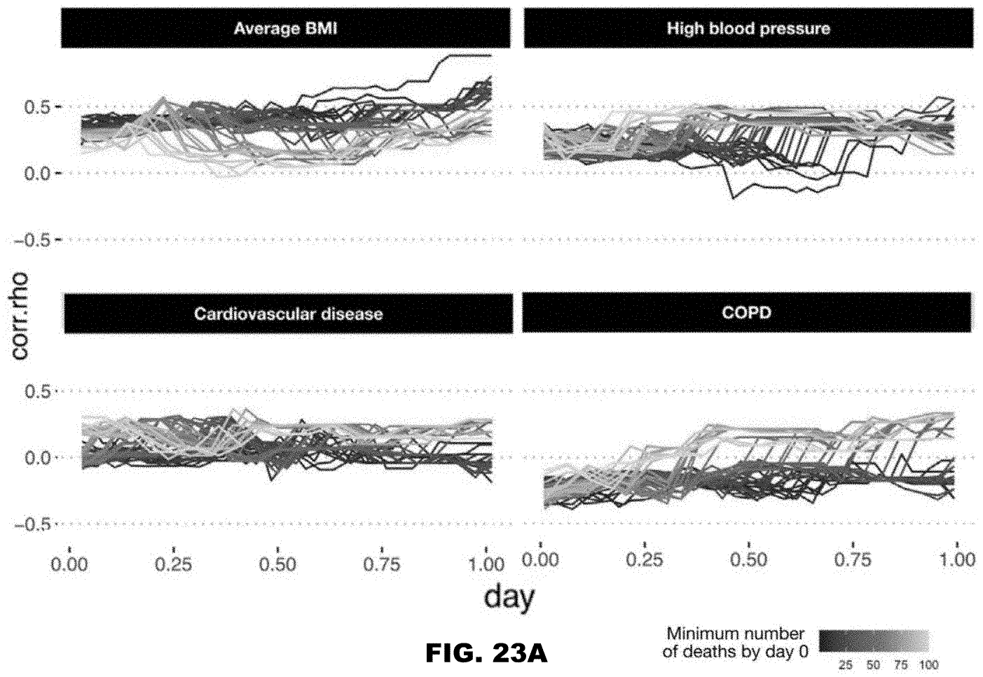

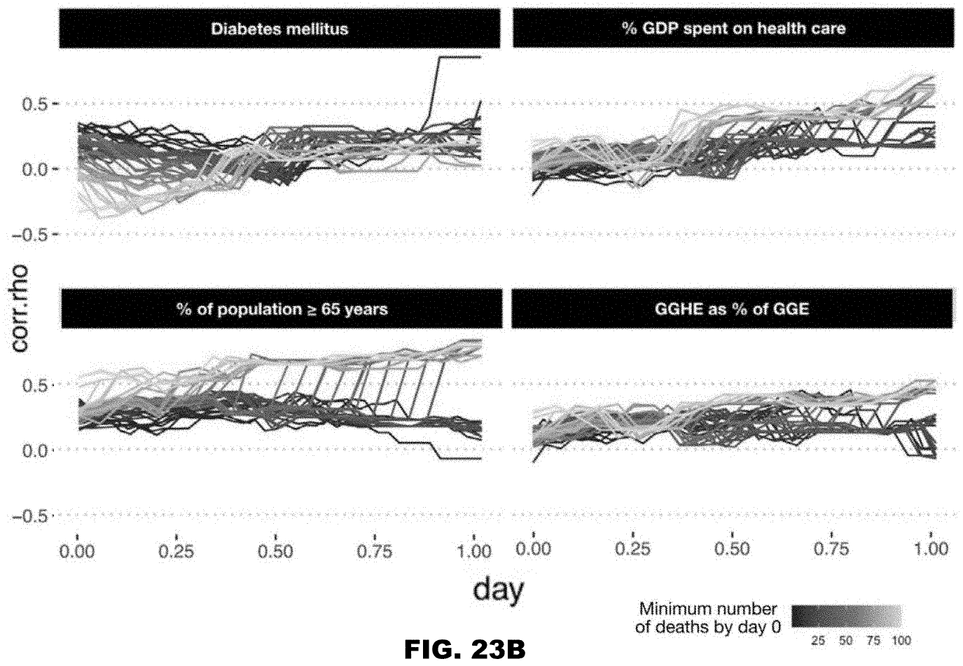

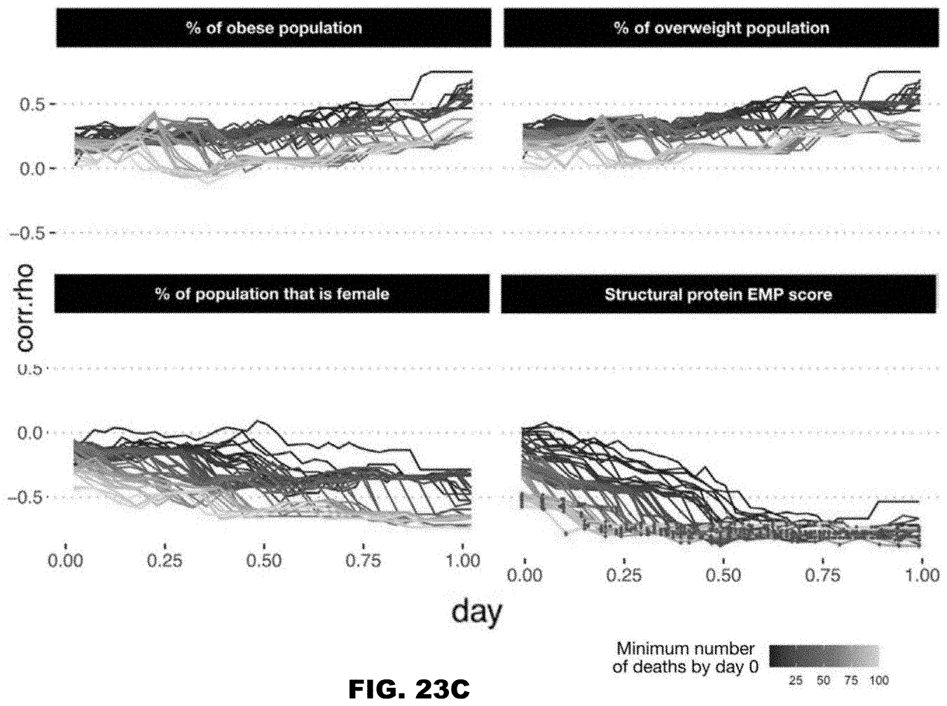

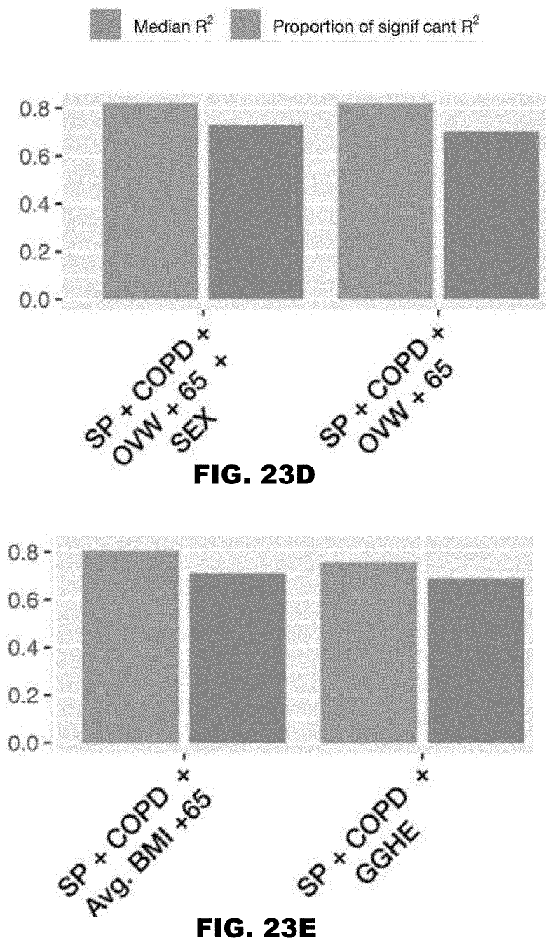

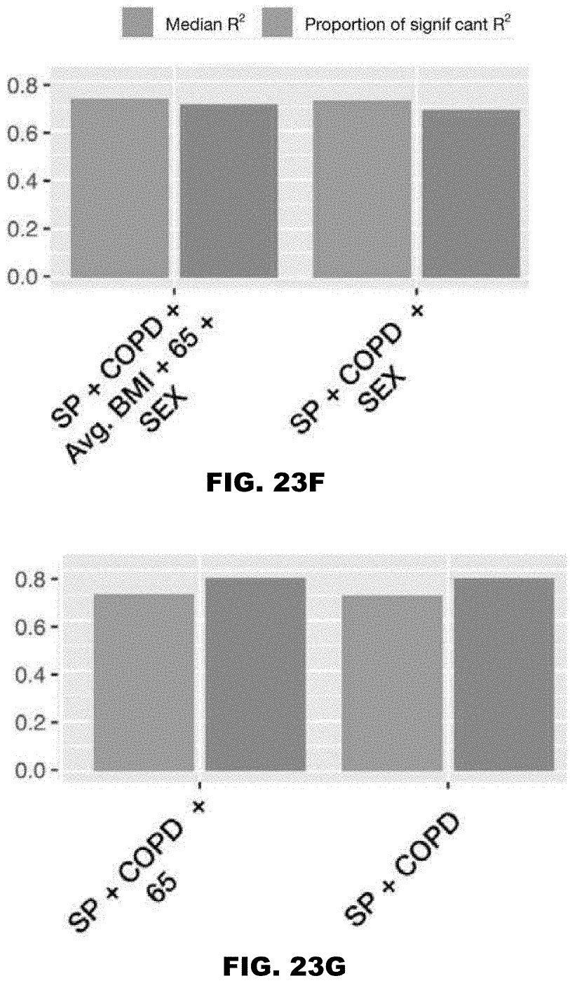

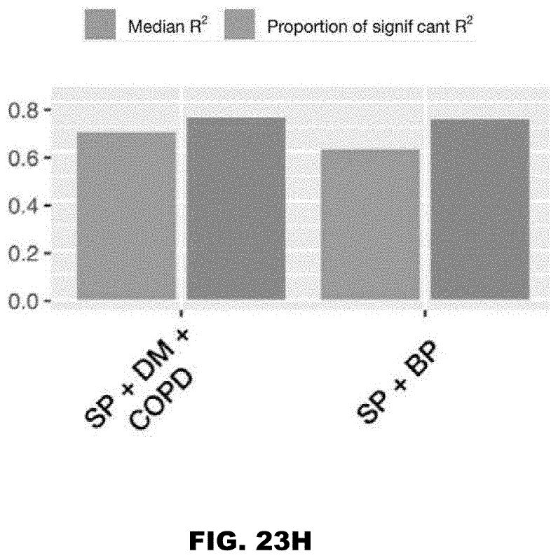

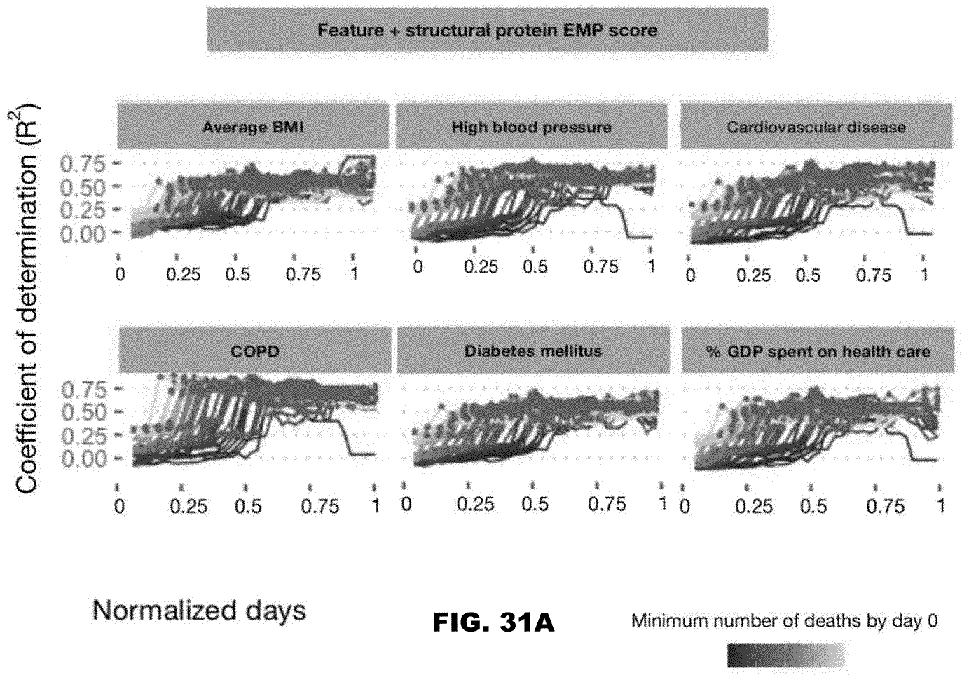

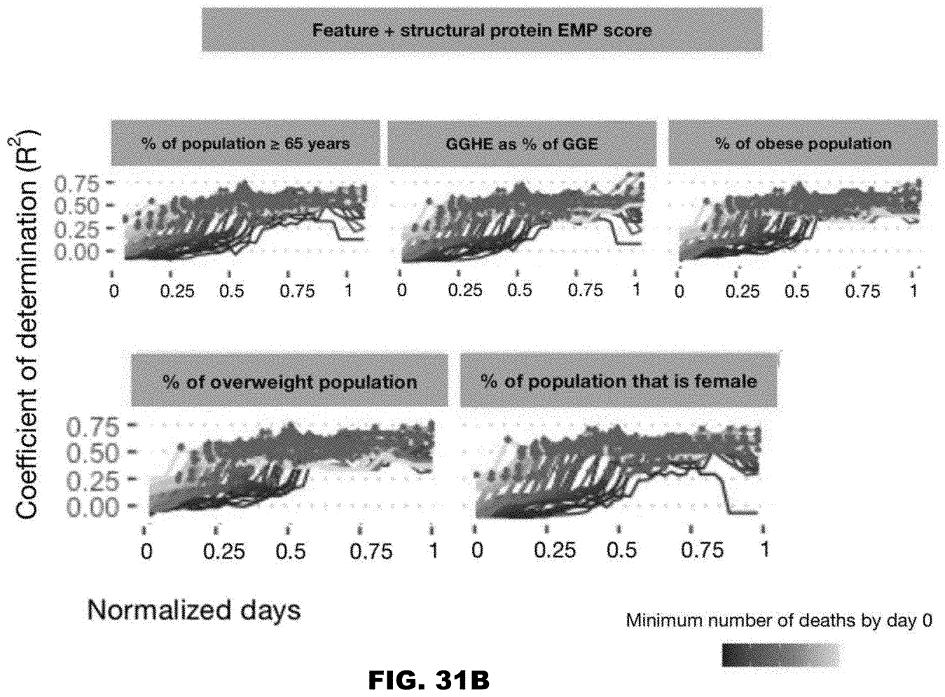

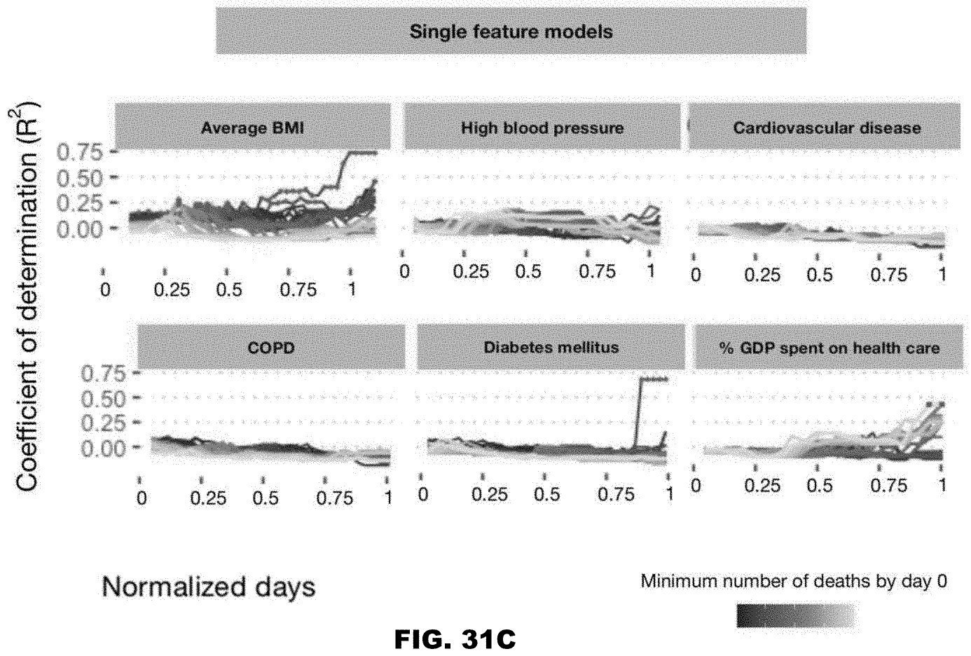

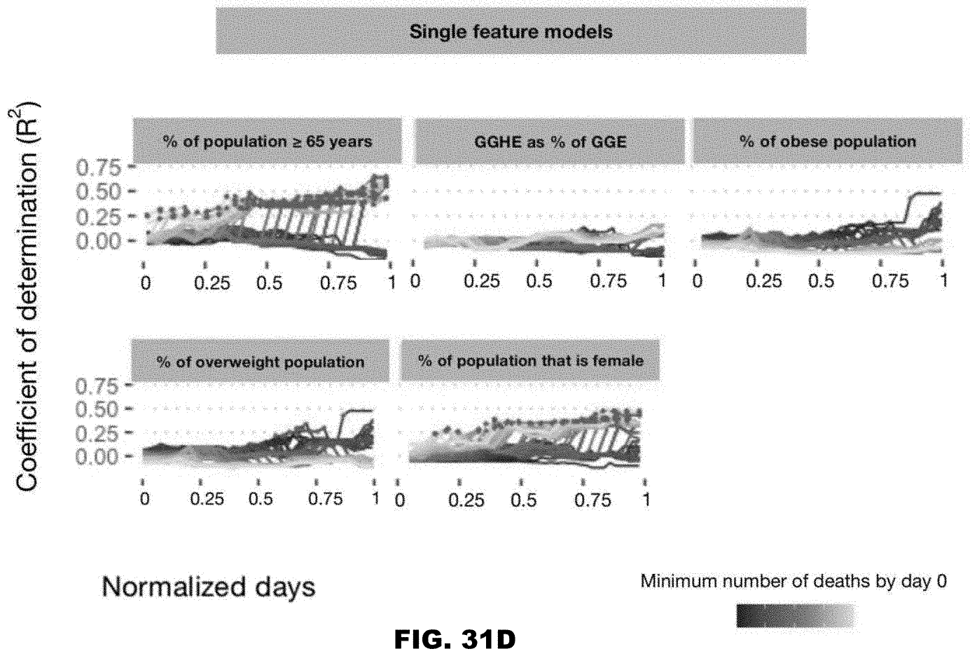

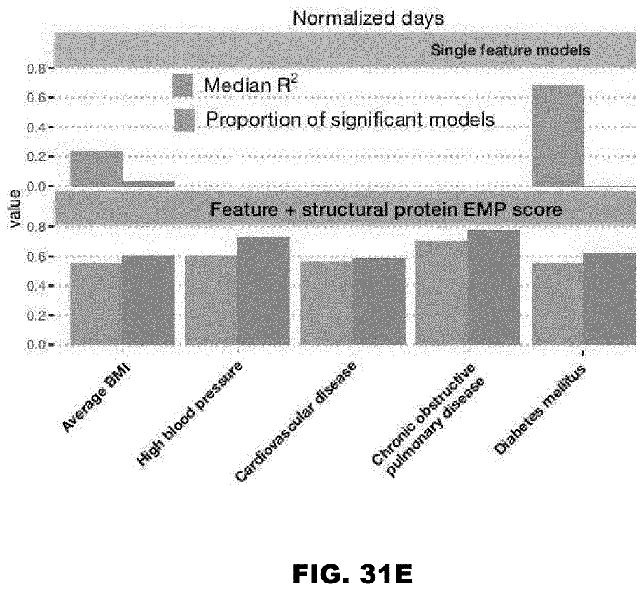

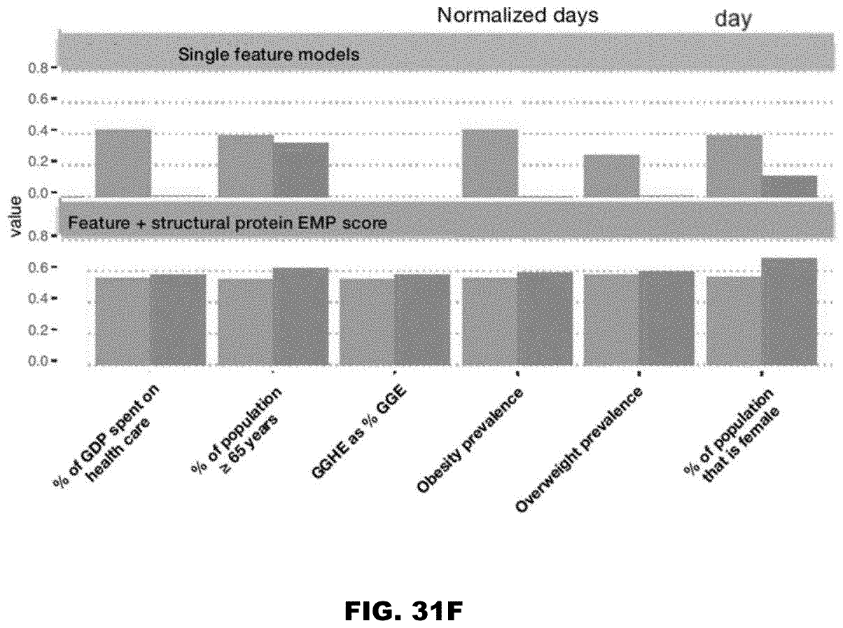

Overall, the structural protein EMP scores produced a significantly stronger association with population SARS-CoV-2 mortality compared to 12 other descriptors (FIG. 23A). While various effect size trends were observed, all of the additional covariates failed to produce statistically significant correlations. To determine whether the modeling of the SARS-CoV-2 mortality rate could be improved by the combination of single socioeconomic or health-related risk factors with structural protein EMP scores, a set of linear models consisting of either a single risk factor (single-feature model) or that factor combined with structural protein EMP scores (combination model) were generated for every time point across each minimum death threshold (STAR Methods). Following model generation, the adjusted coefficient of determination (R2) and significance level of each individual model was extracted and aggregated by dependent variable (FIGS. 31A and 31B. Single-feature models were characterized by low R2Qt=0.0262), while combination models showed significant improvement (X=0.496). Similarly, combination models demonstrated a substantially higher proportion of statistical significance (FIG. 31B). To determine the set of features that produce the best-fitting model, all possible combinations of explanatory factors (risk factors and structural protein EMP score) were tested. Subsequently, the top 10 performing models, ranked by adjusted R2 value, were selected for analysis (FIG. 23B). The identified models were found to be largely significant (average proportion of significant regressions=72%) and produce strong fits to the data (average R2=0.7).

Analysis of the dependent variables included in the top-performing models revealed that all models used structural protein EMP scores followed by deaths per million due to complications from chronic obstructive pulmonary disease (COPD) (90% of models). The median model size included 3 features, with a maximum of 5 features and a minimum of 2 features. The model producing the best fit (median R2=0.791) consisted of structural protein EMP scores, gender demographics, number of deaths due to COPD complications, the proportion of the population older than age 65 years, and proportion of the population that is overweight FIG. 23B). These results further indicate the robustness of the structural protein EMP score as a population-level risk descriptor.

### Discussion

In the present study, evidence supporting an association between population SARS-CoV-2 infection outcome and MHC-I genotype was uncovered. In line with related work highlighting the relationship between total epitope load and HIV viral control, a working model was arrived at, that MHC-I alleles presenting more unique SARS-CoV-2 epitopes will be associated with lower mortality due to a higher number of potential T cell targets. The SARS-CoV-2 binding capacities of 52 common MHC-I alleles were assessed using the EnsembleMHC prediction platform. These predictions identified 971 high-confidence MHC-I peptides out of a candidate pool of nearly 3.5 million. In agreement with other in silica studies, the assignment of the predicted peptides to their respective MHC-I alleles revealed an uneven distribution in the number of peptides attributed to each allele. It was discovered that the MHC-I peptide-allele distribution originating from the full SARS-CoV-2 proteome undergoes a notable rearrangement when considering only peptides derived from viral structural proteins. The structural protein-specific peptide-allele distribution produced a distinct hierarchy of allele-binding capacities. This finding has important clinical implications as a majority of SARS-CoV-2 specific CD8+-T cell response is directed toward SARS-CoV-2 structural proteins. Therefore, patients who express MHC-I alleles enriched with a large potential repertoire of SARS-CoV-2 structural proteins peptides may benefit from a broader CD8+-T cell immune response.

The variations in SARS-CoV-2 peptide-allele distributions were analyzed at epidemiological scale to track its impact on country-specific mortality. Each of the 23 countries were assigned a population SARS-CoV-2 binding capacity (or EMP score) based on the individual binding capacities of the selected 52 MHC-I alleles weighted by their endemic population frequencies. This hierarchization revealed a strong inverse correlation between EMP score and observed population mortality, indicating that populations enriched with high SARS-CoV-2 binding capacity MHC-I alleles may be better protected. The correlation was shown to be stronger when calculating the EMP scores with respect to only structural proteins, reinforcing their relevance to viral immunity. Finally, the molecular origin of the 108 predicted peptides specific to SARS-CoV-2 structural proteins revealed that they are derived from enriched regions with a minimal predicted impact from amino acid sequence polymorphisms.

The utility of structural protein EMP scores was further supported by a multivariate analysis of additional SARS-CoV-2 risk factors. These results emphasized the relative robustness of structural protein EMP scores as a population risk assessment tool. Furthermore, a linear model based on the combination of structural protein EMP scores and select population-level risk factors was identified as a potential candidate for a predictive model for pandemic population severity. As such, the incorporation of the structural protein EMP score in more sophisticated models will likely improve epidemiological modeling.

To achieve the highest level of accuracy in MHC-I predictions, the most up-to-date versions of each component algorithm were used. However, this meant that several of the algorithms (MHCflurry, netMHCpan-EL-4.0, and MixMHCpred) were benchmarked against subsets of mass spectrometry data that were used in the original training of these MHC-I prediction models. While this could result in an unfair weight applied to these algorithms in peptide^{FDR} calculation, the individual FDRs of MHCflurry, netMHCpan-EL-4.0, and MixMHCpred were comparable to algorithms without this advantage (FIG. 24C). Furthermore, the peptide selection of SARS-CoV-2 peptides was shown to be highly cooperative within EnsembleMHC (FIG. 26A), and individual algorithms failed to replicate the strong observed correlations between population-binding capacity and observed SARS-CoV-2 mortality (FIG. 30E).

The presented model can be applied to predict individual T cell capacity to mount a robust SARS-CoV-2 immune response. Evolutionary divergence of patient MHC-I genotypes has been shown to be predictive of the response to immune checkpoint therapy in cancer and HIV. The versatility of the proposed model will be improved by the consideration of additional MHC-I alleles. To reduce the presence of confounding factors, EnsembleMHC was parameterized on only a subset of common MHC-I alleles that had strong existing experimental validation. While the selected MHC-I alleles are among some of the most common, personalized risk assessment will require consideration of the full patient MHC-I genotype. The continued mass spectrometry-based characterization of MHC-I peptide-binding motifs will help in this regard. However, due to the large potential sequence space of the MHC-I protein, extension of this model will likely require the inference of binding motifs based on MHC variant clustering.

### Method Details

### EnsembleMHC Component Binding and Processing Prediction Algorithms.

EnsembleMHC incorporates MHC-I binding and processing predictions from 7 publicly available algorithms: MHCflurry-affinity-1.6.0, MHCflurry-presentation-1.6.0, netMHC-4.0, netMHCpan-4.0-EL, netMHCstabpan-1.0, PickPocket-1.1, and, MixMHCpred-2.0.2. These algorithms were chosen based on the criteria of providing a free academic license, bash command line integration, and demonstrated accuracy for predicting SARS-CoV-2 MHC-I peptides with experimentally validated binding stability.

Each of the selected algorithms cover components of MHC-I binding and antigen processing that roughly fall into two categories: ones based primarily on MHC-I binding affinity predictions and others that incorporate antigen presentation. To this end, MHCflurry-affinity, netMHC, PickPocket, and netMHCstabpan predict binding affinity based on quantitative peptide binding affinity measurements. netMHCstabpan also incorporates peptide-MHC stability measurements and PickPocket performs prediction based on binding pocket structural extrapolation. To model the effects of antigen presentation, MixMHCpred, netMHCpan-EL, and MHCflurry—presentation are trained on naturally eluted MHC-I ligands. Additionally, MHCflurry-presentation incorporates an antigen processing term.

### Parameterization of EnsembleMHC Using Mass Spectrometry Data.

EnsembleMHC is able to achieve high levels of precision in peptide selection through the use of allele and algorithm-specific binding affinity thresholds. These binding affinity thresholds were identified through the parameterization of each algorithm on high-quality mass spectrometry datasets.17 The mass spectrometry datasets used for algorithm parameterization were collected in the largest single laboratory MS-based characterization of MHC-I peptides presented by single MHC allele cell lines. These characteristics significantly reduces the number of artifacts introduced by differences in peptide isolation methods, mass spectrometry acquisition, and convolution of peptides in multiallelic cell lines. An overview of the EnsembleMHC parameterization is provided in supplemental figures (Figure S1A).

Fifty-two common MHC-I alleles were selected for parameterization based on the criteria that they were characterized in Sarkizova et al. datasets and that all 7 component algorithms could perform peptide binding affinity predictions for that allele. Each target peptide (observed in the MS dataset) was paired with 100 length-matched randomly sampled decoy peptides (not observed in the MS dataset) derived from the same source proteins. If a protein was less than 100 amino acids in length, then every potential peptide from that protein was extracted.

Each of the seven algorithms were independently applied to each of the 52 allele datasets. For each allele dataset, the minimum score threshold was determined for each algorithm that recovered 50% of the allele repertoire size (the total number of target peptides observed in the MS dataset for that allele). Additionally, the expected accuracy of each algorithm was assessed by calculating the observed false detection rate (the fraction of identified peptides that were decoy peptides) using the identified algorithm- and allele-specific scoring threshold. The parameterization process was repeated 1000 times for each allele through bootstrap sampling of half of the peptides in each single MHC allele dataset. The final FDR and score threshold for each algorithm at each allele was determined by taking the median value of both quantities reported during bootstrap sampling.

### Peptide^{FDR} Calculation

Peptide confidence is assigned by calculating the peptide^{FDR}. This quantity is defined as the product of the empirical FDRs of each individual algorithm that detected a given peptide. The peptide^{FDR} is calculated using Equation 1,

$${peptide}^{FDR} = {\prod }_{i = 1 , i \neq ND}^{N} {algorithm}_{i}^{FDR} \qquad ( 1 )$$

where N is the number of MHC-I binding and processing algorythms, ND represents an algorithm that did not detect a given peptide, and algorithm^{FDR} represents the allele specific FDR of the Nth algorithm. The peptide^{FDR} represents the joint probability that all MHC-I binding and processing algorithms that detected a particular peptide did so in error, and therefore returns a probability of false detection. Unless otherwise stated, EnsembleMHC selected peptides based on the criterion of a peptide^{FDR} % 5%.

### Application of EnsembleMHC to Tumor Cell Line Data.

Ten tumor samples were obtained from the Sarkizova et al. datasets. Tumor samples were selected for analysis if at least 50% of the expressed MHC-I alleles for that sample were included in the 52 MHC-I alleles supported by EnsembleMHC. For each dataset, decoy peptides were generated in a manner identical to the method used for algorithm parameterization on single MHC allele data.

Peptide identification by each algorithm was based on restrictive or permissive binding affinities thresholds. These thresholds correspond to commonly used score cutoffs for the identification of strong binders (restrictive) or all binders (permissive) (0.5% (percentile rank) or 50 nM (IC50 value) for strong binders, and 2% (percentile rank) or 500 nM (IC50 value) for all binders). Due to the lack of recommend score thresholds for MHCflurry-presentation-1.6.0, the raw presentation score was converted to a percentile score using presentation scores produced by 100,000 randomly generated peptides.

### SARS-CoV-2 Reference Sequence.

MHC-I peptide predictions for the SARS-CoV-2 proteome were performed using the Wuhan-Hu-1 (GenBank: MN908947.3) reference sequence. All potential 8-14-mer peptides (n=67,207) were derived from the open reading frames in the reported proteome, and each peptide was evaluated by the EnsembleMHC workflow.

### SARS-CoV-2 Polymorphism Analysis and Protein Structure Visualizations.

Polymorphism analysis of SARS-CoV-2 structural proteins were performed using 102,148 full length protein sequences obtained from the COVIDep database. Solved structures for the E (PDB: 5X29) and S (PDB: 6VXX) proteins (worldwideweb.rcsb.org) and predicted structures for the M and N proteins were visualized using VMD.

### Application of EnsembleMHC to Determine Population SARS-CoV-2 Binding Capacity.

The peptides identified by the EnsembleMHC workflow were used to assess the SARS-CoV-2 population binding capacity by weighing individual MHC allele SARS-CoV-2 binding capacities by regional expression (for a schematic representation see FIG. 25).

The selection of countries included in the EnsembleMHC population binding capacity assessment was based on several criteria regarding the underlying MHC-I allele data for that country FIG. 25). The MHC-I allele frequency data used in the instant model was obtained from the Allele Frequency Net Database (AFND), and frequencies were aggregated by country. However, the currently available population-based MHC-I frequency data has specific limitations and variances, which was have addressed as follows:

MHC Allele Data Coverage within Countries

MHC-typing breadth was defined as the diversity of identified MHC-I alleles within a given country, and its depth as the ability to accurately achieve 4-digit MHC-I genotype resolution. High variability was observed in both the MHC-I genotyping breadth and depth (FIG. 25 inset). Consequently, additional filter-measures were introduced to capture potential sources of variance within the analyzed cohort of countries. The thresholds for filtering the country-wide MHC-I allele data were set based on meeting two inclusion criteria: 1) MHC genotyping of at least 1000 individuals have been performed in that population, avoiding skewing of allele frequencies due to small sample size. 2) MHC-I allele frequency data for at least 51 of the 52 (95%) MHC-1 alleles for which the EnsembleMHC was parameterized to predict, ensuring full power of the EnsembleMHC workflow.

Ethnic Communities within Countries

In instances where the MHC-I allele frequencies would pertain to more than one community, the reported frequencies were counted toward both contributing groups. For example, the MHC-I frequency data pertaining to the Chinese minority in Germany would be factored into the population MHC-I frequencies for both China and Germany. In doing so, this treatment resolves both ancestral and demographic MHC-I allele frequencies.

### Normalization of MHC Allele Frequency Data

The focus of this work was to uncover potential differences in SARS-CoV-2 MHC-I peptide presentation dynamics induced by the 52 selected alleles within a population. Accordingly, the MHC-I allele frequency data was carefully processed in order to maintain important differences in the expression of selected alleles, while minimizing the effect of confounding factors.

The MHC-I allele frequency data for a given population was first filtered to the 52 selected alleles. These allele frequencies were then converted to the theoretical total number of copies of that allele within the population (allele count) following,

allele count=allele_{freq}×2×n (2)

where allele_{freq} is the observed allele frequency in a population and n is the population sample size for which that allele frequency was measured. The allele count is then normalized with respect to the total allele count of selected 52 alleles within that population using the following relationship

$$norm allele count = \frac{allele {count}_{i}}{{\sum }_{i = 1}^{52} allele {count}_{i}} \qquad ( 3 )$$

where i is one of the 52 selected alleles. This normalization is required to overcome the potential bias toward hidden alleles (alleles that are either not well characterized or not supported by EnsembleMHC) as would be seen using alternative allele frequency accounting techniques (e.g., sample-weighted mean of selected allele frequencies or normalization with respect to all observed alleles within a population; FIG. 29C). The SARS-CoV-2 binding capacity of these hidden alleles cannot be accurately determined using the EnsembleMHC workflow, and therefore important potential relationships would be obscured.

### EnsembleMHC Population Score

The predicted ability of a given population to present SARS-CoV-2 derived peptides was assessed by calculating the EnsembleMHC Population (EMP) score. After the MHC-I allele frequency data filtering steps, 23 countries were included in the analysis. The calculation of the EnsembleMHC population score is as follows

$$EMP score = \frac{{\sum }_{i = 1}^{52} {peptide}_{trac} \times norm allele {count}_{i}}{{N}_{norm allele count \neq 0}} , \qquad ( 4 )$$

where norm allele count is the observed normalized allele count for a given allele in a population, N_{norm allele count}s≠0 is the number of the 52 select alleles detected in a given population (range 51-52 alleles), and peptide_{frac} is the peptide fraction or the fraction of total predicted peptides expected to be presented by that allele within the total set of predicted peptides with a peptide^{FDR}≤5%.

### Death Rate-Presentation Correlation

The correlation between the EMP score and the observed deaths per million within the cohort of selected countries was calculated as a function of time. SARS-Cov-2 data covering the time dependent global evolution of the SARS-CoV-2 pandemic was obtained from Johns Hopkins University Center for Systems Science and Engineering covering the time frame of January 22nd to Apr. 9th 2020. The temporal variations in occurrence of community spread observed in different countries were accounted for by rescaling the time series data relative to when a certain minimum death threshold was met in a country. This analysis was performed for minimum death thresholds of 1-100 total deaths by day 0, and correlations were calculated at each day sequentially following day 0 until there were fewer than 8 countries remaining at that time point. The upper-limit of 100-deaths was chosen due to a steep decline in average statistical power observed with day 1 death thresholds greater than 100 deaths (FIG. 29E).

The time death correlation was computed using Spearman's rank correlation coefficient (two-sided). This method was chosen due to the small sample size and non-normality of the underlying data (FIG. 29D). The reported correlations of EMP score and deaths per million using other correlation methods can be seen in supplemental FIG. 29A.

The low statistical power for some of the obtained correlations were addressed by calculating the Positive Predictive Value (PPV) of all correlations using the following equation 5,

$$PPV = \frac{1 - β \times R}{1 - β \times R + α} \qquad ( 5 )$$

where 1 is the statistical power of a given correlation, R is the pre-study odds, and a is the significance level. A PPV value of ≥95% is analogous to a p value of ≤0.05. Due to an unknown pre-study odd (probability that probed effect is truly non null), R was set to 1 in the reported correlations. The significance of partitioning high risk and low risk countries based on median EMP score was determined using Mann-Whitney U-test. Significance values were corrected for multiple tests using the Benjamini-Hochberg procedure.

Sub-Sampling of Peptides from the Full SARS-CoV-2 Proteome

108 unique peptides, derived from the Full SARS-CoV-2 proteome and passing the 5% peptide^{FDR} filter, were randomly sampled. Then, the time series EMP score—death per million correlation analysis used to generate FIG. 22 was applied to each sampled peptide set. The sub-sampling procedure was repeated for 1,000 iterations (FIG. 30A). To quantitatively describe the similarity of the distributions, the Kullback-Leibler divergence (KLD), a measure of divergence between two probability distributions, was calculated for the correlation distribution of each sub-sample iteration relative to either the correlation distribution of the Full SARS-CoV-2 proteome or SARS-CoV-2 structural proteins (FIG. 30B).

Additional SARS-CoV-2 Risk Factors

Twelve potential SARS-CoV-2 risk factors (Table S2) were selected for analysis. Country-specific data for each risk factor was obtained from the Global Health Observatory data repository provided by the World Health Organization (https://apps.who.int/gho/data/node.main). Countries were selected for analysis based on the criteria of having reported data in the WHO datasets and inclusion in the set of 23 countries for which EnsembleMHC population scores were assigned (Table S2A). Data regarding the total number of noncommunicable disease-related deaths (Cardiovascular disease, Chronic obstructive pulmonary disease, and Diabetes mellitus) were converted to deaths per million.

Correlation of Additional Risk Factors with Observed Deaths Per Million

Correlation analysis of each additional factor was carried out in a similar manner to that of the EnsembleMHC population score. In short, Spearman's correlation coefficient between each individual factor and observed deaths per million was estimated as a function of time from when a specified minimum death threshold was met (FIG. 23). The significance level was set to p≤0.05 and significant PPV was set to PPV≥0.95.

Linear models of SARS-CoV-2 mortality

For the Single and Combination Models, Individual Linear Models were constructed for each considered death threshold as a function of time (similar to the univariate correlation analysis). Each model consisted of 1 (a single socioeconomic or health-related risk factor) or 2 (a combination of 1 risk factor and structural protein EMP score) dependent variables and deaths per million as the independent variable. The adjusted R² value and statistical significance of the model (F-test) were then extracted from each individual model and aggregated by dependent variable (FIG. 31A).

The best performing models were determined by assessing all possible combinations of factors including structural protein EMP score. This resulted in the consideration of 4,083 different linear models. The top performing models were then selected by ranking each model by median adjusted R².

Immunogenic Viral Peptide Analysis

Individual algorithms were assessed for ability to prioritize viral peptides with known immunogenicity by calculating the precision (experimentally validated peptides/putative non-immunogenic peptides) when selecting n number of top scoring peptides as determined by a given algorithm. For example, if n=25, then the precision of each algorithm would be calculated based on the top 25 highest scoring peptides according to that algorithm. A Viral peptide dataset was generated by extracting all potential 8-14-mer peptides from the Hepatitis-C genome polyprotein (P26664), the Dengue virus genome polyprotein (P14340), and the HIV-1 POL-GAG protein (P03369). The resulting peptides were then checked against the Immune Epitope database (IEDB, worldwideweb.iedb.org/) to identify peptides with experimentally validated immunogenicity. This resulted in the generation of a dataset comprised of 616 experimentally validated immunogenic peptides and 54,663 putative non-immunogenic peptides (this includes peptides experimentally determined to be non-immunogenic or peptides with unknown immunogenicity). To benchmark EnsembleMHC against other Ensemble-based MHC-I peptide prediction algorithms, netCTLpan and MHCcons were included for comparison purposes.

### Quantification and Statistical Analysis

Statistical tests were performed using R 4.0.3. All effect size estimations were performed using Spearman's rank correlation. Mann-Whitney U test was used to test for significant testing of death rate stratification between countries with high and low EnsembleMHC score. The threshold for statistical significance was set to p values of ≤0.05 or positive predictive value of PPV ≥0.95. Where indicated, p value correction for multiple testing was accomplished using the Benjamini-Hochberg procedure.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

As used herein, the terms “polypeptide” or “peptide” are used interchangeably and mean any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. For instance, a peptide can be about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 amino acids in length or longer. A peptide can also be about 5-10, 7-15, 10-15, 10-20, 15-20, 20-25, 20-30, 30-35, 30-40, 35-40, 40-45, 40-50, 45-50, 50-55, 50-60, 55-60, 60-65, 60-70, 65-70, 70-75, 70-80, 75-80, 80-85, 80-90, 85-90, 90-95, 90-100, 95-100, or more than 100 amino acids in length or any individual length within these ranges. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As various changes could be made in the above-described cells and methods without departing from the scope of the invention, it is intended that all matter contained in the above description and in the examples given below, shall be interpreted as illustrative and not in a limiting sense.

### Sequences

TABLE A

| SEQ ID NO | peptide | peptideFDR | gene | HLA |
|---|---|---|---|---|
| SEQ ID NO: 1. | AMDEFIERY | 0.000675 | orf1ab | A01:01 |
| SEQ ID NO: 2. | ATEETFKLSY | 0.00196 | orf1ab | A01:01 |
| SEQ ID NO: 3. | ATSRTLSYY | 0.003896 | M | A01:01 |
| SEQ ID NO: 4. | CSDKAYKIEELFY | 0.018504 | orf1ab | A01:01 |
| SEQ ID NO: 5. | CTDDNALAY | 0.000175 | orf1ab | A01:01 |
| SEQ ID NO: 6. | CTDDNALAYY | 0.000175 | orf1ab | A01:01 |
| SEQ ID NO: 7. | DTDFVNEFY | 3.63E-05 | orf1ab | A01:01 |
| SEQ ID NO: 8. | FADDLNQLTGY | 0.009308 | orf1ab | A01:01 |
| SEQ ID NO: 9. | FIDTKRGVY | 0.014641 | orf1ab | A01:01 |
| SEQ ID NO: 10. | FTSDYYQLY | 0.000175 | ORF3a | A01:01 |
| SEQ ID NO: 11. | GTDLEGNFY | 3.63E-05 | orf1ab | A01:01 |
| SEQ ID NO: 12. | HTDFSSEIIGY | 0.003418 | orf1ab | A01:01 |
| SEQ ID NO: 13. | ISDYDYYRY | 3.63E-05 | orf1ab | A01:01 |
| SEQ ID NO: 14. | ITDVFYKENSY | 0.011145 | orf1ab | A01:01 |
| SEQ ID NO: 15. | IVDTVSALVY | 0.00071 | orf1ab | A01:01 |
| SEQ ID NO: 16. | KSDGTGTIY | 0.002783 | orf1ab | A01:01 |
| SEQ ID NO: 17. | LSDDAVVCFNSTY | 0.01435 | orf1ab | A01:01 |
| SEQ ID NO: 18. | LTDEMIAQY | 3.63E-05 | S | A01:01 |
| SEQ ID NO: 19. | LTENLLLY | 0.018504 | orf1ab | A01:01 |
| SEQ ID NO: 20. | LTNDNTSRY | 0.000866 | orf1ab | A01:01 |
| SEQ ID NO: 21. | MADQAMTQMY | 0.011145 | orf1ab | A01:01 |
| SEQ ID NO: 22. | PTDNYITTY | 0.000866 | orf1ab | A01:01 |
| SEQ ID NO: 23. | STDGNKIADKY | 0.003418 | orf1ab | A01:01 |
| SEQ ID NO: 24. | STECSNLLLQY | 0.000786 | S | A01:01 |
| SEQ ID NO: 25. | TSDYYQLY | 0.018504 | ORF3a | A01:01 |
| SEQ ID NO: 26. | TTDPSFLGRY | 3.63E-05 | orf1ab | A01:01 |
| SEQ ID NO: 27. | TVDSSQGSEY | 0.046784 | orf1ab | A01:01 |
| SEQ ID NO: 28. | VTDVTQLY | 0.000172 | orf1ab | A01:01 |
| SEQ ID NO: 29. | VTDVTQLYL | 0.014437 | orf1ab | A01:01 |
| SEQ ID NO: 30. | VTDVTQLYLGGMSY | 0.004112 | orf1ab | A01:01 |
| SEQ ID NO: 31. | VVDDPCPIHFY | 0.011145 | ORF8 | A01:01 |
| SEQ ID NO: 32. | VVDKYFDCY | 0.000809 | orf1ab | A01:01 |
| SEQ ID NO: 33. | VVDYGARFY | 0.003518 | orf1ab | A01:01 |
| SEQ ID NO: 34. | YADVFHLYLQY | 0.018504 | orf1ab | A01:01 |
| SEQ ID NO: 35. | ALSKGVHFV | 0.003483 | ORF3a | A02:01 |
| SEQ ID NO: 36. | ALWEIQQVV | 0.002799 | orf1ab | A02:01 |
| SEQ ID NO: 37. | FLAFVVFLL | 0.038662 | E | A02:01 |
| SEQ ID NO: 38. | FLAHIQWMV | 0.012808 | orf1ab | A02:01 |
| SEQ ID NO: 39. | FLLNKEMYL | 0.00323 | orf1ab | A02:01 |
| SEQ ID NO: 40. | FLLPSLATV | 0.001154 | orf1ab | A02:01 |
| SEQ ID NO: 41. | FLNRFTTTL | 0.027267 | orf1ab | A02:01 |
| SEQ ID NO: 42. | ILFTRFFYV | 0.038662 | orf1ab | A02:01 |
| SEQ ID NO: 43. | KLIEYTDFA | 0.038662 | orf1ab | A02:01 |
| SEQ ID NO: 44. | KLSYGIATV | 0.038662 | orf1ab | A02:01 |
| SEQ ID NO: 45. | LLLDDFVEI | 0.028465 | orf1ab | A02:01 |
| SEQ ID NO: 46. | LLYDANYFL | 0.012808 | ORF3a | A02:01 |
| SEQ ID NO: 47. | NLSDRVVFV | 0.005778 | orf1ab | A02:01 |
| SEQ ID NO: 48. | SMWALIISV | 0.013811 | orf1ab | A02:01 |
| SEQ ID NO: 49. | TLMNVLTLV | 0.013811 | orf1ab | A02:01 |
| SEQ ID NO: 50. | TMADLVYAL | 0.002564 | orf1ab | A02:01 |
| SEQ ID NO: 51. | VLWAHGFEL | 0.028465 | orf1ab | A02:01 |
| SEQ ID NO: 52. | YLATALLTL | 0.004575 | orf1ab | A02:01 |
| SEQ ID NO: 53. | YLDAYNMMI | 0.031068 | orf1ab | A02:01 |
| SEQ ID NO: 54. | YLFDESGEFKL | 0.014017 | orf1ab | A02:01 |
| SEQ ID NO: 55. | YLNTLTLAV | 0.038662 | orf1ab | A02:01 |
| SEQ ID NO: 56. | YLQPRTFLL | 0.007178 | S | A02:01 |
| SEQ ID NO: 57. | YLYALVYFL | 0.013811 | ORF3a | A02:01 |
| SEQ ID NO: 58. | ALSKGVHFV | 0.005288 | ORF3a | A02:02 |
| SEQ ID NO: 59. | FIAGLIAIV | 0.031693 | S | A02:02 |
| SEQ ID NO: 60. | FLAHIQWMV | 0.011838 | orf1ab | A02:02 |
| SEQ ID NO: 61. | FLLNKEMYL | 0.008149 | orf1ab | A02:02 |
| SEQ ID NO: 62. | FLLPSLATV | 0.00759 | orf1ab | A02:02 |
| SEQ ID NO: 63. | FLNRFTTTL | 0.011838 | orf1ab | A02:02 |
| SEQ ID NO: 64. | FLPRVFSAV | 0.031693 | orf1ab | A02:02 |
| SEQ ID NO: 65. | FVNEFYAYL | 0.011838 | orf1ab | A02:02 |
| SEQ ID NO: 66. | KIADYNYKL | 0.016696 | S | A02:02 |
| SEQ ID NO: 67. | KLSYGIATV | 0.026174 | orf1ab | A02:02 |
| SEQ ID NO: 68. | LLYDANYFL | 0.011838 | ORF3a | A02:02 |
| SEQ ID NO: 69. | NLSDRVVFV | 0.007033 | orf1ab | A02:02 |
| SEQ ID NO: 70. | QMAPISAMV | 0.048822 | orf1ab | A02:02 |
| SEQ ID NO: 71. | SLPGVFCGV | 0.041635 | orf1ab | A02:02 |
| SEQ ID NO: 72. | TLMNVLTLV | 0.031693 | orf1ab | A02:02 |
| SEQ ID NO: 73. | TMADLVYAL | 0.003433 | orf1ab | A02:02 |
| SEQ ID NO: 74. | VLNDILSRL | 0.011838 | S | A02:02 |
| SEQ ID NO: 75. | VMVELVAEL | 0.04032 | orf1ab | A02:02 |
| SEQ ID NO: 76. | YLATALLTL | 0.011838 | orf1ab | A02:02 |
| SEQ ID NO: 77. | YLNTLTLAV | 0.031693 | orf1ab | A02:02 |
| SEQ ID NO: 78. | YLQPRTFLL | 0.003433 | S | A02:02 |
| SEQ ID NO: 79. | YLTNDVSFL | 0.011838 | orf1ab | A02:02 |
| SEQ ID NO: 80. | ALSKGVHFV | 0.000533 | ORF3a | A02:03 |
| SEQ ID NO: 81. | FLAHIQWMV | 0.027668 | orf1ab | A02:03 |
| SEQ ID NO: 82. | FLLNKEMYL | 0.041764 | orf1ab | A02:03 |
| SEQ ID NO: 83. | FLLPSLATV | 0.000533 | orf1ab | A02:03 |
| SEQ ID NO: 84. | FLNGSCGSV | 0.027668 | orf1ab | A02:03 |
| SEQ ID NO: 85. | FLNRFTTTL | 0.009167 | orf1ab | A02:03 |
| SEQ ID NO: 86. | FLPRVFSAV | 0.009167 | orf1ab | A02:03 |
| SEQ ID NO: 87. | GLNDNLLEI | 0.014786 | orf1ab | A02:03 |
| SEQ ID NO: 88. | KLKDCVMYA | 0.017475 | orf1ab | A02:03 |
| SEQ ID NO: 89. | KLSYGIATV | 0.002071 | orf1ab | A02:03 |
| SEQ ID NO: 90. | MLAKALRKV | 0.00625 | orf1ab | A02:03 |
| SEQ ID NO: 91. | NLSDRVVFV | 0.001362 | orf1ab | A02:03 |
| SEQ ID NO: 92. | RLANECAQV | 0.043296 | orf1ab | A02:03 |
| SEQ ID NO: 93. | SLPGVFCGV | 0.045086 | orf1ab | A02:03 |
| SEQ ID NO: 94. | TLATHGLAAV | 0.027668 | orf1ab | A02:03 |
| SEQ ID NO: 95. | TLIGDCATV | 0.043296 | orf1ab | A02:03 |
| SEQ ID NO: 96. | TLMNVLTLV | 0.009167 | orf1ab | A02:03 |
| SEQ ID NO: 97. | TMADLVYAL | 0.006029 | orf1ab | A02:03 |
| SEQ ID NO: 98. | VLAWLYAAV | 0.027668 | orf1ab | A02:03 |
| SEQ ID NO: 99. | VLNDILSRL | 0.002071 | S | A02:03 |
| SEQ ID NO: 100. | YLATALLTL | 0.002071 | orf1ab | A02:03 |
| SEQ ID NO: 101. | YLNTLTLAV | 0.009167 | orf1ab | A02:03 |
| SEQ ID NO: 102. | YLQPRTFLL | 0.045086 | S | A02:03 |
| SEQ ID NO: 103. | ALSKGVHFV | 0.042339 | ORF3a | A02:05 |
| SEQ ID NO: 104. | FLLPSLATV | 0.042339 | orf1ab | A02:05 |
| SEQ ID NO: 105. | YLQPRTFLL | 0.047707 | S | A02:05 |
| SEQ ID NO: 106. | ALWEIQQVV | 0.029808 | orf1ab | A02:06 |
| SEQ ID NO: 107. | FLAHIQWMV | 0.048379 | orf1ab | A02:06 |
| SEQ ID NO: 108. | FLLPSLATV | 0.016154 | orf1ab | A02:06 |
| SEQ ID NO: 109. | KIADYNYKL | 0.037881 | S | A02:06 |
| SEQ ID NO: 110. | LLYDANYFL | 0.048379 | ORF3a | A02:06 |
| SEQ ID NO: 111. | SLPGVFCGV | 0.036095 | orf1ab | A02:06 |
| SEQ ID NO: 112. | TLMNVLTLV | 0.048379 | orf1ab | A02:06 |
| SEQ ID NO: 113. | TVYSHLLLV | 0.036095 | ORF3a | A02:06 |
| SEQ ID NO: 114. | YLQPRTFLL | 0.029808 | S | A02:06 |
| SEQ ID NO: 115. | YVWKSYVHV | 0.048379 | orf1ab | A02:06 |
| SEQ ID NO: 116. | ALSKGVHFV | 0.049026 | ORF3a | A02:07 |
| SEQ ID NO: 117. | ALWEIQQVV | 0.028968 | orf1ab | A02:07 |
| SEQ ID NO: 118. | FLAHIQWMV | 0.032251 | orf1ab | A02:07 |
| SEQ ID NO: 119. | FLLPSLATV | 0.002745 | orf1ab | A02:07 |
| SEQ ID NO: 120. | FLPRVFSAV | 0.00823 | orf1ab | A02:07 |
| SEQ ID NO: 121. | FVDGVPFVV | 0.034833 | orf1ab | A02:07 |
| SEQ ID NO: 122. | LLDDFVEII | 0.01323 | orf1ab | A02:07 |
| SEQ ID NO: 123. | SLPGVFCGV | 0.006962 | orf1ab | A02:07 |
| SEQ ID NO: 124. | TMADLVYAL | 0.030378 | orf1ab | A02:07 |
| SEQ ID NO: 125. | YIDIGNYTV | 0.016902 | ORF8 | A02:07 |
| SEQ ID NO: 126. | YLQPRTFLL | 0.019331 | S | A02:07 |
| SEQ ID NO: 127. | ALSKGVHFV | 0.005347 | ORF3a | A02:11 |
| SEQ ID NO: 128. | ALWEIQQVV | 0.006854 | orf1ab | A02:11 |
| SEQ ID NO: 129. | FLAFVVFLL | 0.02643 | E | A02:11 |
| SEQ ID NO: 130. | FLAHIQWMV | 0.010513 | orf1ab | A02:11 |
| SEQ ID NO: 131. | FLLNKEMYL | 0.011887 | orf1ab | AO2:11 |
| SEQ ID NO: 132. | FLLPSLATV | 0.002892 | orf1ab | AO2:11 |
| SEQ ID NO: 133. | ILFTRFFYV | 0.02643 | orf1ab | AO2:11 |
| SEQ ID NO: 134. | KLLEQWNLV | 0.024916 | M | AO2:11 |
| SEQ ID NO: 135. | KLSYGIATV | 0.019436 | orf1ab | AO2:11 |
| SEQ ID NO: 136. | LLYDANYFL | 0.010513 | ORF3a | AO2:11 |
| SEQ ID NO: 137. | NLIDSYFVV | 0.045558 | orf1ab | AO2:11 |
| SEQ ID NO: 138. | NLSDRVVFV | 0.023171 | orf1ab | AO2:11 |
| SEQ ID NO: 139. | SLVKPSFYV | 0.023372 | E | AO2:11 |
| SEQ ID NO: 140. | SMWALIISV | 0.010513 | orf1ab | AO2:11 |
| SEQ ID NO: 141. | TLMNVLTLV | 0.037887 | orf1ab | AO2:11 |
| SEQ ID NO: 142. | TMADLVYAL | 0.005637 | orf1ab | AO2:11 |
| SEQ ID NO: 143. | VLWAHGFEL | 0.023372 | orf1ab | AO2:11 |
| SEQ ID NO: 144. | YLATALLTL | 0.045558 | orf1ab | AO2:11 |
| SEQ ID NO: 145. | YLQPRTFLL | 0.005637 | S | AO2:11 |
| SEQ ID NO: 146. | YLYALVYFL | 0.010513 | ORF3a | AO2:11 |
| SEQ ID NO: 147. | ALAYYNTTK | 0.009539 | orf1ab | A03:01 |
| SEQ ID NO: 148. | ALCTFLLNK | 0.002922 | orf1ab | A03:01 |
| SEQ ID NO: 149. | ASMPTTIAK | 0.007249 | orf1ab | A03:01 |
| SEQ ID NO: 150. | GVAMPNLYK | 0.00319 | orf1ab | A03:01 |
| SEQ ID NO: 151. | GVYFASTEK | 0.004513 | S | A03:01 |
| SEQ ID NO: 152. | GVYYHKNNK | 0.034621 | S | A03:01 |
| SEQ ID NO: 153. | KLFAAETLK | 0.004513 | orf1ab | A03:01 |
| SEQ ID NO: 154. | KLFDRYFKY | 0.002708 | orf1ab | A03:01 |
| SEQ ID NO: 155. | KMADQAMTQMYK | 0.031979 | orf1ab | A03:01 |
| SEQ ID NO: 156. | KMQRMLLEK | 0.031979 | orf1ab | A03:01 |
| SEQ ID NO: 157. | KSAGFPFNK | 0.004513 | orf1ab | A03:01 |
| SEQ ID NO: 158. | KTFPPTEPK | 0.004513 | N | A03:01 |
| SEQ ID NO: 159. | KTFPPTEPKK | 0.012554 | N | A03:01 |
| SEQ ID NO: 160. | KTIQPRVEK | 0.010255 | orf1ab | A03:01 |
| SEQ ID NO: 161. | KVAGFAKFLK | 0.031979 | orf1ab | A03:01 |
| SEQ ID NO: 162. | QTFFKLVNK | 0.015674 | orf1ab | A03:01 |
| SEQ ID NO: 163. | RLFRKSNLK | 0.004513 | S | A03:01 |
| SEQ ID NO: 164. | RLISMMGFK | 0.012554 | orf1ab | A03:01 |
| SEQ ID NO: 165. | RQFHQKLLK | 0.004513 | orf1ab | A03:01 |
| SEQ ID NO: 166. | STFNVPMEK | 0.001147 | orf1ab | A03:01 |
| SEQ ID NO: 167. | TSFGPLVRK | 0.020773 | orf1ab | A03:01 |
| SEQ ID NO: 168. | TTIKPVTYK | 0.002708 | orf1ab | A03:01 |
| SEQ ID NO: 169. | VLSGHNLAK | 0.009539 | orf1ab | A03:01 |
| SEQ ID NO: 170. | VTNNTFTLK | 0.004513 | orf1ab | A03:01 |
| SEQ ID NO: 171. | VTYVPAQEK | 0.029386 | S | A03:01 |
| SEQ ID NO: 172. | WYRGIIIYK | 0.001147 | orf1ab | A03:01 |
| SEQ ID NO: 173. | AIDAYPLTK | 0.026401 | orf1ab | A11:01 |
| SEQ ID NO: 174. | AIVSTIQRK | 0.043911 | orf1ab | A11:01 |
| SEQ ID NO: 175. | ALCTFLLNK | 0.00239 | orf1ab | A11:01 |
| SEQ ID NO: 176. | ASANLAATK | 0.00373 | S | A11:01 |
| SEQ ID NO: 177. | ASMPTTIAK | 0.000873 | orf1ab | A11:01 |
| SEQ ID NO: 178. | ATAEAELAK | 0.009 | orf1ab | A11:01 |
| SEQ ID NO: 179. | ATSRTLSYYK | 0.029639 | M | A11:01 |
| SEQ ID NO: 180. | ATVVIGTSK | 0.006112 | orf1ab | A11:01 |
| SEQ ID NO: 181. | AVAKHDFFK | 0.010827 | orf1ab | A11:01 |
| SEQ ID NO: 182. | AVFDKNLYDK | 0.000873 | orf1ab | A11:01 |
| SEQ ID NO: 183. | AVLQSGFRK | 0.002106 | orf1ab | A11:01 |
| SEQ ID NO: 184. | GTHWFVTQR | 0.041316 | S | A11:01 |
| SEQ ID NO: 185. | GTLSYEQFK | 0.010827 | orf1ab | A11:01 |
| SEQ ID NO: 186. | GTLSYEQFKK | 0.048686 | orf1ab | A11:01 |
| SEQ ID NO: 187. | GVAMPNLYK | 0.002534 | orf1ab | A11:01 |
| SEQ ID NO: 188. | GVYFASTEK | 0.00373 | S | A11:01 |
| SEQ ID NO: 189. | HVVGPNVNK | 0.014905 | orf1ab | A11:01 |
| SEQ ID NO: 190. | IINNTVYTK | 0.026732 | orf1ab | A11:01 |
| SEQ ID NO: 191. | KSAGFPFNK | 0.00373 | orf1ab | A11:01 |
| SEQ ID NO: 192. | KTFPPTEPK | 0.00373 | N | A11:01 |
| SEQ ID NO: 193. | KTFPPTEPKK | 0.00373 | N | A11:01 |
| SEQ ID NO: 194. | KTIQPRVEK | 0.009 | orf1ab | A11:01 |
| SEQ ID NO: 195. | KTQFNYYKK | 0.010212 | orf1ab | A11:01 |
| SEQ ID NO: 196. | KVAGFAKFLK | 0.010827 | orf1ab | A11:01 |
| SEQ ID NO: 197. | MTNRQFHQK | 0.029639 | orf1ab | A11:01 |
| SEQ ID NO: 198. | QTFFKLVNK | 0.002156 | orf1ab | A11:01 |
| SEQ ID NO: 199. | QVVNVVTTK | 0.010276 | orf1ab | A11:01 |
| SEQ ID NO: 200. | SASKIITLK | 0.002156 | ORF3a | A11:01 |
| SEQ ID NO: 201. | SHNNTVYTK | 0.026732 | orf1ab | A11:01 |
| SEQ ID NO: 202. | STFNVPMEK | 0.000873 | orf1ab | A11:01 |
| SEQ ID NO: 203. | STMTNRQFHQK | 0.029272 | orf1ab | A11:01 |
| SEQ ID NO: 204. | TSFGPLVRK | 0.002156 | orf1ab | A11:01 |
| SEQ ID NO: 205. | TTIKPVTYK | 0.000873 | orf1ab | A11:01 |
| SEQ ID NO: 206. | TVIEVQGYK | 0.026732 | orf1ab | A11:01 |
| SEQ ID NO: 207. | VTNNTFTLK | 0.00373 | orf1ab | A11:01 |
| SEQ ID NO: 208. | VTYVPAQEK | 0.009 | S | A11:01 |
| SEQ ID NO: 209. | WYRGIIIYK | 0.010827 | orf1ab | A11:01 |
| SEQ ID NO: 210. | AYANSVFNI | 0.012119 | orf1ab | A23:01 |
| SEQ ID NO: 211. | AYILFTRFF | 0.009729 | orf1ab | A23:01 |
| SEQ ID NO: 212. | AYVNTFSSTF | 0.015233 | orf1ab | A23:01 |
| SEQ ID NO: 213. | CYFGLFCLL | 0.038478 | orf1ab | A23:01 |
| SEQ ID NO: 214. | CYFPLQSYGF | 0.015233 | S | A23:01 |
| SEQ ID NO: 215. | FYLTNDVSF | 0.001186 | orf1ab | A23:01 |
| SEQ ID NO: 216. | GYKSVNITF | 0.014642 | orf1ab | A23:01 |
| SEQ ID NO: 217. | IYLYLTFYL | 0.016358 | orf1ab | A23:01 |
| SEQ ID NO: 218. | IYNDKVAGF | 0.000859 | orf1ab | A23:01 |
| SEQ ID NO: 219. | IYTELEPPCRF | 0.03583 | orf1ab | A23:01 |
| SEQ ID NO: 220. | KFTDGVCLF | 0.038478 | orf1ab | A23:01 |
| SEQ ID NO: 221. | LYDKLVSSF | 0.03444 | orf1ab | A23:01 |
| SEQ ID NO: 222. | LYLYALVYF | 0.015233 | ORF3a | A23:01 |
| SEQ ID NO: 223. | LYNSASFSTF | 0.015233 | S | A23:01 |
| SEQ ID NO: 224. | MYASAVVLL | 0.014642 | orf1ab | A23:01 |
| SEQ ID NO: 225. | MYMGTLSYEQF | 0.015233 | orf1ab | A23:01 |
| SEQ ID NO: 226. | NYLKRRVVF | 0.049921 | orf1ab | A23:01 |
| SEQ ID NO: 227. | NYMPYFFTL | 0.000365 | orf1ab | A23:01 |
| SEQ ID NO: 228. | QYIKWPWYI | 0.001477 | S | A23:01 |
| SEQ ID NO: 229. | RYPANSIVCRF | 0.03583 | orf1ab | A23:01 |
| SEQ ID NO: 230. | SYFIASFRL | 0.016734 | M | A23:01 |
| SEQ ID NO: 231. | SYFIASFRLF | 0.004798 | M | A23:01 |
| SEQ ID NO: 232. | SYFVVKRHTF | 0.046486 | orf1ab | A23:01 |
| SEQ ID NO: 233. | TFFKLVNKF | 0.004508 | orf1ab | A23:01 |
| SEQ ID NO: 234. | TYACWHHSI | 0.016358 | orf1ab | A23:01 |
| SEQ ID NO: 235. | TYKPNTWCI | 0.016358 | orf1ab | A23:01 |
| SEQ ID NO: 236. | TYLEGSVRWTTF | 0.03936 | orf1ab | A23:01 |
| SEQ ID NO: 237. | VYDPLQPEL | 0.029024 | S | A23:01 |
| SEQ ID NO: 238. | VYFLQSINF | 0.002789 | ORF3a | A23:01 |
| SEQ ID NO: 239. | VYIGDPAQL | 0.009142 | orf1ab | A23:01 |
| SEQ ID NO: 240. | VYMPASVWM | 0.002789 | orf1ab | A23:01 |
| SEQ ID NO: 241. | VYMPASWVMRI | 0.030888 | orf1ab | A23:01 |
| SEQ ID NO: 242. | VYSTGSNVF | 0.036985 | S | A23:01 |
| SEQ ID NO: 243. | VYYTSNPTTF | 0.000365 | orf1ab | A23:01 |
| SEQ ID NO: 244. | YFIASFRLF | 0.001477 | M | A23:01 |
| SEQ ID NO: 245. | YFTSDYYQL | 0.049921 | ORF3a | A23:01 |
| SEQ ID NO: 246. | YFVVKRHTF | 0.046486 | orf1ab | A23:01 |
| SEQ ID NO: 247. | YYHTTDPSF | 0.014642 | orf1ab | A23:01 |
| SEQ ID NO: 248. | YYKKDNSYF | 0.049921 | orf1ab | A23:01 |
| SEQ ID NO: 249. | YYTSNPTTF | 0.001477 | orf1ab | A23:01 |
| SEQ ID NO: 250. | AYANSVFNI | 0.023697 | orf1ab | A24:02 |
| SEQ ID NO: 251. | AYVNTFSSTF | 0.00733 | orf1ab | A24:02 |
| SEQ ID NO: 252. | CYFPLQSYGF | 0.029893 | S | A24:02 |
| SEQ ID NO: 253. | FFASFYYVW | 0.029893 | orf1ab | A24:02 |
| SEQ ID NO: 254. | FYLTNDVSF | 0.020402 | orf1ab | A24:02 |
| SEQ ID NO: 255. | GYINVFAFPF | 0.029893 | ORF10 | A24:02 |
| SEQ ID NO: 256. | GYKSVNITF | 0.022128 | orf1ab | A24:02 |
| SEQ ID NO: 257. | IYLYLTFYL | 0.01967 | orf1ab | A24:02 |
| SEQ ID NO: 258. | IYNDKVAGF | 0.00029 | orf1ab | A24:02 |
| SEQ ID NO: 259. | LYALVYFLQSINF | 0.029893 | ORF3a | A24:02 |
| SEQ ID NO: 260. | LYDKLVSSF | 0.004887 | orf1ab | A24:02 |
| SEQ ID NO: 261. | LYIIKLIFLW | 0.029893 | M | A24:02 |
| SEQ ID NO: 262. | LYLTFYLTNDVSF | 0.029893 | orf1ab | A24:02 |
| SEQ ID NO: 263. | LYLYALVYF | 0.01967 | ORF3a | A24:02 |
| SEQ ID NO: 264. | LYNSASFSTF | 0.00733 | S | A24:02 |
| SEQ ID NO: 265. | MYASAVVLL | 0.007427 | orf1ab | A24:02 |
| SEQ ID NO: 266. | MYASAVVLLI | 0.029893 | orf1ab | A24:02 |
| SEQ ID NO: 267. | MYIFFASFYYVW | 0.029893 | orf1ab | A24:02 |
| SEQ ID NO: 268. | MYMGTLSYEQF | 0.029893 | orf1ab | A24:02 |
| SEQ ID NO: 269. | NYMPYFFTL | 9.74E-05 | orf1ab | A24:02 |
| SEQ ID NO: 270. | NYMPYFFTLL | 0.029893 | orf1ab | A24:02 |
| SEQ ID NO: 271. | NYMPYFFTLLL | 0.029893 | orf1ab | A24:02 |
| SEQ ID NO: 272. | NYMPYFFTLLLQL | 0.029893 | orf1ab | A24:02 |
| SEQ ID NO: 273. | QYIKWPWYI | 0.000431 | S | A24:02 |
| SEQ ID NO: 274. | QYIKWPWYIW | 0.029893 | S | A24:02 |
| SEQ ID NO: 275. | QYIKWPWYIWLGF | 0.029893 | S | A24:02 |
| SEQ ID NO: 276. | RYLALYNKYKYF | 0.029893 | orf1ab | A24:02 |
| SEQ ID NO: 277. | RYPANSIVCRF | 0.00733 | orf1ab | A24:02 |
| SEQ ID NO: 278. | TYACWHHSI | 0.006602 | orf1ab | A24:02 |
| SEQ ID NO: 279. | TYASALWEI | 0.006602 | orf1ab | A24:02 |
| SEQ ID NO: 280. | TYKPNTWCI | 0.029893 | orf1ab | A24:02 |
| SEQ ID NO: 281. | TYLEGSVRVVTTF | 0.023697 | orf1ab | A24:02 |
| SEQ ID NO: 282. | VYFLQSINF | 0.001314 | ORF3a | A24:02 |
| SEQ ID NO: 283. | VYIGDPAQL | 0.004506 | orf1ab | A24:02 |
| SEQ ID NO: 284. | VYMPASWVM | 0.001798 | orf1ab | A24:02 |
| SEQ ID NO: 285. | VYMPASWVMRI | 0.006758 | orf1ab | A24:02 |
| SEQ ID NO: 286. | VYMPASWVMRIMTW | 0.029893 | orf1ab | A24:02 |
| SEQ ID NO: 287. | VYSSANNCTF | 0.007954 | S | A24:02 |
| SEQ ID NO: 288. | VYSTGSNVF | 0.00669 | S | A24:02 |
| SEQ ID NO: 289. | VYSVIYLYL | 0.01967 | orf1ab | A24:02 |
| SEQ ID NO: 290. | VYSVIYLYLTF | 0.029893 | orf1ab | A24:02 |
| SEQ ID NO: 291. | VYYTSNPTTF | 9.74E-05 | orf1ab | A24:02 |
| SEQ ID NO: 292. | YYDSMSYEDQDALF | 0.029893 | orf1ab | A24:02 |
| SEQ ID NO: 293. | YYHTTDPSF | 0.022128 | orf1ab | A24:02 |
| SEQ ID NO: 294. | YYKKDNSYF | 0.018368 | orf1ab | A24:02 |
| SEQ ID NO: 295. | YYSQLMCQPI | 0.029893 | orf1ab | A24:02 |
| SEQ ID NO: 296. | YYTSNPTTF | 0.000431 | orf1ab | A24:02 |
| SEQ ID NO: 297. | DIYNDKVAGF | 0.028538 | orf1ab | A25:01 |
| SEQ ID NO: 298. | DTLKEILVTY | 0.044766 | orf1ab | A25:01 |
| SEQ ID NO: 299. | DTVIEVQGY | 0.046522 | orf1ab | A25:01 |
| SEQ ID NO: 300. | DTYNLWNTF | 0.008987 | orf1ab | A25:01 |
| SEQ ID NO: 301. | DVFHLYLQY | 0.046522 | orf1ab | A25:01 |
| SEQ ID NO: 302. | DVFYKENSY | 0.018144 | orf1ab | A25:01 |
| SEQ ID NO: 303. | DVLLPLTQY | 0.046522 | orf1ab | A25:01 |
| SEQ ID NO: 304. | DVTDVTQLY | 0.008648 | orf1ab | A25:01 |
| SEQ ID NO: 305. | EAIRHVRAW | 0.001802 | orf1ab | A25:01 |
| SEQ ID NO: 306. | EAVMYMGTLSY | 0.039544 | orf1ab | A25:01 |
| SEQ ID NO: 307. | EAVMYMGTLSYEQF | 0.039544 | orf1ab | A25:01 |
| SEQ ID NO: 308. | EIAIILASF | 0.002315 | orf1ab | A25:01 |
| SEQ ID NO: 309. | EIKESVQTF | 4.27E-05 | orf1ab | A25:01 |
| SEQ ID NO: 310. | EIVDTVSAL | 4.27E-05 | orf1ab | A25:01 |
| SEQ ID NO: 311. | ETICAPLTVF | 0.029566 | orf1ab | A25:01 |
| SEQ ID NO: 312. | ETIQITISSF | 4.27E-05 | orf1ab | A25:01 |
| SEQ ID NO: 313. | ETIQITISSFKW | 0.039544 | orf1ab | A25:01 |
| SEQ ID NO: 314. | ETISLAGSY | 0.000289 | orf1ab | A25:01 |
| SEQ ID NO: 315. | ETKAIVSTI | 0.039544 | orf1ab | A25:01 |
| SEQ ID NO: 316. | ETKCTLKSF | 0.004943 | S | A25:01 |
| SEQ ID NO: 317. | ETTADIVVF | 0.029566 | orf1ab | A25:01 |
| SEQ ID NO: 318. | EVARDLSLQF | 0.029566 | orf1ab | A25:01 |
| SEQ ID NO: 319. | EVAVKMFDAY | 0.039544 | orf1ab | A25:01 |
| SEQ ID NO: 320. | EVVDKYFDCY | 0.014413 | orf1ab | A25:01 |
| SEQ ID NO: 321. | FTSDYYQLY | 0.046522 | ORF3a | A25:01 |
| SEQ ID NO: 322. | FVFKNIDGY | 0.046522 | S | A25:01 |
| SEQ ID NO: 323. | FVVEVVDKY | 0.046522 | orf1ab | A25:01 |
| SEQ ID NO: 324. | GVVQLTSQW | 0.047348 | orf1ab | A25:01 |
| SEQ ID NO: 325. | LVAEWFLAY | 0.039544 | orf1ab | A25:01 |
| SEQ ID NO: 326. | NTVKSVGKF | 0.005815 | orf1ab | A25:01 |
| SEQ ID NO: 327. | QTFSVLACY | 0.039544 | orf1ab | A25:01 |
| SEQ ID NO: 328. | QVVDMSMTY | 0.046522 | orf1ab | A25:01 |
| SEQ ID NO: 329. | TILDGISQY | 0.046522 | orf1ab | A25:01 |
| SEQ ID NO: 330. | VVIPDYNTY | 0.046522 | orf1ab | A25:01 |
| SEQ ID NO: 331. | YTPSKLIEY | 0.046522 | orf1ab | A25:01 |
| SEQ ID NO: 332. | CVADYSVLY | 0.022973 | S | A26:01 |
| SEQ ID NO: 333. | DTLKEILVTY | 0.010551 | orf1ab | A26:01 |
| SEQ ID NO: 334. | DTVIEVQGY | 0.00125 | orf1ab | A26:01 |
| SEQ ID NO: 335. | DVFHLYLQY | 0.00024 | orf1ab | A26:01 |
| SEQ ID NO: 336. | DVFYKENSY | 0.010551 | orf1ab | A26:01 |
| SEQ ID NO: 337. | DVLLPLTQY | 0.021407 | orf1ab | A26:01 |
| SEQ ID NO: 338. | DVTDVTQLY | 5.19E-05 | orf1ab | A26:01 |
| SEQ ID NO: 339. | EIAIILASF | 0.005488 | orf1ab | A26:01 |
| SEQ ID NO: 340. | EIKESVQTF | 0.000133 | orf1ab | A26:01 |
| SEQ ID NO: 341. | EIVDTVSAL | 5.19E-05 | orf1ab | A26:01 |
| SEQ ID NO: 342. | EIVDTVSALVY | 0.012799 | orf1ab | A26:01 |
| SEQ ID NO: 343. | ETIQITISSF | 0.000217 | orf1ab | A26:01 |
| SEQ ID NO: 344. | ETISLAGSY | 0.000271 | orf1ab | A26:01 |
| SEQ ID NO: 345. | ETISLAGSYKDWSY | 0.020597 | orf1ab | A26:01 |
| SEQ ID NO: 346. | ETKCTLKSF | 0.020129 | S | A26:01 |
| SEQ ID NO: 347. | EVAVKMFDAY | 0.020597 | orf1ab | A26:01 |
| SEQ ID NO: 348. | EVGPEHSLAEY | 0.043657 | orf1ab | A26:01 |
| SEQ ID NO: 349. | EVVDKYFDCY | 0.003679 | orf1ab | A26:01 |
| SEQ ID NO: 350. | FTSDYYQLY | 0.022973 | ORF3a | A26:01 |
| SEQ ID NO: 351. | FVFKNIDGY | 0.005233 | S | A26:01 |
| SEQ ID NO: 352. | FVVEVVDKY | 0.00024 | orf1ab | A26:01 |
| SEQ ID NO: 353. | NVIPTITQM | 0.022451 | orf1ab | A26:01 |
| SEQ ID NO: 354. | QVVDMSMTY | 0.005233 | orf1ab | A26:01 |
| SEQ ID NO: 355. | TILDGISQY | 0.005114 | orf1ab | A26:01 |
| SEQ ID NO: 356. | TTYPGQGLNGY | 0.043657 | orf1ab | A26:01 |
| SEQ ID NO: 357. | WTAGAAAYY | 0.004978 | S | A26:01 |
| SEQ ID NO: 358. | AIMQLFFSY | 0.015906 | orf1ab | A29:02 |
| SEQ ID NO: 359. | ALCEKALKY | 0.026007 | orf1ab | A29:02 |
| SEQ ID NO: 360. | CVADYSVLY | 0.018202 | S | A29:02 |
| SEQ ID NO: 361. | EYADVFHLY | 0.004571 | orf1ab | A29:02 |
| SEQ ID NO: 362. | FAIGLALYY | 0.000592 | orf1ab | A29:02 |
| SEQ ID NO: 363. | FLFVAAIFY | 0.023638 | orf1ab | A29:02 |
| SEQ ID NO: 364. | FLTENLLLY | 0.001982 | orf1ab | A29:02 |
| SEQ ID NO: 365. | FLYLYALVY | 0.018202 | ORF3a | A29:02 |
| SEQ ID NO: 366. | FTSDYYQLY | 0.001982 | ORF3a | A29:02 |
| SEQ ID NO: 367. | GVYSVIYLY | 0.001982 | orf1ab | A29:02 |
| SEQ ID NO: 368. | HFYWFFSNY | 0.017366 | orf1ab | A29:02 |
| SEQ ID NO: 369. | KLFDRYFKY | 0.001537 | orf1ab | A29:02 |
| SEQ ID NO: 370. | LVAEWFLAY | 0.006122 | orf1ab | A29:02 |
| SEQ ID NO: 371. | MMSAPPAQY | 0.021708 | orf1ab | A29:02 |
| SEQ ID NO: 372. | SFYEDFLEY | 0.000592 | ORF8 | A29:02 |
| SEQ ID NO: 373. | SMMGFKMNY | 0.007652 | orf1ab | A29:02 |
| SEQ ID NO: 374. | VMYMGTLSY | 0.007652 | orf1ab | A29:02 |
| SEQ ID NO: 375. | YANRNRFLY | 0.043926 | M | A29:02 |
| SEQ ID NO: 376. | YFKYWDQTY | 0.041909 | orf1ab | A29:02 |
| SEQ ID NO: 377. | YIFFASFYY | 0.001982 | orf1ab | A29:02 |
| SEQ ID NO: 378. | YILFTRFFY | 0.006122 | orf1ab | A29:02 |
| SEQ ID NO: 379. | AMRPNFTIK | 0.01882 | orf1ab | A30:01 |
| SEQ ID NO: 380. | ASKIITLKK | 0.021738 | ORF3a | A30:01 |
| SEQ ID NO: 381. | ASMPTTIAK | 0.01882 | orf1ab | A30:01 |
| SEQ ID NO: 382. | KTFPPTEPK | 0.007922 | N | A30:01 |
| SEQ ID NO: 383. | KTIQPRVEK | 0.007922 | orf1ab | A30:01 |
| SEQ ID NO: 384. | KVKYLYFIK | 0.04008 | orf1ab | A30:01 |
| SEQ ID NO: 385. | AIMQLFFSY | 0.017758 | orf1ab | A30:02 |
| SEQ ID NO: 386. | ATSRTLSYY | 0.032663 | M | A30:02 |
| SEQ ID NO: 387. | AVKTQFNYY | 0.009507 | orf1ab | A30:02 |
| SEQ ID NO: 388. | GTFTCASEY | 0.040463 | orf1ab | A30:02 |
| SEQ ID NO: 389. | GVYSVIYLY | 0.049516 | orf1ab | A30:02 |
| SEQ ID NO: 390. | HSIGFDYVY | 0.049516 | orf1ab | A30:02 |
| SEQ ID NO: 391. | HSYFTSDYY | 0.049516 | ORF3a | A30:02 |
| SEQ ID NO: 392. | IQYIDIGNY | 0.049516 | ORF8 | A30:02 |
| SEQ ID NO: 393. | KIEELFYSY | 0.032841 | orf1ab | A30:02 |
| SEQ ID NO: 394. | KLFDRYFKY | 0.004649 | orf1ab | A30:02 |
| SEQ ID NO: 395. | KMADQAMTQMY | 0.019673 | orf1ab | A30:02 |
| SEQ ID NO: 396. | KMNYQVNGY | 0.00454 | orf1ab | A30:02 |
| SEQ ID NO: 397. | KNFKSVLYY | 0.037265 | orf1ab | A30:02 |
| SEQ ID NO: 398. | KVNSTLEQY | 0.016355 | orf1ab | A30:02 |
| SEQ ID NO: 399. | LTNDNTSRY | 0.049516 | orf1ab | A30:02 |
| SEQ ID NO: 400. | LVKPSFYVY | 0.015971 | E | A30:02 |
| SEQ ID NO: 401. | MMSAPPAQY | 0.015971 | orf1ab | A30:02 |
| SEQ ID NO: 402. | MSNLGMPSY | 0.049516 | orf1ab | A30:02 |
| SEQ ID NO: 403. | RLYYDSMSY | 0.00454 | orf1ab | A30:02 |
| SEQ ID NO: 404. | RVAGDSGFAAY | 0.029663 | M | A30:02 |
| SEQ ID NO: 405. | RVFSAVGNICY | 0.008431 | orf1ab | A30:02 |
| SEQ ID NO: 406. | RYLALYNKY | 0.01565 | orf1ab | A30:02 |
| SEQ ID NO: 407. | SFYEDFLEY | 0.037265 | ORF8 | A30:02 |
| SEQ ID NO: 408. | SMMGFKMNY | 0.037265 | orf1ab | A30:02 |
| SEQ ID NO: 409. | STNVTIATY | 0.015971 | orf1ab | A30:02 |
| SEQ ID NO: 410. | TLKEILVTY | 0.044826 | orf1ab | A30:02 |
| SEQ ID NO: 411. | VMYMGTLSY | 0.015971 | orf1ab | A30:02 |
| SEQ ID NO: 412. | VVIPDYNTY | 0.032841 | orf1ab | A30:02 |
| SEQ ID NO: 413. | VVYRGIIIY | 0.017693 | orf1ab | A30:02 |
| SEQ ID NO: 414. | WTAGAAAYY | 0.049516 | S | A30:02 |
| SEQ ID NO: 415. | AVHECFVKR | 0.003317 | orf1ab | A31:01 |
| SEQ ID NO: 416. | AVILRGHLR | 0.038363 | M | A31:01 |
| SEQ ID NO: 417. | GTHWFVTQR | 0.003317 | S | A31:01 |
| SEQ ID NO: 418. | GVKHVYQLR | 0.020829 | ORF7a | A31:01 |
| SEQ ID NO: 419. | KSNLKPFER | 0.003317 | S | A31:01 |
| SEQ ID NO: 420. | KTPKYKFVR | 0.028503 | orf1ab | A31:01 |
| SEQ ID NO: 421. | RIAGHHLGR | 0.028503 | M | A31:01 |
| SEQ ID NO: 422. | RVCGVSAAR | 0.011628 | orf1ab | A31:01 |
| SEQ ID NO: 423. | RVKNLNSSR | 0.011628 | E | A31:01 |
| SEQ ID NO: 424. | RVVRSIFSR | 0.011628 | orf1ab | A31:01 |
| SEQ ID NO: 425. | RVYANLGER | 0.011628 | orf1ab | A31:01 |
| SEQ ID NO: 426. | SVSPKLFIR | 0.012145 | ORF7a | A31:01 |
| SEQ ID NO: 427. | SVYAWNRKR | 0.003317 | S | A31:01 |
| SEQ ID NO: 428. | VVSTGYHFR | 0.028503 | orf1ab | A31:01 |
| SEQ ID NO: 429. | VYYPDKVFR | 0.006594 | S | A31:01 |
| SEQ ID NO: 430. | AVHFISNSW | 0.01457 | orf1ab | A32:01 |
| SEQ ID NO: 431. | KAYNVTQAF | 0.001013 | N | A32:01 |
| SEQ ID NO: 432. | KIADYNYKL | 0.008237 | S | A32:01 |
| SEQ ID NO: 433. | KLAKKFDTF | 0.008577 | orf1ab | A32:01 |
| SEQ ID NO: 434. | KLASHMYCSF | 0.047194 | orf1ab | A32:01 |
| SEQ ID NO: 435. | KLFDRYFKY | 0.000494 | orf1ab | A32:01 |
| SEQ ID NO: 436. | KLFDRYFKYW | 0.006388 | orf1ab | A32:01 |
| SEQ ID NO: 437. | KLLHKPIVW | 0.000224 | orf1ab | A32:01 |
| SEQ ID NO: 438. | KLMGHFAWW | 0.012574 | orf1ab | A32:01 |
| SEQ ID NO: 439. | KLMGHFAWWTAF | 0.047194 | orf1ab | A32:01 |
| SEQ ID NO: 440. | KMFDAYVNTF | 0.000973 | orf1ab | A32:01 |
| SEQ ID NO: 441. | KMKDLSPRW | 0.009734 | N | A32:01 |
| SEQ ID NO: 442. | KQFDTYNLW | 0.003802 | orf1ab | A32:01 |
| SEQ ID NO: 443. | KSHNIALIW | 0.047194 | orf1ab | A32:01 |
| SEQ ID NO: 444. | KSYELQTPF | 0.016535 | orf1ab | A32:01 |
| SEQ ID NO: 445. | RLFARTRSMW | 0.006388 | M | A32:01 |
| SEQ ID NO: 446. | RLFARTRSMWSF | 0.047194 | M | A32:01 |
| SEQ ID NO: 447. | RLRAKHYVY | 0.023978 | orf1ab | A32:01 |
| SEQ ID NO: 448. | RMYIFFASF | 0.012574 | orf1ab | A32:01 |
| SEQ ID NO: 449. | RTIKGTHHW | 0.000494 | orf1ab | A32:01 |
| SEQ ID NO: 450. | RTIKVFTTV | 0.020611 | orf1ab | A32:01 |
| SEQ ID NO: 451. | RTNVYLAVF | 0.047194 | orf1ab | A32:01 |
| SEQ ID NO: 452. | RVYSSANNCTF | 0.027784 | S | A32:01 |
| SEQ ID NO: 453. | SMMGFKMNY | 0.037304 | orf1ab | A32:01 |
| SEQ ID NO: 454. | DTANPKTPK | 0.049958 | orf1ab | A66:01 |
| SEQ ID NO: 455. | ETISLAGSYK | 0.003313 | orf1ab | A66:01 |
| SEQ ID NO: 456. | ETKAIVSTIQR | 0.041179 | orf1ab | A66:01 |
| SEQ ID NO: 457. | EVVENPTIQK | 0.007514 | orf1ab | A66:01 |
| SEQ ID NO: 458. | EVVGDIILK | 0.00105 | orf1ab | A66:01 |
| SEQ ID NO: 459. | FTIGTVTLK | 0.017151 | ORF3a | A66:01 |
| SEQ ID NO: 460. | HVVGPNVNK | 0.012178 | orf1ab | A66:01 |
| SEQ ID NO: 461. | LVIGAVILR | 0.041179 | M | A66:01 |
| SEQ ID NO: 462. | QVVNVVTTK | 0.012178 | orf1ab | A66:01 |
| SEQ ID NO: 463. | STFNVPMEK | 0.041345 | orf1ab | A66:01 |
| SEQ ID NO: 464. | ETAHSCNVNR | 0.01042 | orf1ab | A68:01 |
| SEQ ID NO: 465. | ETFVTHSKGLYR | 0.0272 | orf1ab | A68:01 |
| SEQ ID NO: 466. | ETISLAGSYK | 0.001109 | orf1ab | A68:01 |
| SEQ ID NO: 467. | EVNSFSGYLK | 0.0332 | orf1ab | A68:01 |
| SEQ ID NO: 468. | EVVENPTIQK | 0.010811 | orf1ab | A68:01 |
| SEQ ID NO: 469. | EVVGDIILK | 0.017128 | orf1ab | A68:01 |
| SEQ ID NO: 470. | FAVSKGFFK | 0.0412 | orf1ab | A68:01 |
| SEQ ID NO: 471. | FSSEIIGYK | 0.0412 | orf1ab | A68:01 |
| SEQ ID NO: 472. | FTALTQHGK | 0.0412 | N | A68:01 |
| SEQ ID NO: 473. | FTIGTVTLK | 0.010922 | ORF3a | A68:01 |
| SEQ ID NO: 474. | GAMDTTSYR | 0.0412 | orf1ab | A68:01 |
| SEQ ID NO: 475. | GVAPGTAVLR | 0.042655 | orf1ab | A68:01 |
| SEQ ID NO: 476. | HTTDPSFLGR | 0.004184 | orf1ab | A68:01 |
| SEQ ID NO: 477. | HVASCDAIMTR | 0.021211 | orf1ab | A68:01 |
| SEQ ID NO: 478. | HVGEIPVAYR | 0.026005 | orf1ab | A68:01 |
| SEQ ID NO: 479. | LVASIKNFK | 0.0412 | orf1ab | A68:01 |
| SEQ ID NO: 480. | LVIGAVILR | 0.0412 | M | A68:01 |
| SEQ ID NO: 481. | NSASFSTFK | 0.0412 | S | A68:01 |
| SEQ ID NO: 482. | QTMLFTMLR | 0.013332 | orf1ab | A68:01 |
| SEQ ID NO: 483. | QVVNVVTTK | 0.01766 | orf1ab | A68:01 |
| SEQ ID NO: 484. | SASKIITLK | 0.02681 | ORF3a | A68:01 |
| SEQ ID NO: 485. | STFNVPMEK | 0.02681 | orf1ab | A68:01 |
| SEQ ID NO: 486. | STTTNIVTR | 0.001109 | orf1ab | A68:01 |
| SEQ ID NO: 487. | SVSPKLFIR | 0.01766 | ORF7a | A68:01 |
| SEQ ID NO: 488. | TSFGPLVRK | 0.02681 | orf1ab | A68:01 |
| SEQ ID NO: 489. | TTCCSLSHR | 0.021211 | orf1ab | A68:01 |
| SEQ ID NO: 490. | TTFDSEYCR | 0.004184 | orf1ab | A68:01 |
| SEQ ID NO: 491. | TTIKPVTYK | 0.010922 | orf1ab | A68:01 |
| SEQ ID NO: 492. | TTIVNGVRR | 0.031738 | orf1ab | A68:01 |
| SEQ ID NO: 493. | TVIEVQGYK | 0.013332 | orf1ab | A68:01 |
| SEQ ID NO: 494. | TVYDDGARR | 0.01027 | orf1ab | A68:01 |
| SEQ ID NO: 495. | VTFQSAVKR | 0.01293 | orf1ab | A68:01 |
| SEQ ID NO: 496. | VVSTGYHFR | 0.02714 | orf1ab | A68:01 |
| SEQ ID NO: 497. | DISGINASV | 0.018095 | S | A68:02 |
| SEQ ID NO: 498. | EAANFCALI | 0.02851 | orf1ab | A68:02 |
| SEQ ID NO: 499. | EAFEKMVSL | 0.005531 | orf1ab | A68:02 |
| SEQ ID NO: 500. | EAMYTPHTV | 0.002833 | orf1ab | A68:02 |
| SEQ ID NO: 501. | ETAQNSVRV | 0.011023 | orf1ab | A68:02 |
| SEQ ID NO: 502. | ETFKLSYGI | 0.009267 | orf1ab | A68:02 |
| SEQ ID NO: 503. | ETICAPLTV | 0.00424 | orf1ab | A68:02 |
| SEQ ID NO: 504. | ETKAIVSTI | 0.01438 | orf1ab | A68:02 |
| SEQ ID NO: 505. | FIAGLIAIV | 0.036849 | S | A68:02 |
| SEQ ID NO: 506. | FSASTSAFV | 0.014247 | orf1ab | A68:02 |
| SEQ ID NO: 507. | FSYFAVHFI | 0.036849 | orf1ab | A68:02 |
| SEQ ID NO: 508. | FTISVTTEI | 0.014247 | S | A68:02 |
| SEQ ID NO: 509. | FTVLCLTPV | 0.036849 | orf1ab | A68:02 |
| SEQ ID NO: 510. | FTYASALWEI | 0.036849 | orf1ab | A68:02 |
| SEQ ID NO: 511. | FVAAIFYLI | 0.010965 | orf1ab | A68:02 |
| SEQ ID NO: 512. | FVNEFYAYL | 0.004355 | orf1ab | A68:02 |
| SEQ ID NO: 513. | HTIDGSSGV | 0.014247 | ORF3a | A68:02 |
| SEQ ID NO: 514. | MSAFAMMFV | 0.036849 | orf1ab | A68:02 |
| SEQ ID NO: 515. | NATNVVIKV | 0.013526 | S | A68:02 |
| SEQ ID NO: 516. | NTASWFTAL | 0.009519 | N | A68:02 |
| SEQ ID NO: 517. | NTFSSTFNV | 0.00424 | orf1ab | A68:02 |
| SEQ ID NO: 518. | NTQEVFAQV | 0.00424 | S | A68:02 |
| SEQ ID NO: 519. | NVFAFPFTI | 0.009519 | ORF10 | A68:02 |
| SEQ ID NO: 520. | QSSYIVDSV | 0.0468 | orf1ab | A68:02 |
| SEQ ID NO: 521. | STSAFVETV | 0.009267 | orf1ab | A68:02 |
| SEQ ID NO: 522. | SVAALTNNV | 0.001296 | orf1ab | A68:02 |
| SEQ ID NO: 523. | SVVSKVVKV | 0.006927 | orf1ab | A68:02 |
| SEQ ID NO: 524. | TTAAKLMVV | 0.009519 | orf1ab | A68:02 |
| SEQ ID NO: 525. | TTIQTIVEV | 0.001296 | orf1ab | A68:02 |
| SEQ ID NO: 526. | TVASLINTL | 0.002833 | orf1ab | A68:02 |
| SEQ ID NO: 527. | TVYSHLLLV | 0.02328 | ORF3a | A68:02 |
| SEQ ID NO: 528. | YTACSHAAV | 0.014247 | orf1ab | A68:02 |
| SEQ ID NO: 529. | YTMADLVYA | 0.037043 | orf1ab | A68:02 |
| SEQ ID NO: 530. | YTNSFTRGV | 0.037043 | S | A68:02 |
| SEQ ID NO: 531. | YTVELGTEV | 0.014247 | orf1ab | A68:02 |
| SEQ ID NO: 532. | FPRGQGVPI | 0.020166 | N | B07:02 |
| SEQ ID NO: 533. | IPRRNVATL | 0.001726 | orf1ab | B07:02 |
| SEQ ID NO: 534. | KPNELSRVL | 0.005178 | orf1ab | B07:02 |
| SEQ ID NO: 535. | VPMEKLKTL | 0.004098 | orf1ab | B07:02 |
| SEQ ID NO: 536. | DLKGKYVQI | 0.033089 | orf1ab | B08:01 |
| SEQ ID NO: 537. | EAFEKMVSL | 0.009301 | orf1ab | B08:01 |
| SEQ ID NO: 538. | LMIERFVSL | 0.005277 | orf1ab | B08:01 |
| SEQ ID NO: 539. | TPKYKFVRI | 0.033006 | orf1ab | B08:01 |
| SEQ ID NO: 540. | VPMEKLKTL | 0.006992 | orf1ab | B08:01 |
| SEQ ID NO: 541. | YLKLRSDVL | 0.005277 | orf1ab | B08:01 |
| SEQ ID NO: 542. | YLQPRTFLL | 0.011826 | S | B08:01 |
| SEQ ID NO: 543. | AQFAPSASAF | 0.003443 | N | B15:01 |
| SEQ ID NO: 544. | AQYELKHGTF | 0.014004 | orf1ab | B15:01 |
| SEQ ID NO: 545. | ILMTARTVY | 0.015149 | orf1ab | B15:01 |
| SEQ ID NO: 546. | KMFDAYVNTF | 0.026408 | orf1ab | B15:01 |
| SEQ ID NO: 547. | QLYLGGMSY | 0.002903 | orf1ab | B15:01 |
| SEQ ID NO: 548. | RLYYDSMSY | 0.00279 | orf1ab | B15:01 |
| SEQ ID NO: 549. | RQKRTATKAY | 0.01805 | N | B15:01 |
| SEQ ID NO: 550. | TLKEILVTY | 0.002903 | orf1ab | B15:01 |
| SEQ ID NO: 551. | VLNEKCSAY | 0.00903 | orf1ab | B15:01 |
| SEQ ID NO: 552. | VMYMGTLSY | 0.003443 | orf1ab | B15:01 |
| SEQ ID NO: 553. | VQMAPISAM | 0.011017 | orf1ab | B15:01 |
| SEQ ID NO: 554. | VVYRGIIIY | 0.002903 | orf1ab | B15:01 |
| SEQ ID NO: 555. | YLKLTDNVY | 0.035413 | orf1ab | B15:01 |
| SEQ ID NO: 556. | YQKVGMQKY | 0.002903 | orf1ab | B15:01 |
| SEQ ID NO: 557. | FAVDAAKAY | 0.009682 | orf1ab | B15:02 |
| SEQ ID NO: 558. | HVGEIPVAY | 0.03479 | orf1ab | B15:02 |
| SEQ ID NO: 559. | ILMTARTVY | 0.03984 | orf1ab | B15:02 |
| SEQ ID NO: 560. | MMSAPPAQY | 0.016372 | orf1ab | B15:02 |
| SEQ ID NO: 561. | MVMCGGSLY | 0.015133 | orf1ab | B15:02 |
| SEQ ID NO: 562. | NVLEGSVAY | 0.012682 | orf1ab | B15:02 |
| SEQ ID NO: 563. | QLYLGGMSY | 0.002166 | orf1ab | B15:02 |
| SEQ ID NO: 564. | RLYYDSMSY | 0.015945 | orf1ab | B15:02 |
| SEQ ID NO: 565. | SIIQFPNTY | 0.03479 | orf1ab | B15:02 |
| SEQ ID NO: 566. | TILDGISQY | 0.03479 | orf1ab | B15:02 |
| SEQ ID NO: 567. | TLKEILVTY | 0.004193 | orf1ab | B15:02 |
| SEQ ID NO: 568. | VLNEKCSAY | 0.021449 | orf1ab | B15:02 |
| SEQ ID NO: 569. | VMYMGTLSY | 0.016372 | orf1ab | B15:02 |
| SEQ ID NO: 570. | VVIPDYNTY | 0.030862 | orf1ab | B15:02 |
| SEQ ID NO: 571. | VVYRGIIIY | 0.002166 | orf1ab | B15:02 |
| SEQ ID NO: 572. | YLFDESGEF | 0.041505 | orf1ab | B15:02 |
| SEQ ID NO: 573. | YLKLTDNVY | 0.023204 | orf1ab | B15:02 |
| SEQ ID NO: 574. | AQFAPSASAF | 0.037381 | N | B15:03 |
| SEQ ID NO: 575. | AQYELKHGTF | 0.008839 | orf1ab | B15:03 |
| SEQ ID NO: 576. | GEYSHVVAF | 0.016591 | orf1ab | B15:03 |
| SEQ ID NO: 577. | KAYNVTQAF | 0.036481 | N | B15:03 |
| SEQ ID NO: 578. | KKFLPFQQF | 0.002716 | S | B15:03 |
| SEQ ID NO: 579. | KRVDWTIEY | 0.015513 | orf1ab | B15:03 |
| SEQ ID NO: 580. | RKAVFISPY | 0.016457 | orf1ab | B15:03 |
| SEQ ID NO: 581. | RKGGRTIAF | 0.035532 | orf1ab | B15:03 |
| SEQ ID NO: 582. | RLYYDSMSY | 0.032423 | orf1ab | B15:03 |
| SEQ ID NO: 583. | VKNGSIHLY | 0.009167 | orf1ab | B15:03 |
| SEQ ID NO: 584. | YQKVGMQKY | 0.010738 | orf1ab | B15:03 |
| SEQ ID NO: 585. | ATVHTANKW | 0.028352 | orf1ab | B15:17 |
| SEQ ID NO: 586. | FTSDYYQLY | 0.04278 | ORF3a | B15:17 |
| SEQ ID NO: 587. | KAYNVTQAF | 0.004762 | N | B15:17 |
| SEQ ID NO: 588. | KSHNIALIW | 0.010345 | orf1ab | B15:17 |
| SEQ ID NO: 589. | KSVNITFEL | 0.039556 | orf1ab | B15:17 |
| SEQ ID NO: 590. | KSYELQTPF | 0.002006 | orf1ab | B15:17 |
| SEQ ID NO: 591. | LSFKELLVY | 0.011779 | orf1ab | B15:17 |
| SEQ ID NO: 592. | LTYTGAIKL | 0.008405 | N | B15:17 |
| SEQ ID NO: 593. | MSMTYGQQF | 0.017814 | orf1ab | B15:17 |
| SEQ ID NO: 594. | RSFIEDLLF | 0.018499 | S | B15:17 |
| SEQ ID NO: 595. | RTIKGTHHW | 0.002006 | orf1ab | B15:17 |
| SEQ ID NO: 596. | SSLPSYAAF | 0.017814 | orf1ab | B15:17 |
| SEQ ID NO: 597. | VVYRGIIIY | 0.013553 | orf1ab | B15:17 |
| SEQ ID NO: 598. | KRFDNPVLPF | 0.014262 | S | B27:05 |
| SEQ ID NO: 599. | KRVDWTIEY | 0.00764 | orf1ab | B27:05 |
| SEQ ID NO: 600. | KRWQLALSK | 0.011723 | ORF3a | B27:05 |
| SEQ ID NO: 601. | NRFLYIIKL | 0.011654 | M | B27:05 |
| SEQ ID NO: 602. | NRFNVAITR | 0.022422 | orf1ab | B27:05 |
| SEQ ID NO: 603. | QRNAPRITF | 0.025687 | N | B27:05 |
| SEQ ID NO: 604. | RRLISMMGF | 0.020756 | orf1ab | B27:05 |
| SEQ ID NO: 605. | RRVVFNGVSF | 0.02859 | orf1ab | B27:05 |
| SEQ ID NO: 606. | SRYWEPEFY | 0.009969 | orf1ab | B27:05 |
| SEQ ID NO: 607. | VRFPNITNL | 0.00666 | S | B27:05 |
| SEQ ID NO: 608. | CPDGVKHVY | 0.006941 | ORF7a | B35:01 |
| SEQ ID NO: 609. | FACPDGVKHVY | 0.02055 | ORF7a | B35:01 |
| SEQ ID NO: 610. | FAIGLALYY | 0.011862 | orf1ab | B35:01 |
| SEQ ID NO: 611. | FAVDAAKAY | 0.000815 | orf1ab | B35:01 |
| SEQ ID NO: 612. | FPNITNLCPF | 0.045085 | S | B35:01 |
| SEQ ID NO: 613. | FVVEVVDKY | 0.022453 | orf1ab | B35:01 |
| SEQ ID NO: 614. | HVGEIPVAY | 0.036705 | orf1ab | B35:01 |
| SEQ ID NO: 615. | IPFAMQMAY | 0.001061 | S | B35:01 |
| SEQ ID NO: 616. | IPIGAGICASY | 0.035284 | S | B35:01 |
| SEQ ID NO: 617. | IPIQASLPF | 0.004209 | ORF3a | B35:01 |
| SEQ ID NO: 618. | IPMDSTVKNY | 0.027529 | orf1ab | B35:01 |
| SEQ ID NO: 619. | LPFAMGHAM | 0.045085 | orf1ab | B35:01 |
| SEQ ID NO: 620. | LPFFSNVTW | 0.01684 | S | B35:01 |
| SEQ ID NO: 621. | LPFNDGVYF | 0.00024 | S | B35:01 |
| SEQ ID NO: 622. | LPGVYSVIY | 0.013872 | orf1ab | B35:01 |
| SEQ ID NO: 623. | LPSLATVAY | 0.00024 | orf1ab | B35:01 |
| SEQ ID NO: 624. | LPVNVAFEL | 0.011368 | orf1ab | B35:01 |
| SEQ ID NO: 625. | MPYFFTLLL | 0.045085 | orf1ab | B35:01 |
| SEQ ID NO: 626. | NVLEGSVAY | 0.036705 | orf1ab | B35:01 |
| SEQ ID NO: 627. | QPTESIVRF | 0.002073 | S | B35:01 |
| SEQ ID NO: 628. | SANNCTFEY | 0.035672 | S | B35:01 |
| SEQ ID NO: 629. | TPAFDKSAF | 0.011697 | orf1ab | B35:01 |
| SEQ ID NO: 630. | TPSGTWLTY | 0.014274 | N | B35:01 |
| SEQ ID NO: 631. | VASQSIIAY | 0.036705 | S | B35:01 |
| SEQ ID NO: 632. | VPFVVSTGY | 0.00024 | orf1ab | B35:01 |
| SEQ ID NO: 633. | VPFWITIAY | 0.00024 | orf1ab | B35:01 |
| SEQ ID NO: 634. | VPWDTIANY | 0.001061 | orf1ab | B35:01 |
| SEQ ID NO: 635. | YPGQGLNGY | 0.036705 | orf1ab | B35:01 |
| SEQ ID NO: 636. | YPNASFDNF | 0.014274 | orf1ab | B35:01 |
| SEQ ID NO: 637. | FPFTIYSL | 0.030684 | ORF10 | B35:03 |
| SEQ ID NO: 638. | FPFTIYSLLL | 0.030684 | ORF10 | B35:03 |
| SEQ ID NO: 639. | HPTQAPTHL | 0.01789 | orf1ab | B35:03 |
| SEQ ID NO: 640. | LPVNVAFEL | 0.004572 | orf1ab | B35:03 |
| SEQ ID NO: 641. | AELAKNVSL | 0.01446 | orf1ab | B37:01 |
| SEQ ID NO: 642. | GEYSHVVAF | 0.012854 | orf1ab | B37:01 |
| SEQ ID NO: 643. | NELSRVLGL | 0.04237 | orf1ab | B37:01 |
| SEQ ID NO: 644. | SEFDRDAAM | 0.049296 | orf1ab | B37:01 |
| SEQ ID NO: 645. | EHFIETISL | 0.015567 | orf1ab | B38:01 |
| SEQ ID NO: 646. | FHLDGEVITF | 0.001151 | orf1ab | B38:01 |
| SEQ ID NO: 647. | MHAASGNLL | 0.000242 | orf1ab | B38:01 |
| SEQ ID NO: 648. | QHEETIYNL | 0.000374 | orf1ab | B38:01 |
| SEQ ID NO: 649. | QHEETIYNLL | 0.001202 | orf1ab | B38:01 |
| SEQ ID NO: 650. | SHFAIGLAL | 0.005605 | orf1ab | B38:01 |
| SEQ ID NO: 651. | SHSQLGGLHL | 0.029037 | orf1ab | B38:01 |
| SEQ ID NO: 652. | SHSQLGGLHLL | 0.029037 | orf1ab | B38:01 |
| SEQ ID NO: 653. | SHVVAFNTL | 0.000374 | orf1ab | B38:01 |
| SEQ ID NO: 654. | THLSVDTKF | 0.001701 | orf1ab | B38:01 |
| SEQ ID NO: 655. | YHNESGLKTIL | 0.016795 | orf1ab | B38:01 |
| SEQ ID NO: 656. | YHTTDPSFL | 0.008652 | orf1ab | B38:01 |
| SEQ ID NO: 657. | AEAELAKNVSL | 0.007677 | orf1ab | B40:01 |
| SEQ ID NO: 658. | AEIRASANL | 0.041511 | S | B40:01 |
| SEQ ID NO: 659. | AEIVDTVSAL | 0.000883 | orf1ab | B40:01 |
| SEQ ID NO: 660. | AELAKNVSL | 0.000245 | orf1ab | B40:01 |
| SEQ ID NO: 661. | AEWFLAYIL | 0.046453 | orf1ab | B40:01 |
| SEQ ID NO: 662. | CEFCGTENL | 0.037064 | orf1ab | B40:01 |
| SEQ ID NO: 663. | GEAANFCAL | 0.011327 | orf1ab | B40:01 |
| SEQ ID NO: 664. | GECPNFVFPL | 0.046453 | orf1ab | B40:01 |
| SEQ ID NO: 665. | GETLPTEVL | 0.000245 | orf1ab | B40:01 |
| SEQ ID NO: 666. | GEVITFDNL | 0.041511 | orf1ab | B40:01 |
| SEQ ID NO: 667. | GEYSHVVAF | 0.003515 | orf1ab | B40:01 |
| SEQ ID NO: 668. | HEETIYNLL | 0.041511 | orf1ab | B40:01 |
| SEQ ID NO: 669. | HEFCSQHTM | 0.003995 | orf1ab | B40:01 |
| SEQ ID NO: 670. | HEGKTFYVL | 0.041511 | orf1ab | B40:01 |
| SEQ ID NO: 671. | HEVLLAPLL | 0.010429 | orf1ab | B40:01 |
| SEQ ID NO: 672. | LEFGATSAAL | 0.046453 | orf1ab | B40:01 |
| SEQ ID NO: 673. | LEYHDVRVVL | 0.030557 | ORF8 | B40:01 |
| SEQ ID NO: 674. | NETLVTMPL | 0.046453 | orf1ab | B40:01 |
| SEQ ID NO: 675. | QEYADVFHL | 0.028134 | orf1ab | B40:01 |
| SEQ ID NO: 676. | REAVGTNLPL | 0.046453 | orf1ab | B40:01 |
| SEQ ID NO: 677. | REVGFWPGL | 0.046453 | orf1ab | B40:01 |
| SEQ ID NO: 678. | SELVIGAVIL | 0.046453 | M | B40:01 |
| SEQ ID NO: 679. | SEVGPEHSL | 0.001901 | orf1ab | B40:01 |
| SEQ ID NO: 680. | TEEVGHTDL | 0.037064 | orf1ab | B40:01 |
| SEQ ID NO: 681. | TEVVGDIIL | 0.041511 | orf1ab | B40:01 |
| SEQ ID NO: 682. | YENFNQHEVL | 0.046453 | orf1ab | B40:01 |
| SEQ ID NO: 683. | AEAELAKNVSL | 0.039917 | orf1ab | B40:02 |
| SEQ ID NO: 684. | AELAKNVSL | 0.001747 | orf1ab | B40:02 |
| SEQ ID NO: 685. | AEWFLAYIL | 0.019301 | orf1ab | B40:02 |
| SEQ ID NO: 686. | GEAANFCAL | 0.049325 | orf1ab | B40:02 |
| SEQ ID NO: 687. | GETLPTEVL | 0.003962 | orf1ab | B40:02 |
| SEQ ID NO: 688. | GEYSHVVAF | 0.001747 | orf1ab | B40:02 |
| SEQ ID NO: 689. | HEFCSQHTM | 0.016033 | orf1ab | B40:02 |
| SEQ ID NO: 690. | HEGKTFYVL | 0.014227 | orf1ab | B40:02 |
| SEQ ID NO: 691. | KEIDRLNEV | 0.012121 | S | B40:02 |
| SEQ ID NO: 692. | KENSYTTTI | 0.01638 | orf1ab | B40:02 |
| SEQ ID NO: 693. | QEYADVFHL | 0.037143 | orf1ab | B40:02 |
| SEQ ID NO: 694. | SEVGPEHSL | 0.007022 | orf1ab | B40:02 |
| SEQ ID NO: 695. | VEKGVLPQL | 0.048718 | orf1ab | B40:02 |
| SEQ ID NO: 696. | YENFNQHEV | 0.049325 | orf1ab | B40:02 |
| SEQ ID NO: 697. | AEWFLAYILF | 0.030057 | orf1ab | B44:02 |
| SEQ ID NO: 698. | EEAIRHVRAW | 0.00493 | orf1ab | B44:02 |
| SEQ ID NO: 699. | EEFEPSTQY | 0.004276 | orf1ab | B44:02 |
| SEQ ID NO: 700. | EELKKLLEQW | 0.018127 | M | B44:02 |
| SEQ ID NO: 701. | GEYSHVVAF | 0.018034 | orf1ab | B44:02 |
| SEQ ID NO: 702. | KEIKESVQTF | 0.007041 | orf1ab | B44:02 |
| SEQ ID NO: 703. | MEVTPSGTW | 0.019179 | N | B44:02 |
| SEQ ID NO: 704. | QEILGTVSW | 0.000277 | orf1ab | B44:02 |
| SEQ ID NO: 705. | QELGKYEQY | 0.018127 | S | B44:02 |
| SEQ ID NO: 706. | QEYADVFHLY | 0.000404 | orf1ab | B44:02 |
| SEQ ID NO: 707. | REHEHEIAW | 0.000949 | orf1ab | B44:02 |
| SEQ ID NO: 708. | SEFSSLPSY | 0.001823 | orf1ab | B44:02 |
| SEQ ID NO: 709. | SEMVMCGGSLY | 0.030057 | orf1ab | B44:02 |
| SEQ ID NO: 710. | VENPDILRVY | 0.013268 | orf1ab | B44:02 |
| SEQ ID NO: 711. | VENPHLMGW | 0.000277 | orf1ab | B44:02 |
| SEQ ID NO: 712. | AEWFLAYILF | 0.040774 | orf1ab | B44:03 |
| SEQ ID NO: 713. | EEAIRHVRAW | 0.028292 | orf1ab | B44:03 |
| SEQ ID NO: 714. | EEFEPSTQY | 0.006215 | orf1ab | B44:03 |
| SEQ ID NO: 715. | GEFKLASHMY | 0.040774 | orf1ab | B44:03 |
| SEQ ID NO: 716. | GEYSHVVAF | 0.001418 | orf1ab | B44:03 |
| SEQ ID NO: 717. | KEIKESVQTF | 0.00771 | orf1ab | B44:03 |
| SEQ ID NO: 718. | MEVTPSGTW | 0.026628 | N | B44:03 |
| SEQ ID NO: 719. | QEILGTVSW | 0.000405 | orf1ab | B44:03 |
| SEQ ID NO: 720. | QELGKYEQY | 0.00771 | S | B44:03 |
| SEQ ID NO: 721. | QEYADVFHLY | 0.000584 | orf1ab | B44:03 |
| SEQ ID NO: 722. | QEYADVFHLYLQY | 0.040774 | orf1ab | B44:03 |
| SEQ ID NO: 723. | REHEHEIAW | 0.000559 | orf1ab | B44:03 |
| SEQ ID NO: 724. | SEFSSLPSY | 0.000763 | orf1ab | B44:03 |
| SEQ ID NO: 725. | TEISFMLW | 0.040774 | orf1ab | B44:03 |
| SEQ ID NO: 726. | TELEPPCRF | 0.02867 | orf1ab | B44:03 |
| SEQ ID NO: 727. | VENPDILRVY | 0.014318 | orf1ab | B44:03 |
| SEQ ID NO: 728. | VENPHLMGW | 0.001418 | orf1ab | B44:03 |
| SEQ ID NO: 729. | AEIRASANLA | 0.030847 | S | B45:01 |
| SEQ ID NO: 730. | AEIVDTVSA | 0.000168 | orf1ab | B45:01 |
| SEQ ID NO: 731. | MEIDFLELA | 0.009692 | orf1ab | B45:01 |
| SEQ ID NO: 732. | MELPTGVHA | 0.02287 | orf1ab | B45:01 |
| SEQ ID NO: 733. | QEAYEQAVA | 0.02918 | orf1ab | B45:01 |
| SEQ ID NO: 734. | QESPFVMMSA | 0.030847 | orf1ab | B45:01 |
| SEQ ID NO: 735. | REAACCHLA | 0.030847 | orf1ab | B45:01 |
| SEQ ID NO: 736. | SEFRVYSSA | 0.002754 | S | B45:01 |
| SEQ ID NO: 737. | TEVPVAIHA | 0.019028 | S | B45:01 |
| SEQ ID NO: 738. | YENAFLPFA | 0.030847 | orf1ab | B45:01 |
| SEQ ID NO: 739. | FAFPFTIYSL | 0.030701 | ORF10 | B46:01 |
| SEQ ID NO: 740. | FAIGLALYY | 0.002681 | orf1ab | B46:01 |
| SEQ ID NO: 741. | FAVDAAKAY | 0.00106 | orf1ab | B46:01 |
| SEQ ID NO: 742. | KAYNVTQAF | 0.004272 | N | B46:01 |
| SEQ ID NO: 743. | LAKDTTEAF | 0.004482 | orf1ab | B46:01 |
| SEQ ID NO: 744. | LTKHPNQEY | 0.023187 | orf1ab | B46:01 |
| SEQ ID NO: 745. | TLKEILVTY | 0.007729 | orf1ab | B46:01 |
| SEQ ID NO: 746. | VARDLSLQF | 0.004482 | orf1ab | B46:01 |
| SEQ ID NO: 747. | VATSRTLSY | 0.024672 | M | B46:01 |
| SEQ ID NO: 748. | VVIPDYNTY | 0.025688 | orf1ab | B46:01 |
| SEQ ID NO: 749. | VVYRGIIIY | 0.034518 | orf1ab | B46:01 |
| SEQ ID NO: 750. | YAFEHIVY | 0.049998 | orf1ab | B46:01 |
| SEQ ID NO: 751. | YLFDESGEF | 0.034012 | orf1ab | B46:01 |
| SEQ ID NO: 752. | YTPSKLIEY | 0.036216 | orf1ab | B46:01 |
| SEQ ID NO: 753. | YVNTFSSTF | 0.033781 | orf1ab | B46:01 |
| SEQ ID NO: 754. | CPAEIVDTV | 0.024829 | orf1ab | B51:01 |
| SEQ ID NO: 755. | DAPAHISTI | 0.001091 | orf1ab | B51:01 |
| SEQ ID NO: 756. | FPFTIYSLL | 0.027061 | ORF10 | B51:01 |
| SEQ ID NO: 757. | FPLCANGQV | 0.031524 | orf1ab | B51:01 |
| SEQ ID NO: 758. | FPLNSIIKTI | 0.031524 | orf1ab | B51:01 |
| SEQ ID NO: 759. | FPRGQGVPI | 0.031524 | N | B51:01 |
| SEQ ID NO: 760. | IPTNFTISV | 0.024463 | S | B51:01 |
| SEQ ID NO: 761. | IPYNSVTSSI | 0.031524 | ORF3a | B51:01 |
| SEQ ID NO: 762. | LPFAMGII | 0.031524 | orf1ab | B51:01 |
| SEQ ID NO: 763. | LPIDKCSRI | 0.006712 | orf1ab | B51:01 |
| SEQ ID NO: 764. | LPIDKCSRII | 0.031524 | orf1ab | B51:01 |
| SEQ ID NO: 765. | LPLVSSQCV | 0.006712 | S | B51:01 |
| SEQ ID NO: 766. | LPQNAVVKI | 0.000365 | orf1ab | B51:01 |
| SEQ ID NO: 767. | LPWNVVRI | 0.000365 | orf1ab | B51:01 |
| SEQ ID NO: 768. | LPWNVVRIKI | 0.031524 | orf1ab | B51:01 |
| SEQ ID NO: 769. | LPYPDPSRI | 8.82E-05 | orf1ab | B51:01 |
| SEQ ID NO: 770. | MPASWVMRI | 0.001715 | orf1ab | B51:01 |
| SEQ ID NO: 771. | MPLKAPKEI | 0.007617 | orf1ab | B51:01 |
| SEQ ID NO: 772. | MPLSAPTL | 0.005999 | orf1ab | B51:01 |
| SEQ ID NO: 773. | MPLSAPTLV | 0.000376 | orf1ab | B51:01 |
| SEQ ID NO: 774. | VPFWITIAYI | 0.031524 | orf1ab | B51:01 |
| SEQ ID NO: 775. | VPQEHYVRI | 0.027061 | orf1ab | B51:01 |
| SEQ ID NO: 776. | VPYCYDTNV | 0.006712 | orf1ab | B51:01 |
| SEQ ID NO: 777. | VPYNMRVI | 0.007391 | orf1ab | B51:01 |
| SEQ ID NO: 778. | YPSLETIQI | 0.024463 | orf1ab | B51:01 |
| SEQ ID NO: 779. | CPIHFYSKW | 0.015948 | ORF8 | B53:01 |
| SEQ ID NO: 780. | DPYEDFQENW | 0.03223 | orf1ab | B53:01 |
| SEQ ID NO: 781. | FAMQMAYRF | 0.015948 | S | B53:01 |
| SEQ ID NO: 782. | FAVHFISNSW | 0.039507 | orf1ab | B53:01 |
| SEQ ID NO: 783. | FPFTIYSL | 0.046479 | ORF10 | B53:01 |
| SEQ ID NO: 784. | FPFTIYSLL | 0.045745 | ORF10 | B53:01 |
| SEQ ID NO: 785. | FPFTIYSLLL | 0.039507 | ORF10 | B53:01 |
| SEQ ID NO: 786. | FPNITNLCPF | 0.039507 | S | B53:01 |
| SEQ ID NO: 787. | HADQLTPTW | 0.00209 | S | B53:01 |
| SEQ ID NO: 788. | IPFAMQMAY | 0.015948 | S | B53:01 |
| SEQ ID NO: 789. | IPIQASLPF | 0.015948 | ORF3a | B53:01 |
| SEQ ID NO: 790. | IPLMYKGLPW | 0.045745 | orf1ab | B53:01 |
| SEQ ID NO: 791. | LAAVNSVPW | 0.045745 | orf1ab | B53:01 |
| SEQ ID NO: 792. | LPFFSNVTW | 0.000294 | S | B53:01 |
| SEQ ID NO: 793. | LPFFSNVTWF | 0.039507 | S | B53:01 |
| SEQ ID NO: 794. | LPFNDGVYF | 0.000294 | S | B53:01 |
| SEQ ID NO: 795. | LPNNTASW | 0.046479 | N | B53:01 |
| SEQ ID NO: 796. | LPNNTASWF | 0.045745 | N | B53:01 |
| SEQ ID NO: 797. | LPSLATVAY | 0.003013 | orf1ab | B53:01 |
| SEQ ID NO: 798. | LPVNVAFEL | 0.046479 | orf1ab | B53:01 |
| SEQ ID NO: 799. | LPVNVAFELW | 0.004302 | orf1ab | B53:01 |
| SEQ ID NO: 800. | LPYGANKDGIIW | 0.007465 | N | B53:01 |
| SEQ ID NO: 801. | MPASWVMRI | 0.010996 | orf1ab | B53:01 |
| SEQ ID NO: 802. | MPASWVMRIMTW | 0.015948 | orf1ab | B53:01 |
| SEQ ID NO: 803. | MPYFFTLLL | 0.015948 | orf1ab | B53:01 |
| SEQ ID NO: 804. | NPFMIDVQQW | 0.03223 | orf1ab | B53:01 |
| SEQ ID NO: 805. | QPTESIVRF | 0.006428 | S | B53:01 |
| SEQ ID NO: 806. | TAFGLVAEW | 0.025336 | orf1ab | B53:01 |
| SEQ ID NO: 807. | TASDTYACW | 0.030566 | orf1ab | B53:01 |
| SEQ ID NO: 808. | TPLGIDLDEW | 0.03223 | orf1ab | B53:01 |
| SEQ ID NO: 809. | VPANSTVLSF | 0.032264 | orf1ab | B53:01 |
| SEQ ID NO: 810. | VPFWITIAY | 0.046479 | orf1ab | B53:01 |
| SEQ ID NO: 811. | VPWDTIANY | 0.03402 | orf1ab | B53:01 |
| SEQ ID NO: 812. | WPWYIWLGF | 0.045745 | S | B53:01 |
| SEQ ID NO: 813. | YPANSIVCRF | 0.039507 | orf1ab | B53:01 |
| SEQ ID NO: 814. | YPKLQSSQAW | 0.03223 | orf1ab | B53:01 |
| SEQ ID NO: 815. | YPNASFDNF | 0.001223 | orf1ab | B53:01 |
| SEQ ID NO: 816. | CPFGEVFNA | 0.030216 | S | B54:01 |
| SEQ ID NO: 817. | FPFNKWGKA | 0.012714 | orf1ab | B54:01 |
| SEQ ID NO: 818. | FPLKLRGTA | 0.012714 | orf1ab | B54:01 |
| SEQ ID NO: 819. | FPQSAPHGV | 0.027428 | S | B54:01 |
| SEQ ID NO: 820. | FPRGQGVPI | 0.036691 | N | B54:01 |
| SEQ ID NO: 821. | IPIGAGICA | 0.00731 | S | B54:01 |
| SEQ ID NO: 822. | KPTVVVNAA | 0.026619 | orf1ab | B54:01 |
| SEQ ID NO: 823. | LPFAMGHA | 0.000206 | orf1ab | B54:01 |
| SEQ ID NO: 824. | LPFFSNVTWFHA | 0.033454 | S | B54:01 |
| SEQ ID NO: 825. | LPFKLTCA | 0.026763 | orf1ab | B54:01 |
| SEQ ID NO: 826. | LPFKLTCAT | 0.033454 | orf1ab | B54:01 |
| SEQ ID NO: 827. | LPFNDGVYFA | 0.036691 | S | B54:01 |
| SEQ ID NO: 828. | LPSYAAFATA | 0.011592 | orf1ab | B54:01 |
| SEQ ID NO: 829. | LPYPDPSRILGA | 0.023577 | orf1ab | B54:01 |
| SEQ ID NO: 830. | MPILTLTRA | 0.000918 | orf1ab | B54:01 |
| SEQ ID NO: 831. | MPILTLTRALTA | 0.033454 | orf1ab | B54:01 |
| SEQ ID NO: 832. | MPLSAPTLV | 0.003246 | orf1ab | B54:01 |
| SEQ ID NO: 833. | MPNMLRIMA | 0.000808 | orf1ab | B54:01 |
| SEQ ID NO: 834. | MPVCVETKA | 0.00296 | orf1ab | B54:01 |
| SEQ ID NO: 835. | SPIFLIVAA | 0.000206 | ORF7a | B54:01 |
| SEQ ID NO: 836. | TPLIQPIGA | 0.028901 | orf1ab | B54:01 |
| SEQ ID NO: 837. | VPNQPYPNA | 0.032806 | orf1ab | B54:01 |
| SEQ ID NO: 838. | YPSARIVYTA | 0.033454 | orf1ab | B54:01 |
| SEQ ID NO: 839. | AIKITEHSW | 0.026099 | orf1ab | B57:01 |
| SEQ ID NO: 840. | ASFRLFARTRSMW | 0.032603 | M | B57:01 |
| SEQ ID NO: 841. | ASWVMRIMTW | 0.032603 | orf1ab | B57:01 |
| SEQ ID NO: 842. | ATVHTANKW | 0.000584 | orf1ab | B57:01 |
| SEQ ID NO: 843. | ATYKPNTW | 0.030792 | orf1ab | B57:01 |
| SEQ ID NO: 844. | ATYKPNTWCIRCLW | 0.032603 | orf1ab | B57:01 |
| SEQ ID NO: 845. | IAIAMACLVGLMW | 0.032603 | M | B57:01 |
| SEQ ID NO: 846. | IAYIICISTKHFYW | 0.032603 | orf1ab | B57:01 |
| SEQ ID NO: 847. | ISMDNSPNLAW | 0.026263 | orf1ab | B57:01 |
| SEQ ID NO: 848. | KALNLGETF | 0.026867 | orf1ab | B57:01 |
| SEQ ID NO: 849. | KATYKPNTW | 0.002545 | orf1ab | B57:01 |
| SEQ ID NO: 850. | KAYKIEELF | 0.002545 | orf1ab | B57:01 |
| SEQ ID NO: 851. | KAYNVTQAF | 0.030792 | N | B57:01 |
| SEQ ID NO: 852. | KSAGFPFNKW | 0.000152 | orf1ab | B57:01 |
| SEQ ID NO: 853. | KSHKPPISF | 0.026099 | orf1ab | B57:01 |
| SEQ ID NO: 854. | KSHNIALIW | 0.000152 | orf1ab | B57:01 |
| SEQ ID NO: 855. | KSPNFSKLINIIIW | 0.032603 | orf1ab | B57:01 |
| SEQ ID NO: 856. | KSYELQTPF | 0.032603 | orf1ab | B57:01 |
| SEQ ID NO: 857. | KTTLPVNVAFELW | 0.032603 | orf1ab | B57:01 |
| SEQ ID NO: 858. | LSDRELHLSW | 0.032603 | orf1ab | B57:01 |
| SEQ ID NO: 859. | LSDRVVFVLW | 0.032603 | orf1ab | B57:01 |
| SEQ ID NO: 860. | LTAFGLVAEW | 0.027014 | orf1ab | B57:01 |
| SEQ ID NO: 861. | LTNDNTSRYW | 0.006855 | orf1ab | B57:01 |
| SEQ ID NO: 862. | LTNDVSFLAHIQW | 0.032603 | orf1ab | B57:01 |
| SEQ ID NO: 863. | MACLVGLMW | 0.027014 | M | B57:01 |
| SEQ ID NO: 864. | MSALNHTKKW | 0.002171 | orf1ab | B57:01 |
| SEQ ID NO: 865. | RSFIEDLLF | 0.032603 | S | B57:01 |
| SEQ ID NO: 866. | RTFKVSIW | 0.009752 | ORF6 | B57:01 |
| SEQ ID NO: 867. | RTIKGTHHW | 0.000152 | orf1ab | B57:01 |
| SEQ ID NO: 868. | RTVYDDGARRVW | 0.001608 | orf1ab | B57:01 |
| SEQ ID NO: 869. | SALNHTKKW | 0.002109 | orf1ab | B57:01 |
| SEQ ID NO: 870. | TAFGLVAEW | 0.008266 | orf1ab | B57:01 |
| SEQ ID NO: 871. | TTFTYASALW | 0.032603 | orf1ab | B57:01 |
| SEQ ID NO: 872. | TTLPVNVAFELW | 0.008318 | orf1ab | B57:01 |
| SEQ ID NO: 873. | VAIKITEHSW | 0.002488 | orf1ab | B57:01 |
| SEQ ID NO: 874. | VSFLAHIQW | 0.000649 | orf1ab | B57:01 |
| SEQ ID NO: 875. | VTCGTTTLNGLW | 0.026263 | orf1ab | B57:01 |
| SEQ ID NO: 876. | FAQDGNAAI | 0.030568 | orf1ab | 003:03 |
| SEQ ID NO: 877. | FASEAARVV | 0.004023 | orf1ab | 003:03 |
| SEQ ID NO: 878. | FAVDAAKAY | 0.024038 | orf1ab | 003:03 |
| SEQ ID NO: 879. | FGADPIHSL | 0.014693 | orf1ab | 003:03 |
| SEQ ID NO: 880. | FVSDADSTL | 0.037317 | orf1ab | 003:03 |
| SEQ ID NO: 881. | HANEYRLYL | 0.008643 | orf1ab | 003:03 |
| SEQ ID NO: 882. | ITFDNLKTL | 0.031568 | orf1ab | 003:03 |
| SEQ ID NO: 883. | VADAVIKTL | 0.017086 | orf1ab | 003:03 |
| SEQ ID NO: 884. | YADVFHLYL | 0.009465 | orf1ab | 003:03 |
| SEQ ID NO: 885. | LFDESGEF | 0.037316 | orf1ab | 004:01 |
| SEQ ID NO: 886. | LYDKLVSSF | 0.00203 | orf1ab | 004:01 |
| SEQ ID NO: 887. | MFDAYVNTF | 0.000225 | orf1ab | 004:01 |
| SEQ ID NO: 888. | MYDPKTKNV | 0.00203 | orf1ab | 004:01 |
| SEQ ID NO: 889. | TFDNLKTLL | 0.000225 | orf1ab | 004:01 |
| SEQ ID NO: 890. | VYDDGARRV | 0.019803 | orf1ab | 004:01 |
| SEQ ID NO: 891. | VYDPLQPEL | 0.000225 | S | 004:01 |
| SEQ ID NO: 892. | FADDLNQL | 0.020959 | orf1ab | 005:01 |
| SEQ ID NO: 893. | FGDDTVIEV | 0.002666 | orf1ab | 005:01 |
| SEQ ID NO: 894. | FVDGVPFVV | 0.002666 | orf1ab | 005:01 |
| SEQ ID NO: 895. | IADKYVRNL | 0.004811 | orf1ab | 005:01 |
| SEQ ID NO: 896. | ISDEFSSNV | 0.02867 | orf1ab | 005:01 |
| SEQ ID NO: 897. | IVDEPEEHV | 0.002666 | ORF3a | 005:01 |
| SEQ ID NO: 898. | IVDTVSALV | 0.046313 | orf1ab | 005:01 |
| SEQ ID NO: 899. | KVDGVDVEL | 0.001599 | orf1ab | 005:01 |
| SEQ ID NO: 900. | KVDGVVQQL | 0.006968 | orf1ab | 005:01 |
| SEQ ID NO: 901. | LSDDAVVCF | 0.02867 | orf1ab | 005:01 |
| SEQ ID NO: 902. | MADQAMTQM | 0.000612 | orf1ab | 005:01 |
| SEQ ID NO: 903. | MLDMYSVML | 0.046313 | orf1ab | 005:01 |
| SEQ ID NO: 904. | SSDNIALLV | 0.046313 | M | 005:01 |
| SEQ ID NO: 905. | TLDSKTQSL | 0.006968 | S | 005:01 |
| SEQ ID NO: 906. | VADAVIKTL | 0.007772 | orf1ab | 005:01 |
| SEQ ID NO: 907. | VSDIDITFL | 0.009531 | orf1ab | 005:01 |
| SEQ ID NO: 908. | VTDVTQLYL | 0.007772 | orf1ab | 005:01 |
| SEQ ID NO: 909. | VVDSYYSLL | 0.046313 | orf1ab | 005:01 |
| SEQ ID NO: 910. | VYDPLQPEL | 0.047243 | S | 005:01 |
| SEQ ID NO: 911. | YADVFHLYL | 0.000612 | orf1ab | 005:01 |
| SEQ ID NO: 912. | YGDFSHSQL | 0.004811 | orf1ab | 005:01 |
| SEQ ID NO: 913. | YIDIGNYTV | 0.010631 | ORF8 | 005:01 |
| SEQ ID NO: 914. | YLDAYNMMI | 0.046313 | orf1ab | 005:01 |
| SEQ ID NO: 915. | YSDVENPHL | 0.000612 | orf1ab | 005:01 |
| SEQ ID NO: 916. | YVDNSSLTI | 0.012953 | orf1ab | 005:01 |
| SEQ ID NO: 917. | FASEAARGG | 0.01274 | orf1ab | C06:02 |
| SEQ ID NO: 918. | IRQEEVQEL | 0.021133 | ORF7a | C06:02 |
| SEQ ID NO: 919. | KRVDWTIEY | 0.04568 | orf1ab | 006:02 |
| SEQ ID NO: 920. | NRFLYHKL | 0.006221 | M | 006:02 |
| SEQ ID NO: 921. | VRFPNITNL | 0.006221 | S | C06:02 |
| SEQ ID NO: 922. | VRIKIVQML | 0.021133 | orf1ab | C06:02 |
| SEQ ID NO: 923. | VRSIFSRTL | 0.021133 | orf1ab | C06:02 |
| SEQ ID NO: 924. | IRQEEVQEL | 0.026909 | ORF7a | C07:01 |
| SEQ ID NO: 925. | KRVDWTIEY | 0.008073 | orf1ab | C07:01 |
| SEQ ID NO: 926. | NRFLYHKL | 0.010858 | M | C07:01 |
| SEQ ID NO: 927. | SRYWEPEFY | 0.048749 | orf1ab | C07:01 |
| SEQ ID NO: 928. | VRFPNITNL | 0.008073 | S | C07:01 |
| SEQ ID NO: 929. | YRGIIIYKL | 0.026909 | orf1ab | C07:01 |
| SEQ ID NO: 930. | IYNDKVAGF | 0.047819 | orf1ab | C07:02 |
| SEQ ID NO: 931. | KRVDWTIEY | 0.010724 | orf1ab | C07:02 |
| SEQ ID NO: 932. | NYMPYFFTL | 0.018982 | orf1ab | C07:02 |
| SEQ ID NO: 933. | VRFPNITNL | 0.005399 | S | C07:02 |
| SEQ ID NO: 934. | VYMPASWVM | 0.049054 | orf1ab | C07:02 |
| SEQ ID NO: 935. | WRNTNPIQL | 0.049054 | orf1ab | C07:02 |
| SEQ ID NO: 936. | YYPSARIVY | 0.043688 | orf1ab | C07:02 |
| SEQ ID NO: 937. | YYTSNPTTF | 0.022181 | orf1ab | C07:02 |
| SEQ ID NO: 938. | FASEAARW | 0.012299 | orf1ab | C12:03 |
| SEQ ID NO: 939. | FAVDAAKAY | 0.007999 | orf1ab | C12:03 |
| SEQ ID NO: 940. | FAYANRNRF | 0.035776 | M | C12:03 |
| SEQ ID NO: 941. | FAYTKRNVI | 0.01524 | orf1ab | C12:03 |
| SEQ ID NO: 942. | HANEYRLYL | 0.040438 | orf1ab | C12:03 |
| SEQ ID NO: 943. | ITFDNLKTL | 0.018974 | orf1ab | C12:03 |
| SEQ ID NO: 944. | KAYNVTQAF | 0.01524 | N | C12:03 |
| SEQ ID NO: 945. | VAYFNMVYM | 0.04454 | orf1ab | C12:03 |
| SEQ ID NO: 946. | YAKPFLNKV | 0.01524 | orf1ab | C12:03 |
| SEQ ID NO: 947. | YVYSRVKNL | 0.018974 | E | C12:03 |
| SEQ ID NO: 948. | FFITGNTL | 0.01964 | orf1ab | C14:02 |
| SEQ ID NO: 949. | FYLTNDVSF | 0.026691 | orf1ab | C14:02 |
| SEQ ID NO: 950. | IFFITGNTL | 0.029014 | orf1ab | C14:02 |
| SEQ ID NO: 951. | IYNDKVAGF | 0.007092 | orf1ab | C14:02 |
| SEQ ID NO: 952. | MFDAYVNTF | 0.029014 | orf1ab | C14:02 |
| SEQ ID NO: 953. | NYMPYFFTL | 0.03943 | orf1ab | C14:02 |
| SEQ ID NO: 954. | SFSASTSAF | 0.028688 | orf1ab | C14:02 |
| SEQ ID NO: 955. | SFYEDFLEY | 0.017213 | ORF8 | C14:02 |
| SEQ ID NO: 956. | SYSGQSTQL | 0.028688 | orf1ab | C14:02 |
| SEQ ID NO: 957. | TYFTQSRNL | 0.038987 | orf1ab | C14:02 |
| SEQ ID NO: 958. | VYDPLQPEL | 0.017409 | S | C14:02 |
| SEQ ID NO: 959. | VYIGDPAQL | 0.029014 | orf1ab | C14:02 |
| SEQ ID NO: 960. | VYMPASWVM | 0.021292 | orf1ab | C14:02 |
| SEQ ID NO: 961. | YFMRFRRAF | 0.021053 | orf1ab | C14:02 |
| SEQ ID NO: 962. | YFVVKRHTF | 0.028688 | orf1ab | C14:02 |
| SEQ ID NO: 963. | YYHTTDPSF | 0.031101 | orf1ab | C14:02 |
| SEQ ID NO: 964. | YYPSARIVY | 0.001879 | orf1ab | C14:02 |
| SEQ ID NO: 965. | YYQLYSTQL | 0.007602 | ORF3a | C14:02 |
| SEQ ID NO: 966. | YYTSNPTTF | 0.012671 | orf1ab | C14:02 |
| SEQ ID NO: 967. | HANEYRLYL | 0.009751 | orf1ab | C15:02 |
| SEQ ID NO: 968. | RAMPNMLRI | 0.030067 | orf1ab | C15:02 |
| SEQ ID NO: 969. | RTIKVFTTV | 0.008434 | orf1ab | C15:02 |
| SEQ ID NO: 970. | TVYSHLLLV | 0.045209 | ORF3a | C15:02 |
| SEQ ID NO: 971. | YADVFHLYL | 0.020093 | orf1ab | C15:02 |

### REFERENCES

1. Zu, Z. Y., Jiang, M. D., Xu, P. P., Chen, W., Ni, Q. Q., Lu, G. M., and Zhang, L. J. (2020} Coronavirus disease 2019 (COVID-19): a perspective from China. Radiology 296, E15-E25.
2. Li, Q., Guan, X, Wu, P., Wang, X, Zhou, L., Tong, Y., Ren, R., Leung, K. S. M., Lau, E. H. Y., Wong, J. Y., et al. (2020). Early transmission dynamics in Wuhan, China, of novel coronavirus-infected pneumonia. N. Engl. J. Med. 382, 1199-1207.
3. Guo, Y.-R., Cao, Q.-D., Hong, Z.-S., Tan, Y.-Y., Chen, S.-D., Jin, H.-J., Tan, K-S., Wang, D.-Y., and Yan, Y. (2020). The origin, transmission and clinical therapies on coronavirus disease 2019 (COVID-19) outbreak—an update on the status. Mil. Med. Res. 7, 11.
4. Channappanavar, R., Zhao, J., and Perlman, S. (2014). T cell-mediated immune response to respiratory coronaviruses. Immunol. Res. 59, 118-128.
5. Janice Oh, H.-L., Ken-En Gan, S., Bertoletti, A., and Tan, Y. J. p012). Understanding the T cell immune response in SARS coronavirus infection. Emerg. Microbes Infect. 1, e23.
6. Ng, O.-W., Chia, A., Tan, A. T., Jadi, R. S., Leong, H. N., Bertoletti, A., and Tan, Y. J. (2016). Memory T cell responses targeting the SARS coronavirus persist up to 11 years post-infection. Vaccine 34, 2008-2014.
7. Le Bert, N., Tan, A. T., Kunasegaran, K., Tham, C. Y. L., Hafezi, M., Chia, A., Chng, M. H. Y., Lin, M., Tan, N., Linster, M., et al. (2020). SARS-CoV-2-spe-cific T cell immunity in cases of COVID-19 and SARS, and uninfected controls. Nature 584, 457-462.
8. Grifoni, A., Weiskopf, D., Ramirez, S. I., Mateus, J., Dan, J. M., Moder-bacher, C. R., Rawlings, S. A., Sutherland, A., Premkumar, L., Jadi, R. S., et al. (2020). Targets of T cell responses to SARS-CoV-2 coronavirus in humans with COVID-19 disease and unexposed individuals. Cell 181, 14891501.e15.
9. Matzaraki, V., Kumar, V., Wijmenga, C., and Zhemakova, A. (2017). The MHC locus and genetic susceptibility to autoimmune and infectious diseases. Genome Biol. 18, 76.
10. Lin, M., Tseng, H.-K., Trejaut, J. A., Lee, H.-L., Loo, J.-H., Chu, C.-C., Chen, P.-J., Su, Y.-W., Lim, K. H., Tsai, Z.-U., et al. (2003). Association of HLA class I with severe acute respiratory syndrome coronavirus infection. BMC Med. Genet. 4, 9.
11. Wang, S.-F., Chen, K. H., Chen, M., Li, W. Y., Chen, Y. J., Tsao, C. H., Yen, M X., Huang, J. C., and Chen, Y. M. (2011). Human-leukocyte antigen class I Cw 1502 and class II DR 0301 genotypes are associated with resistance to severe acute respiratory syndrome (SARS) infection. Viral Immunol. 24, 421-426.
12. Ng, M. H., Lau, K M., Li, L., Cheng, S. H., Chan, W. Y., Hui, P. K., Zee, B., Leung, C. B., and Sung, J. J. (2004). Association of human-leukocyte-antigen class I (B*0703) and class II (DRB1*0301) genotypes with susceptibility and resistance to the development of severe acute respiratory syndrome. J. Infect. Dis. 190, 515-518.
13. Ng, M., Cheng, S. H., Lau, K. M., Leung, G. M., Khoo, U. S., Zee, B. C. W., and Sung, J. J. Y. (2010). Immunogenetics in SARS: a case-control study. Hong Kong Med. J. 16 (5 Suppl 4), 29-33.
14. Sanchez-Mazas, A. (2020). HLA studies in the context of coronavirus outbreaks. Swiss Med. Wkly. 150, w20248.
15. Nguyen, A., David, J. K., Maden, S. K., Wood, M A., Weeder, B. R., Nellore, A., and Thompson, R. F. (2020). Human leukocyte antigen susceptibility map for SARS-CoV-2. J. Virol. 94, e00510-20.
16. Zhao, W., and Sher, X. p018). Systematically benchmarking peptide-MHC binding predictors: from synthetic to naturally processed epitopes. PLoS Comput. Biol. 14, e1006457.
17. Sarkizova, S., Klaeger, S., Le, P. M., Li, L. W., Oliveira, G., Keshishian, H., Hartigan, C. R., Zhang, W., Braun, D. A., Ligon, K. L., et al. (2020). A large peptidome dataset improves HLA class I epitope prediction across most of the human population. Nat. Biotechnol. 38, 199-209.
18. Gonzalez-Galarza, F. F., Takeshita, L. Y., Santos, E. J., Kempson, F., Maia, M. H., da Silva, A. L., Teles e Silva, A. L., Ghattaoraya, G. S., Alfirevic, A., Jones, A. R., and Middleton, D. (2015). Allele frequency net 2015 update: new features for HLA epitopes, KIR and disease and HLA adverse drug reaction associations. Nucleic Acids Res. 43 (D1), D784-0788.
19. O'Donnell, T. J., Rubinsteyn, A., and Laserson, U. (2020). MHCflurry 2.0: Improved Pan-Allele Prediction of MHC Class I-Presented Peptides by Incorporating Antigen Processing. Cell Syst. 11, 42-48.e7.
20. Jurtz, V., Paul, S., Andreatta, M., Marcatili, P., Peters, B., and Nielsen, M. (2017). NetMHCpan-4.0: improved peptide-MHC class I interaction predictions integrating eluted ligand and peptide binding affinity data. J. Immunol. 199, 3360-3368.
21. Andreatta, M., and Nielsen, M. (2016). Gapped sequence alignment using artificial neural networks: application to the MHC class I system. Bioinformatics 32, 511-517.
22. Bassani-Sternberg, M., Chong, C., Guillaume, P., Solleder, M., Pak, H., O Gannon, P., Kandalaft, L. E., Coukos, G., and Gfeller, D. (2017). Deciphering HLA-I motifs across HLA peptidomes improves neo-antigen predictions and identifies allostery regulating HLA specificity. PLoS Comput. Biol. 13, e1005725.
23. Zhang, H., Lund, O., and Nielsen, M. (2009). The PickPocket method for predicting binding specificities for receptors based on receptor pocket similarities: application to MHC-peptide binding. Bioinformatics 25, 1293-1299.
24. Rasmussen, M., Fenoy, E., Hamdahl, M., Kristensen, A. B., Nielsen, I. K., Nielsen, M., and Buus, S. (2016). Pan-specific prediction of peptide-MHC class I complex stability, a correlate of T cell immunogenicity. J. Immunol. 197, 1517-1524.
25. Paul, S., Weiskopf, D., Angelo, M. A., Sidney, J., Peters, B., and Sette, A. (2013). HLA class I alleles are associated with peptide-binding repertoires of different size, affinity, and immunogenicity. J. Immunol. 191, 58315839.
26. Nichols, K. (2007). False discovery rate procedures. In Statistical Parametric Mapping, W. Penny, K. Friston, J. Ashbumer, S. Kiebel, and T. Nichols, eds. (Elsevier), pp. 246-252.
27. Nielsen, M., Andreatta, M., Peters, B., and Buus, S. (2020). Immunoinformatics: Predicting Peptide-MHC Binding. Annu. Rev. Biomed. Data Sci. 3, 191-215.
28. Trolle, T., McMurtrey, C. P., Sidney, J., Bardet, W., Osbom, S. C., Kaever, T., Sette, A., Hildebrand, W. H., Nielsen, M., and Peters, B. (2016). The length distribution of class I-restricted T cell epitopes is determined by both peptide supply and MHC allele-specific binding preference. J. Immunol. 196, 1480-1487.
29. Rapin, N., Hoof, I., Lund, O., and Nielsen, M. polo). The MHC motif viewer: a visualization tool for MHC binding motifs. Curr. Protoc. Immunol. Chapter 18, 17.
30. Benjamini, Y., and Hochberg, Y. (1995). Controlling the false discovery rate: a practical and powerful approach to multiple testing. J. R. Stat. Soc. B 57, 289-300.
31. Williamson, E. J., Walker, A. J., Bhaskara, K., Bacon, S., Bates, C., Morton, C. E., Curtis, H. J., Mehrkar, A., Evans, D., Inglesby, P., et al. (2020). Factors associated with COVID-19 death using OpenSAFELY. Nature 584, 430-436.
32. de Lusignan, S., Dorward, J., Correa, A., Jones, N., Akinyemi, O., Amirtha-lingam, G., Andrews, N., Byford, R., Dabrera, G., Elliot, A, et al. (2020). Risk factors for SARS-CoV-2 among patients in the Oxford Royal College of General Practitioners Research and Surveillance Centre primary care network: a cross-sectional study. Lancet Infect. Dis. 20, 1034-1042.
33. Rolland, M., Heckerman, D., Deng, W., Rousseau, C. M., Coovadia, H., Bishop, K., Goulder, P. J., Walker, B. D., Brander, C., and Mullins, J. I. (2008). Broad and Gag-biased HIV-1 epitope repertoires are associated with lower viral loads. PLoS ONE 3, e1424.
34. Campbell, K M., Steiner, G., Wells, D. K., Ribas, A., and Kalbasi, A. (2020). Prediction of SARS-CoV-2 epitopes across 9360 HLA class I alleles. bio-Rxiv. https://doi/org/10.1101/2020.03.30.016931.
35. Chowell, D., Krishna, C., Pierini, F., Makarov, V., Rizvi, N A., Kuo, F., Morris, L. G. T., Riaz, N., Lenz, T. L., and Chan, T. A. (2019). Evolutionary divergence of HLA class I genotype impacts efficacy of cancer immunotherapy. Nat. Med. 25, 1715-1720.
36. Arora, J., Pierini, F., McLaren, P. J., Carrington, M., Fellay, J., and Lenz, T. L. (2020). HLA heterozygote advantage against HIV-1 is driven by quantitative and qualitative differences in HLA allele-specific peptide presentation. Mol. Biol. Evol. 37, 639-650.
37. Croft, N. P., Smith, S. A, Pickering, J., Sidney, J., Peters, B., Faridi, P., Witney, M. J., Sebastian, P., Flesch, I. E. A., Heading, S. L., et al. (2019). Most viral peptides displayed by class I MHC on infected cells are immunogenic. Proc. Natl. Acad. Sci. USA 116, 3112-3117.
38. Cao, Y., Li, L., Feng, Z., Wan, S., Huang, P., Sun, X, Wen, F., Huang, X, Ning, G., and Wang, W. (2020). Comparative genetic analysis of the novel coronavirus (2019-nCoV/SARS-CoV-2) receptor ACE2 in different populations. Cell Discov. 6, 11.
39. Wu, F., Zhao, S., Yu, B., Chen, Y. M., Wang, W., Song, Z. G., Hu, Y., Tao, Z. W., Tian, J. H., Pei, Y. Y., et al. (2020). A new coronavirus associated with human respiratory disease in China. Nature 579, 265-269.
40. Ahmed, S. F., Quadeer, A. A., and McKay, M. R. (2020). COVIDep: a web-based platform for real-time reporting of vaccine target recommendations for SARS-CoV-2. Nat. Protoc. 15, 2141-2142.
41. Zhang, C., Zheng, W., Huang, X, Bell, E. W., Zhou, X., and Zhang, Y. (2020). Protein structure and sequence re-analysis of 2019-nCoVgenome refutes snakes as its intermediate host or the unique similarity between its spike protein insertions and HIV-1. J. Proteome Res. 19, 1351-1360.
42. Dong, E., Du, H., and Gardner, L. (2020). An interactive web-based dashboard to track COVID-19 in real time. Lancet Infect. Dis. 20, 533-534.
43. R Core Team (2020). R: A language and environment for statistical computing. R Foundation for Statistical Computing, Vienna, Austria. https://www.R-project.org/44.
44. Humphrey, W., Dalke, A., and Schulten, K. (1996). VMD: dynamics. J. Mol. Graph. 14, 33-38, 27-28.
45. Prachar, M., Justesen, S., Bisgaard Steen-Jensen, D., Thorgrimsen, S., Jurgons, E., Winther, O., and Bagger, F. O. (2020). COVID-19 Vaccine Candidates: Prediction and Validation of 174 SARS-CoV-2 Epitopes. bioRxiv 10.1101/2020.03.20.000794.
46. Berman, H. M., Westbrook, J., Feng, Z., Gilliland, G., Bhat, T. N., Weissig, H., Shindyalov, I. N., and Boume, P. E. (2000). The protein data bank. Nucleic Acids Res. 28, 235-242.
47. Button, K S., Ioannidis, J. P., Mokrysz, C., Nosek, B A, Flint, J., Robinson, E S., and Munafo, M. R. (2013). Power failure: why small sample size undermines the reliability of neuroscience. Nat. Rev. Neurosci. 14, 365-376.
48. Vita, R., Mahajan, S., Overton, J. A, Dhanda, S. K., Martini, S., Cantrell, J. R., Wheeler, D. K., Sette, A., and Peters, B. (2019). The immune epitope database (IEDB): 2018 update. Nucleic Acids Res. 47 (D1), D339-0343.
49. Stranzl, T., Larsen, M M., Lundegaard, C., and Nielsen, M. polo). NetCTL-pan: pan-specific MHC class I pathway epitope predictions. Immunogenetics 62, 357-368.
50. Karosiene, E., Lundegaard, C., Lund, O., and Nielsen, M. (2012). NetMHC-cons: a consensus method for the major histocompatibility complex class I predictions. Immunogenetics 64, 177-186.

What is claimed is:

1. A method for predicting consensus MHC-I binding by one or more candidate peptides to an MHC-I protein expressed by a cell, the method comprising:
providing a processor in communication with a memory, the memory including instructions, which, when executed, cause the processor to:
(a) obtain, at the processor, or having obtained training data comprising binding affinity data, for each of a plurality of candidate peptides in a data set, wherein each peptide in the data set is identified by mass spectrometry to be presented by a MHC-I protein as expressed in a mono-allelic cell line;
(b) train, at the processor, or having trained a plurality of machine learning HLA-peptide presentation prediction models using the training data;
(c) generate, at the processor, a presentation prediction for each candidate peptide based on the binding affinity data of the plurality of candidate peptides and using the plurality of machine learning HLA-peptide presentation prediction models, wherein each presentation prediction is indicative of a likelihood of an associated candidate peptide of the plurality of candidate peptides binding to an MHC-I protein expressed in the mono-allelic cell line.

2. The method of claim 1, wherein the memory further includes instructions, which, when executed, further cause the processor to:
select, at the processor, one or more selected peptides of the plurality of candidate peptides for preparing a vaccine composition against a target antigen comprising a polypeptide comprising one or more of the selected peptides, wherein the one or more selected peptides are predicted at the processor to be presented by the MHC-I protein expressed in the mono-allelic cell line.

3. The method of claim 1, wherein the memory further includes instructions, which, when executed, further cause the processor to:
determine, at the processor, population fitness of a selected population against a target antigen by:
factoring, at the processor, observed MHC-I allele preferences for selected target antigen peptides and regional expression of the MHC-I alleles within the selected population;
wherein fitness of a population is inversely associated with observed allele preferences for the selected target antigen peptides; and
wherein the one or more selected target antigen peptides are predicted to be presented by the MHC-I allele expressed in the mono-allelic cell line.

4. The method of claim 3, wherein fitness of a population comprises mortality rate from the target antigen.

5. The method of claim 1, wherein the processor generates the presentation prediction for each candidate peptide based on the binding affinity data of the plurality of candidate peptides using the plurality of machine learning HLA-peptide presentation prediction models and wherein the memory includes instructions which, when executed, further cause the processor to:
(a) observe, at the processor, or having observed performance of each model on a mass spectrometry (MS) data set of naturally presented MHC-I peptides from a mono-allelic cell lines; and
(b) based on the performance, parameterize, at the processor, allele and algorithm specific score thresholds and expected false detection rates (FDR) for each model.

6. The method of claim 1, wherein the training data comprises data relating to a target antigen, which comprises at least two of peptide binding affinity measurements for the target antigen, MHC-peptide stability data and MHC-I pocket architecture.

7. The method of claim 1, wherein each of the plurality of machine learning HLA-peptide presentation prediction models have a previously demonstrated accuracy of peptide calls for a target antigen, wherein accuracy is determined by generating an ROC curve and determining an area under the curve (AUC) measurement of at least 80, 85, 90 or 95 for at least one allotype.

8. The method of claim 1, wherein the plurality of machine learning HLA-peptide presentation prediction models comprises at least 2, 3, 4, 5, 6 or all of:
(i) MHCflurry-binding_percentile,
(ii) MHCflurry_presentation,
(iii) netMHC-4.0,
(iv) netMHCpan-EL-4.0,
(v) netMHCstabpan,
(vi) Pick-pocket; and
(vii) MixMHCpred.

9. The method of claim 8, wherein the plurality of machine learning HLA-peptide presentation prediction models comprises all of (i) through (vii) and wherein the memory further includes instructions, which, when executed, further cause the processor to:
predict, at the processor, a binding affinity for a target antigen using peptide binding affinity measurements for the target antigen including MHCflurry-affinity_percentile and netMHC-4.0;
wherein -MixMHCpred, netMHCpan-EL, and MHCflurry-presentation are trained on naturally eluted MHC-I ligands;
wherein MHCflurry-presentation incorporates antigen processing prediction;
wherein -netMHCstabpan is trained on MHC-peptide stability data; and
wherein PickPocket is trained on quantitative binding affinity data and extrapolates binding based on MHC-I pocket architecture.

10. The method of claim 2, wherein the memory further includes instructions, which, when executed, cause the processor to:
formulate, at the processor, a vaccine composition comprising a polypeptide comprising one or more of the selected peptide sequences, or a polynucleotide encoding the polypeptide.

11. The method of claim 10, wherein the vaccine composition is a cell-mediated immune vaccine, or a T-cell vaccine.

12. The method of claim 2, wherein the target antigen is a pathogen.

13. The method of claim 12, wherein the pathogen is a human immunodeficiency virus (HIV), Hepatitis C virus, Dengue virus, or a coronavirus.

14. The method of claim 12, wherein the target antigen is SARS-CoV-2.

15. The method of claim 12, wherein the target antigen is SARS-CoV2 and the vaccine composition is a SARS-CoV2 vaccine composition.

16. The method of claim 2, wherein the target antigen is a cancer antigen or an immune modulation antigen.

17. (canceled)

18. The method of claim 1, wherein the candidate peptides are selected from any one or any combination of potential 5-mers, 6-mers, 7-mers, 8-mers, 9-mers, 10-mers, 11-mers, 12-mers, 13-mers, 14-mers, 15-mers, 16-mers, 17-mers, 18-mers, 19mers and 20-mers with respect to a target antigen.

19. The method of claim 1, wherein the memory further includes instructions, which, when executed cause the processor to:
determine, at the processor, one or more possible immunotherapy targets.

20. A method of determining population fitness of a selected population against a target antigen, the method comprising:
providing a processor in communication with a memory, the memory including instructions, which, when executed, cause the processor to:
(a) provide, at the processor, a presentation prediction for each candidate peptide of a plurality of candidate peptides with respect to a target antigen using an ensemble presentation prediction model combining the presentation prediction output of each of a plurality of machine learning HLA-peptide presentation prediction models, wherein the presentation prediction represents the likelihood of the candidate peptide binding to an MHC-I protein expressed in a mono-allelic cell line;
(b) at the processor, one or more selected peptides from the candidate peptides based on the presentation prediction for each candidate peptide; and
(c) assessing, at the processor, a fitness of a selected population against the target antigen using the selected peptides, by:
factoring observed allele preferences for the selected target antigen peptides and regional expression of those MHC-I alleles within the selected population.

21. The method of claim 20, wherein fitness of a population comprises mortality rate from the target antigen.

22. The method of claim 20, wherein fitness of a population is inversely associated with observed allele preferences for the selected target antigen peptides.

23. The method of claim 20, wherein each of the plurality of machine learning HLA-peptide presentation prediction models have a previously demonstrated accuracy of peptide calls for the target antigen, wherein accuracy is determined by generating an ROC curve and determining an area under the curve (AUC) measurement of at least 80, 85, 90 or 95 for at least one allotype.

24. The method of claim 20, wherein the target antigen is a pathogen.

25. The method of claim 24, wherein the pathogen is a human immunodeficiency virus (HIV), Hepatitis C virus, Dengue virus, or a coronavirus.

26. The method of claim 20, wherein the target antigen is SARS-CoV-2.

27. The method of claim 26, wherein the target antigen is SARS-CoV2 and the vaccine composition is a SARS-CoV2 vaccine composition.

28. The method of claim 20, wherein the target antigen is a cancer antigen.

29. The method of claim 20, wherein the plurality of machine learning HLA-peptide presentation prediction models comprises at least 2, 3, 4, 5, 6 or all of:
(i) MHCflurry-binding_percentile,
(ii) MHCflurry_presentation,
(iii) netMHC-4.0,
(iv) netMHCpan-EL-4.0,
(v) netMHCstabpan,
(vi) Pick-pocket, and
(vii) MixMHCpred.

30. The method of claim 29, wherein the plurality of the plurality of machine learning HLA-peptide presentation prediction models comprises all of (i) through (vii) and wherein the memory further includes instructions, which, when executed, cause the processor to:
predict a binding affinity for the target antigen using peptide binding affinity measurements for the target antigen including MHCflurry-affinity_percentile and netMHC-4.0;
wherein -MixMHCpred, netMHCpan-EL, and MHCflurry-presentation are trained on naturally eluted MHC-I ligands;
wherein -MHCflurry-presentation incorporates antigen processing prediction;
wherein netMHCstabpan is trained on MHC-peptide stability data; and
wherein -PickPocket is trained on quantitative binding affinity data and extrapolates binding based on MHC-I pocket architecture.

31. (canceled)

32. A peptide library comprising a plurality of library members and stored at a memory accessible by a processor, wherein each library member is a 5-20mer peptide having a predetermined likelihood of binding to a target antigen, and is restricted to a predetermined number of common MHC-I alleles, wherein each library member is selected from a plurality of candidate peptides based on a presentation prediction for each peptide with respect to the target antigen, wherein the presentation prediction represents the likelihood of the candidate peptide binding to an MHC-I protein expressed in a mono-allelic cell line, and wherein the presentation prediction is an output of an ensemble presentation prediction model combining the presentation prediction output of each of a plurality machine learning HLA-peptide presentation prediction models to provide a single presentation prediction for each of a plurality of candidate peptides with respect to the target antigen.

33. The peptide library of claim 32, comprising the 8-14mer peptides of Table A.

34. A vaccine composition comprising a polypeptide comprising any one or more of the library member peptide sequences of the peptide library of claim 32, or a polynucleotide encoding the one or more polypeptides, and an adjuvant, pharmaceutically acceptable carrier, excipient, or any combination thereof.

35. The vaccine composition of claim 34, further comprising a T-cell vaccination.

36. A method of treating or preventing a viral infection in a subject in need thereof, comprising administering to the subject a vaccination composition of claim 34.

37. A method of treating or preventing a cancer infection in a subject in need thereof, comprising administering to the subject a vaccination composition of claim 34.

38. The method of claim 36, wherein a target antigen for administration of the vaccination composition is a pathogen.

39. A vaccine composition comprising a polypeptide comprising any one or more of the peptides in Table A, or a polynucleotide encoding the polypeptide, and an adjuvant, pharmaceutically acceptable carrier, excipient, or any combination thereof.

40. The vaccine composition of claim 39, further comprising a T-cell vaccination.

41. A method of treating or preventing a SARS-COv2 infection in a subject in need thereof, comprising administering to the subject a vaccination composition of claim 39.

42. The method of claim 36, wherein a target antigen for administration of the vaccination composition is a cancer.
